(19) United States
(12) Patent Application Publication
(10) Pub. No.: US 2025/0019668 A1
(43) Pub. Date: Jan. 16, 2025

(54) ENGINEERED CELL LINES FOR INCREASED PRODUCTION OF RECOMBINANT ADENO-ASSOCIATED VIRUS (RAAV)

(71) Applicant: Ultragenyx Pharmaceutical Inc., Novato, CA (US)

(72) Inventors: Nicholas Richards, Chelmsford, MA (US); Justin Tedeschi, Hingham, MA (US); Caroline Adams, Vimoutiers (FR); Charles Pugh, Watertown, MA (US); Matthew Scott Fuller, Millis, MA (US)

(21) Appl. No.: 18/705,200

(22) PCT Filed: Oct. 28, 2022
(86) PCT No.: PCT/US2022/078894

§ 371 Date: Apr. 26, 2024

Related U.S. Application Data
(60) Provisional application No. 63/273,508, filed on Oct. 29, 2021.

(51) Int. Cl. C12N 7/00; C12N 5/00; C12N 15/113

(52) U.S. Cl. CPC C12N 7/00; C12N 5/00; C12N 15/113; C12N 2310/14; C12N 2510/02; C12N 2750/14121; C12N 2750/14152

(57) ABSTRACT

This application relates to recombinant adeno-associated virus (rAAV) packaging and/or producer cell lines which have been engineered to reduce expression and/or activity of one or more genes and/or proteins to increase rAAV titers. The methods of generating the engineered cell lines have also been described herein.

### CROSS-REFERENCE TO RELATED APPLICATION

This application claims the benefit of and priority to U.S. Provisional Application No. 63/273,508, filed on Oct. 29, 2021, the entire disclosure of which is hereby incorporated by reference herein in its entirety for all purposes.

### REFERENCE TO A SEQUENCE LISTING XML

This application contains a Sequence Listing which has been submitted electronically in XML format. The Sequence Listing XML is incorporated herein by reference. Said XML file, created on Oct. 27, 2022, is named ULP-014WO and is 522,739 bytes in size.

### FIELD OF THE DISCLOSURE

This application relates generally to engineered producer and/or packaging cell lines and methods of generating the engineered producer and/or packaging cell lines to reduce or eliminate expression of one or more proteins, which results in increasing recombinant adeno-associated virus (rAAV) titer.

### BACKGROUND

rAAV-based vectors are one of the most promising vehicles for human gene therapy. rAAV vectors are under consideration for a wide variety of gene therapy applications. In particular, rAAV vectors can deliver therapeutic genes to dividing and nondividing cells, and these genes can persist for extended periods without integrating into the genome of the targeted cell. Although systems for producing rAAV have evolved over the last two decades, several issues remain to be solved. One limitation of rAAV production systems is the low titer yield of rAAV particles. Pharmaceutical development of rAAV-based gene products at preclinical stage require large amounts of rAAV vectors for studies in larger species to enable complete toxicology and biodistribution studies that are helpful in predicting dosages in humans. Furthermore, because current rAAV production systems result in low titer yields, manufacturing sufficient levels of rAAV for use in human trials and commercial applications is challenging. Researchers have explored numerous ways to generate adequately high titers of rAAV particles, but there is still a need for addressing this issue. In particular, there is a need for efficient cell lines that are able to produce high quality rAAV with high titer yields. Production of high titer rAAV by the engineered cell lines described herein advances the application of this vector system for gene therapy use in vivo.

### SUMMARY

The present disclosure addresses the need for obtaining improved rAAV titers for gene therapy applications by providing rAAV packaging and/or producer cell lines comprising cells in which one or more genes and/or proteins have been modified.

In one aspect, provided herein are recombinant adeno-associated virus (rAAV) packaging and/or producer cell lines comprising cells in which the expression of a gene selected from the group consisting of RIG-1 (DDX58), IFIT3, MDA5 (IFIH1), CGAS (cGAS), CHUK (IKK-α), DDX41, DHX58 (LGP2), IFI6, IKBKB (IKK-β), IRF3, IRF7, MAVS, MYD88, NFKB1, NFKB2, TBK1, TRIF, and TRIM25, and any combination thereof is reduced compared to that in control parental cells.

In some embodiments, the expression is reduced using a nuclease, a double stranded RNA (dsRNA), a small interfering RNA (siRNA), a small hairpin RNA (shRNA), a microRNA (miRNA), or an antisense RNA oligonucleotide (ASO).

In some embodiments, the expression is reduced with an siRNA comprising a nucleotide sequence selected from any one of SEQ ID NOs: 1-36.

For example, the expression of CGAS (cGAS) may be reduced, and the siRNA may comprise the nucleotide sequence of SEQ ID NO: 1 in the sense strand and the nucleotide sequence of SEQ ID NO: 2 in the anti-sense strand.

For example, the expression of CHUK (IKKα) may be reduced, and the siRNA may comprise the nucleotide sequence of SEQ ID NO: 3 in the sense strand and the nucleotide sequence of SEQ ID NO: 4 in the anti-sense strand.

For example, the expression of DDX41 may be reduced, and the siRNA may comprise the nucleotide sequence of SEQ ID NO: 5 in the sense strand and the nucleotide sequence of SEQ ID NO: 6 in the anti-sense strand.

For example, the expression of DHX58 (LGP2) may be reduced, and the siRNA may comprise the nucleotide sequence of SEQ ID NO: 7 in the sense strand and the nucleotide sequence of SEQ ID NO: 8 in the anti-sense strand.

For example, the expression of IFI6 may be reduced, and the siRNA may comprise the nucleotide sequence of SEQ ID NO: 9 in the sense strand and the nucleotide sequence of SEQ ID NO: 10 in the anti-sense strand.

For example, the expression of IFIT3 may be reduced, and the siRNA may comprise the nucleotide sequence of SEQ ID NO: 11 in the sense strand and the nucleotide sequence of SEQ ID NO: 12 in the anti-sense strand.

For example, the expression of IKBKB (IKK-β) may be reduced, and the siRNA may comprise the nucleotide sequence of SEQ ID NO: 13 in the sense strand and the nucleotide sequence of SEQ ID NO: 14 in the anti-sense strand.

For example, the expression of IRF3 may be reduced, and the siRNA may comprise the nucleotide sequence of SEQ ID NO: 15 in the sense strand and the nucleotide sequence of SEQ ID NO: 16 in the anti-sense strand.

For example, the expression of IRF7 may be reduced, and the siRNA may comprise the nucleotide sequence of SEQ ID NO: 17 in the sense strand and the nucleotide sequence of SEQ ID NO: 18 in the anti-sense strand.

For example, the expression of MAVS may be reduced, and the siRNA may comprise the nucleotide sequence of SEQ ID NO: 19 in the sense strand and the nucleotide sequence of SEQ ID NO: 20 in the anti-sense strand.

For example, the expression of MDA5 (IFIH1) may be reduced, and the siRNA may comprise the nucleotide sequence of SEQ ID NO: 21 in the sense strand and the nucleotide sequence of SEQ ID NO: 22 in the anti-sense strand.

For example, the expression of MYD88 may be reduced, and the siRNA may comprise the nucleotide sequence of SEQ ID NO: 23 in the sense strand and the nucleotide sequence of SEQ ID NO: 24 in the anti-sense strand.

For example, the expression of NFKB1 may be reduced, and the siRNA may comprise the nucleotide sequence of SEQ ID NO: 25 in the sense strand and the nucleotide sequence of SEQ ID NO: 26 in the anti-sense strand.

For example, the expression of NFKB2 may be reduced, and the siRNA may comprise the nucleotide sequence of SEQ ID NO: 27 in the sense strand and the nucleotide sequence of SEQ ID NO: 28 in the anti-sense strand.

For example, the expression of RIG-1 (DDX58) may be reduced, and the siRNA may comprise the nucleotide sequence of SEQ ID NO: 29 in the sense strand and the nucleotide sequence of SEQ ID NO: 30 in the anti-sense strand.

For example, the expression of TBK1 may be reduced, and the siRNA may comprise the nucleotide sequence of SEQ ID NO: 31 in the sense strand and the nucleotide sequence of SEQ ID NO: 32 in the anti-sense strand.

For example, the expression of TRIF may be reduced, and the siRNA may comprise the nucleotide sequence of SEQ ID NO: 33 in the sense strand and the nucleotide sequence of SEQ ID NO: 34 in the anti-sense strand.

For example, the expression of TRIM25 may be reduced, and the siRNA may comprise the nucleotide sequence of SEQ ID NO: 35 in the sense strand and the nucleotide sequence of SEQ ID NO: 36 in the anti-sense strand.

In some embodiments, the nuclease is selected from the group consisting of a Zinc Finger nuclease (ZFN), a meganuclease, a transcription activator-like effector nuclease (TALEN), and a clustered regularly interspaced short palindromic repeats (CRISPR) associated protein.

In some embodiments, the nuclease is a CRISPR associated protein and the expression is reduced using CRISPR genome editing.

In some embodiments, the expression is reduced using CRISPR genome editing, e.g., using a guide RNA that:
(a) comprises a sequence selected from SEQ ID NOs: 37-99, and/or
(b) targets a DNA sequence selected from SEQ ID NOs: 100-162.

For example, the gRNA may be used to target CGAS (cGAS) and may (a) comprise a sequence selected from SEQ ID NOs: 37-40 or any combination(s) thereof and/or (b) target a DNA sequence selected from SEQ ID NOs: 100-103 or any combination(s) thereof.

For example, the gRNA may be used to target CHUK (IKK-α) and may (a) comprise a sequence selected from SEQ ID NOs: 41-44 or any combination(s) thereof and/or (b) target a DNA sequence selected from SEQ ID NOs: 104-107 or any combination(s) thereof.

For example, the gRNA may be used to target DDX41 and may (a) comprise a sequence selected from SEQ ID NOs: 45-48 or any combination(s) thereof and/or (b) target a DNA sequence selected from SEQ ID NOs: 108-111 any combination(s) thereof.

For example, the gRNA may be used to target DHX58 (LGP2) and may (a) comprise the sequence of SEQ ID NO: 49 and/or (b) target a DNA sequence of SEQ ID NO: 112.

For example, the gRNA may be used to target IFI6 and may (a) comprise a sequence selected from SEQ ID NOs: 50-53 or any combination(s) thereof and/or (b) target a DNA sequence selected from SEQ ID NOs: 113-116 or any combination(s) thereof.

For example, the gRNA may be used to target IFIT3 and may (a) comprise a sequence selected from SEQ ID NOs: 54-57 or any combination(s) thereof and/or (b) target a DNA sequence selected from SEQ ID NOs: 117-120 or any combination(s) thereof.

For example, the gRNA may be used to target IKBKB (IKK-β) and may (a) comprise a sequence selected from SEQ ID NOs: 58-61 or any combination(s) thereof and/or (b) target a DNA sequence selected from SEQ ID NOs: 121-124 or any combination(s) thereof.

For example, the gRNA may be used to target IRF3 and may (a) comprise a sequence selected from SEQ ID NOs: 62-65 or any combination(s) thereof and/or (b) target a DNA sequence selected from SEQ ID NOs: 125-128 or any combination(s) thereof.

For example, the gRNA may be used to target IRF7 and may (a) comprise a sequence selected from SEQ ID NOs: 66-69 or any combination(s) thereof and/or (b) target a DNA sequence selected from SEQ ID NOs: 129-132 or any combination(s) thereof.

For example, the gRNA may be used to target MAVS and may (a) comprise a sequence selected from SEQ ID NOs: 70-73 or any combination(s) thereof and/or (b) target a DNA sequence selected from SEQ ID NOs: 133-136 or any combination(s) thereof.

For example, the gRNA may be used to target MDA5 (IFIH1) and may (a) comprise the sequence of SEQ ID NO: 74 and/or (b) target a DNA sequence of SEQ ID NO: 137.

For example, the gRNA may be used to target MYD88 and may (a) comprise a sequence selected from SEQ ID NOs: 75-78 or any combination(s) thereof and/or (b) target a DNA sequence selected from SEQ ID NOs: 138-141 or any combination(s) thereof.

For example, the gRNA may be used to target NFKB1 and may (a) comprise a sequence selected from SEQ ID NOs: 79-82 or any combination(s) thereof and/or (b) target a DNA sequence selected from SEQ ID NOs: 142-145 or any combination(s) thereof.

For example, the gRNA may be used to target NFKB2 and may (a) comprise a sequence selected from SEQ ID NOs: 83-86 or any combination(s) thereof and/or (b) target a DNA sequence selected from SEQ ID NOs: 146-149 or any combination(s) thereof.

For example, the gRNA may be used to target RIG-1 (DDX58) and may (a) comprise the sequence of SEQ ID NO: 87 and/or (b) target a DNA sequence of SEQ ID NO: 150.

For example, the gRNA may be used to target TBK1 and may (a) comprise a sequence selected from SEQ ID NOs: 88-91 or any combination(s) thereof and/or (b) target a DNA sequence selected from SEQ ID NOs: 151-154 or any combination(s) thereof.

For example, the gRNA may be used to target TRIF and may (a) comprise (a) a sequence selected from SEQ ID NOs: 92-95 or any combination(s) thereof and/or (b) target a DNA sequence selected from SEQ ID NOs: 155-158 or any combination(s) thereof.

For example, the gRNA may be used to target TRIM25 and may (a) comprise a sequence selected from SEQ ID NOs: 96-99 or any combination(s) thereof and/or (b) target a DNA sequence selected from SEQ ID NOs: 159-162 or any combination(s) thereof.

In some embodiments, the expression of RGMA is reduced compared to that in control parental cells, for example, using a nuclease, a double stranded RNA (dsRNA), a small interfering RNA (siRNA), a small hairpin RNA (shRNA), a microRNA (miRNA), or an antisense RNA oligonucleotide (ASO). For example, the expression of RGMA may be reduced using an siRNA that comprises the nucleotide sequence of SEQ ID NO: 238 in the sense strand and the nucleotide sequence of SEQ ID NO: 239 in the anti-sense strand.

In some embodiments, the nuclease is selected from the group consisting of a Zinc Finger nuclease (ZFN), a meganuclease, a transcription activator-like effector nuclease (TALEN), and a clustered regularly interspaced short palindromic repeats (CRISPR) associated protein. In some embodiments, the expression is reduced using CRISPR genome editing, e.g., using a guide RNA, wherein the guide RNA: targets a DNA sequence selected from SEQ ID NOs: 240-243.

In certain embodiments, each gRNA molecule is a 2′ O-methyl analog comprising 3″ phosphorothioate internucleotide linkages in the terminal three nucleotides on either or both its 5′ and 3′ ends.

In some embodiments, the expression of KCNN2 is reduced compared to that in control parental cells, for example, using a nuclease, a double stranded RNA (dsRNA), a small interfering RNA (siRNA), a small hairpin RNA (shRNA), a microRNA (miRNA), or an antisense RNA oligonucleotide (ASO). For example, the expression of KCNN2 may be reduced using an siRNA that comprises the nucleotide sequence of SEQ ID NO: 245 in the sense strand and the nucleotide sequence of SEQ ID NO: 246 in the anti-sense strand.

In some embodiments, the expression of KCNN2 is reduced using CRISPR genome editing, e.g., using a guide RNA, wherein the guide RNA: targets a DNA sequence selected from SEQ ID NOs: 251-254. In some embodiments, the gRNA pair used to target KCNN2 comprises a first gRNA molecule comprising the sequence of SEQ ID NO: 247 and a second gRNA molecule comprising the sequence of SEQ ID NO: 248. In some embodiments, the gRNA pair used to target KCNN2 comprises a first gRNA molecule comprising the sequence of SEQ ID NO: 249 and a second gRNA molecule comprising the sequence of SEQ ID NO: 250.

In some embodiments, each gRNA molecule is a 2′ O-methyl analog comprising 3′ phosphorothioate internucleotide linkages in the terminal three nucleotides on either or both its 5′ and 3′ ends.

In some embodiments, the expression of RGMA and/or KCNN2 is reduced compared to the expression of RGMA and KCNN2 in control parental cells.

In some embodiments, the gene expression is eliminated.

In some embodiments, the cell line is a human cell line, e.g., a HeLa cell line or a human embryonic kidney (HEK) 293 cell line.

In one aspect, provided herein is a cell comprising: (a) AAV rep and cap genes and (b) an siRNA comprising a nucleotide sequence selected from any one of SEQ ID NOs: 1-36. In some embodiments, provided herein is an rAAV packaging and/or producer cell line comprising the cell.

In one aspect, provided herein is a cell comprising: (a) AAV rep and cap genes and (b) (i) a guide RNA (gRNA) comprising a nucleotide sequence selected from SEQ ID Nos: 37-99; and/or (ii) a gRNA which targets a DNA sequence selected from SEQ ID NOs: 100-162. In some embodiments, provided herein is an rAAV packaging and/or producer cell line comprising the cell.

In one aspect, provided herein is a cell comprising: (a) AAV rep and cap genes and (b) a nuclease selected from the group consisting of Zinc Finger nuclease (ZFN), a meganuclease, or a transcription activator-like effector nuclease (TALEN), wherein the nuclease targets a gene selected from the group RIG-1 (DDX58), IFIT3, MDA5 (IFIH1), CGAS (cGAS), CHUK (IKK-α), DDX41, DHX58 (LGP2), IFI6, IKBKB (IKK-β), IRF3, IRF7, MAVS, MYD88, NFKB1, NFKB2, TBK1, TRIF, TRIM25, and combinations thereof. In some embodiments, provided herein is an rAAV packaging and/or producer cell line comprising the cell.

In one aspect, provided herein is a cell comprising: (a) AAV rep and cap genes and (b) a knockout version of a gene selected from the group consisting of RIG-1 (DDX58), IFIT3, MDA5 (IFIH1), CGAS (cGAS), CHUK (IKK-α), DDX41, DHX58 (LGP2), IFI6, IKBKB (IKK-β), IRF3, IRF7, MAVS, MYD88, NFKB1, NFKB2, TBK1, TRIF, TRIM25, and combinations thereof. In some embodiments, provided herein is an rAAV packaging and/or producer cell line comprising the cell.

In some embodiments, the AAV rep and cap genes are stably integrated into the genome of the cell.

In some embodiments, provided herein is an rAAV packaging and/or producer cell line comprising the cell.

In certain embodiments, provided are rAAV packaging cell lines according to the present disclosure.

In certain embodiments, provided are rAAV producer cell lines according to the present disclosure. For example, in some embodiments, provided is a cell line capable of producing rAAV or which produce rAAV at a titer that is increased by at least 1.5-fold or at least 2-fold compared to the titer of rAAV produced from a control parental cell line.

In one aspect, provided are lysates of a cell line disclosed herein.

In one aspect, provided are cell culture supernatants from a cell line disclosed herein.

In one aspect, provided are methods of generating a rAAV producer cell line, the method comprising delivering a recombinant adeno-associated virus (rAAV) vector to cells of a rAAV packaging cell line according to the present disclosure.

In one aspect, provided are methods of producing rAAV, the method comprising infecting the cells of a rAAV producer cell line generated by a method disclosed herein with a helper virus.

In one aspect, provided are methods of producing rAAV, the method comprising infecting the cells of a rAAV producer cell line according to the present disclosure with a helper virus. In some embodiments, methods further comprise harvesting the rAAV from the rAAV producer cell line.

In some embodiments, production of rAAV from the rAAV producer cell line is enhanced as compared that of a control parental cell line.

In one aspect, provided are recombinant adeno-associated virus (rAAV) packaging and/or producer cell lines comprising cells which have been engineered to reduce the expression and/or activity of a gene product expressed from a gene selected from the group consisting of RIG-1 (DDX58), IFIT3, MDA5 (IFIH1), CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, and TRIM25, and any combination thereof as compared to corresponding control parental cells.

In some embodiments, the expression and/or activity of a gene product expressed from a gene selected from the group consisting of RIG-1 (DDX58), IFIT3, MDA5 (IFIH1), CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, and TRIM25, and any combination thereof is reduced indefinitely or permanently.

In some embodiments, the cell line has been engineered to comprise a gene disruption or a partial or complete gene deletion in a gene selected from the group consisting of RIG-1 (DDX58), IFIT3, MDA5 (IFIH1), CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, and TRIM25, and any combination thereof.

In some embodiments, the cell line has been engineered to comprise a gene disruption in a gene selected from the group consisting of RIG-1 (DDX58), IFIT3, MDA5 (IFIH1), CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, and TRIM25, and any combination thereof.

In some embodiments, the cell line has been engineered to comprise a partial or complete gene deletion in a gene selected from the group consisting of RIG-1 (DDX58), IFIT3, MDA5 (IFIH1), CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, and TRIM25, and any combination thereof.

In some embodiments, the cells are further engineered to reduce the expression and/or activity of RGMA. In some embodiments, the cells are further engineered to reduce the expression and/or activity of KCNN2. In some embodiments, the cells are further engineered to reduce the expression and/or activity of RGMA and KCNN2.

In some embodiments, the cells are engineered to reduce the expression and/or activity of RIG-1 (DDX58) and RGMA. In some embodiments, the cells are engineered to reduce the expression and/or activity of RIG-1 (DDX58) and KCNN2. In some embodiments, the cells are engineered to reduce the expression and/or activity of RIG-1 (DDX58), RGMA, and KCNN2.

In one aspect, provided are recombinant adeno-associated virus (rAAV) packaging and/or producer cell lines which exhibit reduced expression and/or activity of a polypeptide or polyribonucleotide expressed from a gene selected from the group consisting of RIG-1 (DDX58), IFIT3, MDA5 (IFIH1), CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, and TRIM25, and any combination thereof as compared to that of a control parental cell line.

Other features and advantages of the disclosure will be apparent from the following detailed description and claims.

### BRIEF DESCRIPTION OF THE DRAWINGS

The disclosure can be more completely understood with reference to the following.

FIG. 1 is a schematic showing exemplary methods of generating rAAV packaging and producer cells described herein.

FIG. 2 is a schematic illustrating an exemplary process for gene knockdown using siRNA transfection on producer cell lines (PCLs) and analysis (e.g., by quantitative PCR (qPCR)) for rAAV titer.

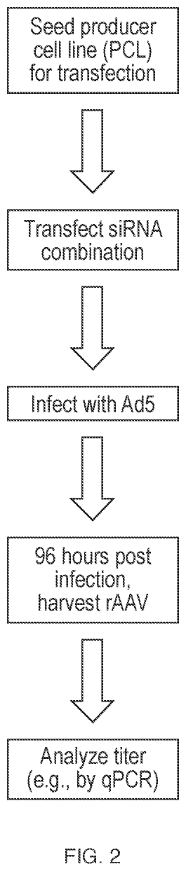

FIG. 3 is a schematic illustrating an exemplary process for gene knockout using CRISPR/Cas9 genome editing technology on producer cell lines (PCLs).

FIG. 4 is a graph depicting changes in rAAV titer from producer cell lines (PCLs) whose expression of cGAS, RIG-1, MDA5, or IFIT3 were knocked down using siRNA. Changes are presented as fold changes relative to rAAV titer from a negative control. Also shown as a positive control is the fold change in rAAV titer from a PCL in which KCNN2 was knocked down using siRNA.

### DETAILED DESCRIPTION OF THE DISCLOSURE

The present disclosure describes a recombinant adeno-associated virus (rAAV) packaging and/or producer cell line comprising cells in which expression of one or more genes and/or proteins is modulated. The modulation of gene expression results in an increased titer yield compared to a cell line in which expression of one or more genes and/or proteins is not modulated.

Unless otherwise noted, technical terms are used according to conventional usage. Definitions of common terms in molecular biology may be found in Benjamin Lewin, Genes V, published by Oxford University Press, 1994 (ISBN 0-19-854287-9): Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8.

The following definitions are included for the purpose of understanding the present subject matter and for construing the appended patent claims. Abbreviations used herein have their conventional meaning within the chemical and biological arts.

### Definitions

As used herein, “modulation” or “modulate” refers to the alteration of the regulation, expression or activity of a gene and/or protein. Modulation may be increasing, reducing (decreasing), or eliminating the expression and/or activity of one or more genes and/or proteins. In cases where multiple genes and/or proteins are modulated, all the expression and/or activity of genes and/or proteins may be increased, or all the expression and/or activity of genes and/or proteins may be decreased, or one or more genes and/or proteins may be increased and others of the genes and/or proteins may be decreased.

As used herein, the term “cell” refers to any cell or cells capable of producing a recombinant adeno-associated virus (rAAV), unless the context clearly indicates otherwise. In some embodiments, the cell is a mammalian cell, for example, a HeLa cell, a COS cell, a HEK293 cell, a A549 cell, a BHK cell, or a Vero cell. In other embodiments, the cell is an insect cell, for example, a Sf9 cell, a Sf-21 cell, a Tn-368 cell, or a BTI-Tn-5B1-4 (High-Five) cell. The term “cell line” refers to a line of cells that comprises a clonal population of cells able to continue to divide and not undergo senescence. In some embodiments, a cell line described in the present disclosure comprises a clonal population of cells. In some embodiments, less than 100% of the cells in the cell line is from a clonal population of cells. Unless otherwise indicated, the terms “cell” or “cell line” are understood to include modified or engineered variants of the indicated cell or cell line.

As used herein, the term “engineered cell line” refers to cell lines that have been modified by one or more means to reduce the expression or other properties (e.g., biological activity) of one or more (e.g., one, two, three, four, five, or more) endogenously expressed genes and/or proteins (e.g., CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, TRIM25, and any combination(s) thereof) so as to augment the production of rAAV.

As used herein, the term “control parental cells” refers to cells that have not been modified by one or more means to reduce the expression or other properties (e.g., biological activity) of one or more (e.g., one, two, three, four, five, or more) endogenously expressed genes and/or proteins (e.g., CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, TRIM25, and any combination(s) thereof) so as to augment the production of rAAV.

As used herein, the term “control parental cell line” refers to a clonal population of control parental cells able to continue to divide and not undergo senescence.

“Lysis” refers to the breaking down of the cell, often by viral, enzymatic, or osmotic mechanisms that compromise its integrity. A “lysed cell” is a cell that has undergone substantial lysis. As used herein, the term “lysate” refers to a fluid containing the contents of lysed cells.

As used herein, the term “higher titer” signifies an increased titer in comparison to titer produced by an unmodified control parental cell line and/or control parental cell.

As used herein, the term “cell culture supernatant” refers to the cell culture media in which cells are suspended and/or cultured.

The term “gene” refers to a transcription unit and may include regulatory regions that are adjacent (e.g., located upstream and downstream), and/or operably linked, to the transcription unit. A transcription unit is a series of nucleotides that are transcribed into an RNA molecule. A transcription unit may include a coding region. A “coding region” is a nucleotide sequence that encodes an unprocessed preRNA (i.e., an RNA molecule that includes both exons and introns) that is subsequently processed to an mRNA. A transcription unit may encode a non-coding RNA. A non-coding RNA is an RNA molecule that is not translated into a protein. Examples of non-coding RNAs include microRNA. The boundaries of a transcription unit are generally determined by an initiation site at its 5′ end and a transcription terminator at its 3′ end. A “regulatory region” is a nucleotide sequence that regulates expression of a transcription unit to which it is operably linked. Nonlimiting examples of regulatory sequences include promoters, enhancers, transcription initiation sites, translation start sites, translation stop sites, transcription terminators, and poly(A) signals. A regulatory region located upstream of a transcription unit may be referred to as a 5′ UTR, and a regulatory region located downstream of a transcription unit may be referred to as a 3′ UTR. A regulatory region may be transcribed and be part of an unprocessed preRNA.

In the context of this document, the term “target” or “target gene” refers to any gene, including protein-encoding genes and genes encoding non-coding RNAs (e.g., miRNA), that when modulated alters some aspect of virus production. Target genes include endogenous genes, viral genes, and transgenes.

As used herein, the term “eliminated,” when used in reference to gene expression, means that the gene expression is not detectable or is below a set threshold known to correspond to no expression in an assay, e.g., an assay to detect mRNA transcript levels and/or an assay to detect protein levels.

As used herein, the term “knockdown,” when used in reference to a gene, refers to a method to reduce but not necessarily eliminate expression of a gene of interest.

As used herein, the term “knockout,” when used in reference to a gene, refers to a method to eliminate expression of a gene of interest. Typically, gene knockout techniques involve deletion of at least a portion of a gene or otherwise modifying the gene such that gene expression is abrogated. In some cases, the deleted portion of the gene is replaced with another genetic element (i.e., a “knock-in”) not normally present in that position in the genome. A “knockout version of a gene” is a gene that has been altered to contain such a deletion or modification that abrogates expression of that gene. For example, in some embodiments, a knockout version of a gene comprises a deletion of at least one exon of the gene.

With regard to gene designations, single genes have often been denoted by multiple symbols. In the context of this document, gene symbols, whether they be human or non-human, may be designated by either upper-case or lower case letters. Neither the use of one particular symbol nor the adoption of lower or upper case symbols is intended to limit the scope of the gene in the context of these disclosures. All gene identification numbers identified herein (GeneID) are derived from the National Center for Biotechnology Information “Entrez Gene” or KEGG web site unless identified otherwise.

As used herein, the terms “about” and “approximately” are used interchangeably and mean in the region of, roughly or around. When the term “about” is used in conjunction with a numerical range, it modifies that range by extending, within permissible value ranges, the boundaries above and/or below the numerical values set forth. In some embodiments, the term “about” or “approximately,” when used in reference to a numerical value, includes values that are within 10%-15% of the numerical value.

As used in the present disclosure, whether in a transitional phrase or in the body of a claim, the terms “comprise(s)” and “comprising” are to be interpreted as having an open-ended meaning. That is, the terms are to be interpreted synonymously with the phrases “having at least” or “including at least.” When used in the context of a method, the term “comprising” means that the method includes at least the recited steps, but may include additional steps. When used in the context of a composition, the term “comprising” means that the composition includes at least the recited features or components, but may also include additional features or components.

For the purposes of promoting an understanding of the embodiments described herein, reference made to preferred embodiments and specific language is used to describe the same. The terminology used herein is for the purpose of describing particular embodiments only, and is not intended to limit the scope of the present disclosure. As used throughout this disclosure, the singular forms “a,” “an,” and “the” include plural reference unless the context clearly dictates otherwise. All percentages and ratios used herein, unless otherwise indicated, are by weight.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. In addition, the materials, methods and examples are illustrative only and are not intended to be limiting. All publications, patent applications, patents and other references mentioned herein are incorporated by reference.

### Adeno-Associated Virus (AAV)

AAV is a small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. AAV includes numerous serologically distinguishable types including serotypes AAV-1 to AAV-13, as well as more than 100 serotypes from nonhuman primates (See, e.g., Srivastava, J. Cell Biochem., 105 (1): 17-24 (2008), and Gao et al., J. Virol., 78 (12), 6381-6388 (2004)). AAV is non-autonomously replicating, and has a life cycle with a latent phase and an infectious phase. In the latent phase, after a cell is infected with an AAV, the AAV site-specifically integrates into the host's genome as a provirus. The infectious phase does not occur unless the cell is also infected with a helper virus (for example, adenovirus (AV) or herpes simplex virus), which allows the AAV to replicate. A recombinant AAV comprises an AAV capsid and a vector genome packaged therein, wherein the vector genome comprises a nucleic acid encoding a protein. The AAV capsid may be from an AAV of serotype 1, 2, 3, 4, 5, 6, 7, 8, 9, 10, 11, 12, rh10, rh74, hu37 (i.e., AAV1, AAV2, AAV3, AAV4, AAV5, AAV6, AAV7, AAV9, AAV10, AAV11, AAV12, AAVrh10, AAVrh74, AAVhu37), or an engineered variant thereof. In an exemplary embodiment, the AAV capsid is selected from an AAV serotype hu37 (AAVhu37) capsid, an AAV serotype 9 (AAV9) capsid, an AAV9 variant capsid, an AAV serotype 8 (AAV8) capsid, or an AAV8 variant capsid. In a further exemplary embodiment, the AAV capsid is the AAVhu37 capsid.

The wild-type AAV genome contains two 145 nucleotide inverted terminal repeats (ITRs), which contain signal sequences directing AAV replication, genome encapsidation and integration. In addition to the ITRs, three AAV promoters, p5, p19, and p40, drive expression of two open reading frames encoding rep and cap genes. Two rep promoters, coupled with differential splicing of the single AAV intron, result in the production of four rep proteins (Rep 78, Rep 68, Rep 52, and Rep 40) from the rep gene. Rep proteins are responsible for genomic replication. The cap gene is expressed from the p40 promoter, and encodes three capsid proteins (VP1, VP2, and VP3) which are splice variants of the cap gene. These proteins form the capsid of the AAV particle.

Because the cis-acting signals for replication, encapsidation, and integration are contained within the ITRs, some or all of the 4.3 kb internal genome may be replaced with foreign DNA, for example, an expression cassette for an exogenous protein of interest. In this case, the rep and cap proteins are provided in trans on, for example, a plasmid. In order to produce an AAV vector, a cell line permissive of AAV replication must express the rep and cap genes, the ITR-flanked expression cassette, and helper functions provided by a helper virus, for example AV genes E1a, E1b55K, E2a, E4orf6, and VA (Weitzman et al., Adeno-associated virus biology. Adeno-Associated Virus: Methods and Protocols, pp. 1-23, 2011). Production of AAV vector can also result in the production of helper virus particles, which must be removed or inactivated prior to use of the AAV vector. Numerous cell types are suitable for producing AAV vectors, including HEK293 cells, COS cells, HeLa cells, BHK cells, Vero cells, as well as insect cells (See e.g. U.S. Pat. Nos. 6,156,303, 5,387,484, 5,741,683, 5,691, 176, 5,688,676, 8,163,543, U.S. Publication No. 20020081721, PCT Publication Nos. WO 00/47757, WO 00/24916, and WO 96/17947). AAV vectors are typically produced in these cell types by one plasmid containing the ITR-flanked expression cassette, and one or more additional plasmids providing the additional AAV and helper virus genes.

AAV of any serotype may be used in the present disclosure. Similarly, it is contemplated that any AV type may be used, and a person of skill in the art will be able to identify AAV and AV types suitable for the production of their desired recombinant AAV vector (rAAV). AAV and AV particles may be purified, for example, by affinity chromatography, iodixanol gradient, or cesium chloride (CsCl) gradient.

The genome of wild-type AAV is single-stranded DNA and is 4.7 kb. AAV vectors may have single-stranded genomes that are 4.7 kb in size, or are larger or smaller than 4.7 kb, including oversized genomes that are as large as 5.2 kb, or as small as 3.0 kb. Further, vector genomes may be substantially self-complementary, so that within the virus the genome is substantially double stranded. AAV vectors containing genomes of all types are suitable for use in the method of the instant disclosure.

As discussed above, AAV requires co-infection with a helper virus in order to enter the infectious phase of its life cycle. Helper viruses include Adenovirus (AV), and herpes simplex virus (HSV), and systems exist for producing AAV in insect cells using baculovirus. It has also been proposed that papilloma viruses may also provide a helper function for AAV (see. e.g., Hermonat et al., Molecular Therapy 9, S289-S290 (2004)). Helper viruses include any virus capable of creating and allowing AAV replication. AV is a nonenveloped nuclear DNA virus with a double-stranded DNA genome of approximately 36 kb. AV is capable of rescuing latent AAV provirus in a cell, by providing E1a, E1b55K, E2a, E4orf6, and VA genes, and allowing AAV replication and encapsidation. HSV is a family of viruses that have a relatively large double-stranded linear DNA genome encapsidated in an icosahedral capsid, which is wrapped in a lipid bilayer envelope. HSV are infectious and highly transmissible. The following HSV-1 replication proteins were identified as necessary for AAV replication: the helicase/primase complex (UL5, UL8, and UL52) and the DNA binding protein ICP8 encoded by the UL29 gene, with other proteins enhancing the helper function. An AAV packaging system serves two purposes: it circumvents the problem of the transfection process, and provide a production technology based on the use of one or several helper functions.

Production of rAAV

General principles of rAAV can be reviewed elsewhere (See. e.g., Carter, 1992, Current Opinions in Biotechnology, 3:533-539; and Muzyczka, 1992, Curr. Topics in Microbiol. and Immunol., 158:97-129). In general terms, to allow for production of rAAV, the cell must be provided with AAV ITRs, which may, for example, flank a heterologous nucleotide sequence of interest, AAV rep and cap gene functions, as well as additional helper functions. These may be provided to the cell using any number of appropriate plasmids or vectors. Additional helper functions can be provided by, for example, an adenovirus (AV) infection, by a plasmid that carries all of the required AV helper function genes, or by other viruses such as HSV or baculovirus. Any genes, gene functions, or genetic material necessary for rAAV production by the cell may transiently exist within the cell, or be stably inserted into the cell genome. rAAV production methods suitable for use with the methods of the current disclosure include those disclosed in Clark et al., Human Gene Therapy 6:1329-1341 (1995), Martin et al., Human Gene Therapy Methods 24:253-269 (2013), Thorne et al., Human Gene Therapy 20:707-714 (2009), Fraser Wright, Human Gene Therapy 20:698-706 (2009), and Virag et al., Human Gene Therapy 20:807-817 (2009). The two main approaches for AAV production systems are recombinant adeno-associated virus (rAAV) packaging cell line and adeno-associated virus (rAAV) producer cell line.

Recombinant Adeno-Associated Virus (rAAV) Packaging and/or Producer Cell Line

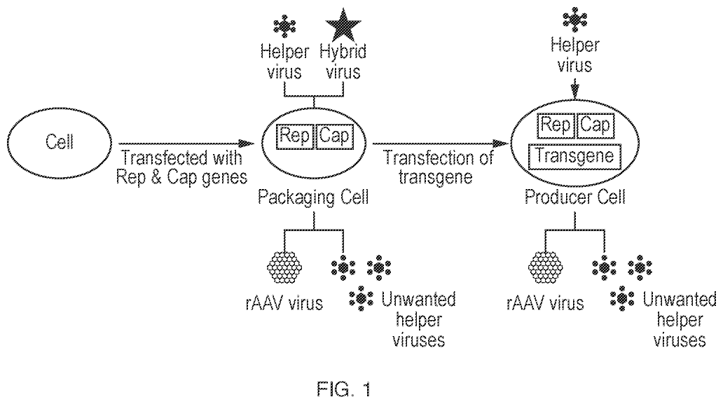

A rAAV packaging cell line can be produced by allowing cellular expression of AAV genetic elements described herein. The stable transfection of a cell line (e.g., HEK293, HeLa) with a plasmid encoding the AAV rep and cap genes can result in production of a packaging cell line. This rAAV packaging cell line can be co-infected with two different adenoviruses (helper virus and hybrid virus that contains the AAV gene-therapy elements) to produce rAAV particles. Alternatively, the stable transfection of the packaging cells with a plasmid containing the rAAV vector or their infection with a rAAV vector leads to a rAAV producer cell line. The infection of the producer cells with a helper virus leads to production of rAAV. FIG. 1 illustrates exemplary methods of generating packaging and producer cell lines.

In certain embodiments of the present disclosure, the rAAV packaging cell line comprising AAV rep and cap gene functions is engineered to increase the rAAV titer.

In one aspect, the present disclosure provides a rAAV packaging cell line comprising cells in which expression of a gene selected from the group consisting of CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, TRIM25, and any combination(s) thereof is reduced compared to control parental cells.

In some embodiments, the present disclosure provides a rAAV packaging cell line comprising cells in which expression of CGAS, RIG-1, MDA5, or IFIT3 is reduced compared to that in control parental cells.

In other embodiments, the present disclosure provides a rAAV producer cell line comprising cells in which expression of one or more (e.g., one, two, three, four, five, or more) genes and/or proteins is reduced compared to control parental cells. For example, expression of a gene selected from the group consisting of CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2. RIG-1 (DDX58), TBK1, TRIF, TRIM25, and any combination(s) thereof is reduced compared to that in control parental cells.

In certain embodiments, the cell line of the present disclosure may be in an adherent or suspension form.

In certain embodiments, the cell line of the present disclosure (e.g., rAAV packaging and/or producer cell line) is a mammalian cell line (e.g., HeLa, human embryonic kidney (HEK) 293, COS, A549, or Vero cell line). In certain embodiments, the cell line is an insect cell line (e.g., Sf9, Sf-21, Tn-368, or BTI-Tn-5B1-4).

Method of Generating a rAAV Producer Cell Line

In some embodiments, the present disclosure provides a method of generating a producer cell line by delivering a rAAV vector to an engineered rAAV packaging cell line comprising cells in which the expression of a gene selected from the group consisting of CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, TRIM25, and any combination(s) thereof is reduced compared to that in control parental cells. In some embodiments, the expression of one gene is reduced. In some embodiments, the expression of a combination of genes is reduced. Examples of combinations of genes whose expression can be reduced together include, but are not limited to, 1) DHX58, MDA5, and RGMA; 2) DHX58 and MDA5; 3) CGAS and DHX50; 5) CGAS and MDA5; 6) CGAS and RGMA; 7) CGAS, DHX58, MDA5, and RGMA; 8) CGAS, DHX58, and MDA5; 9) RGMA and DHX58; 10) RIG and RGMA; 11) RIG and KCNN2; 12) RIG, RGMA, and KCNN2; 13) IFIT3 and RGMA; 14) IFIT3 and KCNN2; 15) IFIT3, RGMA, and KCNN2; 16) MDA5 and RGMA; 17) MDA5 and KCNN2; 18) MDA5, RGMA, and KCNN2; 19) CGAS and RGMA; 14) CGAS and KCNN2; 20) CGAS, RGMA, and KCNN2; 21) CHUK and RGMA; 22) CHUK and KCNN2; 23) CHUK, RGMA, and KCNN2; 24) DDX41 and RGMA; 25) DDX41 and KCNN2; 26) DDX41, RGMA, and KCNN2; 27) DHX58 and RGMA; 28) DHX58 and KCNN2; 29) DHX58, RGMA, and KCNN2; 30) IFI6 and RGMA; 31) IFI6 and KCNN2; 32) IFI6, RGMA, and KCNN2; 33) IKBKB and RGMA; 34) IKBKB and KCNN2; 35) IKBKB, RGMA, and KCNN2; 36) IRF3 and RGMA; 37) IRF3 and KCNN2; 38) IRF3, RGMA, and KCNN2; 39) IRF7 and RGMA; 40) IRF7 and KCNN2; 41) IRF7, RGMA, and KCNN2; 42) MAVS and RGMA; 43) MAVS and KCNN2; 44) MAVS, RGMA, and KCNN2; 45) MYD88 and RGMA; 46) MYD88 and KCNN2; 47) MYD88, RGMA, and KCNN2; 48) NFKB1 and RGMA; 49) NFKB1 and KCNN2; 50) NFKB1, RGMA, and KCNN2; 51) NFKB2 and RGMA; 52) NFKB2 and KCNN2; 53) NFKB2, RGMA, and KCNN2; 54) TBK1 and RGMA; 55) TBK1 and KCNN2; 56) TBK1, RGMA, and KCNN2; 57) TRIF and RGMA; 58) TRIF and KCNN2; 59) TRIF, RGMA, and KCNN2; 60) TRIM25 and RGMA; 61) TRIM25 and KCNN2; and 62) TRIM25, RGMA, and KCNN2.

### Supplements

As used herein, the term “supplements” refers to any compound or other material, whether chemical or biological in origin, which may be used in a media for cell culture to increase rAAV titers or to assay for increases in rAAV titers. Non-limiting examples of supplements include amino acids, salts, metals, sugars, lipids, nucleic acids, hormones, vitamins, fatty acids, proteins, enzymes, nucleosides, metabolites, surfactants, emulsifiers, inorganic salts, and polymers. In certain embodiments, the one or more supplements added to the rAAV packaging and/or producer cell line of the present disclosure is a glucocorticoid analog. In certain embodiments, the one or more supplements added to the rAAV packaging and/or producer cell line includes dexamethasone, hydrocortisone, prednisolone, methylprednisolone, betamethasone, cortisone, prednisone, budesonide, and/or triamcinolone.

In certain embodiments, the concentration of glucocorticoid analog in solution for increasing rAAV titer can be greater than or equal to 1 μM, greater than or equal to 0.1 μM, greater than or equal to 0.01 μM, between 0 and 1 μM, between 0 and 0.1 μM, between 0 and 0.01 μM, between 0.01 and 1 μM, or between 0.01 and 0.1 μM.

As used herein, “supplemented cell line” refers to a cell line (e.g., rAAV packaging and/or producer cell line) in which one or more supplements (e.g., one or more glucocorticoid analogs) have been added to increase rAAV titer. As used herein, “non-supplemented cell line” refers to a cell line (e.g., rAAV packaging and/or producer cell line) not exposed to a supplement or supplements for increasing rAAV titer. As used herein, the terms “non-supplemented” and “unsupplemented” are used interchangeably to refer to culture conditions where the cell line (e.g., rAAV packaging and/or producer cell line) is not exposed to a supplement or supplements for increasing rAAV titer.

Modulated Genes and/or Proteins

In certain embodiments, the present disclosure provides a list of genes that when modulated (individually or in combinations) in a rAAV packaging and/or producer cell line enhance the production of rAAV. In certain embodiments, the modulation is increasing the expression of the one or more gene and/or protein. In certain embodiments, the modulation is decreasing the expression of the one or more gene and/or protein. In cases where multiple genes and/or proteins are modulated, all the gene/proteins may be increased, or all the genes/proteins may be decreased, or one or more genes/proteins may be increased and others of the genes/proteins may be decreased. In certain embodiments, the modulated genes are selected from the group consisting of CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, TRIM25, and combinations thereof.

### Cyclic GMP-AMP Synthase (CGAS)

In some embodiments, the modulated genes comprise Cyclic GMP-AMP Synthase (also known as CGAS or cGAS). CGAS is a nucleotidyltransferase that catalyzes the formation of cyclic GMP-AMP (cGAMP) from ATP and GTP and plays a key role in innate immunity.

An example of a human CGAS nucleotide sequence is available under reference sequence NM_138441.3 (SEQ ID NO: 163) in the NCBI nucleotide database.

### CHUK (IKK-α)

In some embodiments, the modulated genes comprise Component Of Inhibitor Of Nuclear Factor Kappa B Kinase Complex (CHUK) (also known as IKK-α). CHUK is a member of the serine/threonine protein kinase family. The encoded protein is a component of a cytokine-activated protein complex that is an inhibitor of the essential transcription factor NF-kappa-B complex and phosphorylates sites that trigger the degradation of the inhibitor via the ubiquitination pathway, thereby activating the transcription factor.

Examples of human CHUK nucleotide sequences are available under reference sequences NM_001278.5 (SEQ ID NO: 164) and NM_001320928.2 (SEQ ID NO: 165) in the NCBI nucleotide database.

### DDX41

In some embodiments, the modulated genes comprise DEAD-Box Helicase 1 (DDX41). DDX41 is a member of the DEAD box protein family and a putative RNA helicase. RNA helicases are implicated in a number of cellular processes involving alteration of RNA secondary structure, such as translation initiation, nuclear and mitochondrial splicing, and ribosome and spliceosome assembly. Based on their distribution patterns, some members of the DEAD box protein family are believed to be involved in embryogenesis, spermatogenesis, and cellular growth and division. The protein encoded by DDX41 interacts with several spliceosomal proteins. In addition, the encoded protein may recognize the bacterial second messengers cyclic di-GMP and cyclic di-AMP, resulting in the induction of genes involved in the innate immune response.

Examples of human DDX41 nucleotide sequences are available under reference sequences XM_024446109 (SEQ ID NO: 166), NM_001321732.2 (SEQ ID NO: 167), and NM_016222.4 (SEQ ID NO: 168) in the NCBI nucleotide database.

### DExH-Box Helicase 58

In some embodiments, the modulated genes comprise DExH-Box Helicase 58 (also known as DHX58 or LGP2). DHX58 regulates DDX58/RIG-1 and IFIH1/MDA5 mediated antiviral signaling and is involved in the innate immune response to various RNA viruses and some DNA viruses such as poxviruses and coronavirus SARS-COV-2.

An example of a human DHX58 nucleotide sequence is available under reference sequence NM_024119.3 (SEQ ID NO: 169) in the NCBI nucleotide database (nucleotide sequence).

### Interferon Alpha Inducible Protein 6

In some embodiments, the modulated genes comprise Interferon Alpha Inducible Protein 6 (also known as IFI6). IFI6 may play a role in apoptosis by negatively regulating the intrinsic apoptotic signaling pathway and TNSF1-induced apoptosis. IFI6 exhibits antiviral activity toward hepatitis C virus (HCV) by inhibiting the EGFR signaling pathway, which activation is required for entry of HCV into cells.

Examples of human IFI6 nucleotide sequences are available under reference sequences NM_002038.4 (SEQ ID NO: 170), NM_022872.3 (SEQ ID NO: 171), and NM_022873.3 (SEQ ID NO: 172) in the NCBI nucleotide database.

Interferon Induced Protein with Tetratricopeptide Repeats 3

In some embodiments, the modulated genes comprise Interferon Induced Protein with Tetratricopeptide Repeats 3 (also known as IFIT3). IFIT3 is an interferon-induced antiviral protein which acts as an inhibitor of cellular as well as viral processes, cell migration, proliferation, signaling, and viral replication.

Examples of human IFIT3 nucleotide sequences are available under reference sequences NM_001031683.4 (SEQ ID NO: 173), NM_001289758.2 (SEQ ID NO: 174), NM_001289759.2 (SEQ ID NO: 175), and NM_001549.6 (SEQ ID NO: 176) in the NCBI nucleotide database.

### Inhibitor Of Nuclear Factor Kappa B Kinase Subunit Beta IKBKB (IKK-β)

In some embodiments, the modulated genes comprise Inhibitor Of Nuclear Factor Kappa B Kinase Subunit Beta (IKBKB: also known as IKK-β). IKBKB is a serine kinase that plays an essential role in the NF-kappa-B signaling pathway which is activated by multiple stimuli such as inflammatory cytokines, bacterial or viral products. DNA damages or other cellular stresses. IKBKB acts as part of the canonical IKK complex in the conventional pathway of NF-kappa-B activation.

Examples of human IKBKB nucleotide sequences are available under the reference sequence NM_001190720.3 (SEQ ID NO: 177), NM_001242778.2 (SEQ ID NO: 178), and NM_001556.3 (SEQ ID NO: 179) in the NCBI nucleotide database.

### Interferon Regulatory Factor 3

In some embodiments, the modulated genes comprise Interferon Regulatory Factor 3 (IRF3). IRF3 is a key transcriptional regulator of type I interferon (IFN)-dependent immune responses which plays a critical role in the innate immune response against DNA and RNA viruses.

Examples of human IRF3 nucleotide sequences are available under reference sequences NM_001197122.2 (SEQ ID NO: 180), NM_001197123.2 (SEQ ID NO: 181), NM_001197124.2 (SEQ ID NO: 182), NM_001197125.2 (SEQ ID NO: 183), NM_001197126.2 (SEQ ID NO: 184), NM_001197127.2 (SEQ ID NO: 185), NM_001197128.2 (SEQ ID NO: 186), and NM_001571.6 (SEQ ID NO: 187) in the NCBI nucleotide database.

### Interferon Regulatory Factor 7

In some embodiments, the modulated genes comprise Interferon Regulatory Factor 7 (IRF7). IRF7 is a key transcriptional regulator of type I interferon (IFN)-dependent immune responses and plays a critical role in the innate immune response against DNA and RNA viruses.

Examples of human IRF7 nucleotide sequences are available under reference sequences XM_011520066.3 (SEQ ID NO: 188), XM_005252907.3 (SEQ ID NO: 189), XM_005252909.3 (SEQ ID NO: 190), and XM_017017674.1 (SEQ ID NO: 191) in the NCBI nucleotide database.

### Mitochondrial Antiviral Signaling Protein

In some embodiments, the modulated genes comprise Mitochondrial Antiviral Signaling Protein (MAVS). MAVS is required for innate immune defenses against viruses. MAVS acts downstream of DHX33, DDX58/RIG-I and IFIH1/MDA5, which detect intracellular dsRNA produced during viral replication, to coordinate pathways leading to the activation of NF-kappa-B. IRF3 and IRF7, and to the subsequent induction of antiviral cytokines such as IFNB and RANTES (CCL5). Peroxisomal and mitochondrial MAVS act sequentially to create an antiviral cellular state. Upon viral infection, peroxisomal MAVS induces the rapid interferon-independent expression of defense factors that provide short-term protection, whereas mitochondrial MAVS activates an interferon-dependent signaling pathway with delayed kinetics, which amplifies and stabilizes the antiviral response.

Examples of human MAVS nucleotide sequences are available under reference sequences NM_001206491.2 (SEQ ID NO: 192), NM_001385663.1 (SEQ ID NO: 193), and NM_020746.5 (SEQ ID NO: 194) in the NCBI nucleotide database.

Melanoma Differentiation-Associated Protein 5, Encoded by Interferon Induced with Helicase C Domain 1

In some embodiments, the modulated genes comprise Melanoma differentiation-associated protein 5 (MDA5). Interferon induced with helicase C domain 1 (IFIH1) encodes MDA5, which is an intracellular sensor of viral RNA that triggers the innate immune response. Sensing RNA length and secondary structure, MDA5 binds dsRNA oligonucleotides with a modified DExD/H-box helicase core and a C-terminal domain, thus leading to a proinflammatory response that includes interferons. It has been shown that Coronaviruses (CoVs) as well as various other virus families, are capable of evading the MDA5-dependent interferon response, thus impeding the activation of the innate immune response to infection. MDA5 has also been shown to play an important role in enhancing natural killer cell function in malaria infection. In addition to its protective role in antiviral responses, MDA5 has been implicated in autoimmune and autoinflammatory diseases such as type 1 diabetes, systemic lupus erythematosus, and Aicardi-Goutieres syndrome

An example of a human IFIH1 nucleotide sequence is available under reference sequence NM_022168.4 (SEQ ID NO: 195) in the NCBI nucleotide database.

### MYD88 Innate Immune Signal Transduction Adaptor

In some embodiments, the modulated genes comprise MYD88 Innate Immune Signal Transduction Adaptor (MYD88). MYD88 is a cytosolic adapter protein that plays a central role in the innate and adaptive immune response. MYD88 functions as an essential signal transducer in the interleukin-1 and Toll-like receptor signaling pathways. These pathways regulate that activation of numerous proinflammatory genes.

Examples of a human MYD88 nucleotide sequences are available under reference sequences NM_001172566.2 (SEQ ID NO: 196), NM_001172567.2 (SEQ ID NO: 197), NM_001172568.2 (SEQ ID NO: 198), NM_001172569.3 (SEQ ID NO: 199), NM_001365876.1 (SEQ ID NO: 200), NM_001365877.1 (SEQ ID NO: 201), NM_001374787.1 (SEQ ID NO: 202), NM_001374788.1 (SEQ ID NO:203), and NM_002468.5 (SEQ ID NO: 204) in the NCBI nucleotide database.

### Pleiotropic Transcription Factor and Immune Response Regulator NFKB

In some embodiments, the modulated genes comprise NFKB (also known as nuclear factor kappa-light-chain-enhancer of activated B cells or NF-kappa-B). NFKB is a pleiotropic transcription factor protein complex that regulates and potentiates an array of stimuli including, but not limited to, inflammation, innate and adaptive immunity, apoptosis and tumorigenicity. NFKB, which is reported to regulate the expression of at least 400 human genes, refers to the gene product of NFKB1, producing the immature p105 protein and processed, mature p50 protein, or NFKB2, producing the immature p100 protein and processed, mature p52 protein, complexed with additional proteins generated from other genes.

Examples of human NFKB1 nucleotide sequences are available under reference sequences NM_003998.4 (SEQ ID NO: 205), NM_001165412.2 (SEQ ID NO: 206), NM_001319226.2 (SEQ ID NO: 207), NM_001382625.1 (SEQ ID NO: 208), NM_001382626.1 (SEQ ID NO: 209), NM_001382627.1 (SEQ ID NO: 210), and NM_001382628.1 (SEQ ID NO: 211) in the NCBI nucleotide database.

Examples of human NFKB2 nucleotide sequences are available under reference sequences NM_001322934.2 (SEQ ID NO: 212), NM_002502.6 (SEQ ID NO: 213), NM_001077494.3 (SEQ ID NO: 214), NM_001261403.3 (SEQ ID NO: 215), NM_001288724.1 (SEQ ID NO: 216), and NM_001322935.1 (SEQ ID NO: 217) in the NCBI nucleotide database.

### RGMA (Repulsive Guidance Molecule BMP Co-Receptor A)

In some embodiments, the modulated genes comprise RGMA (Repulsive Guidance Molecule BMP Co-Receptor A). RGMA is a glycosylphosphatidylinositol-anchored glycoprotein that functions as an axon guidance protein in the developing and adult central nervous system. RGMA is a member of the repulsive guidance molecule (RGM) family that performs several functions in the developing and adult nervous system. Functions include regulation of cephalic neural tube closure, inhibition of neurite outgrowth and cortical neuron branching, and formation of mature synapses. Binding to its receptor NEO1/neogenin induces activation of RHOA-ROCK1/Rho-kinase signaling pathway through UNC5B-ARHGEF12/LARG-PTK2/FAK1 cascade, leading to collapse of the neuronal growth cone and neurite outgrowth inhibition. Furthermore, RGMA binding to NEO1/neogenin leads to HRAS inactivation by influencing HRAS-PTK2/FAK1-AKT1 pathway. RGMA also functions as a bone morphogenetic protein (BMP) coreceptor that may signal through SMAD1, SMAD5, and SMAD8.

Examples of human RGMA nucleotide sequences are available under reference sequences NM_001166283.2 (SEQ ID NO: 232), NM_001166286.2 (SEQ ID NO: 233), NM_001166287.2 (SEQ ID NO:234), NM_001166288.2 (SEQ ID NO:235), NM_001166289.2 (SEQ ID NO:236), and NM_020211.3 (SEQ ID NO:237) in the NCBI nucleotide database.

### Potassium Calcium-Activated Channel Subfamily N Member 2

Potassium Calcium-Activated Channel Subfamily N Member 2 (also known as KCNN2) gene is a member of the KCNN family of potassium channel genes. The encoded protein is an integral membrane protein that forms a voltage-independent calcium-activated channel with three other calmodulin-binding subunits. Alternate splicing of this gene results in multiple transcript variants. Examples of KCNN2 sequences are available under the reference sequence NM_170775.2 (SEQ ID NO: 255) or NM_001278204.1 (SEQ ID NO: 256) in the NCBI nucleotide database (nucleotide sequence).

### Antiviral Innate Immune Response Receptor RIG-1

In some embodiments, the modulated genes comprise RIG-1 (also known as DexD-H-Box Helicase 58 (DDX58)). RIG-1 is an innate immune receptor that senses cytoplasmic viral nucleic acids and activates a downstream signaling cascade leading to the production of type I interferons and proinflammatory cytokines. RIG-1 is a member of the DEAD box protein family, characterized by the conserved motif Asp-Glu-Ala-Asp (DEAD) (SEQ ID NO: 244). DEAD box proteins are putative RNA helicases which are implicated in a number of cellular processes involving RNA binding and alteration of RNA secondary structure.

Examples of human RIG-1 nucleotide sequences are available under reference sequences NM_001385907.1 (SEQ ID NO: 218), NM_001385909.1 (SEQ ID NO: 219), NM_001385910.1 (SEQ ID NO: 220), NM_001385912.1 (SEQ ID NO: 221), NM_001385913.1 (SEQ ID NO: 222), NM_001385914.1 (SEQ ID NO: 223), and NM_014314.4 (SEQ ID NO: 224) in the NCBI nucleotide database.

### Tank Binding Kinase 1

In some embodiments, the modulated genes comprise Tank Binding Kinase 1 (TBK1). TBK1 is a serine/threonine kinase that plays an essential role in regulating inflammatory responses to foreign agents. Following activation of toll-like receptors by viral or bacterial components, TBK1 associates with TRAF3 and TANK and phosphorylates interferon regulatory factors (IRFs) IRF3 and IRF7 as well as DDX3X. This activity allows subsequent homodimerization and nuclear translocation of the IRFs, leading to transcriptional activation of pro-inflammatory and antiviral genes including IFNA and IFNB.

Examples of human TBK1 nucleotide sequences are available under reference sequence XM_005268809.1 (SEQ ID NO: 225) and XM_005268810.1 (SEQ ID NO: 226) in the NCBI nucleotide database.

### TIR-Domain-Containing Adapter-Inducing Interferon β

In some embodiments, the modulated genes comprise TIR-domain-containing adapter-inducing interferon β (TRIF) (also known as Toll Like Receptor Adaptor Molecule 1 (TICAM1)). TRIF is an adaptor protein that contains an intracellular signaling domain that mediates protein-protein interactions between the Toll-like receptors (TLRs) and signal-transduction components. TRIF is involved in native immunity against invading pathogens. TRIF specifically interacts with toll-like receptor 3, but not with other TLRs, and this association mediates dsRNA induction of interferon-beta through activation of nuclear factor kappa-B, during an antiviral immune response.

Examples of human TRIF nucleotide sequences are available under reference sequence NM_001385678.1 (SEQ ID NO: 227), NM_001385679.1 (SEQ ID NO: 228), NM_001385680.1 (SEQ ID NO: 229), and NM_182919.4 (SEQ ID NO: 230) in the NCBI nucleotide database.

### Tripartite Motif Containing 25

In some embodiments, the modulated genes comprise Tripartite Motif Containing 25 (TRIM25) is a member of the tripartite (TRIM) family. The TRIM motif includes three zinc-binding domains, a RING, a B-box type 1 and a B-box type 2, and a coiled-coil region.

TRIM25 functions as a ubiquitin E3 ligase and as an ISG15 E3 ligase. TRIM25 is involved in innate immune defense against viruses by mediating ubiquitination of DDX58 and IFIH1.

An example of a human TRIM25 nucleotide sequence is available under the reference sequence NM_005082.5 (SEQ ID NO: 231) in the NCBI nucleotide database.

In certain embodiments, the present disclosure provides a rAAV packaging and/or producer cell line comprising cells in which the expression of CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, TRIM25, or any combination(s) thereof is reduced compared to control parental cells.

In certain embodiments, the present disclosure provides a list of genes that when modulated individually in a rAAV packaging and/or producer cell line enhance the production of rAAV compared to a control parental cell line. In some aspects, the modulation of different combination of genes in a rAAV packaging and/or producer cell line increases the production of rAAV. In some aspects, modulating the expression of at least 1, at least 2, at least 3, at least 4, at least 5, at least 6, at least 7, at least 8, at least 9, at least 10, or at least 11 genes in a rAAV packaging and/or producer cell line results in increased rAAV production compared to a control parental cell line.

Methods of Modulating One or More Genes and/or Protein

Modulating (e.g., reducing) the expression or activity of a gene (e.g., CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, TRIM25, or any combination(s) thereof) can be achieved by different mechanisms, including, but not limited to, altering one or more of the following: 1) gene copy number, 2) transcription or translation of a gene, 3) transcript stability or longevity, 4) the number of copies of an mRNA or miRNA, 5) the availability of a non-coding RNA or non-coding RNA target site, 6) the position or degree of post-translational modifications on a protein, or 7) the activity of a protein. Tools that can be used to modulate gene expression include but are not limited to a nuclease, a double stranded RNA (dsRNA), a small interfering RNA (siRNA), a small hairpin RNA (shRNA), a microRNA (miRNA), and an antisense RNA oligonucleotide (ASO).

### Nuclease

In certain embodiments, a nuclease may be used to modulate expression of one or more genes in a cell line described herein (e.g., a rAAV packaging and/or producer cell line). In certain embodiments, gene modulation is achieved using zinc finger nucleases (ZFNs). Synthetic ZFNs are composed of a zinc finger binding domain fused with, e.g., a FokI DNA cleavage domain. ZFNs can be designed/engineered for editing the genome of a cell, including, but not limited to, knock out or knock in gene expression, in a wide range of organisms. Meganucleases, transcription activator-like effector nucleases (TALENs), or clustered regularly interspaced short palindromic repeats (CRISPR) associated proteins (e.g., Cas nucleases), and triplexes can also be used for genome engineering in a wide array of cell types. The described reagents can be used to target promoters, protein-encoding regions (exons), introns, 5′ and 3′ UTRs, and more.

Double Stranded RNA (dsRNA) Molecules for Modulation

In certain embodiments, double-stranded RNA (dsRNA) molecules may be used to modulate expression of one or more genes in a cell line described herein (e.g., a rAAV packaging and/or producer cell line). dsRNA molecules can be designed to antagonize one or more genes by sequence homology-based targeting of the corresponding RNA sequence. Such dsRNAs can be small interfering RNAs (siRNAs), small hairpin RNAs (shRNAs), or micro-RNAs (miRNAs). The sequence of such dsRNAs will comprise a complementary portion of the mRNA encoding the one or more genes to be modulated. This portion can be 100% complementary to the target portion within the mRNA, but lower levels of complementarity (e.g., 90% or more or 95% or more) can also be used. Typically the percent complementarity is determined over a length of contiguous nucleic acid residues. A dsRNA molecule of the disclosure may, for example, have at least 80% complementarity to the target portion within the mRNA measured over at least 10, at least 20, at least 30, at least 40, at least 50, at least 60, at least 70, at least 80, at least 90, or more nucleic acid residues. In some instances, a dsRNA molecule has at least 80% complementarity to the target portion of mRNA over the entire length of the dsRNA molecule.

Another gene targeting reagent that uses RNA interference (RNAi) pathways is small hairpin RNA, also referred to as shRNA. shRNAs delivered to cells via, e.g., expression constructs (e.g., plasmids, lentiviruses) have the ability to provide long term reduction of gene expression in a constitutive or regulated manner, depending upon the type of promoter employed. In one embodiment, the genome of a lentiviral particle is modified to include one or more shRNA expression cassettes that target a gene (or genes) of interest. Such lentiviruses can infect a cell, stably integrate their viral genome into the host genome, and express a shRNA in a constitutive, regulated, or (in the case where multiple shRNA are being expressed) constitutive and regulated fashion. Thus, in some embodiments, shRNA can be designed to target individual variants of a single gene or multiple closely related gene family members. Individual shRNA can modulate collections of targets having similar or redundant functions or sequence motifs. The skilled person will recognize that lentiviral constructs can also incorporate cloned DNA, or ORF expression constructs.

In some embodiments described herein, gene targeting reagents including small interfering RNAs (siRNA) as well as microRNAs (miRNA) can be used to modulate gene function. siRNAs and miRNAs can incorporate a wide range of chemical modifications, levels of complementarity to the target transcript of interest, and designs (see U.S. Pat. No. 8,188,060) to enhance stability, cellular delivery, specificity, and functionality. In addition, such reagents can be designed to target diverse regions of a gene (including the 5′ UTR, the open reading frame, the 3′ UTR of the mRNA), or (in some cases) the promoter/enhancer regions of the genomic DNA encoding the gene of interest. Gene modulation (e.g., reduction of gene expression, knockdown) can be achieved by introducing (into a cell) a single siRNA or miRNA or multiple siRNAs or pools of miRNAs targeting different regions of the same mRNA transcript. Synthetic siRNA/miRNA delivery can be achieved by any number of methods including but not limited to 1) self-delivery, 2) lipid-mediated delivery, 3) electroporation, or 4) vector/plasmid-based expression systems. An introduced RNA molecule may be referred to as an exogenous nucleotide sequence or polynucleotide. In some embodiments, siRNA can be designed to target individual variants of a single gene or multiple closely related gene family members.

siRNA can be used to reduce the expression of one or more genes (e.g., CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, TRIM25, or any combination(s) thereof). In some embodiments, a siRNA which comprises a nucleotide sequence selected from SEQ ID NOs: 1-36, or a variant thereof, is used to reduce the expression of a target gene.

TABLE 1
Exemplary siRNA sequences for reducing expression of genes.

| | SEQ ID | |
|---|---|---|
| Target gene | NO: | siRNA Sequence* |
| CGAS | 1 | Sense: uuucugcaccuaaugaauuu |
| (cGAS) | 2 | Antisense: aaauucauuaggugcagaaa |
| CHUK | 3 | Sense: aaaguguauauuugcaugug |
| (IKK-α) | 4 | Antisense: cacaugcaaauauacacuuu |
| DDX41 | 5 | Sense: uucugccuggaacaagagaa |
| | 6 | Antisense: uucucuuguuccaggcagaa |
| DHX58 | 7 | Sense: uaugagcagcagguggugaa |
| (LGP2) | 8 | Antisense: uucaccaccugcugcucaua |
| IFI6 | 9 | Sense: aggaacagacucagccuccc |
| | 10 | Antisense: gggaggcugagucuguuccu |
| IFIT3 | 11 | Sense: gugucucaagggaucuagaa |
| | 12 | Antisense: uucuagaucccuugagacac |
| IKBKB | 13 | Sense: uuugcacaucauucgugggg |
| (IKK-β) | 14 | Antisense: ccccacgaaugaugugcaaa |
| IRF3 | 15 | Sense: ucuacgaguuugugaacuca |
| | 16 | Antisense: ugaguucacaaacucguaga |
| IRF7 | 17 | Sense: ccaucauguacaagggccgc |
| | 18 | Antisense: gcggcccuuguacaugaugg |
| MAVS | 19 | Sense: ccuaccaccuugaugccugu |
| | 20 | Antisense: acaggcaucaaggugguagg |
| MDA5 | 21 | Sense: aaguuauaguucuugucaau |
| (IFIH1) | 22 | Antisense: auugacaagaacuauaacuu |
| MYD88 | 23 | Sense: uaucgacugaaguugugugu |
| | 24 | Antisense: acacacaacuucagucgaua |
| NFKB1 | 25 | Sense: uuuacagcuuuucuuccgg |
| | 26 | Antisense: ccggaagaaaagcuguaaa |
| NFKB2 | 27 | Sense: gcuacaacccaggucuggau |
| | 28 | Antisense: auccagaccuggguuguagc |
| RIG-1 | 29 | Sense: aucccaaccgauaucauuuc |
| (DDX58) | 30 | Antisense: gaaaugauaucgguugggau |
| TBK1 | 31 | Sense: auuagcuuauaaugaagaac |
| | 32 | Antisense: guucuucauuauaagcuaau |
| TRIF | 33 | Sense: uguagaagauaccaccucuc |
| | 34 | Antisense: gagaggugguaucuucuaca |
| TRIM25 | 35 | Sense: auacacggaaaugaaggcuc |
| | 36 | Antisense: gagccuucauuuccguguau |
| RGMA | 238 | Sense: cgcucaucgacaauaauuatt |
| | 239 | Antisense: uaauuauugucgaugagcggc |
| KCNN2 | 245 | Sense: gaagcuagaacuuaccaaatt |
| | 246 | Antisense: uuugguaaguucuagcuucct |

*siRNA sequences (sense and antisense) used for reducing expression of genes.

In some embodiments, the siRNA used to reduce the expression of CGAS (cGAS) comprises the nucleotide sequence of SEQ ID NO: 1 and/or SEQ ID NO: 2, or a variant thereof. For example, in some embodiments, the siRNA comprises the nucleotide sequence of SEQ ID NO: 1 in the sense strand and the nucleotide sequence of SEQ ID NO: 2 in the anti-sense strand. 5

In some embodiments, the siRNA used to reduce the expression of CHUK (IKK-α) comprises the nucleotide sequence of SEQ ID NO: 3 and/or SEQ ID NO: 4, or a variant thereof. For example, in some embodiments, the siRNA comprises the nucleotide sequence of SEQ ID NO: 3 in the sense strand and the nucleotide sequence of SEQ ID NO: 4 in the anti-sense strand.

In some embodiments, the siRNA used to reduce the expression of DDX41 comprises the nucleotide sequence of SEQ ID NO: 5 and/or SEQ ID NO: 6, or a variant thereof. For example, in some embodiments, the siRNA comprises the nucleotide sequence of SEQ ID NO: 5 in the sense strand and the nucleotide sequence of SEQ ID NO: 6 in the anti-sense strand.

In some embodiments, the siRNA used to reduce the expression of DHX58 (LGP2) comprises the nucleotide sequence of SEQ ID NO: 7 and/or SEQ ID NO: 8, or a variant thereof. For example, in some embodiments, the siRNA comprises the nucleotide sequence of SEQ ID NO: 7 in the sense strand and the nucleotide sequence of SEQ ID NO: 8 in the anti-sense strand.

In some embodiments, the siRNA used to reduce the expression of IFI6 comprises the nucleotide sequence of SEQ ID NO: 9 and/or SEQ ID NO: 10, or a variant thereof. For example, in some embodiments, the siRNA comprises the nucleotide sequence of SEQ ID NO: 9 in the sense strand and the nucleotide sequence of SEQ ID NO: 10 in the anti-sense strand.

In some embodiments, the siRNA used to reduce the expression of IFIT3 comprises the nucleotide sequence of SEQ ID NO: 11 and/or SEQ ID NO: 12, or a variant thereof. For example, in some embodiments, the siRNA comprises the nucleotide sequence of SEQ ID NO: 11 in the sense strand and the nucleotide sequence of SEQ ID NO: 12 in the anti-sense strand.

In some embodiments, the siRNA used to reduce the expression of IKBKB (IKK-β) comprises the nucleotide sequence of SEQ ID NO: 13 and/or SEQ ID NO: 14, or a variant thereof. For example, in some embodiments, the siRNA comprises the nucleotide sequence of SEQ ID NO: 13 in the sense strand and the nucleotide sequence of SEQ ID NO: 14 in the anti-sense strand.

In some embodiments, the siRNA used to reduce the expression of IRF3 comprises the nucleotide sequence of SEQ ID NO: 15 and/or SEQ ID NO: 16, or a variant thereof. For example, in some embodiments, the siRNA comprises the nucleotide sequence of SEQ ID NO: 15 in the sense strand and the nucleotide sequence of SEQ ID NO: 16 in the anti-sense strand.

In some embodiments, the siRNA used to reduce the expression of IRF7 comprises the nucleotide sequence of SEQ ID NO: 17 and/or SEQ ID NO: 18, or a variant thereof. For example, in some embodiments, the siRNA comprises the nucleotide sequence of SEQ ID NO: 17 in the sense strand and the nucleotide sequence of SEQ ID NO: 18 in the anti-sense strand.

In some embodiments, the siRNA used to reduce the expression of MAVS comprises the nucleotide sequence of SEQ ID NO: 19 and/or SEQ ID NO: 20, or a variant thereof. For example, in some embodiments, the siRNA comprises the nucleotide sequence of SEQ ID NO: 19 in the sense strand and the nucleotide sequence of SEQ ID NO: 20 in the anti-sense strand.

In some embodiments, the siRNA used to reduce the expression of MDA5 (IFIH1) comprises the nucleotide sequence of SEQ ID NO: 21 and/or SEQ ID NO: 22, or a variant thereof. For example, in some embodiments, the siRNA comprises the nucleotide sequence of SEQ ID NO: 21 in the sense strand and the nucleotide sequence of SEQ ID NO: 22 in the anti-sense strand.

In some embodiments, the siRNA used to reduce the expression of MYD88 comprises the nucleotide sequence of SEQ ID NO: 23 and/or SEQ ID NO: 24, or a variant thereof. For example, in some embodiments, the siRNA comprises the nucleotide sequence of SEQ ID NO: 23 in the sense strand and the nucleotide sequence of SEQ ID NO: 24 in the anti-sense strand.

In some embodiments, the siRNA used to reduce the expression of NFKB1 comprises the nucleotide sequence of SEQ ID NO: 25 and/or SEQ ID NO: 26, or a variant thereof. For example, in some embodiments, the siRNA comprises the nucleotide sequence of SEQ ID NO: 25 in the sense strand and the nucleotide sequence of SEQ ID NO: 26 in the anti-sense strand.

In some embodiments, the siRNA used to reduce the expression of NFKB2 comprises the nucleotide sequence of SEQ ID NO: 27 and/or SEQ ID NO: 28, or a variant thereof. For example, in some embodiments, the siRNA comprises the nucleotide sequence of SEQ ID NO: 27 in the sense strand and the nucleotide sequence of SEQ ID NO: 28 in the anti-sense strand.

In some embodiments, the siRNA used to reduce the expression of RIG-1 (DDX58) comprises the nucleotide sequence of SEQ ID NO: 29 and/or SEQ ID NO: 30, or a variant thereof. For example, in some embodiments, the siRNA comprises the nucleotide sequence of SEQ ID NO: 29 in the sense strand and the nucleotide sequence of SEQ ID NO: 30 in the anti-sense strand.

In some embodiments, the siRNA used to reduce the expression of TBK1 comprises the nucleotide sequence of SEQ ID NO: 31 and/or SEQ ID NO: 32, or a variant thereof. For example, in some embodiments, the siRNA comprises the nucleotide sequence of SEQ ID NO: 31 in the sense strand and the nucleotide sequence of SEQ ID NO: 32 in the anti-sense strand.

In some embodiments, the siRNA used to reduce the expression of TRIF comprises the nucleotide sequence of SEQ ID NO: 33 and/or SEQ ID NO: 34, or a variant thereof. For example, in some embodiments, the siRNA comprises the nucleotide sequence of SEQ ID NO: 33 in the sense strand and the nucleotide sequence of SEQ ID NO: 34 in the anti-sense strand.

In some embodiments, the siRNA used to reduce the expression of TRIM25 comprises the nucleotide sequence of SEQ ID NO: 35 and/or SEQ ID NO: 36, or a variant thereof. For example, in some embodiments, the siRNA comprises the nucleotide sequence of SEQ ID NO: 35 in the sense strand and the nucleotide sequence of SEQ ID NO: 36 in the anti-sense strand.

In some embodiments, the siRNA used to reduce the expression of RGMA comprises the nucleotide sequence of SEQ ID NO: 238 and/or SEQ ID NO: 239, or a variant thereof. For example, in some embodiments, the siRNA comprises the nucleotide sequence of SEQ ID NO: 238 in the sense strand and the nucleotide sequence of SEQ ID NO: 239 in the anti-sense strand.

In some embodiments, the siRNA used to reduce the expression of KCNN2 comprises the nucleotide sequence of SEQ ID NO: 245 and/or SEQ ID NO: 246, or a variant thereof. For example, in some embodiments, the siRNA comprises the nucleotide sequence of SEQ ID NO: 245 in the sense strand and the nucleotide sequence of SEQ ID NO: 246 in the anti-sense strand. Antisense RNA Oligonucleotide (ASO))

Antisense RNA oligonucleotide (ASO), can be used to modulate expression of one or more genes in a rAAV packaging and/or producer cell line. Typically, ASOs are used to reduce expression of one or more genes. Using known techniques and based on a knowledge of the sequence of the one or more gene to be modulated, ASO molecules can be designed to antagonize the one or more genes by sequence homology-based targeting of the corresponding RNA. The ASO sequence can comprise nucleotide sequence that is complementary to a target portion of the mRNA or long non-coding RNA (lncRNA) produced from the one or more genes. This portion can be 100% complementary to the target portion within the mRNA or lncRNA but lower levels of complementarity (e.g., 90% or more or 95% or more) can also be used.

In some embodiments, the ASO can be an antisense RNA oligonucleotide wherein at least one nucleoside linkage of the sequence is a phosphorothioate linkage, a phosphorodithioate linkage, a phosphotriester linkage, an alkylphosphonate linkage, an aminoalkyl phosphotriester linkage, an alkylene phosphonate linkage, a phosphinate linkage, a phosphoramidate linkage, an aminoalkyl phosphoramidate linkage, a thiophosphoramidate linkage, a thionoalkylphosphonate linkage, a thionoalkylphosphotriester linkage, a thiophosphate linkage, a selenophosphate linkage, or a boranophosphate linkage. In some embodiments, at least one internucleoside linkage of the antisense RNA oligonucleotide sequence is a phosphorothioate linkage. In some embodiments, all of the internucleoside linkages of the antisense RNA oligonucleotide sequence are phosphorothioate linkages.

### CRISPR Genome Editing

In some embodiments, modulation of gene expression in a rAAV packaging and/or producer cell line is carried out using CRISPR genome editing. The CRISPR genome editing typically comprises two distinct components: (1) a guide RNA (gRNA) and (2) an endonuclease, specifically a CRISPR associated (Cas) nuclease (e.g., Cas9). The guide RNA can be an RNA that combines the endogenous bacterial crispr RNA (crRNA) and a tracrRNA into a single chimeric guide RNA transcript. Without being bound by theory, it is believed that when gRNA and the Cas are expressed in the cell, the genomic target sequence can be modified or permanently disrupted.

The gRNA/Cas complex is recruited to the target sequence by base-pairing between the gRNA sequence and the complement to the target DNA sequence in the gene for reduction (e.g., CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, TRIM25, or any combination(s) thereof). For successful binding of Cas, the genomic target sequence typically also contains the Protospacer Adjacent Motif (PAM) sequence immediately following the target sequence. The binding of the gRNA/Cas complex localizes the Cas to the genomic target sequence in the one or more genes of the present disclosure so that the wild-type Cas can cut both strands of DNA causing a double strand break. This can be repaired through one of two general repair pathways: (1) the non-homologous end joining DNA repair pathway or (2) the homology directed repair pathway. The non-homologous end joining DNA repair pathway can result in inserts/deletions at the double strand break that can lead to frameshifts and/or premature stop codons, effectively disrupting the open reading frame of the target gene. The homology directed repair pathway requires the presence of a repair template, which is used to fix the double strand break.

Any appropriate gRNA, or a combination of gRNAs, may be used for CRISPR genome editing. Typically, one or more gRNAs are used to reduce expression of one or more (e.g., one, two, three, four, five, or more) genes (e.g., CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, TRIM25, or any combination(s) thereof). In some embodiments described herein, one or more gRNAs is used to modulate (e.g., reduce or eliminate/knockout) expression of a gene selected from the group consisting of CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, TRIM25, and any combination(s) thereof. CRISPR genome editing can involve making single edits at each locus or multiple edits (e.g., a pair of edits) at each locus. In embodiments in which multiple edits are made at a single locus, multiple gRNAs (e.g., a pair of gRNAs) may be used.

One or more gRNAs can be designed using known techniques and based on a knowledge of the sequence of the one or more genes to be modulated, typically using any publicly available appropriate computer program (examples of a few design tools include, but not limited to, synthego design tool, Broad Institute GPP sgRNA Designer, CRISPOR, CHOPCHOP, Off-Spotter, Cas-OFFinder, CRISPR-Era, Benchling CRISPR guide RNA design tool, and E-CRISPR). Knock out packaging and/or producer cells may be generated using any appropriate technique, with standard techniques being known in the art and suitable kits being commercially available.

One or more gRNAs can be delivered to a producer cell line of the disclosure by any appropriate means. Suitable techniques are known in the art and include the use of plasmid, viral and bacterial vectors to deliver the one or more gRNAs to the producer cell line. Typically, one or more gRNAs is delivered using plasmid DNA.

One or more gRNAs may be used to reduce the expression of a gene selected from the group consisting of CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, TRIM25, and any combination(s) thereof. One gRNA or multiple gRNAs may be used to modulate the expression of a gene. In some embodiments described herein, one or more gRNAs are used to reduce the expression of a gene selected from the group consisting of CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, TRIM25, and any combination(s) thereof. Multiple gRNAs may be used to modulate the expression of multiple genes together, and can include, e.g., combinations with other genes not included in the aforementioned list, such as RGMA (Repulsive Guidance Molecule BMP Co-Receptor A) and KCNN2 (Potassium Calcium-Activated Channel Subfamily N Member 2). Examples of combinations genes that may be knocked out together in various embodiments include, but are not limited to, 1) DHX58, MDA5, and RGMA; 2) DHX58 and MDA5; 3) CGAS and DHX50; 5) CGAS and MDA5; 6) CGAS and RGMA; 7) CGAS, DHX58, MDA5, and RGMA; 8) CGAS, DHX58, and MDA5; 9) RGMA and DHX58; 10) RIG and RGMA; 11) RIG and KCNN2; 12) RIG, RGMA, and KCNN2; 13) IFIT3 and RGMA; 14) IFIT3 and KCNN2; 15) IFIT3, RGMA, and KCNN2; 16) MDA5 and RGMA; 17) MDA5 and KCNN2; 18) MDA5, RGMA, and KCNN2; 19) CGAS and RGMA; 14) CGAS and KCNN2; 20) CGAS, RGMA, and KCNN2; 21) CHUK and RGMA; 22) CHUK and KCNN2; 23) CHUK, RGMA, and KCNN2; 24) DDX41 and RGMA; 25) DDX41 and KCNN2; 26) DDX41, RGMA, and KCNN2; 27) DHX58 and RGMA; 28) DHX58 and KCNN2; 29) DHX58, RGMA, and KCNN2; 30) IFI6 and RGMA; 31) IFI6 and KCNN2; 32) IFI6, RGMA, and KCNN2; 33) IKBKB and RGMA; 34) IKBKB and KCNN2; 35) IKBKB, RGMA, and KCNN2; 36) IRF3 and RGMA; 37) IRF3 and KCNN2; 38) IRF3, RGMA, and KCNN2; 39) IRF7 and RGMA; 40) IRF7 and KCNN2; 41) IRF7, RGMA, and KCNN2; 42) MAVS and RGMA; 43) MAVS and KCNN2; 44) MAVS, RGMA, and KCNN2; 45) MYD88 and RGMA; 46) MYD88 and KCNN2; 47) MYD88, RGMA, and KCNN2; 48) NFKB1 and RGMA; 49) NFKB1 and KCNN2; 50) NFKB1, RGMA, and KCNN2; 51) NFKB2 and RGMA; 52) NFKB2 and KCNN2; 53) NFKB2, RGMA, and KCNN2; 54) TBK1 and RGMA; 55) TBK1 and KCNN2; 56) TBK1, RGMA, and KCNN2; 57) TRIF and RGMA; 58) TRIF and KCNN2; 59) TRIF, RGMA, and KCNN2; 60) TRIM25 and RGMA; 61) TRIM25 and KCNN2; and 62) TRIM25, RGMA, and KCNN2.

In some embodiments, gRNAs may be modified to enhance editing efficiency by increasing binding to the target site and inhibiting nuclease degradation. In certain embodiments, these modifications may be 2′ O-methyl analogs and 3′ phosphorothioate internucleotide linkages in the terminal three nucleotides on both 5′ and 3′ ends of the gRNA.

Exemplary target region sequences within gRNA(s) used to modulate gene expression of one or more genes are shown in Table 2. Variants of such sequences are also contemplated. Exemplary target DNA sequences targeted by gRNA(s) used to modulate gene expression of one or more genes are shown in Table 3. Variants of such sequences are also contemplated.

TABLE 2
Exemplary target region sequences of gRNAs.

| | |
|---|---|
| SEQ ID NO: | Sequence |

CGAS (cGAS)

| | |
|---|---|
| SEQ ID NO: 37 | GAACUUUCCCGCCUUAGGCA |
| SEQ ID NO: 38 | UGCAUGGCCUUUCCGUGCCA |
| SEQ ID NO: 39 | CUGCCUAAGGCGGGAAAGUU |
| SEQ ID NO: 40 | CGAACUUUCCCGCCUUAGGC |

CHUK (IKK-α)

| | |
|---|---|
| SEQ ID NO: 41 | ACGUCUGUCUGUACCAGCAU |
| SEQ ID NO: 42 | GCAAUGGAAUACUGUUCUGG |
| SEQ ID NO: 43 | UGAUGUGCCUCUUCUAGCAA |
| SEQ ID NO: 44 | AGGCCUUUACAACAUUGGCA |

DDX41

| | |
|---|---|
| SEQ ID NO: 45 | ACGUGCCCUAUGUGCCGUUA |
| SEQ ID NO: 46 | GAUGAGGACGACAUCCCGCU |
| SEQ ID NO: 47 | GAUCCUGGAGAGUGUUGCCG |
| SEQ ID NO: 48 | UAAGGUGCUGGUGCUGAUCC |

DHX58 (LGP2)

| | |
|---|---|
| SEQ ID NO: 49 | UGGAGCUUCGGUCCUACCAA |

IFI6

| | |
|---|---|
| SEQ ID NO: 50 | CAAGUAUCUCGAUAGUGAGG |
| SEQ ID NO: 51 | CUGGUCUGCGAUCCUGAAUG |
| SEQ ID NO: 52 | GGUAAUAUUGGUGCCCUGAU |
| SEQ ID NO: 53 | GAAAAAGUGCUCGGAGAGCU |

IFIT3

| | |
|---|---|
| SEQ ID NO: 54 | CAUCGCAAUUGCCAGUCCAG |
| SEQ ID NO: 55 | GGCAAUUGCGAUGUACCAUC |
| SEQ ID NO: 56 | ACACCUAGAUGGUAACAACG |
| SEQ ID NO: 57 | CGCCUGGGUCUACUAUCACU |

IKBKB (IKK-β)

| | |
|---|---|
| SEQ ID NO: 58 | AUGAAGGUAUCUAAGCGCAG |
| SEQ ID NO: 59 | CAUGUCUGCGUUGUCAGGGA |
| SEQ ID NO: 60 | ACCGUCGACUACUGGAGCUU |
| SEQ ID NO: 61 | GAAGCCCGUGAUGCACUCAA |

IRF3

| | |
|---|---|
| SEQ ID NO: 62 | GCAACCCUUCUUUGCGGUUG |
| SEQ ID NO: 63 | UCUCCGGACACCAAUGGUGG |
| SEQ ID NO: 64 | GGUUGCGUUUAGCAGAGGAC |
| SEQ ID NO: 65 | AUUACCUUCACGGAAGGAAG |

IRF7

| | |
|---|---|
| SEQ ID NO: 66 | CGUGAUGCUGCGGGAUAACU |
| SEQ ID NO: 67 | GCGGAAGUUGGUUUUCCAGC |
| SEQ ID NO: 68 | AUCAGCAGCGGCUGCUAUGA |
| SEQ ID NO: 69 | CUCCUUGGAGAGAUCAGCAG |

MAVS

| | |
|---|---|
| SEQ ID NO: 70 | CUGUGAGCUAGUUGAUCUCG |
| SEQ ID NO: 71 | GUACUUCAUUGCGGCACUGA |
| SEQ ID NO: 72 | UCUUCAAUACCCUUCAGCGG |
| SEQ ID NO: 73 | GGGUAUUGAAGAGAUGCCAG |

MDA5 (IFIH1)

| | |
|---|---|
| SEQ ID NO: 74 | UUGGACUCGGGAAUUCGUGG |

MYD88

| | |
|---|---|
| SEQ ID NO: 75 | CGUUCAAGAACAGAGACAGG |
| SEQ ID NO: 76 | GUUCUUGAACGUGCGGACAC |
| SEQ ID NO: 77 | CUCGAGCUGCUUACCAAGCU |
| SEQ ID NO: 78 | AUGAAGGCAUCGAAACGCUC |

NFKB1

| | |
|---|---|
| SEQ ID NO: 79 | CGUUUCCGUUAUGUAUGUGA |
| SEQ ID NO: 80 | CAACUAUGUGGGACCAGCAA |
| SEQ ID NO: 81 | GGGAUCUGCACUGUAACUGC |
| SEQ ID NO: 82 | GAACAAGAAGUCUUACCCUC |

NFKB2

| | |
|---|---|
| SEQ ID NO: 83 | GUUCCACGAUCACCAGGUAG |
| SEQ ID NO: 84 | CGAUUUCGAUAUGGCUGUGA |
| SEQ ID NO: 85 | CUUUCGGCCCUUCUCACUGG |
| SEQ ID NO: 86 | AGGUCCACCUCGAUCUUGGC |

RIG-1 (DDX58)

| | |
|---|---|
| SEQ ID NO: 87 | GGAUUAUAUCCGGAAGACCC |

TBK1

| | |
|---|---|
| SEQ ID NO: 88 | GGUAGUCCAUAGGCAUUAGA |
| SEQ ID NO: 89 | UUUGAACAUCCACUGGACGA |
| SEQ ID NO: 90 | CAUAAGCUUCCUUCGUCCAG |
| SEQ ID NO: 91 | GCUACUGCAAAUGUCUUUCG |

TRIF

| | |
|---|---|
| SEQ ID NO: 92 | UGCUGCACCUAGAAUGUCGA |
| SEQ ID NO: 93 | GAUCUCUCUAGAGGCAUUGA |
| SEQ ID NO: 94 | GCCAUGGUUCUCCUGAAGCU |
| SEQ ID NO: 95 | CUUGUACCACCUGCUGGCUG |

TRIM25

| | |
|---|---|
| SEQ ID NO: 96 | GUCGUGCCUGAAUGAGACGU |
| SEQ ID NO: 97 | ACCGUGUUCUUGUGCAGCUG |
| SEQ ID NO: 98 | GAACACGGUGCUGUGCAACG |
| SEQ ID NO: 99 | GAAGACGUGCUUGGUGUGCA |

KCNN2

| | |
|---|---|
| SEQ ID NO: 247 | UUGCCACUACAGCUACCACC |
| SEQ ID NO: 248 | CCAAUGUACUCAGGGAAACA |
| SEQ ID NO: 249 | AGUCCACCAAAGUGUUUGCU |
| SEQ ID NO: 250 | AAAGGAGUCUGCUUACUUAC |

TABLE 3
Exemplary target DNA sequences.

| | |
|---|---|
| SEQ ID NO: | Sequence |

CGAS

| | |
|---|---|
| SEQ ID NO: 100 | GAACTTTCCCGCCTTAGGCA |
| SEQ ID NO: 101 | TGCATGGCCTTTCCGTGCCA |
| SEQ ID NO: 102 | CTGCCTAAGGCGGGAAAGTT |
| SEQ ID NO: 103 | CGAACTTTCCCGCCTTAGGC |

CHUK (IKK-α)

| | |
|---|---|
| SEQ ID NO: 104 | ACGTCTGTCTGTACCAGCAT |
| SEQ ID NO: 105 | GCAATGGAATACTGTTCTGG |
| SEQ ID NO: 106 | TGATGTGCCTCTTCTAGCAA |
| SEQ ID NO: 107 | AGGCCTTTACAACATTGGCA |

DDX41

| | |
|---|---|
| SEQ ID NO: 108 | ACGTGCCCTATGTGCCGTTA |
| SEQ ID NO: 109 | GATGAGGACGACATCCCGCT |
| SEQ ID NO: 110 | GATCCTGGAGAGTGTTGCCG |
| SEQ ID NO: 111 | TAAGGTGCTGGTGCTGATCC |

DHX58 (LGP2)

| | |
|---|---|
| SEQ ID NO: 112 | TGGAGCTTCGGTCCTACCAA |

IFI6

| | |
|---|---|
| SEQ ID NO: 113 | CAAGTATCTCGATAGTGAGG |
| SEQ ID NO: 114 | CTGGTCTGCGATCCTGAATG |
| SEQ ID NO: 115 | GGTAATATTGGTGCCCTGAT |
| SEQ ID NO: 116 | GAAAAAGTGCTCGGAGAGCT |

IFIT3

| | |
|---|---|
| SEQ ID NO: 117 | CATCGCAATTGCCAGTCCAG |
| SEQ ID NO: 118 | GGCAATTGCGATGTACCATC |
| SEQ ID NO: 119 | ACACCTAGATGGTAACAACG |
| SEQ ID NO: 120 | CGCCTGGGTCTACTATCACT |

IKBKB (IKK-β)

| | |
|---|---|
| SEQ ID NO: 121 | ATGAAGGTATCTAAGCGCAG |
| SEQ ID NO: 122 | CATGTCTGCGTTGTCAGGGA |
| SEQ ID NO: 123 | ACCGTCGACTACTGGAGCTT |
| SEQ ID NO: 124 | GAAGCCCGTGATGCACTCAA |

IRF3

| | |
|---|---|
| SEQ ID NO: 125 | GCAACCCTTCTTTGCGGTTG |
| SEQ ID NO: 126 | TCTCCGGACACCAATGGTGG |
| SEQ ID NO: 127 | GGTTGCGTTTAGCAGAGGAC |
| SEQ ID NO: 128 | ATTACCTTCACGGAAGGAAG |

IRF7

| | |
|---|---|
| SEQ ID NO: 129 | CGTGATGCTGCGGGATAACT |
| SEQ ID NO: 130 | GCGGAAGTTGGTTTTCCAGC |
| SEQ ID NO: 131 | ATCAGCAGCGGCTGCTATGA |
| SEQ ID NO: 132 | CTCCTTGGAGAGATCAGCAG |

MAVS

| | |
|---|---|
| SEQ ID NO: 133 | CTGTGAGCTAGTTGATCTCG |
| SEQ ID NO: 134 | GTACTTCATTGCGGCACTGA |
| SEQ ID NO: 135 | TCTTCAATACCCTTCAGCGG |
| SEQ ID NO: 136 | GGGTATTGAAGAGATGCCAG |

MDA5 (IFIH1)

| | |
|---|---|
| SEQ ID NO: 137 | TTGGACTCGGGAATTCGTGG |

MYD88

| | |
|---|---|
| SEQ ID NO: 138 | CGTTCAAGAACAGAGACAGG |
| SEQ ID NO: 139 | GTTCTTGAACGTGCGGACAC |
| SEQ ID NO: 140 | CTCGAGCTGCTTACCAAGCT |
| SEQ ID NO: 141 | ATGAAGGCATCGAAACGCTC |

NFKB1 (NF-kB)

| | |
|---|---|
| SEQ ID NO: 142 | CGTTTCCGTTATGTATGTGA |
| SEQ ID NO: 143 | CAACTATGTGGGACCAGCAA |
| SEQ ID NO: 144 | GGGATCTGCACTGTAACTGC |
| SEQ ID NO: 145 | GAACAAGAAGTCTTACCCTC |

NFKB2

| | |
|---|---|
| SEQ ID NO: 146 | GTTCCACGATCACCAGGTAG |
| SEQ ID NO: 147 | CGATTTCGATATGGCTGTGA |
| SEQ ID NO: 148 | CTTTCGGCCCTTCTCACTGG |
| SEQ ID NO: 149 | AGGTCCACCTCGATCTTGGC |

RIG-1 (DDX58)

| | |
|---|---|
| SEQ ID NO: 150 | GGATTATATCCGGAAGACCC |

TBK1

| | |
|---|---|
| SEQ ID NO: 151 | GGTAGTCCATAGGCATTAGA |
| SEQ ID NO: 152 | TTTGAACATCCACTGGACGA |
| SEQ ID NO: 153 | CATAAGCTTCCTTCGTCCAG |
| SEQ ID NO: 154 | GCTACTGCAAATGTCTTTCG |

TRIF

| | |
|---|---|
| SEQ ID NO: 155 | TGCTGCACCTAGAATGTCGA |
| SEQ ID NO: 156 | GATCTCTCTAGAGGCATTGA |
| SEQ ID NO: 157 | GCCATGGTTCTCCTGAAGCT |
| SEQ ID NO: 158 | CTTGTACCACCTGCTGGCTG |

TRIM25

| | |
|---|---|
| SEQ ID NO: 159 | GTCGTGCCTGAATGAGACGT |
| SEQ ID NO: 160 | ACCGTGTTCTTGTGCAGCTG |
| SEQ ID NO: 161 | GAACACGGTGCTGTGCAACG |
| SEQ ID NO: 162 | GAAGACGTGCTTGGTGTGCA |

RGMA

| | |
|---|---|
| SEQ ID NO: 240 | CTTCTCGTAATGGCAGATCT |
| SEQ ID NO: 241 | GCACTTGAGGATCTTGCACG |
| SEQ ID NO: 242 | GAGGTCCTCTATGCCATGGA |
| SEQ ID NO: 243 | CCATACCCATCCATCCAGCT |

KCNN2

| | |
|---|---|
| SEQ ID NO: 251 | TTGCCACTACAGCTACCACC |
| SEQ ID NO: 252 | CCAATGTACTCAGGGAAACA |
| SEQ ID NO: 253 | AGTCCACCAAAGTGTTTGCT |
| SEQ ID NO: 254 | AAAGGAGTCTGCTTACTTAC |

For example, one or more gRNAs used to target CGAS (cGAS) can comprise a sequence selected from SEQ ID NOs: 37-40 (shown in Table 2), or any combination(s) thereof, or a sequence that targets a nucleotide sequence selected from SEQ ID NOs: 100-103 (shown in Table 3), or any combination(s) thereof.

For example, one or more gRNAs used to target CHUK (IKK-α) can comprise a sequence selected from the SEQ ID NOs: 41-44 (shown in Table 2), or any combination(s) thereof, or a sequence that targets a nucleotide sequence selected from SEQ ID NOs: 104-107 (shown in Table 3), or any combination(s) thereof.

For example, one or more gRNAs used to target DDX41 can comprise a sequence selected from SEQ ID NOs: 45-48 (shown in Table 2), or any combination(s) thereof, or a sequence that targets a nucleotide sequence selected from SEQ ID NOs: 108-111 (shown in Table 3), or any combination(s) thereof.

For example, one or more gRNAs used to target DHX58 (LGP2) can comprise the sequence of SEQ ID NO: 49 (shown in Table 2) or a sequence that targets the nucleotide sequence of SEQ ID NO: 112 (shown in Table 3).

For example, one or more gRNAs used to target IFI6 can comprise a sequence selected from SEQ ID NOs: 50-53 (shown in Table 2), or any combination(s) thereof, or a sequence that targets a nucleotide sequence selected from SEQ ID NOs: 113-116 (shown in Table 3), or any combination(s) thereof.

For example, one or more gRNAs used to target IFIT3 can comprise a sequence selected from SEQ ID NOs: 54-57 (shown in Table 2), or any combination(s) thereof, or a sequence that targets a nucleotide sequence selected from SEQ ID NOs: 117-120 (shown in Table 3), or any combination(s) thereof.

For example, one or more gRNAs used to target IKBKB (IKK-β) can comprise a sequence selected from SEQ ID NOs: 58-61 (shown in Table 2), or any combination(s) thereof, or a sequence that targets a nucleotide sequence selected from SEQ ID NOs: 121-124 (shown in Table 3), or any combination(s) thereof.

For example, one or more gRNAs used to target IRF3 can comprise a sequence selected from SEQ ID NOs: 62-65 (shown in Table 2), or any combination(s) thereof, or a sequence that targets a nucleotide sequence selected from SEQ ID NOs: 125-128 (shown in Table 3), or any combination(s) thereof.

For example, one or more gRNAs used to target IRF7 can comprise a sequence selected from SEQ ID NOs: 66-69 (shown in Table 2), or any combination(s) thereof, or a sequence that targets a nucleotide sequence selected from SEQ ID NOs: 129-132 (shown in Table 3), or any combination(s) thereof.

For example, one or more gRNAs used to target MAVS can comprise a sequence selected from SEQ ID NOs: 70-73 (shown in Table 2) or any combination(s) thereof, or a sequence that targets a nucleotide sequence selected from SEQ ID NOs: 133-136 (shown in Table 3), or any combination(s) thereof.

For example, one or more gRNAs used to target MDA5 (IFIH1) can comprise the sequence of SEQ ID NO: 74 (shown in Table 2) or a sequence that targets the nucleotide sequence of SEQ ID NO: 137 (shown in Table 3).

For example, one or more gRNAs pairs used to target MYD88 can comprise a sequence selected from SEQ ID NOs: 75-78 (shown in Table 2), or any combination(s) thereof, or a sequence that targets a nucleotide sequence selected from SEQ ID NOs: 138-141 (shown in Table 3), or any combination(s) thereof.

For example, one or more gRNAs used to target NFKB1 can comprise a sequence selected from SEQ ID NOs: 79-82 (shown in Table 2) or any combination(s) thereof, or a sequence that targets a nucleotide sequence selected from SEQ ID NOs: 142-145 (shown in Table 3), or any combination(s) thereof.

For example, one or more gRNAs used to target NFKB2 can comprise a sequence selected from SEQ ID NOs: 83-86 (shown in Table 2) or any combination(s) thereof, or a sequence that targets a nucleotide sequence selected from SEQ ID NOs: 146-149 (shown in Table 3), or any combination(s) thereof.

For example, one or more gRNAs used to target RIG-1 (DDX58) can comprise the sequence of SEQ ID NO: 87 (shown in Table 2) or a sequence that targets a nucleotide sequence of SEQ ID NO: 150 (shown in Table 3).

For example, one or more gRNAs used to target TBK1 can comprise a sequence selected from SEQ ID NOs: 88-91 (shown in Table 2), or any combination(s) thereof, or a sequence that targets a nucleotide sequence selected from SEQ ID NOs: 151-154 (shown in Table 3), or any combination(s) thereof.

For example, one or more gRNAs used to target TRIF can comprise a sequence selected from SEQ ID NOs: 92-95 (shown in Table 2), or any combination(s) thereof, or a sequence that targets a nucleotide sequence selected from SEQ ID NOs: 155-158 (shown in Table 3), or any combination(s) thereof.

For example, gRNAs used to target TRIM25 can comprise a sequence selected from SEQ ID NOs: 96-99 (shown in Table 2), or any combination(s) thereof, or a sequence that targets a nucleotide sequence selected from SEQ ID NOs: 159-162 (shown in Table 3), or any combination(s) thereof.

For example, gRNAs used to target KCNN2 can comprise a sequence selected from SEQ ID NOs: 247-250 (shown in Table 2), or any combination(s) thereof, or a sequence that targets a nucleotide sequence selected from SEQ ID NOs: 251-254 (shown in Table 3), or any combination(s) thereof.

In some embodiments, a gRNA molecule used to target a gene is a 2′ O-methyl analog comprising 3′ phosphorothioate internucleotide linkages in the terminal three nucleotides on either or both its 5′ and 3′.

A variant gRNA sequence may have at least 80% sequence identity to a sequence of the present disclosure, measured over any appropriate length of sequence. Typically the percent sequence identity is determined over a length of contiguous nucleic acids. A variant gRNA sequence of the present disclosure can, for example, have at least 80% sequence identity to a sequence of the present disclosure measured over at least 10, at least 11, at least 12, at least 13, at least 14, at least 15, at least 16, at least 17, at least 18, at least 19, at least 20, at least 21, at least 22, at least 23, at least 24, or more nucleic acid residues. In some embodiments, the variant gRNA molecule has at least 80% sequence identity with the gRNA molecule of the present disclosure over the entire length of the variant gRNA molecule. In some embodiments, a variant gRNA molecule of the present disclosure can be a variant of one or more of the gRNA molecules whose target regions are complementary to a target sequence of one of SEQ ID NOs: 37-99. gRNAs of the present disclosure may comprise a variant of one or both of two gRNA sequences in the pair targeting a gene, e.g., a gene selected from CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, and TRIM25.

### Modulation at Protein Level

In another embodiment, modulation of expression and/or activity of a gene takes place at the protein (e.g., polypeptide) level. By way of example, reduction of gene function at the protein level can be achieved by methods including, but not limited to, targeting the protein with a small molecule, a peptide, an aptamer, destabilizing domains, or other methods that can e.g., down-regulate the activity or enhance the rate of degradation of a gene product. Alternatively, the expressed protein may be modified to reduce or eliminate biological activity through site-directed mutagenesis and/or the incorporation of missense or nonsense mutations. In some embodiments, a small molecule that binds, e.g., an active site and inhibits the function of a target protein can be added to, e.g., the cell culture media and thereby be introduced into a packaging and/or producer cell. Alternatively, target protein function can be modulated by introducing, e.g., a peptide into a cell (e.g., a packaging and/or producer cell) that for instance prevents protein-protein interactions (see Shangary et. al., (2009) Annual Review of Pharmacology and Toxicology 49:223). Such peptides can be introduced into a cell (e.g., a packaging and/or producer cell) by, for example, transfection or electroporation, or via an expression construct. Alternatively, peptides can be introduced into a cell (e.g., a packaging and/or producer cell) by adding (e.g., through conjugation) one or more moieties that facilitate cellular delivery, or supercharging molecules to enhance self-delivery. Techniques for expressing a peptide include, but are not limited to, fusion of the peptide to a scaffold, or attachment of a signal sequence, to stabilize or direct the peptide to a position or compartment of interest, respectively. In certain embodiments, a rAAV packaging and/or producer cell line comprises cells which have been engineered to reduce the expression and/or activity of a gene product expressed from a gene selected from the group consisting of CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, TRIM25, and any combination(s) thereof using any of the aforementioned methods.

Effect of Modulation on Expression of One or More Genes and/or Proteins

In certain embodiments, methods of modulations described in the present disclosure can be utilized to generate a rAAV packaging and/or producer cell line that produces high titers of rAAV. In certain embodiments, methods of modulations described in the present disclosure can result in a significant reduction in expression of a gene selected from the group consisting of CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, TRIM25, and any combination(s) thereof; and/or a significant reduction in the activity of a protein expressed by the aforementioned genes (e.g., a reduction of at least 5%, at least 10%, at least 20%, or greater reduction). In certain embodiments, expression of a target gene is reduced from about 40% to about 100% (for example, from about 40% to about 95%, from about 40% to about 90%, from about 40% to about 85%, from about 40% to about 80%, from about 40% to about 75%, from about 40% to about 70%, from about 40% to about 65%, from about 40% to about 60%, from about 40% to about 55%, from about 40% to about 50%, from about 40% to about 45%, from about 45% to about 100%, from about 50% to about 100%, from about 55% to about 100%, from about 60% to about 100%, from about 65% to about 100%, from about 70% to about 100%, from about 75% to about 100%, from about 80% to about 100%, from about 85% to about 100%, from about 90% to about 100%, from about 95% to about 100%; or about 40%, about 50%, about 60%, about 70%, about 80%, about 90%, about 100%).

In certain embodiments, methods of modulation described in the present disclosure can result in a significant reduction in activity of a protein or RNA expressed by a target gene (e.g., CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF. TRIM25, or any combinations thereof). For example, methods described herein can result in at least 5%, at least 10%, at least 20% or greater reduction in activity of a protein or RNA expressed by a target gene. In certain embodiments, target gene protein or RNA activity is reduced from about 40% to about 100% (for example, from about 40% to about 95%, from about 40% to about 90%, from about 40% to about 85%, from about 40% to about 80%, from about 40% to about 75%, from about 40% to about 70%, from about 40% to about 65%, from about 40% to about 60%, from about 40% to about 55%, from about 40% to about 50%, from about 40% to about 45%, from about 45% to about 100%, from about 50% to about 100%, from about 55% to about 100%, from about 60% to about 100%, from about 65% to about 100%, from about 70% to about 100%, from about 75% to about 100%, from about 80% to about 100%, from about 85% to about 100%, from about 90% to about 100%, from about 95% to about 100%; or about 40%, about 50%, about 60%, about 70%, about 80%, about 90%, about 100%). Furthermore, modulation of one or more genes can result in modulation of multiple genes (e.g., by miRNAs).

In certain embodiments, methods of modulation described in the present disclosure can result in a significant reduction in expression of gene product (e.g., a gene product of CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, TRIM25, or any combination(s) thereof) (e.g., at least 5%, at least 10%, at least 20% or greater reduction). In certain embodiments, expression of a gene product is reduced from about 40% to about 100% (for example, from about 40% to about 95%, from about 40% to about 90%, from about 40% to about 85%, from about 40% to about 80%, from about 40% to about 75%, from about 40% to about 70%, from about 40% to about 65%, from about 40% to about 60%, from about 40% to about 55%, from about 40% to about 50%, from about 40% to about 45%, from about 45% to about 100%, from about 50% to about 100%, from about 55% to about 100%, from about 60% to about 100%, from about 65% to about 100%, from about 70% to about 100%, from about 75% to about 100%, from about 80% to about 100%, from about 85% to about 100%, from about 90% to about 100%, from about 95% to about 100%; or about 40%, about 50%, about 60%, about 70%, about 80%, about 90%, about 100%).

In certain embodiments, methods of modulation described in the present disclosure can result in a significant reduction in expression of polypeptide or polyribonucleotide expressed from a gene selected from the group consisting of CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, TRIM25, and any combination(s) thereof (e.g., at least 5%, at least 10%, at least 20% or greater reduction). In certain embodiments, expression of polypeptide or polyribonucleotide is reduced from about 40% to about 100% (for example, from about 40% to about 95%, from about 40% to about 90%, from about 40% to about 85%, from about 40% to about 80%, from about 40% to about 75%, from about 40% to about 70%, from about 40% to about 65%, from about 40% to about 60%, from about 40% to about 55%, from about 40% to about 50%, from about 40% to about 45%, from about 45% to about 100%, from about 50% to about 100%, from about 55% to about 100%, from about 60% to about 100%, from about 65% to about 100%, from about 70% to about 100%, from about 75% to about 100%, from about 80% to about 100%, from about 85% to about 100%, from about 90% to about 100%, from about 95% to about 100%; or about 40%, about 50%, about 60%, about 70%, about 80%, about 90%, about 100%).

In certain embodiments, methods of modulation described in the present disclosure can result in a significant reduction in activity of a polypeptide or polyribonucleotide expressed from a gene selected from the group consisting of CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, and TRIM25, and any combination(s) thereof (e.g., at least 5%, at least 10%, at least 20% or greater reduction). In certain embodiments, activity of expressed polypeptide or polyribonucleotide is reduced from about 40% to about 100% (for example, from about 40% to about 95%, from about 40% to about 90%, from about 40% to about 85%, from about 40% to about 80%, from about 40% to about 75%, from about 40% to about 70%, from about 40% to about 65%, from about 40% to about 60%, from about 40% to about 55%, from about 40% to about 50%, from about 40% to about 45%, from about 45% to about 100%, from about 50% to about 100%, from about 55% to about 100%, from about 60% to about 100%, from about 65% to about 100%, from about 70% to about 100%, from about 75% to about 100%, from about 80% to about 100%, from about 85% to about 100%, from about 90% to about 100%, from about 95% to about 100%; or about 40%, about 50%, about 60%, about 70%, about 80%, about 90%, about 100%).

In certain embodiments, reduction in expression and/or activity of one or more genes, proteins, or RNAs in a rAAV packaging and/or producer cell line is maintained for about 5 days or more (e.g., about 6 hours, about 12 hours, about 1 day, about 2 days, about 3 days, about 4 days, about 5 days, about 6 days, about 7 days, about 8 days, about 9 days, about 10 days or more).

In certain embodiments, reduction in expression and/or activity of one or more genes, proteins, or RNAs in a rAAV packaging and/or producer cell line is intended to be maintained indefinitely or permanently, e.g., through the use of a gene disruption or a partial or complete gene deletion.

In certain embodiments, reduction in expression and/or activity of one or more genes, proteins, or RNAs in a rAAV packaging and/or producer cell line is maintained for at least one, at least two, at least three, at least four, at least five, at least ten, at least 20, at least 30, at least 40 or more passages of the rAAV packaging and/or producer cell line in culture.

Effect of Modulation on rAAV Production

Modulation of one or more genes and/or proteins in a rAAV packaging and/or producer cell line may result in an increase in the titer of rAAV. In some embodiments, modulation results in an increase in the titer of rAAV produced from the rAAV packaging and/or producer cell line. In some embodiments, the titer of rAAV produced from the rAAV packaging and/or producer cell line is increased about 1.5 fold to about 7 fold (e.g., about 1.5 to about 6.5, about 1.5 to about 6, about 1.5 to about 5.5, about 1.5 to about 5, about 1.5 to about 4.5, about 1.5 to about 4, about 1.5 to about 3.5, about 1.5 to about 3.0, about 1.5 to about 2.5, about 1.5 to about 2.0, about 2 to about 7, about 2.5 to about 7, about 3 to about 7, about 3.5 to about 7, about 4 to about 7, about 4.5 to about 7, about 5 to about 7, about 5.5 to about 7, about 6 to about 7, about 6.5 to about 7, or about 1.5, about 2.0, about 2.5, about 3.0, about 3.5, about 4.0, about 4.5, about 5.0, about 5.5, about 6.0, about 6.5, or about 7.0). In some embodiments, the titer of rAAV produced from the rAAV packaging and/or producer cell line is increased at least 1.5 fold, at least 2 fold, at least 3 fold, at least 4 fold, at least 5 fold, at least 6 fold, at least 7 fold, at least 8 fold, at least 9 fold, at least 10 fold, at least 15 fold, at least 20 fold or more. Any increase in the rAAV titer resulting from modulation of one or more genes and/or protein can be compared with the rAAV titer produced from a control parental cell line.

In some embodiments, modulation of one or more genes and/or proteins in a rAAV packaging and/or producer cell line may increase the rAAV titer production for at least 2 days, at least 5 days, at least 20 days, at least 30 days, at least 40 days, at least 50 days, at least 60 days, at least 70 days, at least 80 days, at least 90 days, at least 100 days or more.

Methods of Producing rAAV

In certain embodiments, the present disclosure describes a method of producing rAAV from rAAV packaging and/or producer cell lines that have been engineered to modulate the expression of one or more genes, proteins, or non-coding RNAs. In certain embodiments, rAAV is produced by infecting a packaging cell line of the present disclosure with a helper virus and a hybrid virus that contains AAV gene-therapy elements. In other embodiments, rAAV is produced by infecting a producer cell line of the present disclosure with a helper virus. In certain embodiments, an rAAV packaging or producer cell line as disclosed herein is characterized in that expression of one or more genes, proteins, or non-coding RNAs have been modulated. In certain embodiments, the production of rAAV from engineered rAAV packaging and/or producer cell line is enhanced as compared to a control parental cell line.

In certain embodiments, cells of the engineered packaging cell line are infected with a helper virus (e.g., adenovirus (AV) or herpes simplex virus), which allows the rAAV to replicate. In some embodiments, cells of the engineered producer cell line are infected with a helper virus (e.g., adenovirus (AV) or herpes simplex virus).

Methods of Harvesting rAAV

rAAV particles may be obtained from engineered rAAV packaging and/or producer cells by lysing the cells. Lysis of engineered rAAV packaging and/or producer cells can be accomplished by methods that chemically or enzymatically treat the cells in order to release infectious viral particles. These methods include the use of nucleases such as benzonase or DNAse, proteases such as trypsin, or detergents or surfactants. Physical disruption, such as homogenization or grinding, or the application of pressure via a microfluidizer pressure cell, or freeze-thaw cycles may also be used. In certain embodiments, lysates from the engineered rAAV packaging and/or producer cells can be used to harvest rAAV particles.

In certain embodiments, cell culture supernatant may be collected from engineered rAAV packaging and/or producer cells without the need for cell lysis. In certain embodiments of the present disclosure, the engineered rAAV packaging and/or producer cells secrete rAAV particles that can be collected from the cell culture supernatant without the need for cell lysis. In certain embodiments, the engineered rAAV packaging and/or producer cell line has a higher rAAV titer than that of a control parental cell line such that more rAAV is harvested from the engineered rAAV packaging and/or producer cell line compared to the control parental cell line.

After harvesting rAAV particles, it may be necessary to purify the sample containing rAAV, to remove, for example, the cellular debris resulting from cell lysis. Methods of minimal purification of AAV particles are known in the art. Two exemplary purification methods are Cesium chloride (CsCl)— and iodixanol-based density gradient purification. Both methods are described in Strobel et al., Human Gene Therapy Methods, 26 (4): 147-157 (2015). Minimal purification can also be accomplished using affinity chromatography using, for example, AVB Sepharose affinity resin (GE Healthcare Bio-Sciences AB, Uppsala, Sweden). Methods of AAV purification using AVB Sepharose affinity resin are described in, for example, Wang et al., Mol Ther Methods Clin Dev., 2:15040 (2015). Following purification, rAAV particles may be filtered and stored at ≤−60° C.

In certain embodiments, the present disclosure provides a method of harvesting rAAV particles that are produced from an engineered rAAV packaging cell line after the cells have been co-infected with two different adenoviruses.

In certain embodiments, the present disclosure provides a method of harvesting rAAV particles that are produced after infection of a rAAV producer cell line generated from an engineered rAAV packaging cell line.

In certain embodiments, the present disclosure provides a method of harvesting rAAV particles that are produced after infection of an engineered rAAV producer cell line with a helper virus.

Quantification of rAAV Particles

Quantification of rAAV particles is complicated by the fact that AAV infection does not result in cytopathic effects in vitro, and therefore plaque assays cannot be used to determine infectious titers. rAAV particles can be quantified using a number of methods, however, including quantitative polymerase chain reaction (qPCR) (Clark et al., Hum. Gene Ther. 10, 1031-1039 (1999)), dot-blot hybridization (Samulski et al., J. Virol. 63, 3822-3828 (1989)), and by optical density of highly purified vector preparations (Sommer et al., Mol. Ther. 7, 122-128 (2003)). DNase-resistant particles (DRP) can be quantified by real-time quantitative gene expression reduced polymerase chain reaction (qPCR) (DRP-qPCR) in a thermocycler (for example, an iCycler iQ 96-well block format thermocycler (Bio-Rad, Hercules, CA)). Samples containing rAAV particles can be incubated in the presence of DNase I (100 U/ml: Promega, Madison, WI) at 37° C. for 60 min, followed by proteinase K (Invitrogen, Carlsbad, CA) digestion (10 U/ml) at 50° C. for 60 min, and then denatured at 95° C. for 30 min. The primer-probe set used should be specific to a non-native portion of the rAAV vector genome, for example, the poly(A) sequence of the protein of interest. The PCR product can be amplified using any appropriate set of cycling parameters, based on the length and composition of the primers, probe, and amplified sequence. Alternative protocols are disclosed in, for example, Lock et al., Human Gene Therapy Methods 25 (2): 115-125 (2014).

Viral genome amplification can also be measured using qPCR techniques similar to those described above. However, in order to quantify total genome amplification within producer cells, only intracellular samples are collected and the samples are not treated with DNase I in order to measure both packaged and unpackaged viral genomes. Viral genome amplification may be calculated on a per-host-cell basis by concomitantly measuring a host cell housekeeping gene, for example, RNase P.

The infectivity of rAAV particles can be determined using a TCID50 (tissue culture infectious dose at 50%) assay, as described for example in Zhen et al., Human Gene Therapy 15:709-715 (2004). In this assay, rAAV vector particles are serially diluted and used to co-infect a Rep/Cap-expressing cell line along with AV particles in 96-well plates. 48 hours post-infection, total cellular DNA from infected and control wells is extracted. rAAV vector replication is then measured using qPCR with transgene-specific probe and primers. TCID50 infectivity per milliliter (TCID50/ml) is calculated with the Kärber equation, using the ratios of wells positive for AAV at 10-fold serial dilutions.

### Therapeutic Applications

The rAAV produced from the engineered rAAV packaging and/or producer cell lines described herein can be used, e.g., for gene therapy in mammals. The rAAV produced from the engineered cells described herein can be used for ex vivo and/or in vivo gene therapy applications. The rAAV produced from the engineered cells described herein can be used, e.g., to deliver small molecules (e.g., siRNAs or sgRNAs), peptides, and/or proteins.

In some embodiments, the rAAV generated from the engineered cell lines described herein can be used to treat a disease or a disorder in a human subject in need. In certain embodiments, the rAAV generated from the engineered cell lines described herein can be administered in conjunction with a pharmaceutically acceptable carrier.

Any suitable method or route can be used to administer a rAAV or a rAAV-containing composition produced from the engineered packaging and/or producer cell lines described herein. Routes of administration include, for example, systemic, oral, inhalation, intranasal, intratracheal, intraarterial, intraocular, intravenous, intramuscular, subcutaneous, intradermal, and other parenteral routes of administration. In some embodiments, the rAAV or a composition comprising a rAAV produced from the engineered packaging and/or producer cell line is administered intravenously.

Practice of the disclosure will be more fully understood from the foregoing examples, which are presented herein for illustrative purposes only, and should not be construed as limiting the disclosure in any way.

### EXAMPLES

### Example 1: Effect of Gene Knockdown of cGAS, RIG-1, MDA5, or IFIT3 on rAAV Productivity in HeLa Producer Cell Lines

The purpose of this example is to assess the effect of siRNA-mediated knockdown of specific innate immune genes (here, cGAS, RIG-1, MDA5, or IFIT3) on rAAV productivity in HeLa producer cell lines (PCLs).

Knockdown experiments were performed by individually knocking down genes in HeLa producer cell lines based on an optimized protocol described, e.g., in PCT Publication No. WO 2020/210507, the contents of which are incorporated by reference in their entirety. siRNA nucleotide sequences targeting each gene (see Table 1) were chosen from a library of pre-designed siRNAs.

In this example, a HeLa PCL engineered to produce rAAV (AAV serotype 9 (AAV9) capsid) was transfected with siRNAs to cGAS, RIG-1, MDA5, or IFIT3 for 24 hours. Following siRNA transfection, rAAV production was activated via addition of wildtype Adenovirus serotype 5. Five days post-infection, supernatants were collected and assayed via DNase resistant particle (DRP) qPCR assay. Briefly, supernatants were treated with Salt Active Nuclease (SAN) to remove non-encapsidated DNA, then treated with Proteinase K to deactivate SAN and release encapsidated DNA. Quantitative polymerase chain reaction (qPCR) was then performed on samples to determine rAAV productivity of PCL.

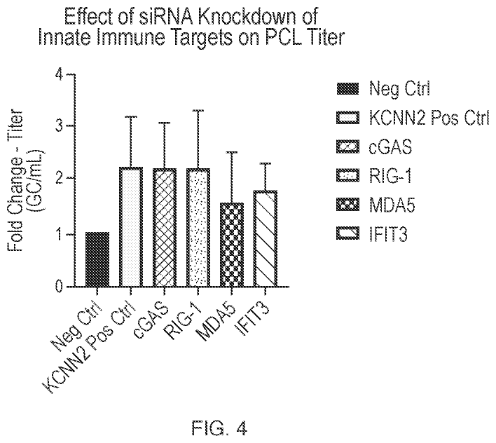

As shown in FIG. 4, siRNA knockdown of cGAS and RIG-1 resulted in 2-3 fold increases in productivity as compared to a scrambled siRNA negative control. siRNA knockdown of MDA5 and IFIT3 resulted in 1.5-2 fold increases in productivity as compared to a scrambled siRNA negative control. Importantly, as a positive control, the siRNA knockdown of KCNN2, which has been previously described as efficacious in increasing productivity (see WO 2020/210507), demonstrated comparable results to the data presented in WO 2020/210507.

### Example 2: Effect of Gene Knockdown on rAAV Productivity in Producer Cell Lines

To assess the effect of siRNA-mediated knockdown of additional innate immune genes and/or combinations of innate immune genes on rAAV production from producer cell lines (PCLs) (e.g., HeLa PCLs), the experiments similar to those described in Example 1 are performed with siRNAs targeting one or more of CHUK (IKK-α), DDX41, DHX58 (LGP2), IFI6, IKBKB (IKK-β), IRF3, IRF7, MAVS, MYD88, NFKB1, NFKB2, TBK1, TRIF, or TRIM25; or one or more combinations of CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, and TRIM25.

Increases in rAAV titer relative to a negative control may indicate that knocking down a particular innate immune gene or combination of innate immune genes can increase productivity in producer cell lines.

### Example 3: Effect of Gene Knockout on rAAV Productivity in HeLa Producer Cell Lines

To assess the effect of gene knockout of innate immune genes and/or combinations of innate immune genes on rAAV production from producer cell lines (PCLs) (e.g., HeLa PCLs), gene knockout experiments are performed using a CRISPR/Cas enzyme and a guide RNA (gRNA) or pair of gRNAs targeting a gene or genes selected from CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, TRIM25, and combinations thereof.

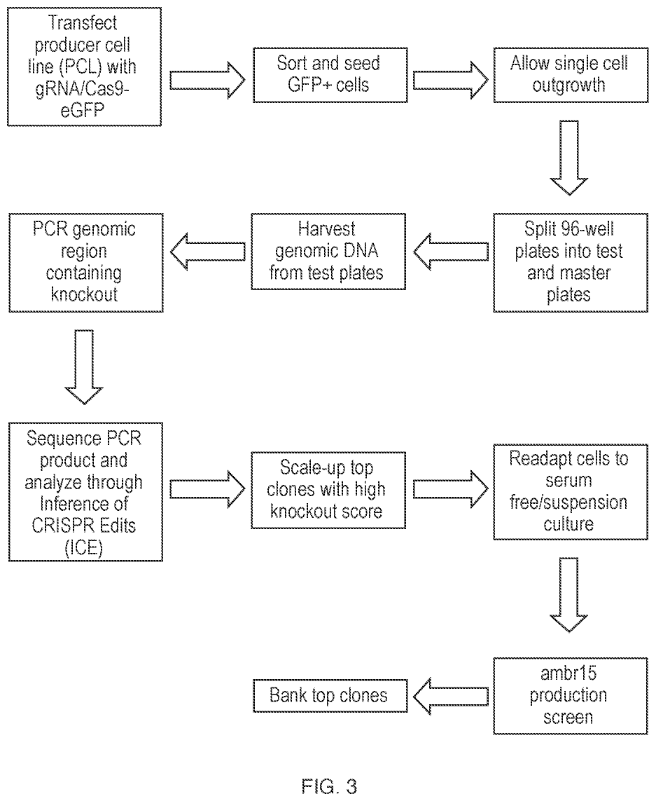

FIG. 3 provides a schematic showing an exemplary procedure for performing these experiments. Cas9-eGFP can be transfected together with the appropriate gRNA(s), and the cells can be sorted for GFP⁺ cells, which are then seeded and grown as colonies forming from single cells. Genome edits in clones can be analyzed by polymerase chain reaction (PCR) and sequencing. Table 2 provides examples of sequences of targeting regions within gRNAs that may be used in these experiments.

Increases in rAAV titer relative to a negative control may indicate that knocking down a particular innate immune gene or combination of innate immune genes can increase productivity in producer cell lines.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A recombinant adeno-associated virus (rAAV) packaging and/or producer cell line comprising cells in which the expression of a gene selected from the group consisting of RIG-1 (DDX58), IFIT3, MDA5 (IFIH1), CGAS (cGAS), CHUK (IKK-α), DDX41, DHX58 (LGP2), IFI6, IKBKB (IKK-β), IRF3, IRF7, MAVS, MYD88, NFKB1, NFKB2, TBK1, TRIF, and TRIM25, and any combination thereof is reduced compared to that in control parental cells.

2. The rAAV packaging and/or producer cell line according to claim 1, wherein the expression is reduced using a nuclease, a double stranded RNA (dsRNA), a small interfering RNA (siRNA), a small hairpin RNA (shRNA), a microRNA (miRNA), or an antisense RNA oligonucleotide (ASO).

3. The rAAV packaging and/or producer cell line according to claim 1 or 2, wherein the expression is reduced with an siRNA comprising a nucleotide sequence selected from any one of SEQ ID NOs: 1-36.

4. The rAAV packaging and/or producer cell line according to claim 3, wherein the expression of RIG-1 (DDX58) is reduced, and the siRNA comprises the nucleotide sequence of SEQ ID NO: 29 in the sense strand and the nucleotide sequence of SEQ ID NO: 30 in the anti-sense strand.

5. The rAAV packaging and/or producer cell line according to claim 3, wherein the expression of IFIT3 is reduced, and the siRNA comprises the nucleotide sequence of SEQ ID NO: 11 in the sense strand and the nucleotide sequence of SEQ ID NO: 12 in the anti-sense strand.

6. The rAAV packaging and/or producer cell line according to claim 3, wherein the expression of MDA5 (IFIH1) is reduced, and the siRNA comprises the nucleotide sequence of SEQ ID NO: 21 in the sense strand and the nucleotide sequence of SEQ ID NO: 22 in the anti-sense strand.

7. The rAAV packaging and/or producer cell line according to claim 3, wherein the expression of CGAS (cGAS) is reduced, and the siRNA comprises the nucleotide sequence of SEQ ID NO: 1 in the sense strand and the nucleotide sequence of SEQ ID NO: 2 in the anti-sense strand.

8. The rAAV packaging and/or producer cell line according to claim 3, wherein the expression of CHUK (IKKα) is reduced, and the siRNA comprises the nucleotide sequence of SEQ ID NO: 3 in the sense strand and the nucleotide sequence of SEQ ID NO: 4 in the anti-sense strand.

9. The rAAV packaging and/or producer cell line according to claim 3, wherein the expression of DDX41 is reduced, and the siRNA comprises the nucleotide sequence of SEQ ID NO: 5 in the sense strand and the nucleotide sequence of SEQ ID NO: 6 in the anti-sense strand.

10. The rAAV packaging and/or producer cell line according to claim 3, wherein the expression of DHX58 (LGP2) is reduced, and the siRNA comprises the nucleotide sequence of SEQ ID NO: 7 in the sense strand and the nucleotide sequence of SEQ ID NO: 8 in the anti-sense strand.

11. The rAAV packaging and/or producer cell line according to claim 3, wherein the expression of IFI6 is reduced, and the siRNA comprises the nucleotide sequence of SEQ ID NO: 9 in the sense strand and the nucleotide sequence of SEQ ID NO: 10 in the anti-sense strand.

12. The rAAV packaging and/or producer cell line according to claim 3, wherein the expression of IKBKB (IKK-β) is reduced, and the siRNA comprises the nucleotide sequence of SEQ ID NO: 13 in the sense strand and the nucleotide sequence of SEQ ID NO: 14 in the anti-sense strand.

13. The rAAV packaging and/or producer cell line according to claim 3, wherein the expression of IRF3 is reduced, and the siRNA comprises the nucleotide sequence of SEQ ID NO: 15 in the sense strand and the nucleotide sequence of SEQ ID NO: 16 in the anti-sense strand.

14. The rAAV packaging and/or producer cell line according to claim 3, wherein the expression of IRF7 is reduced, and the siRNA comprises the nucleotide sequence of SEQ ID NO: 17 in the sense strand and the nucleotide sequence of SEQ ID NO: 18 in the anti-sense strand.

15. The rAAV packaging and/or producer cell line according to claim 3, wherein the expression of MAVS is reduced, and the siRNA comprises the nucleotide sequence of SEQ ID NO: 19 in the sense strand and the nucleotide sequence of SEQ ID NO: 20 in the anti-sense strand.

16. The rAAV packaging and/or producer cell line according to claim 3, wherein the expression of MYD88 is reduced, and the siRNA comprises the nucleotide sequence of SEQ ID NO: 23 in the sense strand and the nucleotide sequence of SEQ ID NO: 24 in the anti-sense strand.

17. The rAAV packaging and/or producer cell line according to claim 3, wherein the expression of NFKB1 is reduced, and the siRNA comprises the nucleotide sequence of SEQ ID NO: 25 in the sense strand and the nucleotide sequence of SEQ ID NO: 26 in the anti-sense strand.

18. The rAAV packaging and/or producer cell line according to claim 3, wherein the expression of NFKB2 is reduced, and the siRNA comprises the nucleotide sequence of SEQ ID NO: 27 in the sense strand and the nucleotide sequence of SEQ ID NO: 28 in the anti-sense strand.

19. The rAAV packaging and/or producer cell line according to claim 3, wherein the expression of TBK1 is reduced, and the siRNA comprises the nucleotide sequence of SEQ ID NO: 31 in the sense strand and the nucleotide sequence of SEQ ID NO: 32 in the anti-sense strand.

20. The rAAV packaging and/or producer cell line according to claim 3, wherein the expression of TRIF is reduced, and the siRNA comprises the nucleotide sequence of SEQ ID NO: 33 in the sense strand and the nucleotide sequence of SEQ ID NO: 34 in the anti-sense strand.

21. The rAAV packaging and/or producer cell line according to claim 3, wherein the expression of TRIM25 is reduced, and the siRNA comprises the nucleotide sequence of SEQ ID NO: 35 in the sense strand and the nucleotide sequence of SEQ ID NO: 36 in the anti-sense strand.

22. The rAAV packaging and/or producer cell line according to claim 1 or 2, wherein the nuclease is selected from the group consisting of a Zinc Finger nuclease (ZFN), a meganuclease, a transcription activator-like effector nuclease (TALEN), and a clustered regularly interspaced short palindromic repeats (CRISPR) associated protein.

23. The rAAV packaging and/or producer cell line according to claim 22, wherein the nuclease is a CRISPR associated protein and the expression is reduced using CRISPR genome editing.

24. The rAAV packaging and/or producer cell line according to claim 23, wherein the expression is reduced using a guide RNA (gRNA), wherein the gRNA:
(a) comprises a nucleotide sequence selected from SEQ ID NOs: 37-99, and/or
(b) targets a DNA sequence selected from SEQ ID NOs: 100-162.

25. The rAAV packaging and/or producer cell line according to claim 24, wherein the gRNA is used to target RIG-1 (DDX58) and (a) comprises the sequence of SEQ ID NO: 87 and/or (b) targets the DNA sequence of SEQ ID NO: 150.

26. The rAAV packaging and/or producer cell line according to claim 24, wherein the gRNA is used to target IFIT3 and (a) comprises a sequence selected from SEQ ID NOs: 54-57 or any combination(s) thereof and/or (b) targets a DNA sequence selected from SEQ ID NOs: 117-120 or any combination(s) thereof.

27. The rAAV packaging and/or producer cell line according to claim 24, wherein the gRNA is used to target MDA5 (IFIH1) and (a) comprises the sequence of SEQ ID NO: 74 and/or (b) targets the DNA sequence of SEQ ID NO: 137.

28. The rAAV packaging and/or producer cell line according to claim 24, wherein the gRNA is used to target CGAS (cGAS) and (a) comprises a sequence selected from SEQ ID NOs: 37-40 or any combination(s) thereof and/or (b) targets a DNA sequence selected from SEQ ID NO: 100-103 or any combination(s) thereof.

29. The rAAV packaging and/or producer cell line according to claim 24, wherein the gRNA is used to target CHUK (IKK-α) and (a) comprises a sequence selected from SEQ ID NOs: 41-44 or any combination(s) thereof and/or (b) targets a DNA sequence selected from SEQ ID NO: 104-107 or any combination(s) thereof.

30. The rAAV packaging and/or producer cell line according to claim 24, wherein the gRNA is used to target DDX41 and (a) comprises a sequence selected from SEQ ID NOs: 45-48 or any combination(s) thereof and/or (b) targets a DNA sequence selected from SEQ ID NOs: 108-111 or any combination(s) thereof.

31. The rAAV packaging and/or producer cell line according to claim 24, wherein the gRNA is used to target DHX58 (LGP2) and (a) comprises the sequence of SEQ ID NO: 49 and/or (b) targets a DNA sequence of SEQ ID NO: 112.

32. The rAAV packaging and/or producer cell line according to claim 24, wherein the gRNA is used to target IFI6 and (a) comprises a sequence selected from SEQ ID NOs: 50-53 or any combination(s) thereof and/or (b) targets a DNA sequence selected from SEQ ID NOs: 113-116 or any combination(s) thereof.

33. The rAAV packaging and/or producer cell line according to claim 24, wherein the gRNA is used to target IKBKB (IKK-β) and (a) comprises a sequence selected from SEQ ID NOs: 58-61 or any combination(s) thereof and/or (b) targets a DNA sequence selected from SEQ ID NOs: 121-124 or any combination(s) thereof.

34. The rAAV packaging and/or producer cell line according to claim 24, wherein the gRNA is used to target IRF3 and (a) comprises a sequence selected from SEQ ID NOs: 62-65 or any combination(s) thereof and/or (b) targets a DNA sequence selected from SEQ ID NOs: 125-128 or any combination(s) thereof.

35. The rAAV packaging and/or producer cell line according to claim 24, wherein the gRNA is used to target IRF7 and (a) comprises a sequence selected from SEQ ID NOs: 66-69 or any combination(s) thereof and/or (b) targets a DNA sequence selected from SEQ ID NOs: 129-132 or any combination(s) thereof.

36. The rAAV packaging and/or producer cell line according to claim 24, wherein the gRNA is used to target MAVS and (a) comprises a sequence selected from SEQ ID NOs: 70-73 or any combination(s) thereof and/or (b) targets a DNA sequence selected from SEQ ID NOs: 133-136 or any combination(s) thereof.

37. The rAAV packaging and/or producer cell line according to claim 24, wherein the gRNA is used to target MYD88 and (a) comprises a sequence selected from SEQ ID NOs: 75-78 or any combination(s) thereof and/or (b) targets a DNA sequence selected from SEQ ID NOs: 138-141 or any combination(s) thereof.

38. The rAAV packaging and/or producer cell line according to claim 24, wherein the gRNA is used to target NFKB1 and (a) comprises a sequence selected from SEQ ID NOs: 79-82 or any combination(s) thereof and/or (b) targets a DNA sequence selected from SEQ ID NOs: 142-145 or any combination(s) thereof.

39. The rAAV packaging and/or producer cell line according to claim 24, wherein the gRNA is used to target NFKB2 and (a) comprises a sequence selected from SEQ ID NOs: 83-86 or any combination(s) thereof and/or (b) targets a DNA sequence selected from SEQ ID NOs: 146-149 or any combination(s) thereof.

40. The rAAV packaging and/or producer cell line according to claim 24, wherein the gRNA is used to target TBK1 and (a) comprises a sequence selected from SEQ ID NOs: 88-91 or any combination(s) thereof and/or (b) targets a DNA sequence selected from SEQ ID NOs: 151-154 or any combination(s) thereof.

41. The rAAV packaging and/or producer cell line according to claim 24, wherein the gRNA is used to target TRIF and (a) comprises a sequence selected from SEQ ID NOs: 92-95 or any combination(s) thereof and/or (b) targets a DNA sequence selected from SEQ ID NOs: 155-158 or any combination(s) thereof.

42. The rAAV packaging and/or producer cell line according to claim 24, wherein the gRNA is used to target TRIM25 and (a) comprises a sequence selected from SEQ ID NOs: 96-99 or any combination(s) thereof and/or (b) targets a DNA sequence selected from SEQ ID NOs: 159-162 or any combination(s) thereof.

43. The rAAV packaging and/or producer cell line according to any one of claims 1-42, wherein the expression of RGMA and/or KCNN2 is reduced compared to the expression of RGMA and KCNN2 in control parental cells.

44. The rAAV packaging and/or producer cell line according to claim 43, wherein the expression of RGMA and/or KCNN2 is reduced using a nuclease, a double stranded RNA (dsRNA), a small interfering RNA (siRNA), a small hairpin RNA (shRNA), a microRNA (miRNA), or an antisense RNA oligonucleotide (ASO).

45. The rAAV packaging and/or producer cell line according to claim 44, wherein the expression of RGMA is reduced using an siRNA that comprises the nucleotide sequence of SEQ ID NO: 238 in the sense strand and the nucleotide sequence of SEQ ID NO: 239 in the anti-sense strand.

46. The rAAV packaging and/or producer cell line according to claim 44 or 45, wherein the expression of KCNN2 is reduced using an siRNA that comprises the nucleotide sequence of SEQ ID NO: 245 in the sense strand and the nucleotide sequence of SEQ ID NO: 246 in the anti-sense strand.

47. The rAAV packaging and/or producer cell line according to claim 44, wherein the nuclease is selected from the group consisting of a Zinc Finger nuclease (ZFN), a meganuclease, a transcription activator-like effector nuclease (TALEN), and a clustered regularly interspaced short palindromic repeats (CRISPR) associated protein.

48. The rAAV packaging and/or producer cell line according to claim 47, wherein the nuclease is a CRISPR associated protein and the expression is reduced using CRISPR genome editing.

49. The rAAV packaging and/or producer cell line according to claim 48, wherein the expression of RGMA is reduced using a guide RNA (gRNA) that targets a DNA sequence selected from SEQ ID NOs: 240-243.

50. The rAAV packaging and/or producer cell line according to claim 48, wherein the expression of KCNN2 is reduced using a guide RNA (gRNA) that targets a DNA sequence selected from SEQ ID NOs: 251-254.

51. The rAAV packaging and/or producer cell line of any one of claim 24-42 or 50, wherein each gRNA molecule is a 2′ O-methyl analog comprising 3′ phosphorothioate internucleotide linkages in the terminal three nucleotides on either or both its 5′ and 3′ ends.

52. The rAAV packaging and/or producer cell line according to any one of claims 1-51, wherein the gene expression is eliminated.

53. The rAAV packaging and/or producer cell line according to any one of claims 1-52, wherein the cell line is a human cell line.

54. The rAAV packaging and/or producer cell line according to claim 53, wherein the human cell line is a HeLa cell line or a human embryonic kidney (HEK) 293 cell line.

55. A cell comprising:
(a) AAV rep and cap genes and
(b) an siRNA comprising a nucleotide sequence selected from any one of SEQ ID NOs: 1-36.

56. A cell comprising:
(a) AAV rep and cap genes and
(b) (i) a guide RNA (gRNA) comprising a nucleotide sequence selected from SEQ ID Nos: 37-99; and/or
(ii) a gRNA which targets a DNA sequence selected from SEQ ID NOs: 100-162.

57. A cell comprising:
(a) AAV rep and cap genes and
(b) a nuclease selected from the group consisting of Zinc Finger nuclease (ZFN), a meganuclease, or a transcription activator-like effector nuclease (TALEN), wherein the nuclease targets a gene selected from the group RIG-1 (DDX58), IFIT3, MDA5 (IFIH1), CGAS (cGAS), CHUK (IKK-α), DDX41, DHX58 (LGP2), IFI6, IKBKB (IKK-β), IRF3, IRF7, MAVS, MYD88, NFKB1, NFKB2, TBK1, TRIF, TRIM25, and combinations thereof.

58. A cell comprising:
(a) AAV rep and cap genes and
(b) a knockout version of a gene selected from the group consisting of RIG-1 (DDX58), IFIT3, MDA5 (IFIH1), CGAS (cGAS), CHUK (IKK-α), DDX41, DHX58 (LGP2), IFI6, IKBKB (IKK-β), IRF3, IRF7, MAVS, MYD88, NFKB1, NFKB2, TBK1, TRIF, TRIM25, and
combinations thereof.

59. The cell of claim 58, wherein the AAV rep and cap genes are stably integrated into the genome of the cell.

60. An recombinant adeno-associated virus (rAAV) packaging and/or producer cell line comprising the cell of any one of claims 55-59.

61. An rAAV packaging cell line according to the rAAV packaging cell line of any one of claims 1-54 and 60.

62. An rAAV producer cell line according to the rAAV producer cell line of any one of claims 1-54 and 60.

63. The rAAV producer cell line according to claim 62, wherein the cell line is capable of producing or produces rAAV at a titer that is increased by at least 1.5-fold compared to the titer of rAAV produced from a control parental cell line.

64. The rAAV producer cell line according to claim 63, wherein the cell line is capable of producing or produces rAAV at a titer that is increased by at least 2-fold compared to the titer of rAAV produced from a control parental cell line.

65. A lysate of the cell line according to any one of claims 1-64.

66. A cell culture supernatant from a cell line according to any one of claims 1-64.

67. A method of generating a rAAV producer cell line, the method comprising delivering a rAAV vector to the cells of a rAAV packaging cell line according to any one of claims 1-61.

68. A method of producing rAAV, the method comprising infecting the cells of a rAAV producer cell line generated by the method of claim 67 with a helper virus.

69. A method of producing rAAV, the method comprising infecting the cells of a rAAV producer cell line according to any one of claims 62 to 64 with a helper virus.

70. The method of claim 68 or 69, further comprising harvesting the rAAV from the rAAV producer cell line.

71. A method of any one of claims 68 to 70, wherein production of rAAV from the rAAV producer cell line is enhanced as compared that of a control parental cell line.

72. A recombinant adeno-associated virus (rAAV) packaging and/or producer cell line comprising cells which have been engineered to reduce the expression and/or activity of a gene product expressed from a gene selected from the group consisting of RIG-1 (DDX58), IFIT3, MDA5 (IFIH1), CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, and TRIM25, and any combination thereof as compared to corresponding control parental cells.

73. The rAAV packaging and/or producer cell line of claim 72, wherein the expression and/or activity of a gene product expressed from a gene selected from the group consisting of RIG-1 (DDX58), IFIT3, MDA5 (IFIH1), CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, and TRIM25, and any combination thereof is reduced indefinitely or permanently.

74. The rAAV packaging and/or producer cell line of claim 73, wherein the cell line has been engineered to comprise a gene disruption or a partial or complete gene deletion in a gene selected from the group consisting of RIG-1 (DDX58), IFIT3, MDA5 (IFIH1), CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, and TRIM25, and any combination thereof.

75. The rAAV packaging and/or producer cell line of claim 74, wherein the cell line has been engineered to comprise a gene disruption in a gene selected from the group consisting of RIG-1 (DDX58), IFIT3, MDA5 (IFIH1), CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, and TRIM25, and any combination thereof.

76. The rAAV packaging and/or producer cell line of claim 74, wherein the cell line has been engineered to comprise a partial or complete gene deletion in a gene selected from the group consisting of RIG-1 (DDX58), IFIT3, MDA5 (IFIH1), CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, and TRIM25, and any combination thereof.

77. The rAAV packaging and/or producer cell line of any one of claims 72-76, wherein the cells are further engineered to reduce the expression and/or activity of RGMA and/or KCNN2.

78. A recombinant adeno-associated virus (rAAV) packaging and/or producer cell line, wherein said cell line exhibits reduced expression and/or activity of a polypeptide or polyribonucleotide expressed from a gene selected from the group consisting of RIG-1 (DDX58), IFIT3, MDA5 (IFIH1), CGAS (cGAS), CHUK (IKK-α), DDX41, DHX58 (LGP2), IFI6, IFIT3, IKBKB (IKK-β), IRF3, IRF7, MAVS, MDA5 (IFIH1), MYD88, NFKB1, NFKB2, RIG-1 (DDX58), TBK1, TRIF, and TRIM25, and any combination thereof as compared to that of a control parental cell line.


SEQUENCE LISTING (256 sequences)

SEQ ID NO: 1 - RNA, 20 nt, synthetic construct
  source 1..20: mol_type = other RNA
tttctgcacctaatgaattt

SEQ ID NO: 2 - RNA, 20 nt, synthetic construct
  source 1..20: mol_type = other RNA
aaattcattaggtgcagaaa

SEQ ID NO: 3 - RNA, 20 nt, synthetic construct
  source 1..20: mol_type = other RNA
aaagtgtatatttgcatgtg

SEQ ID NO: 4 - RNA, 20 nt, synthetic construct
  source 1..20: mol_type = other RNA
cacatgcaaatatacacttt

SEQ ID NO: 5 - RNA, 20 nt, synthetic construct
  source 1..20: mol_type = other RNA
ttctgcctggaacaagagaa

SEQ ID NO: 6 - RNA, 20 nt, synthetic construct
  source 1..20: mol_type = other RNA
ttctcttgttccaggcagaa

SEQ ID NO: 7 - RNA, 20 nt, synthetic construct
  source 1..20: mol_type = other RNA
tatgagcagcaggtggtgaa

SEQ ID NO: 8 - RNA, 20 nt, synthetic construct
  source 1..20: mol_type = other RNA
ttcaccacctgctgctcata

SEQ ID NO: 9 - RNA, 20 nt, synthetic construct
  source 1..20: mol_type = other RNA
aggaacagactcagcctccc

SEQ ID NO: 10 - RNA, 20 nt, synthetic construct
  source 1..20: mol_type = other RNA
gggaggctgagtctgttcct

SEQ ID NO: 11 - RNA, 20 nt, synthetic construct
  source 1..20: mol_type = other RNA
gtgtctcaagggatctagaa

SEQ ID NO: 12 - RNA, 20 nt, synthetic construct
  source 1..20: mol_type = other RNA
ttctagatcccttgagacac

SEQ ID NO: 13 - RNA, 20 nt, synthetic construct
  source 1..20: mol_type = other RNA
tttgcacatcattcgtgggg

SEQ ID NO: 14 - RNA, 20 nt, synthetic construct
  source 1..20: mol_type = other RNA
ccccacgaatgatgtgcaaa

SEQ ID NO: 15 - RNA, 20 nt, synthetic construct
  source 1..20: mol_type = other RNA
tctacgagtttgtgaactca

SEQ ID NO: 16 - RNA, 20 nt, synthetic construct
  source 1..20: mol_type = other RNA
tgagttcacaaactcgtaga

SEQ ID NO: 17 - RNA, 20 nt, synthetic construct
  source 1..20: mol_type = other RNA
ccatcatgtacaagggccgc

SEQ ID NO: 18 - RNA, 20 nt, synthetic construct
  source 1..20: mol_type = other RNA
gcggcccttgtacatgatgg

SEQ ID NO: 19 - RNA, 20 nt, synthetic construct
  source 1..20: mol_type = other RNA
cctaccaccttgatgcctgt

SEQ ID NO: 20 - RNA, 20 nt, synthetic construct
  source 1..20: mol_type = other RNA
acaggcatcaaggtggtagg

SEQ ID NO: 21 - RNA, 20 nt, synthetic construct
  source 1..20: mol_type = other RNA
aagttatagttcttgtcaat

SEQ ID NO: 22 - RNA, 20 nt, synthetic construct
  source 1..20: mol_type = other RNA
attgacaagaactataactt

SEQ ID NO: 23 - RNA, 20 nt, synthetic construct
  source 1..20: mol_type = other RNA
tatcgactgaagttgtgtgt

SEQ ID NO: 24 - RNA, 20 nt, synthetic construct
  source 1..20: mol_type = other RNA
acacacaacttcagtcgata

SEQ ID NO: 25 - RNA, 19 nt, synthetic construct
  source 1..19: mol_type = other RNA
tttacagcttttcttccgg

SEQ ID NO: 26 - RNA, 19 nt, synthetic construct
  source 1..19: mol_type = other RNA
ccggaagaaaagctgtaaa

SEQ ID NO: 27 - RNA, 20 nt, synthetic construct
  source 1..20: mol_type = other RNA
gctacaacccaggtctggat

SEQ ID NO: 28 - RNA, 20 nt, synthetic construct
  source 1..20: mol_type = other RNA
atccagacctgggttgtagc

SEQ ID NO: 29 - RNA, 20 nt, synthetic construct
  source 1..20: mol_type = other RNA
atcccaaccgatatcatttc

SEQ ID NO: 30 - RNA, 20 nt, synthetic construct
  source 1..20: mol_type = other RNA
gaaatgatatcggttgggat

SEQ ID NO: 31 - RNA, 20 nt, synthetic construct
  source 1..20: mol_type = other RNA
attagcttataatgaagaac

SEQ ID NO: 32 - RNA, 20 nt, synthetic construct
  source 1..20: mol_type = other RNA
gttcttcattataagctaat

SEQ ID NO: 33 - RNA, 20 nt, synthetic construct
  source 1..20: mol_type = other RNA
tgtagaagataccacctctc

SEQ ID NO: 34 - RNA, 20 nt, synthetic construct
  source 1..20: mol_type = other RNA
gagaggtggtatcttctaca

SEQ ID NO: 35 - RNA, 20 nt, synthetic construct
  source 1..20: mol_type = other RNA
atacacggaaatgaaggctc

SEQ ID NO: 36 - RNA, 20 nt, synthetic construct
  source 1..20: mol_type = other RNA
gagccttcatttccgtgtat

SEQ ID NO: 37 - RNA, 20 nt, synthetic construct
  source 1..20: mol_type = other RNA
gaactttcccgccttaggca

SEQ ID NO: 38 - RNA, 20 nt, synthetic construct
  source 1..20: mol_type = other RNA
tgcatggcctttccgtgcca

SEQ ID NO: 39 - RNA, 20 nt, synthetic construct
  source 1..20: mol_type = other RNA
ctgcctaaggcgggaaagtt

SEQ ID NO: 40 - RNA, 20 nt, synthetic construct
  source 1..20: mol_type = other RNA
cgaactttcccgccttaggc

SEQ ID NO: 41 - RNA, 20 nt, synthetic construct
  source 1..20: mol_type = other RNA
acgtctgtctgtaccagcat

SEQ ID NO: 42 - RNA, 20 nt, synthetic construct
  source 1..20: mol_type = other RNA
gcaatggaatactgttctgg

SEQ ID NO: 43 - RNA, 20 nt, synthetic construct
  source 1..20: mol_type = other RNA
tgatgtgcctcttctagcaa

SEQ ID NO: 44 - RNA, 20 nt, synthetic construct
  source 1..20: mol_type = other RNA
aggcctttacaacattggca

SEQ ID NO: 45 - RNA, 20 nt, synthetic construct
  source 1..20: mol_type = other RNA
acgtgccctatgtgccgtta

SEQ ID NO: 46 - RNA, 20 nt, synthetic construct
  source 1..20: mol_type = other RNA
gatgaggacgacatcccgct

SEQ ID NO: 47 - RNA, 20 nt, synthetic construct
  source 1..20: mol_type = other RNA
gatcctggagagtgttgccg

SEQ ID NO: 48 - RNA, 20 nt, synthetic construct
  source 1..20: mol_type = other RNA
taaggtgctggtgctgatcc

SEQ ID NO: 49 - RNA, 20 nt, synthetic construct
  source 1..20: mol_type = other RNA
tggagcttcggtcctaccaa

SEQ ID NO: 50 - RNA, 20 nt, synthetic construct
  source 1..20: mol_type = other RNA
caagtatctcgatagtgagg

SEQ ID NO: 51 - RNA, 20 nt, synthetic construct
  source 1..20: mol_type = other RNA
ctggtctgcgatcctgaatg

SEQ ID NO: 52 - RNA, 20 nt, synthetic construct
  source 1..20: mol_type = other RNA
ggtaatattggtgccctgat

SEQ ID NO: 53 - RNA, 20 nt, synthetic construct
  source 1..20: mol_type = other RNA
gaaaaagtgctcggagagct

SEQ ID NO: 54 - RNA, 20 nt, synthetic construct
  source 1..20: mol_type = other RNA
catcgcaattgccagtccag

SEQ ID NO: 55 - RNA, 20 nt, synthetic construct
  source 1..20: mol_type = other RNA
ggcaattgcgatgtaccatc

SEQ ID NO: 56 - RNA, 20 nt, synthetic construct
  source 1..20: mol_type = other RNA
acacctagatggtaacaacg

SEQ ID NO: 57 - RNA, 20 nt, synthetic construct
  source 1..20: mol_type = other RNA
cgcctgggtctactatcact

SEQ ID NO: 58 - RNA, 20 nt, synthetic construct
  source 1..20: mol_type = other RNA
atgaaggtatctaagcgcag

SEQ ID NO: 59 - RNA, 20 nt, synthetic construct
  source 1..20: mol_type = other RNA
catgtctgcgttgtcaggga

SEQ ID NO: 60 - RNA, 20 nt, synthetic construct
  source 1..20: mol_type = other RNA
accgtcgactactggagctt

SEQ ID NO: 61 - RNA, 20 nt, synthetic construct
  source 1..20: mol_type = other RNA
gaagcccgtgatgcactcaa

SEQ ID NO: 62 - RNA, 20 nt, synthetic construct
  source 1..20: mol_type = other RNA
gcaacccttctttgcggttg

SEQ ID NO: 63 - RNA, 20 nt, synthetic construct
  source 1..20: mol_type = other RNA
tctccggacaccaatggtgg

SEQ ID NO: 64 - RNA, 20 nt, synthetic construct
  source 1..20: mol_type = other RNA
ggttgcgtttagcagaggac

SEQ ID NO: 65 - RNA, 20 nt, synthetic construct
  source 1..20: mol_type = other RNA
attaccttcacggaaggaag

SEQ ID NO: 66 - RNA, 20 nt, synthetic construct
  source 1..20: mol_type = other RNA
cgtgatgctgcgggataact

SEQ ID NO: 67 - RNA, 20 nt, synthetic construct
  source 1..20: mol_type = other RNA
gcggaagttggttttccagc

SEQ ID NO: 68 - RNA, 20 nt, synthetic construct
  source 1..20: mol_type = other RNA
atcagcagcggctgctatga

SEQ ID NO: 69 - RNA, 20 nt, synthetic construct
  source 1..20: mol_type = other RNA
ctccttggagagatcagcag

SEQ ID NO: 70 - RNA, 20 nt, synthetic construct
  source 1..20: mol_type = other RNA
ctgtgagctagttgatctcg

SEQ ID NO: 71 - RNA, 20 nt, synthetic construct
  source 1..20: mol_type = other RNA
gtacttcattgcggcactga

SEQ ID NO: 72 - RNA, 20 nt, synthetic construct
  source 1..20: mol_type = other RNA
tcttcaatacccttcagcgg

SEQ ID NO: 73 - RNA, 20 nt, synthetic construct
  source 1..20: mol_type = other RNA
gggtattgaagagatgccag

SEQ ID NO: 74 - RNA, 20 nt, synthetic construct
  source 1..20: mol_type = other RNA
ttggactcgggaattcgtgg

SEQ ID NO: 75 - RNA, 20 nt, synthetic construct
  source 1..20: mol_type = other RNA
cgttcaagaacagagacagg

SEQ ID NO: 76 - RNA, 20 nt, synthetic construct
  source 1..20: mol_type = other RNA
gttcttgaacgtgcggacac

SEQ ID NO: 77 - RNA, 20 nt, synthetic construct
  source 1..20: mol_type = other RNA
ctcgagctgcttaccaagct

SEQ ID NO: 78 - RNA, 20 nt, synthetic construct
  source 1..20: mol_type = other RNA
atgaaggcatcgaaacgctc

SEQ ID NO: 79 - RNA, 20 nt, synthetic construct
  source 1..20: mol_type = other RNA
cgtttccgttatgtatgtga

SEQ ID NO: 80 - RNA, 20 nt, synthetic construct
  source 1..20: mol_type = other RNA
caactatgtgggaccagcaa

SEQ ID NO: 81 - RNA, 20 nt, synthetic construct
  source 1..20: mol_type = other RNA
gggatctgcactgtaactgc

SEQ ID NO: 82 - RNA, 20 nt, synthetic construct
  source 1..20: mol_type = other RNA
gaacaagaagtcttaccctc

SEQ ID NO: 83 - RNA, 20 nt, synthetic construct
  source 1..20: mol_type = other RNA
gttccacgatcaccaggtag

SEQ ID NO: 84 - RNA, 20 nt, synthetic construct
  source 1..20: mol_type = other RNA
cgatttcgatatggctgtga

SEQ ID NO: 85 - RNA, 20 nt, synthetic construct
  source 1..20: mol_type = other RNA
ctttcggcccttctcactgg

SEQ ID NO: 86 - RNA, 20 nt, synthetic construct
  source 1..20: mol_type = other RNA
aggtccacctcgatcttggc

SEQ ID NO: 87 - RNA, 20 nt, synthetic construct
  source 1..20: mol_type = other RNA
ggattatatccggaagaccc

SEQ ID NO: 88 - RNA, 20 nt, synthetic construct
  source 1..20: mol_type = other RNA
ggtagtccataggcattaga

SEQ ID NO: 89 - RNA, 20 nt, synthetic construct
  source 1..20: mol_type = other RNA
tttgaacatccactggacga

SEQ ID NO: 90 - RNA, 20 nt, synthetic construct
  source 1..20: mol_type = other RNA
cataagcttccttcgtccag

SEQ ID NO: 91 - RNA, 20 nt, synthetic construct
  source 1..20: mol_type = other RNA
gctactgcaaatgtctttcg

SEQ ID NO: 92 - RNA, 20 nt, synthetic construct
  source 1..20: mol_type = other RNA
tgctgcacctagaatgtcga

SEQ ID NO: 93 - RNA, 20 nt, synthetic construct
  source 1..20: mol_type = other RNA
gatctctctagaggcattga

SEQ ID NO: 94 - RNA, 20 nt, synthetic construct
  source 1..20: mol_type = other RNA
gccatggttctcctgaagct

SEQ ID NO: 95 - RNA, 20 nt, synthetic construct
  source 1..20: mol_type = other RNA
cttgtaccacctgctggctg

SEQ ID NO: 96 - RNA, 20 nt, synthetic construct
  source 1..20: mol_type = other RNA
gtcgtgcctgaatgagacgt

SEQ ID NO: 97 - RNA, 20 nt, synthetic construct
  source 1..20: mol_type = other RNA
accgtgttcttgtgcagctg

SEQ ID NO: 98 - RNA, 20 nt, synthetic construct
  source 1..20: mol_type = other RNA
gaacacggtgctgtgcaacg

SEQ ID NO: 99 - RNA, 20 nt, synthetic construct
  source 1..20: mol_type = other RNA
gaagacgtgcttggtgtgca

SEQ ID NO: 100 - DNA, 20 bp, synthetic construct
  source 1..20: mol_type = other DNA
gaactttcccgccttaggca

SEQ ID NO: 101 - DNA, 20 bp, synthetic construct
  source 1..20: mol_type = other DNA
tgcatggcctttccgtgcca

SEQ ID NO: 102 - DNA, 20 bp, synthetic construct
  source 1..20: mol_type = other DNA
ctgcctaaggcgggaaagtt

SEQ ID NO: 103 - DNA, 20 bp, synthetic construct
  source 1..20: mol_type = other DNA
cgaactttcccgccttaggc

SEQ ID NO: 104 - DNA, 20 bp, synthetic construct
  source 1..20: mol_type = other DNA
acgtctgtctgtaccagcat

SEQ ID NO: 105 - DNA, 20 bp, synthetic construct
  source 1..20: mol_type = other DNA
gcaatggaatactgttctgg

SEQ ID NO: 106 - DNA, 20 bp, synthetic construct
  source 1..20: mol_type = other DNA
tgatgtgcctcttctagcaa

SEQ ID NO: 107 - DNA, 20 bp, synthetic construct
  source 1..20: mol_type = other DNA
aggcctttacaacattggca

SEQ ID NO: 108 - DNA, 20 bp, synthetic construct
  source 1..20: mol_type = other DNA
acgtgccctatgtgccgtta

SEQ ID NO: 109 - DNA, 20 bp, synthetic construct
  source 1..20: mol_type = other DNA
gatgaggacgacatcccgct

SEQ ID NO: 110 - DNA, 20 bp, synthetic construct
  source 1..20: mol_type = other DNA
gatcctggagagtgttgccg

SEQ ID NO: 111 - DNA, 20 bp, synthetic construct
  source 1..20: mol_type = other DNA
taaggtgctggtgctgatcc

SEQ ID NO: 112 - DNA, 20 bp, synthetic construct
  source 1..20: mol_type = other DNA
tggagcttcggtcctaccaa

SEQ ID NO: 113 - DNA, 20 bp, synthetic construct
  source 1..20: mol_type = other DNA
caagtatctcgatagtgagg

SEQ ID NO: 114 - DNA, 20 bp, synthetic construct
  source 1..20: mol_type = other DNA
ctggtctgcgatcctgaatg

SEQ ID NO: 115 - DNA, 20 bp, synthetic construct
  source 1..20: mol_type = other DNA
ggtaatattggtgccctgat

SEQ ID NO: 116 - DNA, 20 bp, synthetic construct
  source 1..20: mol_type = other DNA
gaaaaagtgctcggagagct

SEQ ID NO: 117 - DNA, 20 bp, synthetic construct
  source 1..20: mol_type = other DNA
catcgcaattgccagtccag

SEQ ID NO: 118 - DNA, 20 bp, synthetic construct
  source 1..20: mol_type = other DNA
ggcaattgcgatgtaccatc

SEQ ID NO: 119 - DNA, 20 bp, synthetic construct
  source 1..20: mol_type = other DNA
acacctagatggtaacaacg

SEQ ID NO: 120 - DNA, 20 bp, synthetic construct
  source 1..20: mol_type = other DNA
cgcctgggtctactatcact

SEQ ID NO: 121 - DNA, 20 bp, synthetic construct
  source 1..20: mol_type = other DNA
atgaaggtatctaagcgcag

SEQ ID NO: 122 - DNA, 20 bp, synthetic construct
  source 1..20: mol_type = other DNA
catgtctgcgttgtcaggga

SEQ ID NO: 123 - DNA, 20 bp, synthetic construct
  source 1..20: mol_type = other DNA
accgtcgactactggagctt

SEQ ID NO: 124 - DNA, 20 bp, synthetic construct
  source 1..20: mol_type = other DNA
gaagcccgtgatgcactcaa

SEQ ID NO: 125 - DNA, 20 bp, synthetic construct
  source 1..20: mol_type = other DNA
gcaacccttctttgcggttg

SEQ ID NO: 126 - DNA, 20 bp, synthetic construct
  source 1..20: mol_type = other DNA
tctccggacaccaatggtgg

SEQ ID NO: 127 - DNA, 20 bp, synthetic construct
  source 1..20: mol_type = other DNA
ggttgcgtttagcagaggac

SEQ ID NO: 128 - DNA, 20 bp, synthetic construct
  source 1..20: mol_type = other DNA
attaccttcacggaaggaag

SEQ ID NO: 129 - DNA, 20 bp, synthetic construct
  source 1..20: mol_type = other DNA
cgtgatgctgcgggataact

SEQ ID NO: 130 - DNA, 20 bp, synthetic construct
  source 1..20: mol_type = other DNA
gcggaagttggttttccagc

SEQ ID NO: 131 - DNA, 20 bp, synthetic construct
  source 1..20: mol_type = other DNA
atcagcagcggctgctatga

SEQ ID NO: 132 - DNA, 20 bp, synthetic construct
  source 1..20: mol_type = other DNA
ctccttggagagatcagcag

SEQ ID NO: 133 - DNA, 20 bp, synthetic construct
  source 1..20: mol_type = other DNA
ctgtgagctagttgatctcg

SEQ ID NO: 134 - DNA, 20 bp, synthetic construct
  source 1..20: mol_type = other DNA
gtacttcattgcggcactga

SEQ ID NO: 135 - DNA, 20 bp, synthetic construct
  source 1..20: mol_type = other DNA
tcttcaatacccttcagcgg

SEQ ID NO: 136 - DNA, 20 bp, synthetic construct
  source 1..20: mol_type = other DNA
gggtattgaagagatgccag

SEQ ID NO: 137 - DNA, 20 bp, synthetic construct
  source 1..20: mol_type = other DNA
ttggactcgggaattcgtgg

SEQ ID NO: 138 - DNA, 20 bp, synthetic construct
  source 1..20: mol_type = other DNA
cgttcaagaacagagacagg

SEQ ID NO: 139 - DNA, 20 bp, synthetic construct
  source 1..20: mol_type = other DNA
gttcttgaacgtgcggacac

SEQ ID NO: 140 - DNA, 20 bp, synthetic construct
  source 1..20: mol_type = other DNA
ctcgagctgcttaccaagct

SEQ ID NO: 141 - DNA, 20 bp, synthetic construct
  source 1..20: mol_type = other DNA
atgaaggcatcgaaacgctc

SEQ ID NO: 142 - DNA, 20 bp, synthetic construct
  source 1..20: mol_type = other DNA
cgtttccgttatgtatgtga

SEQ ID NO: 143 - DNA, 20 bp, synthetic construct
  source 1..20: mol_type = other DNA
caactatgtgggaccagcaa

SEQ ID NO: 144 - DNA, 20 bp, synthetic construct
  source 1..20: mol_type = other DNA
gggatctgcactgtaactgc

SEQ ID NO: 145 - DNA, 20 bp, synthetic construct
  source 1..20: mol_type = other DNA
gaacaagaagtcttaccctc

SEQ ID NO: 146 - DNA, 20 bp, synthetic construct
  source 1..20: mol_type = other DNA
gttccacgatcaccaggtag

SEQ ID NO: 147 - DNA, 20 bp, synthetic construct
  source 1..20: mol_type = other DNA
cgatttcgatatggctgtga

SEQ ID NO: 148 - DNA, 20 bp, synthetic construct
  source 1..20: mol_type = other DNA
ctttcggcccttctcactgg

SEQ ID NO: 149 - DNA, 20 bp, synthetic construct
  source 1..20: mol_type = other DNA
aggtccacctcgatcttggc

SEQ ID NO: 150 - DNA, 20 bp, synthetic construct
  source 1..20: mol_type = other DNA
ggattatatccggaagaccc

SEQ ID NO: 151 - DNA, 20 bp, synthetic construct
  source 1..20: mol_type = other DNA
ggtagtccataggcattaga

SEQ ID NO: 152 - DNA, 20 bp, synthetic construct
  source 1..20: mol_type = other DNA
tttgaacatccactggacga

SEQ ID NO: 153 - DNA, 20 bp, synthetic construct
  source 1..20: mol_type = other DNA
cataagcttccttcgtccag

SEQ ID NO: 154 - DNA, 20 bp, synthetic construct
  source 1..20: mol_type = other DNA
gctactgcaaatgtctttcg

SEQ ID NO: 155 - DNA, 20 bp, synthetic construct
  source 1..20: mol_type = other DNA
tgctgcacctagaatgtcga

SEQ ID NO: 156 - DNA, 20 bp, synthetic construct
  source 1..20: mol_type = other DNA
gatctctctagaggcattga

SEQ ID NO: 157 - DNA, 20 bp, synthetic construct
  source 1..20: mol_type = other DNA
gccatggttctcctgaagct

SEQ ID NO: 158 - DNA, 20 bp, synthetic construct
  source 1..20: mol_type = other DNA
cttgtaccacctgctggctg

SEQ ID NO: 159 - DNA, 20 bp, synthetic construct
  source 1..20: mol_type = other DNA
gtcgtgcctgaatgagacgt

SEQ ID NO: 160 - DNA, 20 bp, synthetic construct
  source 1..20: mol_type = other DNA
accgtgttcttgtgcagctg

SEQ ID NO: 161 - DNA, 20 bp, synthetic construct
  source 1..20: mol_type = other DNA
gaacacggtgctgtgcaacg

SEQ ID NO: 162 - DNA, 20 bp, synthetic construct
  source 1..20: mol_type = other DNA
gaagacgtgcttggtgtgca

SEQ ID NO: 163 - DNA, 3201 bp, Homo sapiens
  source 1..3201: mol_type = genomic DNA
gcagactcttgtgtgcccgccagtagtgcttggtttccaacagctgctgctggctcttcctcttgcggccttttcctgaaacggattcttctttcggggaacagaaagcgccagccatgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccagaattcaactagaagaatattccaacactcgtgcatattactttgtgaaatttaaaagaaatccgaaagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttgagattgtatttttagaaagatctaagaactagagtcaccctaaatcctggagaatacaagaaaaatttgaaaaggggccagacgctgtggctcacacctgtaatcccagctctttggaggccgaggcaggcggatcacttgaggtcaggagtttgagaccagcctgaccaacatggtgaaactccatctctactaaaaatataaaaattagccgggcatggtgatgcatgcctgtaatcccagctactcgggaggcttagacatgagaatcacttgaacccaggaggtggaggttgcagtgagtcaagatggcaccactgcactccatcctgggtgacagagcaagacttcctctcaaaaaataaataaataaataagaaaaataaattaggaaattattaaaataattttttagaaaagcaacaaaataacaaaaattagtgactgtaataattggaatgtttgaaatttgtcacagcaataattgactaaaatgaatgtaaataattattcagttttcagtttgttgtaataaactctaatcagtataaaggattgactatcctaggaaaaagattagtcaaggaataagaagcaaaagtagtatttgaaatctaaaaacattaccatgtttgctttatcatgcaggcaaataaataaataaatgacatttttaggttggaataaaaaaagataaagtacatattgccttaattaaattcatcaagttcttactgaaaaacagctctccgccgtgcctggccaggaaatacataattttttttttttttttttagatggagtctcgctctgtcgcccaggctggagtccagtggcatgatcttggctcactgcaagctctgcttcctgggttcatgccattctcctgcctcagccttccgagtagctgggactacaggtgcccgccaccacatccggctaattttttgtatttttagtaaagatggggtttcaccatgttagccaggatggtctcgatctccttaccttgtgatccgcccgccttggcctcccaaagtgctgggattacaggtgtgagccaccacgcctggctgaaatacataatcttaaaagaaaacataagatactttattttaatatacgtgactaaatgtaaaacctaacttattttctgttatctatttatttttactttcagtaacactttttttattttaggtagcattcagcctagaggcaactgctgtttgttaaatatttcctgttcatatattttgcacattttcttatgggttagttttcttctcattgttttgggaagttcttaatatatttggggtatttatctttcattcgttgtctgtgtaacaaataacttctgccatatgggttgtctgcacattttttggtgtcttttagtaaacaaggtttttttgttttgtattgttttgtttattgtaaagatttttaaattttaatggagttgatttcttttctcattcaagcttttgagaataaattggagttgaattttt

SEQ ID NO: 164 - DNA, 3600 bp, Homo sapiens
  source 1..3600: mol_type = genomic DNA
atcggcggcccggaaccggccttggaacaactgtggaacctgaggccgcttgccctcccgccccatggagcggcccccggggctgcggccgggcgcgggcgggccctgggagatgcgggagcggctgggcaccggcggcttcgggaacgtctgtctgtaccagcatcgggaacttgatctcaaaatagcaattaagtcttgtcgcctagagctaagtaccaaaaacagagaacgatggtgccatgaaatccagattatgaagaagttgaaccatgccaatgttgtaaaggcctgtgatgttcctgaagaattgaatattttgattcatgatgtgcctcttctagcaatggaatactgttctggaggagatctccgaaagctgctcaacaaaccagaaaattgttgtggacttaaagaaagccagatactttctttactaagtgatatagggtctgggattcgatatttgcatgaaaacaaaattatacatcgagatctaaaacctgaaaacatagttcttcaggatgttggtggaaagataatacataaaataattgatctgggatatgccaaagatgttgatcaaggaagtctgtgtacatcttttgtgggaacactgcagtatctggccccagagctctttgagaataagccttacacagccactgttgattattggagctttgggaccatggtatttgaatgtattgctggatataggccttttttgcatcatctgcagccatttacctggcatgagaagattaagaagaaggatccaaagtgtatatttgcatgtgaagagatgtcaggagaagttcggtttagtagccatttacctcaaccaaatagcctttgtagtttagtagtagaacccatggaaaactggctacagttgatgttgaattgggaccctcagcagagaggaggacctgttgaccttactttgaagcagccaagatgttttgtattaatggatcacattttgaatttgaagatagtacacatcctaaatatgacttctgcaaagataatttcttttctgttaccacctgatgaaagtcttcattcactacagtctcgtattgagcgtgaaactggaataaatactggttctcaagaacttctttcagagacaggaatttctctggatcctcggaaaccagcctctcaatgtgttctagatggagttagaggctgtgatagctatatggtttatttgtttgataaaagtaaaactgtatatgaagggccatttgcttccagaagtttatctgattgtgtaaattatattgtacaggacagcaaaatacagcttccaattatacagctgcgtaaagtgtgggctgaagcagtgcactatgtgtctggactaaaagaagactatagcaggctctttcagggacaaagggcagcaatgttaagtcttcttagatataatgctaacttaacaaaaatgaagaacactttgatctcagcatcacaacaactgaaagctaaattggagttttttcacaaaagcattcagcttgacttggagagatacagcgagcagatgacgtatgggatatcttcagaaaaaatgctaaaagcatggaaagaaatggaagaaaaggccatccactatgctgaggttggtgtcattggatacctggaggatcagattatgtctttgcatgctgaaatcatggagctacagaagagcccctatggaagacgtcagggagacttgatggaatctctggaacagcgtgccattgatctatataagcagttaaaacacagaccttcagatcactcctacagtgacagcacagagatggtgaaaatcattgtgcacactgtgcagagtcaggaccgtgtgctcaaggagctgtttggtcatttgagcaagttgttgggctgtaagcagaagattattgatctactccctaaggtggaagtggccctcagtaatatcaaagaagctgacaatactgtcatgttcatgcagggaaaaaggcagaaagaaatatggcatctccttaaaattgcctgtacacagagttctgcccggtcccttgtaggatccagtctagaaggtgcagtaacccctcagacatcagcatggctgcccccgacttcagcagaacatgatcattctctgtcatgtgtggtaactcctcaagatggggagacttcagcacaaatgatagaagaaaatttgaactgccttggccatttaagcactattattcatgaggcaaatgaggaacagggcaatagtatgatgaatcttgattggagttggttaacagaatgagttgtcacttgttcactgtccccaaacctatggaagttgttgctatacatgttggaaatgtgtttttcccccatgaaaccattcttcagacatcagtcaatggaagaaatggctatgaacagaaactacatttctactatgatcagaagaacatgattttacaagtataacagttttgagtaattcaagcctctaaacagacaggaatttagaaaaagtcaatgtacttgtttgaatatttgttttaataccacagctatttagaagcatcatcacgacacatttgccttcagtcttggtaaaacattacttatttaactgattaaaaataccttctatgtattagtgtcaacttttaacttttgggcgtaagaccaaatgtagttttgtatacagagaagaaaacctcaagtaataggcattttaagtaaaagtctacctgtgtttttttctaaaaaggctgctcacaagttctatttcttgaagaataaattctacctccttgtgttgcactgaacaggttctcttcctggcatcataaggagttggtgtaatcattttaaattccactgaaaatttaacagtatccccttctcatcgaagggattgtgtatctgtgcttctaatattagttggctttcataaatcatgttgttgtgtgtatatgtatttaagatgtacatttaataatatcaaagagaagatgcctgttaatttataatgtatttgaaaattacatgttttttcatttgtaaaaatgagtcatttgtttaaacaatctttcatgtcttgtcatacaaatttataaaggtctgcactcctttatctgtaattgtaattccaaaatccaaaaagctctgaaaacaaggtttccataagcttggtgacaaaattcatttgcttgcaatctaatctgaactgaccttgaatctttttatcccatttagtgtgaatattcctttattttgctgcttgatgatgagagggagggctgctgccacagactgtggtgagggctggttaatgtagtatggtatatgcacaaaactacttttctaaaatctaaaatttcataattctgaaacaacttgccccaagggtttcagagaaaggactgtggacctctatcatctgctaagtaatttagaagatattatttgtcttaaaaaatgtgaaatgcttttatattctaatagtttttcactttgtgtattaaatggtttttaaattactttcttgatctctattcattataaaaatcagattataataaaacagttgaatatggcttaggaaaa

SEQ ID NO: 165 - DNA, 3583 bp, Homo sapiens
  source 1..3583: mol_type = genomic DNA
atcggcggcccggaaccggccttggaacaactgtggaacctgaggccgcttgccctcccgccccatggagcggcccccggggctgcggccgggcgcgggcgggccctgggagatgcgggagcggctgggcaccggcggcttcgggaacgtctgtctgtaccagcatcgggaacttgatctcaaaatagcaattaagtcttgtcgcctagagctaagtaccaaaaacagagaacgatggtgccatgaaatccagattatgaagaagttgaaccatgccaatgttgtaaaggcctgtgatgttcctgaagaattgaatattttgattcatgatgtgcctcttctagcaatggaatactgttctggaggagatctccgaaagctgctcaacaaaccagaaaattgttgtggacttaaagaaagccagatactttctttactaagtgatatagggtctgggattcgatatttgcatgaaaacaaaattatacatcgagatctaaaacctgaaaacatagttcttcaggatgttggtggaaagataatacataaaataattgatctgggatatgccaaagatgttgatcaaggaagtctgtgtacatcttttgtgggaacactgcagtatctggccccagagctctttgagaataagccttacacagccactgttgattattggagctttgggaccatggtatttgaatgtattgctggatataggccttttttgcatcatctgcagccatttacctggcatgagaagattaagaagaaggatccaaagtgtatatttgcatgtgaagagatgtcaggagaagttcggtttagtagccatttacctcaaccaaatagcctttgtagtttagtagtagaacccatggaaaactggctacagttgatgttgaattgggaccctcagcagagaggaggacctgttgaccttactttgaagcagccaagatgttttgtattaatggatcacattttgaatttgaagatagtacacatcctaaatatgacttctgcaaagataatttcttttctgttaccacctgatgaaagtcttcattcactacagtctcgtattgagcgtgaaactggaataaatactggttctcaagaacttctttcagagacaggaatttctctggatcctcggaaaccagcctctcaatgtgttctagatggagttagaggctgtgatagctatatggtttatttgtttgataaaagtaaaactgtatatgaagggccatttgcttccagaagtttatctgattgtgtaaattatattgtacaggacagcaaaatacagcttccaattatacagctgcgtaaagtgtgggctgaagcagtgcactatgtgtctggactaaaagaagactatagcaggctctttcagggacaaagggcagcaatgttaagtcttcttagatataatgctaacttaacaaaaatgaagaacactttgatctcagcatcacaacaactgaaagctaaattggagttttttcacaaaagcattcagcttgacttggagagatacagcgagcagatgacgtatgggatatcttcagaaaaaatgctaaaagcatggaaagaaatggaagaaaaggccatccactatgctgaggttggtgtcattggatacctggaggatcagattatgtctttgcatgctgaaatcatggagctacagaagagcccctatggaagacgtcagggagacttgatggaatctctggaacagcgtgccattgatctatataagcagttaaaacacagaccttcagatcactcctacagtgacagcacagagatggtgaaaatcattgtgcacactgtgcagagtcaggaccgtgtgctcaaggagctgtttggtcatttgagcaagttgttgggctgtaagcagaagattattgatctactccctaaggtggaagtggccctcagtaatatcaaagaagctgacaatactgtcatgttcatgcagggaaaaaggcagaaagaaatatggcatctccttaaaattgcctgtacacagagttctgcccggtcccttgtaggatccagtctagaaggtgcagtaacccctcagacatcagcatggctgcccccgacttcagcagaacatgatcattctctgtcatgtgtgggagacttcagcacaaatgatagaagaaaatttgaactgccttggccatttaagcactattattcatgaggcaaatgaggaacagggcaatagtatgatgaatcttgattggagttggttaacagaatgagttgtcacttgttcactgtccccaaacctatggaagttgttgctatacatgttggaaatgtgtttttcccccatgaaaccattcttcagacatcagtcaatggaagaaatggctatgaacagaaactacatttctactatgatcagaagaacatgattttacaagtataacagttttgagtaattcaagcctctaaacagacaggaatttagaaaaagtcaatgtacttgtttgaatatttgttttaataccacagctatttagaagcatcatcacgacacatttgccttcagtcttggtaaaacattacttatttaactgattaaaaataccttctatgtattagtgtcaacttttaacttttgggcgtaagaccaaatgtagttttgtatacagagaagaaaacctcaagtaataggcattttaagtaaaagtctacctgtgtttttttctaaaaaggctgctcacaagttctatttcttgaagaataaattctacctccttgtgttgcactgaacaggttctcttcctggcatcataaggagttggtgtaatcattttaaattccactgaaaatttaacagtatccccttctcatcgaagggattgtgtatctgtgcttctaatattagttggctttcataaatcatgttgttgtgtgtatatgtatttaagatgtacatttaataatatcaaagagaagatgcctgttaatttataatgtatttgaaaattacatgttttttcatttgtaaaaatgagtcatttgtttaaacaatctttcatgtcttgtcatacaaatttataaaggtctgcactcctttatctgtaattgtaattccaaaatccaaaaagctctgaaaacaaggtttccataagcttggtgacaaaattcatttgcttgcaatctaatctgaactgaccttgaatctttttatcccatttagtgtgaatattcctttattttgctgcttgatgatgagagggagggctgctgccacagactgtggtgagggctggttaatgtagtatggtatatgcacaaaactacttttctaaaatctaaaatttcataattctgaaacaacttgccccaagggtttcagagaaaggactgtggacctctatcatctgctaagtaatttagaagatattatttgtcttaaaaaatgtgaaatgcttttatattctaatagtttttcactttgtgtattaaatggtttttaaattactttcttgatctctattcattataaaaatcagattataataaaacagttgaatatggcttaggaaaa

SEQ ID NO: 166 - DNA, 2286 bp, Homo sapiens
  source 1..2286: mol_type = genomic DNA
aaagaatggaggagtcggaacccgaacggaagcgggctcgcaccgacgaggtgcctgccggaggaagccgctccgaggcggaagatgaggacgacgaggactacgtgccctatgtgccgttacggcagcgccggcagctactgccaggatcctgacgctgaccttgtggactccatgcccatcctatctgtggatcccgggtggaacctcagctccagaagctgctgcagcgaagacgcaagggagctgcggaggaagagcagcaggacagcggtagtgaaccccggggagatgaggacgacatcccgctaggccctcagtccaacgtcagcctcctggatcagcaccagcaccttaaagagaaggctgaagcgcgcaaagagtctgccaaggagaagcagctgaaggaagaagagaagatcctggagagtgttgccgagggccgaggtatggttgtagccaggatagtgagaaggtggactgagtcttctcctcccagccccaggcatgttatatctcaagggatctggcttctcagaagcaggagccagccctgagccatgtctctctcttcagcattgatgtcagtgaaggagatggctaagggcattacgtatgatgaccccatcaaaaccagctggactccaccccgttatgttctgagcatgtctgaagagcgacatgagcgcgtgcggaagaaataccacatcctggtggagggagacggtatcccaccacccatcaagagcttcaaggaaatgaagtttcctgcagccatcctgagaggcctgaagaagaaaggcattcaccacccaacacccattcagatccagggcatccccaccattctatctggccgtgacatgataggcatcgctttcacgggttcaggcaagacactggtgttcacgttgcccgtcatcatgttctgcctggaacaagagaagaggttacccttctcaaagcgcgaggggccctatggactcatcatctgcccctcgcgggagctggcccggcagacccatggcatcctggagtactactgccgcctgctgcaggaggacagctcaccactcctgcgctgcgccctctgcattgggggcatgtccgtgaaagagcagatggagaccatccgacacggtgtacacatgatggtggccaccccggggcgcctcatggatttgctgcagaagaagatggtcagcctagacatctgtcgctacctggccctggacgaggctgaccgcatgatcgacatgggcttcgagggtgacatccgtaccatcttctcctacttcaagggccagcgacagaccctgctcttcagtgccaccatgccgaagaagattcagaactttgctaagagtgcccttgtaaagcctgtgaccatcaatgtggggcgcgctggggctgccagcctggatgtcatccaggaggtagaatatgtgaaggaggaggccaagatggtgtacctgctcgagtgcctgcagaagacacccccgcctgtactcatctttgcagagaagaaggcagacgtggacgccatccacgagtacctgctgctcaagggggttgaggccgtagccatccatgggggcaaagaccaggaggaacggactaaggccatcgaggcattccgggagggcaagaaggatgtcctagtagccacagacgttgcctccaagggcctggacttccctgccatccagcacgtcatcaattatgacatgccagaggagattgagaactatgtacaccggattggccgcaccgggcgctcgggaaacacaggcatcgccactaccttcatcaacaaagcgtgtgatgagtcagtgctgatggacctcaaagcgctgctgctagaagccaagcagaaggtgccgcccgtgctgcaggtgctgcattgcggggatgagtccatgctggacattggaggagagcgcggctgtgccttctgcgggggcctgggtcatcggatcactgactgccccaaactcgaggctatgcagaccaagcaggtcagcaacatcggtcgcaaggactacctggcccacagctccatggacttctgagccgacagtcttcccttctctccaagaggcctcagtccccaagactgccaccagtctacacatacagcagccccctggacagaatcagcatttcagctcagctggcctgggatgggccaggctggtcctggctgcctgttccctgtgctcttcagaattactgtttttgtttccttttaccccagctgccattaaagcccaaacctctagccca

SEQ ID NO: 167 - DNA, 2376 bp, Homo sapiens
  source 1..2376: mol_type = genomic DNA
atgcgtgcagcaaagaatggaggagtcggaacccgaacggaagcgggctcgcaccgacgaggtgcctgccggaggaagccgctccgaggcggaagatgaggacgacgaggactacgtgccctatgtgccgttacggcagcgccggcagctactggtgaggggcacgcgtccggggagggatggggccgtgaccgcggggtcgcagggggcgggcgtggcctcggcctccgtggggaggagggcagtctgaggagtgagcttcagtacaccagggcgcggcccccagacaaacgagggaccgacggcttgatctgaggggtagggcagctttgcgcctcgggcacaggatcctcgccccaaagccaggatcctgacgctgaccttgtggactccatgcccatcctatctgtggatcccgggtggaacctcagctccagaagctgctgcagcgaagacgcaagggagctgcggaggaagagcagcaggacagcggtagtgaaccccggggagatgaggacgacatcccgctaggccctcagtccaacgtcagcctcctggatcagcaccagcaccttaaagagaaggctgaagcgcgcaaagagtctgccaaggagaagcagctgaaggaagaagagaagatcctggagagtgttgccgagggccgagcattgatgtcagtgaaggagatggctaagggcattacgtatgatgaccccatcaaaaccagctggactccaccccgttatgttctgagcatgtctgaagagcgacatgagcgcgtgcggaagaaataccacatcctggtggagggagacggtatcccaccacccatcaagagcttcaaggaaatgaagtttcctgcagccatcctgagaggcctgaagaagaaaggcattcaccacccaacacccattcagatccagggcatccccaccattctatctggccgtgacatgataggcatcgctttcacgggttcaggcaagacactggtgttcacgttgcccgtcatcatgttctgcctggaacaagagaagaggttacccttctcaaagcgcgaggggccctatggactcatcatctgcccctcgcgggagctggcccggcagacccatggcatcctggagtactactgccgcctgctgcaggaggacagctcaccactcctgcgctgcgccctctgcattgggggcatgtccgtgaaagagcagatggagaccatccgacacggtgtacacatgatggtggccaccccggggcgcctcatggatttgctgcagaagaagatggtcagcctagacatctgtcgctacctggccctggacgaggctgaccgcatgatcgacatgggcttcgagggtgacatccgtaccatcttctcctacttcaagggccagcgacagaccctgctcttcagtgccaccatgccgaagaagattcagaactttgctaagagtgcccttgtaaagcctgtgaccatcaatgtggggcgcgctggggctgccagcctggatgtcatccaggaggtagaatatgtgaaggaggaggccaagatggtgtacctgctcgagtgcctgcagaagacacccccgcctgtactcatctttgcagagaagaaggcagacgtggacgccatccacgagtacctgctgctcaagggggttgaggccgtagccatccatgggggcaaagaccaggaggaacggactaaggccatcgaggcattccgggagggcaagaaggatgtcctagtagccacagacgttgcctccaagggcctggacttccctgccatccagcacgtcatcaattatgacatgccagaggagattgagaactatgtacaccggattggccgcaccgggcgctcgggaaacacaggcatcgccactaccttcatcaacaaagcgtgtgatgagtcagtgctgatggacctcaaagcgctgctgctagaagccaagcagaaggtgccgcccgtgctgcaggtgctgcattgcggggatgagtccatgctggacattggaggagagcgcggctgtgccttctgcgggggcctgggtcatcggatcactgactgccccaaactcgaggctatgcagaccaagcaggtcagcaacatcggtcgcaaggactacctggcccacagctccatggacttctgagccgacagtcttcccttctctccaagaggcctcagtccccaagactgccaccagtctacacatacagcagccccctggacagaatcagcatttcagctcagctggcctgggatgggccaggctggtcctggctgcctgttccctgtgctcttcagaattactgtttttgtttccttttaccccagctgccattaaagcccaaacctctagccca

SEQ ID NO: 168 - DNA, 2099 bp, Homo sapiens
  source 1..2099: mol_type = genomic DNA
atgcgtgcagcaaagaatggaggagtcggaacccgaacggaagcgggctcgcaccgacgaggtgcctgccggaggaagccgctccgaggcggaagatgaggacgacgaggactacgtgccctatgtgccgttacggcagcgccggcagctactgctccagaagctgctgcagcgaagacgcaagggagctgcggaggaagagcagcaggacagcggtagtgaaccccggggagatgaggacgacatcccgctaggccctcagtccaacgtcagcctcctggatcagcaccagcaccttaaagagaaggctgaagcgcgcaaagagtctgccaaggagaagcagctgaaggaagaagagaagatcctggagagtgttgccgagggccgagcattgatgtcagtgaaggagatggctaagggcattacgtatgatgaccccatcaaaaccagctggactccaccccgttatgttctgagcatgtctgaagagcgacatgagcgcgtgcggaagaaataccacatcctggtggagggagacggtatcccaccacccatcaagagcttcaaggaaatgaagtttcctgcagccatcctgagaggcctgaagaagaaaggcattcaccacccaacacccattcagatccagggcatccccaccattctatctggccgtgacatgataggcatcgctttcacgggttcaggcaagacactggtgttcacgttgcccgtcatcatgttctgcctggaacaagagaagaggttacccttctcaaagcgcgaggggccctatggactcatcatctgcccctcgcgggagctggcccggcagacccatggcatcctggagtactactgccgcctgctgcaggaggacagctcaccactcctgcgctgcgccctctgcattgggggcatgtccgtgaaagagcagatggagaccatccgacacggtgtacacatgatggtggccaccccggggcgcctcatggatttgctgcagaagaagatggtcagcctagacatctgtcgctacctggccctggacgaggctgaccgcatgatcgacatgggcttcgagggtgacatccgtaccatcttctcctacttcaagggccagcgacagaccctgctcttcagtgccaccatgccgaagaagattcagaactttgctaagagtgcccttgtaaagcctgtgaccatcaatgtggggcgcgctggggctgccagcctggatgtcatccaggaggtagaatatgtgaaggaggaggccaagatggtgtacctgctcgagtgcctgcagaagacacccccgcctgtactcatctttgcagagaagaaggcagacgtggacgccatccacgagtacctgctgctcaagggggttgaggccgtagccatccatgggggcaaagaccaggaggaacggactaaggccatcgaggcattccgggagggcaagaaggatgtcctagtagccacagacgttgcctccaagggcctggacttccctgccatccagcacgtcatcaattatgacatgccagaggagattgagaactatgtacaccggattggccgcaccgggcgctcgggaaacacaggcatcgccactaccttcatcaacaaagcgtgtgatgagtcagtgctgatggacctcaaagcgctgctgctagaagccaagcagaaggtgccgcccgtgctgcaggtgctgcattgcggggatgagtccatgctggacattggaggagagcgcggctgtgccttctgcgggggcctgggtcatcggatcactgactgccccaaactcgaggctatgcagaccaagcaggtcagcaacatcggtcgcaaggactacctggcccacagctccatggacttctgagccgacagtcttcccttctctccaagaggcctcagtccccaagactgccaccagtctacacatacagcagccccctggacagaatcagcatttcagctcagctggcctgggatgggccaggctggtcctggctgcctgttccctgtgctcttcagaattactgtttttgtttccttttaccccagctgccattaaagcccaaacctctagccca

SEQ ID NO: 169 - DNA, 2591 bp, Homo sapiens
  source 1..2591: mol_type = genomic DNA
gatttctgctctctgcgctgagcacagcggcaccaggctgagctaagcagggccgccttgggcaggcctacgtggtggtgcaggcgagacccaggctgggcaaggcgcagtttcagtttccatcttgggtctctgagctgagcagagtggcaccaggctgagttaagtgggactgccctgggcagacctacctactagagcagaatggagcttcggtcctaccaatgggaggtgatcatgcctgccctggagggcaagaatatcatcatctggctgcccacgggtgccgggaagacccgggcggctgcttatgtggccaagcggcacctagagactgtggatggagccaaggtggttgtattggtcaacagggtgcacctggtgacccagcatggtgaagagttcaggcgcatgctggatggacgctggaccgtgacaaccctgagtggggacatgggaccacgtgctggctttggccacctggcccggtgccatgacctgctcatctgcacagcagagcttctgcagatggcactgaccagccccgaggaggaggagcacgtggagctcactgtcttctccctgatcgtggtggatgagtgccaccacacgcacaaggacaccgtctacaacgtcatcatgagccagtacctagaacttaaactccagagggcacagccgctaccccaggtgctgggtctcacagcctccccaggcactggcggggcctccaaactcgatggggccatcaaccacgtcctgcagctctgtgccaacttggacacgtggtgcatcatgtcaccccagaactgctgcccccagctgcaggagcacagccaacagccttgcaaacagtacaacctctgccacaggcgcagccaggatccgtttggggacttgctgaagaagctcatggaccaaatccatgaccacctggagatgcctgagttgagccggaaatttgggacgcaaatgtatgagcagcaggtggtgaagctgagtgaggctgcggctttggctgggcttcaggagcaacgggtgtatgcgcttcacctgaggcgctacaatgacgcgctgctcatccatgacaccgtccgcgccgtggatgccttggctgcgctgcaggatttctatcacagggagcacgtcactaaaacccagatcctgtgtgccgagcgccggctgctggccctgttcgatgaccgcaagaatgagctggcccacttggcaactcatggcccagagaatccaaaactggagatgctggaaaagatcctgcaaaggcagttcagtagctctaacagccctcggggtatcatcttcacccgcacccgccaaagcgcacactccctcctgctctggctccagcagcagcagggcctgcagactgtggacatccgggcccagctactgattggggctgggaacagcagccagagcacccacatgacccagagggaccagcaagaagtgatccagaagttccaagatggaaccctgaaccttctggtggccacgagtgtggcggaggaggggctggacatcccacattgcaatgtggtggtgcgttatgggctcttgaccaatgaaatctccatggtccaggccaggggccgtgcccgggccgatcagagtgtatacgcgtttgtagcaactgaaggtagccgggagctgaagcgggagctgatcaacgaggcgctggagacgctgatggagcaggcagtggctgctgtgcagaaaatggaccaggccgagtaccaggccaagatccgggatctgcagcaggcagccttgaccaagcgggcggcccaggcagcccagcgggagaaccagcggcagcagttcccagtggagcacgtgcagctactctgcatcaactgcatggtggctgtgggccatggcagcgacctgcggaaggtggagggcacccaccatgtcaatgtgaaccccaacttctcgaactactataatgtctccagggatcctgtggtcatcaacaaagtcttcaaggactggaagcctgggggtgtcatcagctgcaggaactgtggggaggtctggggtctgcagatgatctacaagtcagtgaagctgccagtgctcaaagtccgcagcatgctgctggagacccctcaggggcggatccaggccaaaaagtggtcccgcgtgcccttctccgtgcctgactttgacttcctgcagcattgtgccgagaacttgtcggacctctccctggactgaccacctcattgctgcagtgcccggtttgggctgtagggggcgggagagtctgcagcagactccaggcccctccttcctgaatcatcagctgtgggcatcaggcccaccagccacacaggagtcctgggcaccctggcttaggctcccgcaatgggaaaacaaccggagggccagagcttagtccagacctaccttgtacgcacatagacattttcatatgcactggatggagttagggaaactgaggcaaaagaatttgccatactgtactcagaatcacgacattccttccctaccaaggccacttctattttttgaggctcctcataaaaataaatgaaaaaatggga

SEQ ID NO: 170 - DNA, 812 bp, Homo sapiens
  source 1..812: mol_type = genomic DNA
gtttactcgctgctgtgcccatctatcagcaggctccgggctgaagattgcttctcttctctcctccaaggtctagtgacggagcccgcgcgcggcgccaccatgcggcagaaggcggtatcgcttttcttgtgctacctgctgctcttcacttgcagtggggtggaggcaggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgcctttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaaca

SEQ ID NO: 171 - DNA, 824 bp, Homo sapiens
  source 1..824: mol_type = genomic DNA
gtttactcgctgctgtgcccatctatcagcaggctccgggctgaagattgcttctcttctctcctccaaggtctagtgacggagcccgcgcgcggcgccaccatgcggcagaaggcggtatcgcttttcttgtgctacctgctgctcttcacttgcagtggggtggaggcaggtgagaatgcgggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgcctttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaaca

SEQ ID NO: 172 - DNA, 836 bp, Homo sapiens
  source 1..836: mol_type = genomic DNA
gtttactcgctgctgtgcccatctatcagcaggctccgggctgaagattgcttctcttctctcctccaaggtctagtgacggagcccgcgcgcggcgccaccatgcggcagaaggcggtatcgcttttcttgtgctacctgctgctcttcacttgcagtggggtggaggcaggtgagaatgcgggtaaggatgcaggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgcctttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaaca

SEQ ID NO: 173 - DNA, 2407 bp, Homo sapiens
  source 1..2407: mol_type = genomic DNA
gtggaaacctcttcagcatttgcttggaatcagtaagctaaaaacaaaatcaaccgggaccccagcttttcagaactgcagggaaacagccatcatgagtgaggtcaccaagaattccctggagaaaatccttccacagctgaaatgccatttcacctggaacttattcaaggaagacagtgtctcaagggatctagaagatagagtgtgtaaccagattgaatttttaaacactgagttcaaagctacaatgtacaacttgttggcctacataaaacacctagatggtaacaacgaggcagccctggaatgcttacggcaagctgaagagttaatccagcaagaacatgctgaccaagcagaaatcagaagtctagtcacttggggaaactacgcctgggtctactatcacttgggcagactctcagatgctcagatttatgtagataaggtgaaacaaacctgcaagaaattttcaaatccatacagtattgagtattctgaacttgactgtgaggaagggtggacacaactgaagtgtggaagaaatgaaagggcgaaggtgtgttttgagaaggctctggaagaaaagcccaacaacccagaattctcctctggactggcaattgcgatgtaccatctggataatcacccagagaaacagttctctactgatgttttgaagcaggccattgagctgagtcctgataaccaatacgtcaaggttctcttgggcctgaaactgcagaagatgaataaagaagctgaaggagagcagtttgttgaagaagccttggaaaagtctccttgccaaacagatgtcctccgcagtgcagccaaattttacagaagaaaaggtgacctagacaaagctattgaactgtttcaacgggtgttggaatccacaccaaacaatggctacctctatcaccagattgggtgctgctacaaggcaaaagtaagacaaatgcagaatacaggagaatctgaagctagtggaaataaagagatgattgaagcactaaagcaatatgctatggactattcgaataaagctcttgagaagggactgaatcctctgaatgcatactccgatctcgctgagttcctggagacggaatgttatcagacaccattcaataaggaagtccctgatgctgaaaagcaacaatcccatcagcgctactgcaaccttcagaaatataatgggaagtctgaagacactgctgtgcaacatggtttagagggtttgtccataagcaaaaaatcaactgacaaggaagagatcaaagaccaaccacagaatgtatctgaaaatctgcttccacaaaatgcaccaaattattggtatcttcaaggattaattcataagcagaatggagatctgctgcaagcagccaaatgttatgagaaggaactgggccgcctgctaagggatgccccttcaggcataggcagtattttcctgtcagcatctgagcttgaggatggtagtgaggaaatgggccagggcgcagtcagctccagtcccagagagctcctctctaactcagagcaactgaactgagacagaggaggaaaacagagcatcagaagcctgcagtggtggttgtgacgggtaggacgataggaagacagggggccccaacctgggattgctgagcagggaagctttgcatgttgctctaaggtacatttttaaagagttgttttttggccgggcgcagtggctcatgcctgtaatcccagcactttgggaggccgaggtgggcggatcacgaggtctggagtttgagaccatcctggctaacacagtgaaatcccgtctctactaaaaatacaaaaaattagccaggcgtggtggctggcacctgtagtcccagctacttgggaggctgaggcaggagaatggcgtgaacctggaaggaagaggttgcagtgagccaagattgcgcccctgcactccagcctgggcaacagagcaagactccatctcaaaaaaaaaaaaaaaaaaaaaaaagagttgttttctcatgttcattatagttcattacagttacatagtccgaaggtcttacaactaatcactggtagcaataaatgcttcaggcccacatgatgctgattagttctcagttttcattcagttcacaatataaccaccattcctgccctccctgccaagggtcataaatggtgactgcctaacaacaaaatttgcagtctcatctcattttcatccagacttctggaactcaaagattaacttttgactaaccctggaatatctcttatctcacttatagcttcaggcatgtatttatatgtattcttgatagcaataccataatcaatgtgtattcctgatagtaatgctacaataaatccaaacatttcaactctgtta

SEQ ID NO: 174 - DNA, 2508 bp, Homo sapiens
  source 1..2508: mol_type = genomic DNA
gcagacaggaagacttctgaagaacaaatcagcctggtcaccagcttttcggaacagcagagacacagagggcagtcatgagggtcccactctatcaccctggctggagtgtaatggtgtgatctcagctcactacaacttttgcctcctgggctcaagtgatcctcctgtctcagcctcccgaatggcgaggactacagtgaggtcaccaagaattccctggagaaaatccttccacagctgaaatgccatttcacctggaacttattcaaggaagacagtgtctcaagggatctagaagatagagtgtgtaaccagattgaatttttaaacactgagttcaaagctacaatgtacaacttgttggcctacataaaacacctagatggtaacaacgaggcagccctggaatgcttacggcaagctgaagagttaatccagcaagaacatgctgaccaagcagaaatcagaagtctagtcacttggggaaactacgcctgggtctactatcacttgggcagactctcagatgctcagatttatgtagataaggtgaaacaaacctgcaagaaattttcaaatccatacagtattgagtattctgaacttgactgtgaggaagggtggacacaactgaagtgtggaagaaatgaaagggcgaaggtgtgttttgagaaggctctggaagaaaagcccaacaacccagaattctcctctggactggcaattgcgatgtaccatctggataatcacccagagaaacagttctctactgatgttttgaagcaggccattgagctgagtcctgataaccaatacgtcaaggttctcttgggcctgaaactgcagaagatgaataaagaagctgaaggagagcagtttgttgaagaagccttggaaaagtctccttgccaaacagatgtcctccgcagtgcagccaaattttacagaagaaaaggtgacctagacaaagctattgaactgtttcaacgggtgttggaatccacaccaaacaatggctacctctatcaccagattgggtgctgctacaaggcaaaagtaagacaaatgcagaatacaggagaatctgaagctagtggaaataaagagatgattgaagcactaaagcaatatgctatggactattcgaataaagctcttgagaagggactgaatcctctgaatgcatactccgatctcgctgagttcctggagacggaatgttatcagacaccattcaataaggaagtccctgatgctgaaaagcaacaatcccatcagcgctactgcaaccttcagaaatataatgggaagtctgaagacactgctgtgcaacatggtttagagggtttgtccataagcaaaaaatcaactgacaaggaagagatcaaagaccaaccacagaatgtatctgaaaatctgcttccacaaaatgcaccaaattattggtatcttcaaggattaattcataagcagaatggagatctgctgcaagcagccaaatgttatgagaaggaactgggccgcctgctaagggatgccccttcaggcataggcagtattttcctgtcagcatctgagcttgaggatggtagtgaggaaatgggccagggcgcagtcagctccagtcccagagagctcctctctaactcagagcaactgaactgagacagaggaggaaaacagagcatcagaagcctgcagtggtggttgtgacgggtaggacgataggaagacagggggccccaacctgggattgctgagcagggaagctttgcatgttgctctaaggtacatttttaaagagttgttttttggccgggcgcagtggctcatgcctgtaatcccagcactttgggaggccgaggtgggcggatcacgaggtctggagtttgagaccatcctggctaacacagtgaaatcccgtctctactaaaaatacaaaaaattagccaggcgtggtggctggcacctgtagtcccagctacttgggaggctgaggcaggagaatggcgtgaacctggaaggaagaggttgcagtgagccaagattgcgcccctgcactccagcctgggcaacagagcaagactccatctcaaaaaaaaaaaaaaaaaaaaaaaagagttgttttctcatgttcattatagttcattacagttacatagtccgaaggtcttacaactaatcactggtagcaataaatgcttcaggcccacatgatgctgattagttctcagttttcattcagttcacaatataaccaccattcctgccctccctgccaagggtcataaatggtgactgcctaacaacaaaatttgcagtctcatctcattttcatccagacttctggaactcaaagattaacttttgactaaccctggaatatctcttatctcacttatagcttcaggcatgtatttatatgtattcttgatagcaataccataatcaatgtgtattcctgatagtaatgctacaataaatccaaacatttcaactctgtta

SEQ ID NO: 175 - DNA, 2529 bp, Homo sapiens
  source 1..2529: mol_type = genomic DNA
gtggaaacctcttcagcatttgcttggaatcagtaagctaaaaacaaaatcaaccgggaccccagcttttcagaactgcagggaaacagccatcatgaggcagggtcccactctatcaccctggctggagtgtaatggtgtgatctcagctcactacaacttttgcctcctgggctcaagtgatcctcctgtctcagcctcccgaatggcgaggactacagtgaggtcaccaagaattccctggagaaaatccttccacagctgaaatgccatttcacctggaacttattcaaggaagacagtgtctcaagggatctagaagatagagtgtgtaaccagattgaatttttaaacactgagttcaaagctacaatgtacaacttgttggcctacataaaacacctagatggtaacaacgaggcagccctggaatgcttacggcaagctgaagagttaatccagcaagaacatgctgaccaagcagaaatcagaagtctagtcacttggggaaactacgcctgggtctactatcacttgggcagactctcagatgctcagatttatgtagataaggtgaaacaaacctgcaagaaattttcaaatccatacagtattgagtattctgaacttgactgtgaggaagggtggacacaactgaagtgtggaagaaatgaaagggcgaaggtgtgttttgagaaggctctggaagaaaagcccaacaacccagaattctcctctggactggcaattgcgatgtaccatctggataatcacccagagaaacagttctctactgatgttttgaagcaggccattgagctgagtcctgataaccaatacgtcaaggttctcttgggcctgaaactgcagaagatgaataaagaagctgaaggagagcagtttgttgaagaagccttggaaaagtctccttgccaaacagatgtcctccgcagtgcagccaaattttacagaagaaaaggtgacctagacaaagctattgaactgtttcaacgggtgttggaatccacaccaaacaatggctacctctatcaccagattgggtgctgctacaaggcaaaagtaagacaaatgcagaatacaggagaatctgaagctagtggaaataaagagatgattgaagcactaaagcaatatgctatggactattcgaataaagctcttgagaagggactgaatcctctgaatgcatactccgatctcgctgagttcctggagacggaatgttatcagacaccattcaataaggaagtccctgatgctgaaaagcaacaatcccatcagcgctactgcaaccttcagaaatataatgggaagtctgaagacactgctgtgcaacatggtttagagggtttgtccataagcaaaaaatcaactgacaaggaagagatcaaagaccaaccacagaatgtatctgaaaatctgcttccacaaaatgcaccaaattattggtatcttcaaggattaattcataagcagaatggagatctgctgcaagcagccaaatgttatgagaaggaactgggccgcctgctaagggatgccccttcaggcataggcagtattttcctgtcagcatctgagcttgaggatggtagtgaggaaatgggccagggcgcagtcagctccagtcccagagagctcctctctaactcagagcaactgaactgagacagaggaggaaaacagagcatcagaagcctgcagtggtggttgtgacgggtaggacgataggaagacagggggccccaacctgggattgctgagcagggaagctttgcatgttgctctaaggtacatttttaaagagttgttttttggccgggcgcagtggctcatgcctgtaatcccagcactttgggaggccgaggtgggcggatcacgaggtctggagtttgagaccatcctggctaacacagtgaaatcccgtctctactaaaaatacaaaaaattagccaggcgtggtggctggcacctgtagtcccagctacttgggaggctgaggcaggagaatggcgtgaacctggaaggaagaggttgcagtgagccaagattgcgcccctgcactccagcctgggcaacagagcaagactccatctcaaaaaaaaaaaaaaaaaaaaaaaagagttgttttctcatgttcattatagttcattacagttacatagtccgaaggtcttacaactaatcactggtagcaataaatgcttcaggcccacatgatgctgattagttctcagttttcattcagttcacaatataaccaccattcctgccctccctgccaagggtcataaatggtgactgcctaacaacaaaatttgcagtctcatctcattttcatccagacttctggaactcaaagattaacttttgactaaccctggaatatctcttatctcacttatagcttcaggcatgtatttatatgtattcttgatagcaataccataatcaatgtgtattcctgatagtaatgctacaataaatccaaacatttcaactctgtta

SEQ ID NO: 176 - DNA, 2390 bp, Homo sapiens
  source 1..2390: mol_type = genomic DNA
gcagacaggaagacttctgaagaacaaatcagcctggtcaccagcttttcggaacagcagagacacagagggcagtcatgagtgaggtcaccaagaattccctggagaaaatccttccacagctgaaatgccatttcacctggaacttattcaaggaagacagtgtctcaagggatctagaagatagagtgtgtaaccagattgaatttttaaacactgagttcaaagctacaatgtacaacttgttggcctacataaaacacctagatggtaacaacgaggcagccctggaatgcttacggcaagctgaagagttaatccagcaagaacatgctgaccaagcagaaatcagaagtctagtcacttggggaaactacgcctgggtctactatcacttgggcagactctcagatgctcagatttatgtagataaggtgaaacaaacctgcaagaaattttcaaatccatacagtattgagtattctgaacttgactgtgaggaagggtggacacaactgaagtgtggaagaaatgaaagggcgaaggtgtgttttgagaaggctctggaagaaaagcccaacaacccagaattctcctctggactggcaattgcgatgtaccatctggataatcacccagagaaacagttctctactgatgttttgaagcaggccattgagctgagtcctgataaccaatacgtcaaggttctcttgggcctgaaactgcagaagatgaataaagaagctgaaggagagcagtttgttgaagaagccttggaaaagtctccttgccaaacagatgtcctccgcagtgcagccaaattttacagaagaaaaggtgacctagacaaagctattgaactgtttcaacgggtgttggaatccacaccaaacaatggctacctctatcaccagattgggtgctgctacaaggcaaaagtaagacaaatgcagaatacaggagaatctgaagctagtggaaataaagagatgattgaagcactaaagcaatatgctatggactattcgaataaagctcttgagaagggactgaatcctctgaatgcatactccgatctcgctgagttcctggagacggaatgttatcagacaccattcaataaggaagtccctgatgctgaaaagcaacaatcccatcagcgctactgcaaccttcagaaatataatgggaagtctgaagacactgctgtgcaacatggtttagagggtttgtccataagcaaaaaatcaactgacaaggaagagatcaaagaccaaccacagaatgtatctgaaaatctgcttccacaaaatgcaccaaattattggtatcttcaaggattaattcataagcagaatggagatctgctgcaagcagccaaatgttatgagaaggaactgggccgcctgctaagggatgccccttcaggcataggcagtattttcctgtcagcatctgagcttgaggatggtagtgaggaaatgggccagggcgcagtcagctccagtcccagagagctcctctctaactcagagcaactgaactgagacagaggaggaaaacagagcatcagaagcctgcagtggtggttgtgacgggtaggacgataggaagacagggggccccaacctgggattgctgagcagggaagctttgcatgttgctctaaggtacatttttaaagagttgttttttggccgggcgcagtggctcatgcctgtaatcccagcactttgggaggccgaggtgggcggatcacgaggtctggagtttgagaccatcctggctaacacagtgaaatcccgtctctactaaaaatacaaaaaattagccaggcgtggtggctggcacctgtagtcccagctacttgggaggctgaggcaggagaatggcgtgaacctggaaggaagaggttgcagtgagccaagattgcgcccctgcactccagcctgggcaacagagcaagactccatctcaaaaaaaaaaaaaaaaaaaaaaaagagttgttttctcatgttcattatagttcattacagttacatagtccgaaggtcttacaactaatcactggtagcaataaatgcttcaggcccacatgatgctgattagttctcagttttcattcagttcacaatataaccaccattcctgccctccctgccaagggtcataaatggtgactgcctaacaacaaaatttgcagtctcatctcattttcatccagacttctggaactcaaagattaacttttgactaaccctggaatatctcttatctcacttatagcttcaggcatgtatttatatgtattcttgatagcaataccataatcaatgtgtattcctgatagtaatgctacaataaatccaaacatttcaactctgtta

SEQ ID NO: 177 - DNA, 3805 bp, Homo sapiens
  source 1..3805: mol_type = genomic DNA
gtgacgtcagagcaggaagtgtttgaggaagtcgcgccgcgctgcccgcgttaagattcccgcattttaatgttttcaggggggtgtcatagccccgggtttggccgccccagccccgccttccccgccccggggagcccgccccctgccccgcgtccctgccgacaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatgatgtggggggactcgaccccctgacatggggcagcccatagcaggccttgtgcagtggggggactcgaccccctgacatggggctgcctggagcaggccgcgtgacgtggggctgcctggccgcggctctcacatggtggttcctgctgcactgatggcccaggggtctctggtatccagatggagctctcgcttcctcagcagctgtgactttcacccaggacccaggacgcagccctccgtgggcactgccggcgccttgtctgcacactggaggtcctccattacagaggcccagcgcacatcgctggccccacaaacgttcaggggtacagccatggcagctccttcctctgccgtgagaaaagtgcttggagtacggtttgccacacacgtgactggacagtgtccaattcaaatctttcagggcagagtccgagcagcgcttggtgacagcctgtcctctcctgctctccaaaggccctgctccctgtcctctctcactttacagcttgtgtttcttctggattcagcttctcctaaacagacagtttaattatagttgcggcctggccccatcctcacttcctctttttatttcactgctgctaaaattgtgtttttacctactactttggtggttgtcctcttttcggcaaagttggagcgagtgccaagctctccatctgtggtcctttctgccaagagcgactcatagtaaccaggatgggagagcagctgccttattctgaatcccaaaaattacttgggggtgattgtcacagaggagggacagaaagggtatctgctgaccaccagcctgcctacccatgcccatgtctccattcctgctcaagcgtgtgtgctgggccggggagtccctgtctctcacagcatctagcagtattattaaatggattcattttaaaaatagctcctatattttgtaacatgtctcaaacactcatactgggttccacaatccactgttagaatacctatggttagggcttctgaactaaaataatggaaaattttaacaatttgtatagtgcctggatcattactagtgccataaccctgcttcttcaacatttcacagaacttctcttttatataaaggcaagagcacaaaatgagttcagatgatcacaaacaggtgagttttgttggagaagaaagttggagtaggagactttcacaagtggtttccatggagatagaatgaagcattctgtggtcaagtaagtttagggagctattcatgtttcacttgctttgtggagattcacactatgcactgggaaagtatctgaaaagtcttataataaagaaacaggcttaactttgtgtaagaacactgtttatcaatgtcatttggctatagaaacattttctcctgctgattgtgtgtgtgaaacatgtattaacattccaatgaactagcatttaataaagcacaattttggaaa

SEQ ID NO: 178 - DNA, 3843 bp, Homo sapiens
  source 1..3843: mol_type = genomic DNA
gtgacgtcagagcaggaagtgtttgaggaagtcgcgccgcgctgcccgcgttaagattcccgcattttaatgttttcaggggggtgtcatagccccgggtttggccgccccagccccgccttccccgccccggggagcccgccccctgccccgcgtccctgccgacagagttagcacgacatcagtatgagctggtcaccttccctgacaacgcagacatgtggggcctgggaaatgaaagagcgccttgggacagggggatttggaaatgtcatccgatggcacaatcaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatgatgtggggggactcgaccccctgacatggggcagcccatagcaggccttgtgcagtggggggactcgaccccctgacatggggctgcctggagcaggccgcgtgacgtggggctgcctggccgcggctctcacatggtggttcctgctgcactgatggcccaggggtctctggtatccagatggagctctcgcttcctcagcagctgtgactttcacccaggacccaggacgcagccctccgtgggcactgccggcgccttgtctgcacactggaggtcctccattacagaggcccagcgcacatcgctggccccacaaacgttcaggggtacagccatggcagctccttcctctgccgtgagaaaagtgcttggagtacggtttgccacacacgtgactggacagtgtccaattcaaatctttcagggcagagtccgagcagcgcttggtgacagcctgtcctctcctgctctccaaaggccctgctccctgtcctctctcactttacagcttgtgtttcttctggattcagcttctcctaaacagacagtttaattatagttgcggcctggccccatcctcacttcctctttttatttcactgctgctaaaattgtgtttttacctactactttggtggttgtcctcttttcggcaaagttggagcgagtgccaagctctccatctgtggtcctttctgccaagagcgactcatagtaaccaggatgggagagcagctgccttattctgaatcccaaaaattacttgggggtgattgtcacagaggagggacagaaagggtatctgctgaccaccagcctgcctacccatgcccatgtctccattcctgctcaagcgtgtgtgctgggccggggagtccctgtctctcacagcatctagcagtattattaaatggattcattttaaaaatagctcctatattttgtaacatgtctcaaacactcatactgggttccacaatccactgttagaatacctatggttagggcttctgaactaaaataatggaaaattttaacaatttgtatagtgcctggatcattactagtgccataaccctgcttcttcaacatttcacagaacttctcttttatataaaggcaagagcacaaaatgagttcagatgatcacaaacaggtgagttttgttggagaagaaagttggagtaggagactttcacaagtggtttccatggagatagaatgaagcattctgtggtcaagtaagtttagggagctattcatgtttcacttgctttgtggagattcacactatgcactgggaaagtatctgaaaagtcttataataaagaaacaggcttaactttgtgtaagaacactgtttatcaatgtcatttggctatagaaacattttctcctgctgattgtgtgtgtgaaacatgtattaacattccaatgaactagcatttaataaagcacaattttggaaaccctggtaaa

SEQ ID NO: 179 - DNA, 3938 bp, Homo sapiens
  source 1..3938: mol_type = genomic DNA
gtgacgtcagagcaggaagtgtttgaggaagtcgcgccgcgctgcccgcgttaagattcccgcattttaatgttttcaggggggtgtcatagccccgggtttggccgccccagccccgccttccccgccccggggagcccgccccctgccccgcgtccctgccgacagagttagcacgacatcagtatgagctggtcaccttccctgacaacgcagacatgtggggcctgggaaatgaaagagcgccttgggacagggggatttggaaatgtcatccgatggcacaatcaggaaacaggtgagcagattgccatcaagcagtgccggcaggagctcagcccccggaaccgagagcggtggtgcctggagatccagatcatgagaaggctgacccaccccaatgtggtggctgcccgagatgtccctgaggggatgcagaacttggcgcccaatgacctgcccctgctggccatggagtactgccaaggaggagatctccggaagtacctgaaccagtttgagaactgctgtggtctgcgggaaggtgccatcctcaccttgctgagtgacattgcctctgcgcttagataccttcatgaaaacagaatcatccatcgggatctaaagccagaaaacatcgtcctgcagcaaggagaacagaggttaatacacaaaattattgacctaggatatgccaaggagctggatcagggcagtctttgcacatcattcgtggggaccctgcagtacctggccccagagctactggagcagcagaagtacacagtgaccgtcgactactggagcttcggcaccctggcctttgagtgcatcacgggcttccggcccttcctccccaactggcagcccgtgcagtggcattcaaaagtgcggcagaagagtgaggtggacattgttgttagcgaagacttgaatggaacggtgaagttttcaagctctttaccctaccccaataatcttaacagtgtcctggctgagcgactggagaagtggctgcaactgatgctgatgtggcacccccgacagaggggcacggatcccacgtatgggcccaatggctgcttcaaggccctggatgacatcttaaacttaaagctggttcatatcttgaacatggtcacgggcaccatccacacctaccctgtgacagaggatgagagtctgcagagcttgaaggccagaatccaacaggacacgggcatcccagaggaggaccaggagctgctgcaggaagcgggcctggcgttgatccccgataagcctgccactcagtgtatttcagacggcaagttaaatgagggccacacattggacatggatcttgtttttctctttgacaacagtaaaatcacctatgagactcagatctccccacggccccaacctgaaagtgtcagctgtatccttcaagagcccaagaggaatctcgccttcttccagctgaggaaggtgtggggccaggtctggcacagcatccagaccctgaaggaagattgcaaccggctgcagcagggacagcgagccgccatgatgaatctcctccgaaacaacagctgcctctccaaaatgaagaattccatggcttccatgtctcagcagctcaaggccaagttggatttcttcaaaaccagcatccagattgacctggagaagtacagcgagcaaaccgagtttgggatcacatcagataaactgctgctggcctggagggaaatggagcaggctgtggagctctgtgggcgggagaacgaagtgaaactcctggtagaacggatgatggctctgcagaccgacattgtggacttacagaggagccccatgggccggaagcaggggggaacgctggacgacctagaggagcaagcaagggagctgtacaggagactaagggaaaaacctcgagaccagcgaactgagggtgacagtcaggaaatggtacggctgctgcttcaggcaattcagagcttcgagaagaaagtgcgagtgatctatacgcagctcagtaaaactgtggtttgcaagcagaaggcgctggaactgttgcccaaggtggaagaggtggtgagcttaatgaatgaggatgagaagactgttgtccggctgcaggagaagcggcagaaggagctctggaatctcctgaagattgcttgtagcaaggtccgtggtcctgtcagtggaagcccggatagcatgaatgcctctcgacttagccagcctgggcagctgatgtctcagccctccacggcctccaacagcttacctgagccagccaagaagagtgaagaactggtggctgaagcacataacctctgcaccctgctagaaaatgccatacaggacactgtgagggaacaagaccagagtttcacggccctagactggagctggttacagacggaagaagaagagcacagctgcctggagcaggcctcatgatgtggggggactcgaccccctgacatggggcagcccatagcaggccttgtgcagtggggggactcgaccccctgacatggggctgcctggagcaggccgcgtgacgtggggctgcctggccgcggctctcacatggtggttcctgctgcactgatggcccaggggtctctggtatccagatggagctctcgcttcctcagcagctgtgactttcacccaggacccaggacgcagccctccgtgggcactgccggcgccttgtctgcacactggaggtcctccattacagaggcccagcgcacatcgctggccccacaaacgttcaggggtacagccatggcagctccttcctctgccgtgagaaaagtgcttggagtacggtttgccacacacgtgactggacagtgtccaattcaaatctttcagggcagagtccgagcagcgcttggtgacagcctgtcctctcctgctctccaaaggccctgctccctgtcctctctcactttacagcttgtgtttcttctggattcagcttctcctaaacagacagtttaattatagttgcggcctggccccatcctcacttcctctttttatttcactgctgctaaaattgtgtttttacctactactttggtggttgtcctcttttcggcaaagttggagcgagtgccaagctctccatctgtggtcctttctgccaagagcgactcatagtaaccaggatgggagagcagctgccttattctgaatcccaaaaattacttgggggtgattgtcacagaggagggacagaaagggtatctgctgaccaccagcctgcctacccatgcccatgtctccattcctgctcaagcgtgtgtgctgggccggggagtccctgtctctcacagcatctagcagtattattaaatggattcattttaaaaatagctcctatattttgtaacatgtctcaaacactcatactgggttccacaatccactgttagaatacctatggttagggcttctgaactaaaataatggaaaattttaacaatttgtatagtgcctggatcattactagtgccataaccctgcttcttcaacatttcacagaacttctcttttatataaaggcaagagcacaaaatgagttcagatgatcacaaacaggtgagttttgttggagaagaaagttggagtaggagactttcacaagtggtttccatggagatagaatgaagcattctgtggtcaagtaagtttagggagctattcatgtttcacttgctttgtggagattcacactatgcactgggaaagtatctgaaaagtcttataataaagaaacaggcttaactttgtgtaagaacactgtttatcaatgtcatttggctatagaaacattttctcctgctgattgtgtgtgtgaaacatgtattaacattccaatgaactagcatttaataaagcacaattttggaaaccctggtaaa

SEQ ID NO: 180 - DNA, 1611 bp, Homo sapiens
  source 1..1611: mol_type = genomic DNA
ataggggcgggaacagcccagcgggccgtcccatcggcttttgggtctgttacccaaagaatgataaagttggttttatttcaagaagtcgatcaaaaagaaagccccagcgctctagagctcagctgacgggaaagggggtgcgcagcctcgagtttgagagctacccggagctccaagacaggggtgggttccagctgcccgcacgccccgaccttccatcgtaggccggaccatgggaaccccaaagccacggatcctgccctggctggtgtcgcagctggacctggggcaactggagggcgtggcctgggtgaacaagagccgcacgcgcttccgcatcccttggaagcacggcctacggcaggatgcacagcaggaggatttcggaatcttccaggcctgggccgaggccactggtgcatatgttcccgggagggataagccagacctgccaacctggaagaggaatttccgctctgccctcaaccgcaaagaagggttgcgtttagcagaggaccggagcaaggaccctcacgacccacataaaatctacgagtttgtgaactcaggagttggggacttttcccagccagacacctctccggacaccaatggtggaggcagtacttctgatacccaggaagacattctggatgagttactgggtaacatggtgttggccccactcccagatccgggacccccaagcctggctgtagcccctgagccctgccctcagcccctgcggagccccagcttggacaatcccactcccttcccaaacctggggccctctgagaacccactgaagcggctgttggtgccgggggaagagtgggagttcgaggtgacagccttctaccggggccgccaagtcttccagcagaccatctcctgcccggagggcctgcggctggtggggtccgaagtgggagacaggacgctgcctggatggccagtcacactgccagaccctggcatgtccctgacagacaggggagtgatgagctacgtgaggcatgtgctgagctgcctgggtgggggactggctctctggcgggccgggcagtggctctgggcccagcggctggggcactgccacacatactgggcagtgagcgaggagctgctccccaacagcgggcatgggcctgatggcgaggtccccaaggacaaggaaggaggcgtgtttgacctggggcccttcattgtaggctcctgggcccccagatctgattaccttcacggaaggaagcggacgctcaccacgctatgccctctggttctgtgtgggggagtcatggccccaggaccagccgtggaccaagaggctcgtgatggtcaaggttgtgcccacgtgcctcagggccttggtagaaatggcccgggtagggggtgcctcctccctggagaatactgtggacctgcacatttccaacagccacccactctccctcacctccgaccagtacaaggcctacctgcaggacttggtggagggcatggatttccagggccctggggagagctgagccctcgctcctcatggtgtgcctccaacccccctgttccccaccacctcaaccaataaactggttcctgctatga

SEQ ID NO: 181 - DNA, 1653 bp, Homo sapiens
  source 1..1653: mol_type = genomic DNA
ataggggcgggaacagcccagcgggccgtcccatcggcttttgggtctgttacccaaagaatgataaagttggttttatttcaagaagtcgatcaaaaagaaagccccagcgctctagagctcagctgacgggaaagggggtgcgcagcctcgagtttgagagctacccggagctccaagacaggggtgggttccagctgcccgcacgccccgaccttccatcgtaggtgcgtgcgacgacctgggacgctggaaacccggagctgcgcgctgggcggaaaggactcctagtgggcccggctgagaaggcggaggtggcgcgcgaggggcgggctcgtgccaacgctggtgggtggggctagaacgggaggggcggggctagccggatccgctgactgatggtggggcgcaagcagggttggagctgcagggttacgggaataaaaccgggaaagccggcctgggccgaggccactggtgcatatgttcccgggagggataagccagacctgccaacctggaagaggaatttccgctctgccctcaaccgcaaagaagggttgcgtttagcagaggaccggagcaaggaccctcacgacccacataaaatctacgagtttgtgaactcaggagttggggacttttcccagccagacacctctccggacaccaatggtggaggcagtacttctgatacccaggaagacattctggatgagttactgggtaacatggtgttggccccactcccagatccgggacccccaagcctggctgtagcccctgagccctgccctcagcccctgcggagccccagcttggacaatcccactcccttcccaaacctggggccctctgagaacccactgaagcggctgttggtgccgggggaagagtgggagttcgaggtgacagccttctaccggggccgccaagtcttccagcagaccatctcctgcccggagggcctgcggctggtggggtccgaagtgggagacaggacgctgcctggatggccagtcacactgccagaccctggcatgtccctgacagacaggggagtgatgagctacgtgaggcatgtgctgagctgcctgggtgggggactggctctctggcgggccgggcagtggctctgggcccagcggctggggcactgccacacatactgggcagtgagcgaggagctgctccccaacagcgggcatgggcctgatggcgaggtccccaaggacaaggaaggaggcgtgtttgacctggggcccttcattgtagatctgattaccttcacggaaggaagcggacgctcaccacgctatgccctctggttctgtgtgggggagtcatggccccaggaccagccgtggaccaagaggctcgtgatggtcaaggttgtgcccacgtgcctcagggccttggtagaaatggcccgggtagggggtgcctcctccctggagaatactgtggacctgcacatttccaacagccacccactctccctcacctccgaccagtacaaggcctacctgcaggacttggtggagggcatggatttccagggccctggggagagctgagccctcgctcctcatggtgtgcctccaacccccctgttccccaccacctcaaccaataaactggttcctgctatga

SEQ ID NO: 182 - DNA, 1214 bp, Homo sapiens
  source 1..1214: mol_type = genomic DNA
ataggggcgggaacagcccagcgggccgtcccatcggcttttgggtctgttacccaaagaatgataaagttggttttatttcaagaagtcgatcaaaaagaaagccccagcgctctagagctcagctgacgggaaagggggtgcgcagcctcgagtttgagagctacccggagctccaagacaggggtgggttccagctgcccgcacgccccgaccttccatcgtaggccggaccatgggaaccccaaagccacggatcctgccctggctggtgtcgcagctggacctggggcaactggagggcgtggcctgggtgaacaagagccgcacgcgcttccgcatcccttggaagcacggcctacggcaggatgcacagcaggaggatttcggaatcttccaggcctgggccgaggccactggtgcatatgttcccgggagggataagccagacctgccaacctggaagaggaatttccgctctgccctcaaccgcaaagaagggttgcgtttagcagaggaccggagcaaggaccctcacgacccacataaaatctacgagtttgtgaactcaggagttggggacttttcccagccagacacctctccggacaccaatggtggaggcagtacttctgatacccaggaagacattctggatgagttactgggtaacatggtgttggccccactcccagatccgggacccccaagcctggctgtagcccctgagccctgccctcagcccctgcggagccccagcttggacaatcccactcccttcccaaacctggggccctctgagaacccactgaagcggctgttggtgccgggggaagatctgattaccttcacggaaggaagcggacgctcaccacgctatgccctctggttctgtgtgggggagtcatggccccaggaccagccgtggaccaagaggctcgtgatggtcaaggttgtgcccacgtgcctcagggccttggtagaaatggcccgggtagggggtgcctcctccctggagaatactgtggacctgcacatttccaacagccacccactctccctcacctccgaccagtacaaggcctacctgcaggacttggtggagggcatggatttccagggccctggggagagctgagccctcgctcctcatggtgtgcctccaacccccctgttccccaccacctcaaccaataaactggttcctgctatga

SEQ ID NO: 183 - DNA, 1422 bp, Homo sapiens
  source 1..1422: mol_type = genomic DNA
ataggggcgggaacagcccagcgggccgtcccatcggcttttgggtctgttacccaaagaatgataaagttggttttatttcaagaagtcgatcaaaaagaaagccccagcgctctagagctcagctgacgggaaagggggtgcgcagcctcgagtttgagagctacccggagctccaagacaggggtgggttccagctgcccgcacgccccgaccttccatcgtaggcctgggccgaggccactggtgcatatgttcccgggagggataagccagacctgccaacctggaagaggaatttccgctctgccctcaaccgcaaagaagggttgcgtttagcagaggaccggagcaaggaccctcacgacccacataaaatctacgagtttgtgaactcaggagttggggacttttcccagccagacacctctccggacaccaatggtggaggcagtacttctgatacccaggaagacattctggatgagttactgggtaacatggtgttggccccactcccagatccgggacccccaagcctggctgtagcccctgagccctgccctcagcccctgcggagccccagcttggacaatcccactcccttcccaaacctggggccctctgagaacccactgaagcggctgttggtgccgggggaagagtgggagttcgaggtgacagccttctaccggggccgccaagtcttccagcagaccatctcctgcccggagggcctgcggctggtggggtccgaagtgggagacaggacgctgcctggatggccagtcacactgccagaccctggcatgtccctgacagacaggggagtgatgagctacgtgaggcatgtgctgagctgcctgggtgggggactggctctctggcgggccgggcagtggctctgggcccagcggctggggcactgccacacatactgggcagtgagcgaggagctgctccccaacagcgggcatgggcctgatggcgaggtccccaaggacaaggaaggaggcgtgtttgacctggggcccttcattgtagatctgattaccttcacggaaggaagcggacgctcaccacgctatgccctctggttctgtgtgggggagtcatggccccaggaccagccgtggaccaagaggctcgtgatggtcaaggttgtgcccacgtgcctcagggccttggtagaaatggcccgggtagggggtgcctcctccctggagaatactgtggacctgcacatttccaacagccacccactctccctcacctccgaccagtacaaggcctacctgcaggacttggtggagggcatggatttccagggccctggggagagctgagccctcgctcctcatggtgtgcctccaacccccctgttccccaccacctcaaccaataaactggttcctgctatga

SEQ ID NO: 184 - DNA, 1250 bp, Homo sapiens
  source 1..1250: mol_type = genomic DNA
ataggggcgggaacagcccagcgggccgtcccatcggcttttgggtctgttacccaaagaatgataaagttggttttatttcaagaagtcgatcaaaaagaaagccccagcgctctagagctcagctgacgggaaagggggtgcgcagcctcgagtttgagagctacccggagctccaagacaggggtgggttccagctgcccgcacgccccgaccttccatcgtaggagttggggacttttcccagccagacacctctccggacaccaatggtggaggcagtacttctgatacccaggaagacattctggatgagttactgggtaacatggtgttggccccactcccagatccgggacccccaagcctggctgtagcccctgagccctgccctcagcccctgcggagccccagcttggacaatcccactcccttcccaaacctggggccctctgagaacccactgaagcggctgttggtgccgggggaagagtgggagttcgaggtgacagccttctaccggggccgccaagtcttccagcagaccatctcctgcccggagggcctgcggctggtggggtccgaagtgggagacaggacgctgcctggatggccagtcacactgccagaccctggcatgtccctgacagacaggggagtgatgagctacgtgaggcatgtgctgagctgcctgggtgggggactggctctctggcgggccgggcagtggctctgggcccagcggctggggcactgccacacatactgggcagtgagcgaggagctgctccccaacagcgggcatgggcctgatggcgaggtccccaaggacaaggaaggaggcgtgtttgacctggggcccttcattgtagatctgattaccttcacggaaggaagcggacgctcaccacgctatgccctctggttctgtgtgggggagtcatggccccaggaccagccgtggaccaagaggctcgtgatggtcaaggttgtgcccacgtgcctcagggccttggtagaaatggcccgggtagggggtgcctcctccctggagaatactgtggacctgcacatttccaacagccacccactctccctcacctccgaccagtacaaggcctacctgcaggacttggtggagggcatggatttccagggccctggggagagctgagccctcgctcctcatggtgtgcctccaacccccctgttccccaccacctcaaccaataaactggttcctgctatga

SEQ ID NO: 185 - DNA, 1041 bp, Homo sapiens
  source 1..1041: mol_type = genomic DNA
ataggggcgggaacagcccagcgggccgtcccatcggcttttgggtctgttacccaaagaatgataaagttggttttatttcaagaagtcgatcaaaaagaaagccccagcgctctagagctcagctgacgggaaagggggtgcgcagcctcgagtttgagagctacccggagctccaagacaggggtgggttccagctgcccgcacgccccgaccttccatcgtaggcctgggccgaggccactggtgcatatgttcccgggagggataagccagacctgccaacctggaagaggaatttccgctctgccctcaaccgcaaagaagggttgcgtttagcagaggaccggagcaaggaccctcacgacccacataaaatctacgagtttgtgaactcaggagttggggacttttcccagccagacacctctccggacaccaatggtggaggcagtacttctgatacccaggaagacattctggatgagttactgggtaacatggtgttggccccactcccagatccgggacccccaagcctggctgtagcccctgagccctgccctcagcccctgcggagccccagcttggacaatcccactcccttcccaaacctggggccctctgagaacccactgaagcggctgttggtgccgggggaagatctgattaccttcacggaaggaagcggacgctcaccacgctatgccctctggttctgtgtgggggagtcatggccccaggaccagccgtggaccaagaggctcgtgatggtcaaggttgtgcccacgtgcctcagggccttggtagaaatggcccgggtagggggtgcctcctccctggagaatactgtggacctgcacatttccaacagccacccactctccctcacctccgaccagtacaaggcctacctgcaggacttggtggagggcatggatttccagggccctggggagagctgagccctcgctcctcatggtgtgcctccaacccccctgttccccaccacctcaaccaataaactggttcctgctatga

SEQ ID NO: 186 - DNA, 869 bp, Homo sapiens
  source 1..869: mol_type = genomic DNA
ataggggcgggaacagcccagcgggccgtcccatcggcttttgggtctgttacccaaagaatgataaagttggttttatttcaagaagtcgatcaaaaagaaagccccagcgctctagagctcagctgacgggaaagggggtgcgcagcctcgagtttgagagctacccggagctccaagacaggggtgggttccagctgcccgcacgccccgaccttccatcgtaggagttggggacttttcccagccagacacctctccggacaccaatggtggaggcagtacttctgatacccaggaagacattctggatgagttactgggtaacatggtgttggccccactcccagatccgggacccccaagcctggctgtagcccctgagccctgccctcagcccctgcggagccccagcttggacaatcccactcccttcccaaacctggggccctctgagaacccactgaagcggctgttggtgccgggggaagatctgattaccttcacggaaggaagcggacgctcaccacgctatgccctctggttctgtgtgggggagtcatggccccaggaccagccgtggaccaagaggctcgtgatggtcaaggttgtgcccacgtgcctcagggccttggtagaaatggcccgggtagggggtgcctcctccctggagaatactgtggacctgcacatttccaacagccacccactctccctcacctccgaccagtacaaggcctacctgcaggacttggtggagggcatggatttccagggccctggggagagctgagccctcgctcctcatggtgtgcctccaacccccctgttccccaccacctcaaccaataaactggttcctgctatga

SEQ ID NO: 187 - DNA, 1595 bp, Homo sapiens
  source 1..1595: mol_type = genomic DNA
ataggggcgggaacagcccagcgggccgtcccatcggcttttgggtctgttacccaaagaatgataaagttggttttatttcaagaagtcgatcaaaaagaaagccccagcgctctagagctcagctgacgggaaagggggtgcgcagcctcgagtttgagagctacccggagctccaagacaggggtgggttccagctgcccgcacgccccgaccttccatcgtaggccggaccatgggaaccccaaagccacggatcctgccctggctggtgtcgcagctggacctggggcaactggagggcgtggcctgggtgaacaagagccgcacgcgcttccgcatcccttggaagcacggcctacggcaggatgcacagcaggaggatttcggaatcttccaggcctgggccgaggccactggtgcatatgttcccgggagggataagccagacctgccaacctggaagaggaatttccgctctgccctcaaccgcaaagaagggttgcgtttagcagaggaccggagcaaggaccctcacgacccacataaaatctacgagtttgtgaactcaggagttggggacttttcccagccagacacctctccggacaccaatggtggaggcagtacttctgatacccaggaagacattctggatgagttactgggtaacatggtgttggccccactcccagatccgggacccccaagcctggctgtagcccctgagccctgccctcagcccctgcggagccccagcttggacaatcccactcccttcccaaacctggggccctctgagaacccactgaagcggctgttggtgccgggggaagagtgggagttcgaggtgacagccttctaccggggccgccaagtcttccagcagaccatctcctgcccggagggcctgcggctggtggggtccgaagtgggagacaggacgctgcctggatggccagtcacactgccagaccctggcatgtccctgacagacaggggagtgatgagctacgtgaggcatgtgctgagctgcctgggtgggggactggctctctggcgggccgggcagtggctctgggcccagcggctggggcactgccacacatactgggcagtgagcgaggagctgctccccaacagcgggcatgggcctgatggcgaggtccccaaggacaaggaaggaggcgtgtttgacctggggcccttcattgtagatctgattaccttcacggaaggaagcggacgctcaccacgctatgccctctggttctgtgtgggggagtcatggccccaggaccagccgtggaccaagaggctcgtgatggtcaaggttgtgcccacgtgcctcagggccttggtagaaatggcccgggtagggggtgcctcctccctggagaatactgtggacctgcacatttccaacagccacccactctccctcacctccgaccagtacaaggcctacctgcaggacttggtggagggcatggatttccagggccctggggagagctgagccctcgctcctcatggtgtgcctccaacccccctgttccccaccacctcaaccaataaactggttcctgctatga

SEQ ID NO: 188 - DNA, 1969 bp, Homo sapiens
  source 1..1969: mol_type = genomic DNA
gaaactcccgcctggccaccataaaagcgccggccctccgcttccccgcgagacgaaacttcccgtcccggcggctctggcacccagggtccggcctgcgccttcccgccaggcctggacactggttcaacacctgtgacttcatgtgtgcgcgccggccacacctgcagtcacacctgtagccccctctgccaagagatccataccgaggcagcgtcggtggctacaagccctcagtccacacctgtggacacctgtgacacctggccacacgacctgtggccgcggcctggcgtctgctgcgacaggagcccttacctcccctgttataacacctgaccgccacctaactgcccctgcagaaggagcaatggccttggctcctgagagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgggataactcgggggacccggccgacccgcacaaggtgtacgcgctcagccgggagctgtgctggcgaggcccaggcacggaccagactgaggcagaggcccccgcagctgtcccaccaccacagggtgggcccccagggccattcctggcacacacacatgctggactccaagccccaggccccctccctgccccagctggtgacaagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcccagggctccctgctggggagctgtacgggtgggcagtagagacgacccccagccccgggccccagcccgcggcactaacgacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctagaacccagtctaatgagaactccagaaagctggagcagcccacctagagctggccgcggccgcccagtctaataaaaagaactccagaaca

SEQ ID NO: 189 - DNA, 2034 bp, Homo sapiens
  source 1..2034: mol_type = genomic DNA
ataaaagcgccggccctccgcttccccgcgagacgaaacttcccgtcccggcggctctggcacccagggtccggcctgcgccttcccgccaggcctggacactggttcaacacctgtgacttcatgtgtgcgcgccggccacacctgcagtcacacctgtagccccctctgccaagagatccataccgaggcagcgtcggtggctacaagccctcagtccacacctgtggacacctgtgacacctggccacacgacctgtggccgcggcctggcgtctgctgcgacaggagcccttacctcccctgttataacacctgaccgccacctaactgcccctgcagaaggagcaatggccttggctcctgagaggtaagagcccggcccaccctctccagatgccagtccccgagcgccctgcagccggccctgactctccgcggccgggcacccgcagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgggataactcgggggacccggccgacccgcacaaggtgtacgcgctcagccgggagctgtgctggcgaggcccaggcacggaccagactgaggcagaggcccccgcagctgtcccaccaccacagggtgggcccccagggccattcctggcacacacacatgctggactccaagccccaggccccctccctgccccagctggtgacaagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcccagggctccctgctggggagctgtacgggtgggcagtagagacgacccccagccccgggccccagcccgcggcactaacgacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctagaacccagtctaatgagaactccagaaagctggagcagcccacctagagctggccgcggccgcccagtctaataaaaagaactccagaaca

SEQ ID NO: 190 - DNA, 1950 bp, Homo sapiens
  source 1..1950: mol_type = genomic DNA
ataaaagcgccggccctccgcttccccgcgagacgaaacttcccgtcccggcggctctggcacccagggtccggcctgcgccttcccgccaggcctggacactggttcaacacctgtgacttcatgtgtgcgcgccggccacacctgcagtcacacctgtagccccctctgccaagagatccataccgaggcagcgtcggtggctacaagccctcagtccacacctgtggacacctgtgacacctggccacacgacctgtggccgcggcctggcgtctgctgcgacaggagcccttacctcccctgttataacacctgaccgccacctaactgcccctgcagaaggagcaatggccttggctcctgagaggtaagagcccggcccaccctctccagatgccagtccccgagcgccctgcagccggccctgactctccgcggccgggcacccgcagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgggataactcgggggacccggccgacccgcacaaggtgtacgcgctcagccgggagctgtgctggcgagaaggcccaggcacggaccagactgaggcagaggcccccgcagctgtcccaccaccacagggtgggcccccagggccattcctggcacacacacatgctggactccaagccccaggccccctccctgccccagctggtgacaagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctagaacccagtctaatgagaactccagaaagctggagcagcccacctagagctggccgcggccgcccagtctaataaaaagaactccagaaca

SEQ ID NO: 191 - DNA, 850 bp, Homo sapiens
  source 1..850: mol_type = genomic DNA
tcccaccaccacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctagaacccagtctaatgagaactccagaaagctggagcagcccacctagagctggccgcggccgcccagtctaataaaaagaactccagaaca

SEQ ID NO: 192 - DNA, 11556 bp, Homo sapiens
  source 1..11556: mol_type = genomic DNA
ctcctcgctgcgggaagggtcctgggccccgggcggcggtcgccaggtctcagggccgggggtacccgagtctcgtttcctctcagtccatccacccttcatggggccagagccctctctccagaatctgagcagcaatgccgtttgctgaagacaagacctataagtatatctgccgcaatttcagcaatttttgcaatgtggatgttgtagagattctgccttacctgccctgcctcacagcaagagaccagggacctcggaccgtcccccagacccactggagccaccgtcacttcctgctgagaggccagggccccccacacctgctgcggcccacagcatcccctacaacagctgcagagagaaggagccaagttaccccatgcctgtccaggagacccaggcgccagagtccccaggagagaattcagagcaagccctgcagacgctcagccccagagccatcccaaggaatccagatggtggccccctggagtcctcctctgacctggcagccctcagccctctgacctccagcgggcatcaggagcaggacacagaactgggcagtacccacacagcaggtgcgacctccagcctcacaccatcccgtgggcctgtgtctccatctgtctccttccagcccctggcccgttccacccccagggcaagccgcttgcctggacccacagggtcagttgtatctactggcacctccttctcctcctcatcccctggcttggcctctgcaggggctgcagagggtaaacagggtgcagagagtgaccaggccgagcctatcatctgctccagtggggcagaggcacctgccaactctctgccctccaaagtgcctaccaccttgatgcctgtgaacacagtggccctgaaagtgcctgccaacccagcatctgtcagcacagtgccctccaagttgccaactagctcaaagccccctggtgcagtgccttctaatgcgctcaccaatccagcaccatccaaattgcccatcaactcaacccgtgctggcatggtgccatccaaagtgcctactagcatggtgctcaccaaggtgtctgccagcacagtccccactgacgggagcagcagaaatgaggagaccccagcagctccaacacccgccggcgccactggaggcagctcagcctggctagacagcagctctgagaataggggccttgggtcggagctgagtaagcctggcgtgctggcatcccaggtagacagcccgttctcgggctgcttcgaggatcttgccatcagtgccagcacctccttgggcatggggccctgccatggcccagaggagaatgagtataagtccgagggcacctttgggatccacgtggctgagaaccccagcatccagctcctggagggcaaccctgggccacctgcggacccggatggcggccccaggccacaagccgaccggaagttccaggagagggaggtgccatgccacaggccctcacctggggctctgtggctccaggtggctgtgacaggggtgctggtagtcacactcctggtggtgctgtaccggcggcgtctgcactagtgaagccctgggctcttcccaccacccatctgttccgttcctgcagtacacctggcccctctccgaagccccttgtccctttcttggggattgtggaggctgggtcagaggggagttaagggactgcaggcctggcagcaggacatgccttggctgaaccaagtcctgagagcagcatctctgtccccacggtgccttgtgtgggtccccgtccttggctttctgggtcctgggctgcccccagtgctccagaccttccccactggcaatccaggttatcatccatgtcctccagaggagcttcctcctccaggcctcagccctgttggcccaggtggagcaggagggaccactggaacatgtggtgcttgggaatgcctctcctgttgcattggtccctgaaggcctcagggcaggtatgtggtgtgtgggcgactccacaagacctgcctcccatcctggcagcccagcctgagaccgttgcattgaggcaggcaggagcggcagggtggctgctctccaggagcccaactgccttgagttcctgccccactgggccccctcccctgctgggcaatcctgggaaggtctggaggttcctgtggacctcagggaagccaggggcagctgtcaggcctgaggaagacctgtggagctcctctccagcctcctctttccctcccctctggtctccattctcttcagctccctacatgggctggggaggagacacctggtgggcagagctcaggcagaggtttggatttcagctccctcacttccggggctgtgtggctttggcagatgtcagacttctggtcttgcttctccacgtggacagtgagtatctggctcattcttcactgggttcttctgagattgaacctacaggtgtttgccaagtgcctggcccagagcaagtggccactgcttctcccatctctctcctgcccaacctggtagagctgagggcatgagaggcagagtgcacagtggtcaagggtgcagctctgcagcacaggcagcctaggcctgcgtcccaacctgcctctcaccagctctgtgaccttgggcaagggatttatctgtctgtcccttagttttctcacctgtaaaaggaggataagtatatatatatatttcccagtgttgtgaagattaaaggagtttatcgatgtaggtcttaggatgagtcctggcatttaccaagggttggatatatgttattatcactattaagtgttgagggtccaggcatgctgggcaacagggaccccatctctacaaaaaagtttaaaaaattagccaggcgtggtggtgcacctgtcgtcttagctacttgggaggctgaggtgggaggatcacttgagcccagaagcttgaagctgcagtgagctaggatcgtgccactgcactccaacctgggtgagagagcgagaccctgtctcaagaaaaagaaaaatgcagagaaacaggagtcttggctactcctttagaggcagactcagaccctcctgcctcacagctttatctttgtatttgccccttactttatcttgtgccttgagaaattgctggggagagaggtatgtccactgggcagctgtacaggatggaggatatagggcgtttccactcccagcagccaggttccctcaccccaagctcacccactgttggggagattatctacaataacaccagaaacacattggggtggattgggggtatccttatgggttcttttcagggaaccattgctggacaaggcacaggagccacctccatttctgagctctgcaagggacaagaactagagccatcaggggctgggctcactgtggccccaccccaagccgtcagcctccagggatctacaccctgccttggctgctacagctttttcactccactgccctaggggagttcagcaacctaatgatctctatctctgaacatctcttcatcccatgctccaagtccagcaacctgcaccctggaaccaggagtggaccctacccgagctgtctgtattaatccccatcccccaccaccaatcttaaaaagccctctgtccccctaccctaaaccccagttaggtacccatgctgggcaggtcagttaacaatttatgcacaggtactagttttattgtattaccgttccagggtagctttgaaaaaagtatctcaaaaaggcaacatgggccgagcgcagtggctcacgcctgtaatcccagcactttgggaggccaaggtgggcagatcgcctgaggtctggagttcaagaccagcctggccaacagggtgaaaccccgtctctacaaaaataagaaaattagccaggtgtagtggcagacgtctgtaatcccagctattcaggaggctgaggcacgagaattccatgaacccaggatgcggaggttgcagtgagccgagattgtgccactgcgctccagcctgggcgacagagtggtattctgtttcaaaaaaaaaaaaaaaggcagtatgtagccccgaagactgttgcccaagtggtagaatgttagcacactaccagcctaggtaaaaaatacaaaaagtaactgggcatggcggcgcccatctatagtcccagctacatgggaggctgaggtgggaagataagtcacttgagcccgccaggaggcggaggttgtagtgagctgagatcgcaccactgcactccagcctgggtgaccgagtgatactctgtctcaaagaaaaaaaattataattttagcacagtaaccagccatgatgggagataccctgggtaaggcatgtagaaagggttgagggaccttcccagtcccctagccccgcctcccatcctcccatctttttcttttttcttttttttagagaatcacccagcctggagcgaagtggtgcaatcataactcactgtatccttaaactcccgggcttaagcgatcctcctgcctcagccttctgagtaactaggacttcaggtacctgtcaccatgcctggctaattaaatttttttttcttttttttttttgagatggagtcttgctctgtcacccaggctggagtgcagtggcgcgatctcagctcactgcgacctccacctcctgggttcaggccattctcccgcctcagcctccagagtagctgggactacaggcgcctgccaccacgcctggctaatttttttgcacttttagtagagacggggtttcactgtgttagccaggatggtctcgatctcctgaccttgtgatccgcccgcctcggcctcccaaagtgctgggattacaggcgtgagccaccgcgcccagccaaattaaattttttatagagatgaggtcatgctgttatgttgcccaggttggcctcatgagatcttgccttagcctcccaaagtgctgggattacagatgtgagacactgcacccaaaccccaccacttttttttttcctttttctttttttgagacagtcttactccgttgcccaggctggagtgtagtggcatgatctcagctcactgcaacctccgcctcccgggttcaagcaattctcctgcctcagcctcccgagtagctgggattacagaggcctgccaccacacccgactaattttcgtatttttagtagagacggggtttctccatgttggccaggctgttcttgaactcctgacctcaagtgctccacctgcgttggcttcccaaagtgctgggatacaggagtgagccactgcgcctggctgatcccagcacttttcaaatgatgccgctcaaagccgtgacttggcctactttgaacagcaaacttgttgctgctgttgtcaacctgaaggcctctcaaatgccagcttcaagcagggtgtgaattggccagtgtcagatctcaggagtcctgtgttgagagtgtggctttcagctgcggggagctgcacttggtggggaaagccaggcaggtcaccctcacagccagataatgtggaggtcagaacccaaggaagggagtgagacctccactcccagtgggggacctggccacccatccttggggacctgagaaagcgtacttcaccttggggtgaaggctgggtggggccagagggaccagtgccctcctcagtgcttaggggcagagccacctgcagcaatggtatctgcatattagcccctctccaccttctttctcccgctgaatcatttccctcaaagcccaagagctgtcactgcttctttctccctgggaagaatgcgtggactctgcctggtgatagactgaagccagaacagtgccacaccctcgccttaattccttgctaggtgttctcagatttatgagacttcttagtcaaatatgagggaggttggatgtggtggcttgtgcctgtaatcccagcattttgggaagccgaggtgggaggatcccttgaagccaggagtttgagacaagcctgggcaacaaagcaagaccctatctctaaaaaaaaaaaaaaaaaaaaaaaaaaaaaaaaaaaaaaaaaatctaggagatgctctttaccctgcctggcctcaaactattaatagcttcctttgagcaacattatttattatgaactttcaaacacaaaaaagtagagagagtagaataacaaatccccatgagcccatcacccaacttcagtaattatcaattcatggccatcttgttcacccctgcctgcttccctgcttcccctcattctgcagaggttcttttcttttgagacagagtgttgctctgttgcccaggctggagtgcagtggtgcaacttcggctcactgcaacctccgcctcccaggttcaagtgattctcctgcttcagcctctcaagtagctgggattacagatgcccgccaccacacctggctaattttcgtatttttgttagagatggggtttcaccatgttggccaggctggtctcgaactcctgacctcaagtgatccgcccgccttggcctcccaaaatgctgggattacaggtgtgaaccacggtgcctggccactgtacaggttatttatagaagttggagagtgaagggttgagaaagccaaggggcagatgcgggtctggaggattttgtgcctaaggccctctctttgctcccagacagcatgaagtaacaatgaggcatccacctcttggttttgtggcctctgtggatgacgtctctcaccttgaaccagttcagagttggagtagcgcaggatcctgtcttcagaggaggggccgaagcgggttcctctgttgtcaagctctttggaggtgcctggctgctactactgtcccagagaggtgatgatgaatgatgggtgtgtccagtggcagtttgccccactgaggcaggggcttccactaggccctgacagagcccttccagcaggcagaaatccctgtgctaggcaagattcaaactccgtagcatgtctcctgctcccatctcttaggaatggagtccttcaggccttgagtcccacattttccatgatgctccattaagcagctgatagcacccccacctccagggaaagtgagttcagagtccttggtctaatgcatctgtgttgaaattgaggccttcccctgtgttcacctttctgctctttttcttttagcccaaggctatgaaggcctcattcggtgctgggcatggtcactcctagcattcctcactctgttgctaacagcaacagcaataataataagggttacaacttactccataccttactgtctgccaggcattaagctaagtgctttacatatattaagtcatttaatcctcataatgaccctatgaaagagataccatctcaacccaattgacagctggtttgcaagattaggagggatgaaggacccaggggacaatgcgagggaaaactctgaccccggggccccaggctggatgttctttatgcctgtgaaccacagcttatcacatgtctggagttagggaccccacttaaagtgagattttggctggaggtggtggatcatacctataatcccagcactttgggagaccaaggcagaaggactgcttgaggccaggagttcaaaaccagtgtaggtaacagctagaccctatctctacaaaaaatttaaaaattagctgggtgtggtggtatgtgcctcaagttccagctactcaggaggctgaggtgggaggatcacttgagcacaggagtttgaagttacagtgagctatgatggcaccactgcacttcagcctaggcaacagagggagaccctgtctttaaagtacatagaggtttttcacaccaacacatctctgcccagtgtgccaacatctgccacctgctataatagtactataacactcaatatgtaattaatgtagtctcagggatgttatgacaatatgattacaactatcacgtgtgtgcccagccaggctcaatgccccaggctgggcgaggtggggcaggggacacagcctaaaatgccaggcctcaggaagccatttggtttagcagacattgtttattaaaggagttacctatgccagatcgaaggcctaagatgattaagacactatgagtgccttcaagtggttggggacgttcatgattgtggtacagacaaataggctttcacatcattcttttatgtaatcatacaacagatatttgcacctacatgtgcagagcactgtgataggcctcagtgacacagaataatacggcaaagaccccacccgatgagccccctcccaccacccaccagtacagtagggggtggtttaatggagtgttcctggaatatgaagtgggggcaggcattaggggtggcaaagggacaagtgtttatctgatcagttatgtactgtttataataagtaaatcagcagagggggaataatacttagaacctatagagagtaaatctgacaagatgaaatgctgatgaaaatatggaggaaatgaaactctcatgggttttgcagggaatctaagtcagtgctgtgttgtgaatgtaggtgtaccctttgaattcatatgttgaatcctaacccccaaagcaatggcattaagaggtggggcctttggggctgggtatggtggctcatgactgtaatcccagcactttgggatgctggcagggggcagatcacttgaagccaggagtctgagatcagcctggccaacatggtgaaaccccatctgtactaaaaatacaaaaattagccaggtgtgatggcgtacatctgtaatttcagccactcgggaggctgagacaggagaatagcttgaacccagtaggtggagatttcagtgagccgagatcgtgccactgcactccagcctgggtgacagagcgagactccatctcaaaaaaataataaagatgtggggcctgtgggaggtggttaggtcatgagggtggagatcatgaatggggttagcaccttataaaacaggcttgagggagcccttctgtcccttctaccatgtgtggatgcagtgagaaggcaccgtatctctgaagcagagagcccgccctggacactggatctgctggcaccttgatcttggacttcccagcctctagaactgtgagaaataattttttgttgtttacaaattacccaggctaaggtgtttcattgtaacctgaatggaccaagctggtgtgaccctgttggaaaactggcagtatctaccaaaagccgaacatacgtataaactgatccagcagttccactcctgggtatgtacaccacagaaagctatgtccaccgagacattggcaagaatgtttctaaccacacgctgactgtagccccaaacctgaaacaacccaaatgtccatccaccaacccaaatgtccatccacagttgaagctacagtgaagtcacagggtcgaatactactgcacagcaacgaatatgaatgaaaatatcgctatgcacagcaacatggataaatttcacagacatgaggtcaagcaaaagaggtcagagtcctcatcatcaagagagaattcattgtatgattctcttcctacaaaaagtacagaaataagcaaaactgatccatggtgttagaagccaggggaacagttaacaggggagggatactggggaggggcatcctggagtgctggtctacctcatctgggtgttgatttcacgagtattgtcagtttgtttccagactccctgttggagatgtggaaataaaaaccacctaaacaagagcagagaggccatttggtcaaagtttgcaaaggagtcagccatgattgcttgtatttggcaggggtcaaaggcaggcagggactgtgaaatgttatagtggaaaaaaagggaaggctctgggtgtgctgtgattggagattgttggcatggggacagagcggactaactggaggggcatctttggttggttgggggggtatatttggctttctctggttggtctggagttggaagagggggtgtggtggctggggattgggaagaagctggcagccactaagttcagactgttctgggtccgattgctgctgaggctgtggtttggcttccttggcttcccaggctggtcatgggtttctggccagagtctattgtcatatgtggcctggccattgtccagttgtatgttcagtctcttggaaggaagggtattgactctgagaggggccaccatcgctggaatgggggacacacagtacttcctccagctgcctacacccccctagggtcagtggcgcctgcctgtgagggtgagcccaatggctagagggctctgctccaagtcattgcttactacacccacaaacattcttcgttctttaaggcctaacttaaagcccagatcctacaggaaaccttgattagacccctctctttattaagcttcctaagatcaaaccctgcttttgtgtaaatgctgacctccttgcctacattttaaaaacctagagctgggcatgatggccccagcctgtaatcccagtgattcaggagactgaggtgggaggattgctagaagccaggagttcgagaccagcctgggtaacatagctagaccacatctcttaaaataaaatagttaatttagccaggcatgatgatatatgcctgtagtcccaactacttggaaggctgaggtgtgaggatctttgagcccgggaggtcgaggctacagtaagctatgatctcaccactgtactccagcctgggtgacagagcgagacccagactcaaaaaataaaaataaaaaccctgaatatcttccttctacttcttcagtgctgtttttatttaaaaaaaaaaaaaaccagccaaaaccacaactttttactgaagtgtaatgtaaatgctgtaaaaggcagtgaaaggcacaagggaggtggaggggtaggaagggtggaagtggcgggaggaagtggcagggcaggcaaaatgaagggaagccctgggttcttgtcctgcatccgcagccagctcccactttcctcaccctccaggacctgtaaactgtgaggctggaccagttatgtcaaatctgtcctcccccagagctcagtccctctgcccttgggtgtccttggcacaaggcaggctaggctgcaccagcttcctccatctccgtcctgcctcccccatccccaggtgccattcccacaccatctgaatcactgatttcctcgcaatcagacgctatcttccagttaatcacttcgcttgtatttaacataagaaagaaaaaccctttcattatcacatacagctggaaatcggcttcttgcaggaggcgtatccaaaggaattggagaagagataaactggtaattggtgaaagaattactttaattttttttcctacttgctgtcatgatgatgtccttagaattgtgagcccgtggacacttctgtacaataaatctgctattattacttctagaa

SEQ ID NO: 193 - DNA, 11855 bp, Homo sapiens
  source 1..11855: mol_type = genomic DNA
ctcctcgctgcgggaagggtcctgggccccgggcggcggtcgccaggtctcagggccgggggtacccgagtctcgtttcctctcagtccatccacccttcatggggccagagccctctctccagaatctgagcagcaatgccgtttgctgaagacaagacctataagtatatctgccgcaatttcagcaatttttgcaatgtggatgttgtagagattctgccttacctgccctgcctcacagcaagagaccaggatcgactgcgggccacctgcacactctcagggaaccgggacaccctctggcatctcttcaatacccttcagcggcggcccggctgggtggagtacttcattgcggcactgaggggctgtgagctagttgatctcgcggacgaagtggcctctgtctaccagagctaccagcctcagacagagtcttgctgcgccacccaggctgcagtgcagtggtgcaatcatagctcactgcagccttgacctcctggactcaagtgatccttctacctcagcctccagagtagctgggactacagggacctcggaccgtcccccagacccactggagccaccgtcacttcctgctgagaggccagggccccccacacctgctgcggcccacagcatcccctacaacagctgcagagagaaggagccaagttaccccatgcctgtccaggagacccaggcgccagagtccccaggagagaattcagagcaagccctgcagacgctcagccccagagccatcccaaggaatccagatggtggccccctggagtcctcctctgacctggcagccctcagccctctgacctccagcgggcatcaggagcaggacacagaactgggcagtacccacacagcaggtgcgacctccagcctcacaccatcccgtgggcctgtgtctccatctgtctccttccagcccctggcccgttccacccccagggcaagccgcttgcctggacccacagggtcagttgtatctactggcacctccttctcctcctcatcccctggcttggcctctgcaggggctgcagagggtaaacagggtgcagagagtgaccaggccgagcctatcatctgctccagtggggcagaggcacctgccaactctctgccctccaaagtgcctaccaccttgatgcctgtgaacacagtggccctgaaagtgcctgccaacccagcatctgtcagcacagtgccctccaagttgccaactagctcaaagccccctggtgcagtgccttctaatgcgctcaccaatccagcaccatccaaattgcccatcaactcaacccgtgctggcatggtgccatccaaagtgcctactagcatggtgctcaccaaggtgtctgccagcacagtccccactgacgggagcagcagaaatgaggagaccccagcagctccaacacccgccggcgccactggaggcagctcagcctggctagacagcagctctgagaataggggccttgggtcggagctgagtaagcctggcgtgctggcatcccaggtagacagcccgttctcgggctgcttcgaggatcttgccatcagtgccagcacctccttgggcatggggccctgccatggcccagaggagaatgagtataagtccgagggcacctttgggatccacgtggctgagaaccccagcatccagctcctggagggcaaccctgggccacctgcggacccggatggcggccccaggccacaagccgaccggaagttccaggagagggaggtgccatgccacaggccctcacctggggctctgtggctccaggtggctgtgacaggggtgctggtagtcacactcctggtggtgctgtaccggcggcgtctgcactagtgaagccctgggctcttcccaccacccatctgttccgttcctgcagtacacctggcccctctccgaagccccttgtccctttcttggggattgtggaggctgggtcagaggggagttaagggactgcaggcctggcagcaggacatgccttggctgaaccaagtcctgagagcagcatctctgtccccacggtgccttgtgtgggtccccgtccttggctttctgggtcctgggctgcccccagtgctccagaccttccccactggcaatccaggttatcatccatgtcctccagaggagcttcctcctccaggcctcagccctgttggcccaggtggagcaggagggaccactggaacatgtggtgcttgggaatgcctctcctgttgcattggtccctgaaggcctcagggcaggtatgtggtgtgtgggcgactccacaagacctgcctcccatcctggcagcccagcctgagaccgttgcattgaggcaggcaggagcggcagggtggctgctctccaggagcccaactgccttgagttcctgccccactgggccccctcccctgctgggcaatcctgggaaggtctggaggttcctgtggacctcagggaagccaggggcagctgtcaggcctgaggaagacctgtggagctcctctccagcctcctctttccctcccctctggtctccattctcttcagctccctacatgggctggggaggagacacctggtgggcagagctcaggcagaggtttggatttcagctccctcacttccggggctgtgtggctttggcagatgtcagacttctggtcttgcttctccacgtggacagtgagtatctggctcattcttcactgggttcttctgagattgaacctacaggtgtttgccaagtgcctggcccagagcaagtggccactgcttctcccatctctctcctgcccaacctggtagagctgagggcatgagaggcagagtgcacagtggtcaagggtgcagctctgcagcacaggcagcctaggcctgcgtcccaacctgcctctcaccagctctgtgaccttgggcaagggatttatctgtctgtcccttagttttctcacctgtaaaaggaggataagtatatatatatatttcccagtgttgtgaagattaaaggagtttatcgatgtaggtcttaggatgagtcctggcatttaccaagggttggatatatgttattatcactattaagtgttgagggtccaggcatgctgggcaacagggaccccatctctacaaaaaagtttaaaaaattagccaggcgtggtggtgcacctgtcgtcttagctacttgggaggctgaggtgggaggatcacttgagcccagaagcttgaagctgcagtgagctaggatcgtgccactgcactccaacctgggtgagagagcgagaccctgtctcaagaaaaagaaaaatgcagagaaacaggagtcttggctactcctttagaggcagactcagaccctcctgcctcacagctttatctttgtatttgccccttactttatcttgtgccttgagaaattgctggggagagaggtatgtccactgggcagctgtacaggatggaggatatagggcgtttccactcccagcagccaggttccctcaccccaagctcacccactgttggggagattatctacaataacaccagaaacacattggggtggattgggggtatccttatgggttcttttcagggaaccattgctggacaaggcacaggagccacctccatttctgagctctgcaagggacaagaactagagccatcaggggctgggctcactgtggccccaccccaagccgtcagcctccagggatctacaccctgccttggctgctacagctttttcactccactgccctaggggagttcagcaacctaatgatctctatctctgaacatctcttcatcccatgctccaagtccagcaacctgcaccctggaaccaggagtggaccctacccgagctgtctgtattaatccccatcccccaccaccaatcttaaaaagccctctgtccccctaccctaaaccccagttaggtacccatgctgggcaggtcagttaacaatttatgcacaggtactagttttattgtattaccgttccagggtagctttgaaaaaagtatctcaaaaaggcaacatgggccgagcgcagtggctcacgcctgtaatcccagcactttgggaggccaaggtgggcagatcgcctgaggtctggagttcaagaccagcctggccaacagggtgaaaccccgtctctacaaaaataagaaaattagccaggtgtagtggcagacgtctgtaatcccagctattcaggaggctgaggcacgagaattccatgaacccaggatgcggaggttgcagtgagccgagattgtgccactgcgctccagcctgggcgacagagtggtattctgtttcaaaaaaaaaaaaaaaggcagtatgtagccccgaagactgttgcccaagtggtagaatgttagcacactaccagcctaggtaaaaaatacaaaaagtaactgggcatggcggcgcccatctatagtcccagctacatgggaggctgaggtgggaagataagtcacttgagcccgccaggaggcggaggttgtagtgagctgagatcgcaccactgcactccagcctgggtgaccgagtgatactctgtctcaaagaaaaaaaattataattttagcacagtaaccagccatgatgggagataccctgggtaaggcatgtagaaagggttgagggaccttcccagtcccctagccccgcctcccatcctcccatctttttcttttttcttttttttagagaatcacccagcctggagcgaagtggtgcaatcataactcactgtatccttaaactcccgggcttaagcgatcctcctgcctcagccttctgagtaactaggacttcaggtacctgtcaccatgcctggctaattaaatttttttttcttttttttttttgagatggagtcttgctctgtcacccaggctggagtgcagtggcgcgatctcagctcactgcgacctccacctcctgggttcaggccattctcccgcctcagcctccagagtagctgggactacaggcgcctgccaccacgcctggctaatttttttgcacttttagtagagacggggtttcactgtgttagccaggatggtctcgatctcctgaccttgtgatccgcccgcctcggcctcccaaagtgctgggattacaggcgtgagccaccgcgcccagccaaattaaattttttatagagatgaggtcatgctgttatgttgcccaggttggcctcatgagatcttgccttagcctcccaaagtgctgggattacagatgtgagacactgcacccaaaccccaccacttttttttttcctttttctttttttgagacagtcttactccgttgcccaggctggagtgtagtggcatgatctcagctcactgcaacctccgcctcccgggttcaagcaattctcctgcctcagcctcccgagtagctgggattacagaggcctgccaccacacccgactaattttcgtatttttagtagagacggggtttctccatgttggccaggctgttcttgaactcctgacctcaagtgctccacctgcgttggcttcccaaagtgctgggatacaggagtgagccactgcgcctggctgatcccagcacttttcaaatgatgccgctcaaagccgtgacttggcctactttgaacagcaaacttgttgctgctgttgtcaacctgaaggcctctcaaatgccagcttcaagcagggtgtgaattggccagtgtcagatctcaggagtcctgtgttgagagtgtggctttcagctgcggggagctgcacttggtggggaaagccaggcaggtcaccctcacagccagataatgtggaggtcagaacccaaggaagggagtgagacctccactcccagtgggggacctggccacccatccttggggacctgagaaagcgtacttcaccttggggtgaaggctgggtggggccagagggaccagtgccctcctcagtgcttaggggcagagccacctgcagcaatggtatctgcatattagcccctctccaccttctttctcccgctgaatcatttccctcaaagcccaagagctgtcactgcttctttctccctgggaagaatgcgtggactctgcctggtgatagactgaagccagaacagtgccacaccctcgccttaattccttgctaggtgttctcagatttatgagacttcttagtcaaatatgagggaggttggatgtggtggcttgtgcctgtaatcccagcattttgggaagccgaggtgggaggatcccttgaagccaggagtttgagacaagcctgggcaacaaagcaagaccctatctctaaaaaaaaaaaaaaaaaaaaaaaaaaaaaaaaaaaaaaaaaatctaggagatgctctttaccctgcctggcctcaaactattaatagcttcctttgagcaacattatttattatgaactttcaaacacaaaaaagtagagagagtagaataacaaatccccatgagcccatcacccaacttcagtaattatcaattcatggccatcttgttcacccctgcctgcttccctgcttcccctcattctgcagaggttcttttcttttgagacagagtgttgctctgttgcccaggctggagtgcagtggtgcaacttcggctcactgcaacctccgcctcccaggttcaagtgattctcctgcttcagcctctcaagtagctgggattacagatgcccgccaccacacctggctaattttcgtatttttgttagagatggggtttcaccatgttggccaggctggtctcgaactcctgacctcaagtgatccgcccgccttggcctcccaaaatgctgggattacaggtgtgaaccacggtgcctggccactgtacaggttatttatagaagttggagagtgaagggttgagaaagccaaggggcagatgcgggtctggaggattttgtgcctaaggccctctctttgctcccagacagcatgaagtaacaatgaggcatccacctcttggttttgtggcctctgtggatgacgtctctcaccttgaaccagttcagagttggagtagcgcaggatcctgtcttcagaggaggggccgaagcgggttcctctgttgtcaagctctttggaggtgcctggctgctactactgtcccagagaggtgatgatgaatgatgggtgtgtccagtggcagtttgccccactgaggcaggggcttccactaggccctgacagagcccttccagcaggcagaaatccctgtgctaggcaagattcaaactccgtagcatgtctcctgctcccatctcttaggaatggagtccttcaggccttgagtcccacattttccatgatgctccattaagcagctgatagcacccccacctccagggaaagtgagttcagagtccttggtctaatgcatctgtgttgaaattgaggccttcccctgtgttcacctttctgctctttttcttttagcccaaggctatgaaggcctcattcggtgctgggcatggtcactcctagcattcctcactctgttgctaacagcaacagcaataataataagggttacaacttactccataccttactgtctgccaggcattaagctaagtgctttacatatattaagtcatttaatcctcataatgaccctatgaaagagataccatctcaacccaattgacagctggtttgcaagattaggagggatgaaggacccaggggacaatgcgagggaaaactctgaccccggggccccaggctggatgttctttatgcctgtgaaccacagcttatcacatgtctggagttagggaccccacttaaagtgagattttggctggaggtggtggatcatacctataatcccagcactttgggagaccaaggcagaaggactgcttgaggccaggagttcaaaaccagtgtaggtaacagctagaccctatctctacaaaaaatttaaaaattagctgggtgtggtggtatgtgcctcaagttccagctactcaggaggctgaggtgggaggatcacttgagcacaggagtttgaagttacagtgagctatgatggcaccactgcacttcagcctaggcaacagagggagaccctgtctttaaagtacatagaggtttttcacaccaacacatctctgcccagtgtgccaacatctgccacctgctataatagtactataacactcaatatgtaattaatgtagtctcagggatgttatgacaatatgattacaactatcacgtgtgtgcccagccaggctcaatgccccaggctgggcgaggtggggcaggggacacagcctaaaatgccaggcctcaggaagccatttggtttagcagacattgtttattaaaggagttacctatgccagatcgaaggcctaagatgattaagacactatgagtgccttcaagtggttggggacgttcatgattgtggtacagacaaataggctttcacatcattcttttatgtaatcatacaacagatatttgcacctacatgtgcagagcactgtgataggcctcagtgacacagaataatacggcaaagaccccacccgatgagccccctcccaccacccaccagtacagtagggggtggtttaatggagtgttcctggaatatgaagtgggggcaggcattaggggtggcaaagggacaagtgtttatctgatcagttatgtactgtttataataagtaaatcagcagagggggaataatacttagaacctatagagagtaaatctgacaagatgaaatgctgatgaaaatatggaggaaatgaaactctcatgggttttgcagggaatctaagtcagtgctgtgttgtgaatgtaggtgtaccctttgaattcatatgttgaatcctaacccccaaagcaatggcattaagaggtggggcctttggggctgggtatggtggctcatgactgtaatcccagcactttgggatgctggcagggggcagatcacttgaagccaggagtctgagatcagcctggccaacatggtgaaaccccatctgtactaaaaatacaaaaattagccaggtgtgatggcgtacatctgtaatttcagccactcgggaggctgagacaggagaatagcttgaacccagtaggtggagatttcagtgagccgagatcgtgccactgcactccagcctgggtgacagagcgagactccatctcaaaaaaataataaagatgtggggcctgtgggaggtggttaggtcatgagggtggagatcatgaatggggttagcaccttataaaacaggcttgagggagcccttctgtcccttctaccatgtgtggatgcagtgagaaggcaccgtatctctgaagcagagagcccgccctggacactggatctgctggcaccttgatcttggacttcccagcctctagaactgtgagaaataattttttgttgtttacaaattacccaggctaaggtgtttcattgtaacctgaatggaccaagctggtgtgaccctgttggaaaactggcagtatctaccaaaagccgaacatacgtataaactgatccagcagttccactcctgggtatgtacaccacagaaagctatgtccaccgagacattggcaagaatgtttctaaccacacgctgactgtagccccaaacctgaaacaacccaaatgtccatccaccaacccaaatgtccatccacagttgaagctacagtgaagtcacagggtcgaatactactgcacagcaacgaatatgaatgaaaatatcgctatgcacagcaacatggataaatttcacagacatgaggtcaagcaaaagaggtcagagtcctcatcatcaagagagaattcattgtatgattctcttcctacaaaaagtacagaaataagcaaaactgatccatggtgttagaagccaggggaacagttaacaggggagggatactggggaggggcatcctggagtgctggtctacctcatctgggtgttgatttcacgagtattgtcagtttgtttccagactccctgttggagatgtggaaataaaaaccacctaaacaagagcagagaggccatttggtcaaagtttgcaaaggagtcagccatgattgcttgtatttggcaggggtcaaaggcaggcagggactgtgaaatgttatagtggaaaaaaagggaaggctctgggtgtgctgtgattggagattgttggcatggggacagagcggactaactggaggggcatctttggttggttgggggggtatatttggctttctctggttggtctggagttggaagagggggtgtggtggctggggattgggaagaagctggcagccactaagttcagactgttctgggtccgattgctgctgaggctgtggtttggcttccttggcttcccaggctggtcatgggtttctggccagagtctattgtcatatgtggcctggccattgtccagttgtatgttcagtctcttggaaggaagggtattgactctgagaggggccaccatcgctggaatgggggacacacagtacttcctccagctgcctacacccccctagggtcagtggcgcctgcctgtgagggtgagcccaatggctagagggctctgctccaagtcattgcttactacacccacaaacattcttcgttctttaaggcctaacttaaagcccagatcctacaggaaaccttgattagacccctctctttattaagcttcctaagatcaaaccctgcttttgtgtaaatgctgacctccttgcctacattttaaaaacctagagctgggcatgatggccccagcctgtaatcccagtgattcaggagactgaggtgggaggattgctagaagccaggagttcgagaccagcctgggtaacatagctagaccacatctcttaaaataaaatagttaatttagccaggcatgatgatatatgcctgtagtcccaactacttggaaggctgaggtgtgaggatctttgagcccgggaggtcgaggctacagtaagctatgatctcaccactgtactccagcctgggtgacagagcgagacccagactcaaaaaataaaaataaaaaccctgaatatcttccttctacttcttcagtgctgtttttatttaaaaaaaaaaaaaaccagccaaaaccacaactttttactgaagtgtaatgtaaatgctgtaaaaggcagtgaaaggcacaagggaggtggaggggtaggaagggtggaagtggcgggaggaagtggcagggcaggcaaaatgaagggaagccctgggttcttgtcctgcatccgcagccagctcccactttcctcaccctccaggacctgtaaactgtgaggctggaccagttatgtcaaatctgtcctcccccagagctcagtccctctgcccttgggtgtccttggcacaaggcaggctaggctgcaccagcttcctccatctccgtcctgcctcccccatccccaggtgccattcccacaccatctgaatcactgatttcctcgcaatcagacgctatcttccagttaatcacttcgcttgtatttaacataagaaagaaaaaccctttcattatcacatacagctggaaatcggcttcttgcaggaggcgtatccaaaggaattggagaagagataaactggtaattggtgaaagaattactttaattttttttcctacttgctgtcatgatgatgtccttagaattgtgagcccgtggacacttctgtacaataaatctgctattattacttctagaa

SEQ ID NO: 194 - DNA, 11731 bp, Homo sapiens
  source 1..11731: mol_type = genomic DNA
ctcctcgctgcgggaagggtcctgggccccgggcggcggtcgccaggtctcagggccgggggtacccgagtctcgtttcctctcagtccatccacccttcatggggccagagccctctctccagaatctgagcagcaatgccgtttgctgaagacaagacctataagtatatctgccgcaatttcagcaatttttgcaatgtggatgttgtagagattctgccttacctgccctgcctcacagcaagagaccaggatcgactgcgggccacctgcacactctcagggaaccgggacaccctctggcatctcttcaatacccttcagcggcggcccggctgggtggagtacttcattgcggcactgaggggctgtgagctagttgatctcgcggacgaagtggcctctgtctaccagagctaccagcctcggacctcggaccgtcccccagacccactggagccaccgtcacttcctgctgagaggccagggccccccacacctgctgcggcccacagcatcccctacaacagctgcagagagaaggagccaagttaccccatgcctgtccaggagacccaggcgccagagtccccaggagagaattcagagcaagccctgcagacgctcagccccagagccatcccaaggaatccagatggtggccccctggagtcctcctctgacctggcagccctcagccctctgacctccagcgggcatcaggagcaggacacagaactgggcagtacccacacagcaggtgcgacctccagcctcacaccatcccgtgggcctgtgtctccatctgtctccttccagcccctggcccgttccacccccagggcaagccgcttgcctggacccacagggtcagttgtatctactggcacctccttctcctcctcatcccctggcttggcctctgcaggggctgcagagggtaaacagggtgcagagagtgaccaggccgagcctatcatctgctccagtggggcagaggcacctgccaactctctgccctccaaagtgcctaccaccttgatgcctgtgaacacagtggccctgaaagtgcctgccaacccagcatctgtcagcacagtgccctccaagttgccaactagctcaaagccccctggtgcagtgccttctaatgcgctcaccaatccagcaccatccaaattgcccatcaactcaacccgtgctggcatggtgccatccaaagtgcctactagcatggtgctcaccaaggtgtctgccagcacagtccccactgacgggagcagcagaaatgaggagaccccagcagctccaacacccgccggcgccactggaggcagctcagcctggctagacagcagctctgagaataggggccttgggtcggagctgagtaagcctggcgtgctggcatcccaggtagacagcccgttctcgggctgcttcgaggatcttgccatcagtgccagcacctccttgggcatggggccctgccatggcccagaggagaatgagtataagtccgagggcacctttgggatccacgtggctgagaaccccagcatccagctcctggagggcaaccctgggccacctgcggacccggatggcggccccaggccacaagccgaccggaagttccaggagagggaggtgccatgccacaggccctcacctggggctctgtggctccaggtggctgtgacaggggtgctggtagtcacactcctggtggtgctgtaccggcggcgtctgcactagtgaagccctgggctcttcccaccacccatctgttccgttcctgcagtacacctggcccctctccgaagccccttgtccctttcttggggattgtggaggctgggtcagaggggagttaagggactgcaggcctggcagcaggacatgccttggctgaaccaagtcctgagagcagcatctctgtccccacggtgccttgtgtgggtccccgtccttggctttctgggtcctgggctgcccccagtgctccagaccttccccactggcaatccaggttatcatccatgtcctccagaggagcttcctcctccaggcctcagccctgttggcccaggtggagcaggagggaccactggaacatgtggtgcttgggaatgcctctcctgttgcattggtccctgaaggcctcagggcaggtatgtggtgtgtgggcgactccacaagacctgcctcccatcctggcagcccagcctgagaccgttgcattgaggcaggcaggagcggcagggtggctgctctccaggagcccaactgccttgagttcctgccccactgggccccctcccctgctgggcaatcctgggaaggtctggaggttcctgtggacctcagggaagccaggggcagctgtcaggcctgaggaagacctgtggagctcctctccagcctcctctttccctcccctctggtctccattctcttcagctccctacatgggctggggaggagacacctggtgggcagagctcaggcagaggtttggatttcagctccctcacttccggggctgtgtggctttggcagatgtcagacttctggtcttgcttctccacgtggacagtgagtatctggctcattcttcactgggttcttctgagattgaacctacaggtgtttgccaagtgcctggcccagagcaagtggccactgcttctcccatctctctcctgcccaacctggtagagctgagggcatgagaggcagagtgcacagtggtcaagggtgcagctctgcagcacaggcagcctaggcctgcgtcccaacctgcctctcaccagctctgtgaccttgggcaagggatttatctgtctgtcccttagttttctcacctgtaaaaggaggataagtatatatatatatttcccagtgttgtgaagattaaaggagtttatcgatgtaggtcttaggatgagtcctggcatttaccaagggttggatatatgttattatcactattaagtgttgagggtccaggcatgctgggcaacagggaccccatctctacaaaaaagtttaaaaaattagccaggcgtggtggtgcacctgtcgtcttagctacttgggaggctgaggtgggaggatcacttgagcccagaagcttgaagctgcagtgagctaggatcgtgccactgcactccaacctgggtgagagagcgagaccctgtctcaagaaaaagaaaaatgcagagaaacaggagtcttggctactcctttagaggcagactcagaccctcctgcctcacagctttatctttgtatttgccccttactttatcttgtgccttgagaaattgctggggagagaggtatgtccactgggcagctgtacaggatggaggatatagggcgtttccactcccagcagccaggttccctcaccccaagctcacccactgttggggagattatctacaataacaccagaaacacattggggtggattgggggtatccttatgggttcttttcagggaaccattgctggacaaggcacaggagccacctccatttctgagctctgcaagggacaagaactagagccatcaggggctgggctcactgtggccccaccccaagccgtcagcctccagggatctacaccctgccttggctgctacagctttttcactccactgccctaggggagttcagcaacctaatgatctctatctctgaacatctcttcatcccatgctccaagtccagcaacctgcaccctggaaccaggagtggaccctacccgagctgtctgtattaatccccatcccccaccaccaatcttaaaaagccctctgtccccctaccctaaaccccagttaggtacccatgctgggcaggtcagttaacaatttatgcacaggtactagttttattgtattaccgttccagggtagctttgaaaaaagtatctcaaaaaggcaacatgggccgagcgcagtggctcacgcctgtaatcccagcactttgggaggccaaggtgggcagatcgcctgaggtctggagttcaagaccagcctggccaacagggtgaaaccccgtctctacaaaaataagaaaattagccaggtgtagtggcagacgtctgtaatcccagctattcaggaggctgaggcacgagaattccatgaacccaggatgcggaggttgcagtgagccgagattgtgccactgcgctccagcctgggcgacagagtggtattctgtttcaaaaaaaaaaaaaaaggcagtatgtagccccgaagactgttgcccaagtggtagaatgttagcacactaccagcctaggtaaaaaatacaaaaagtaactgggcatggcggcgcccatctatagtcccagctacatgggaggctgaggtgggaagataagtcacttgagcccgccaggaggcggaggttgtagtgagctgagatcgcaccactgcactccagcctgggtgaccgagtgatactctgtctcaaagaaaaaaaattataattttagcacagtaaccagccatgatgggagataccctgggtaaggcatgtagaaagggttgagggaccttcccagtcccctagccccgcctcccatcctcccatctttttcttttttcttttttttagagaatcacccagcctggagcgaagtggtgcaatcataactcactgtatccttaaactcccgggcttaagcgatcctcctgcctcagccttctgagtaactaggacttcaggtacctgtcaccatgcctggctaattaaatttttttttcttttttttttttgagatggagtcttgctctgtcacccaggctggagtgcagtggcgcgatctcagctcactgcgacctccacctcctgggttcaggccattctcccgcctcagcctccagagtagctgggactacaggcgcctgccaccacgcctggctaatttttttgcacttttagtagagacggggtttcactgtgttagccaggatggtctcgatctcctgaccttgtgatccgcccgcctcggcctcccaaagtgctgggattacaggcgtgagccaccgcgcccagccaaattaaattttttatagagatgaggtcatgctgttatgttgcccaggttggcctcatgagatcttgccttagcctcccaaagtgctgggattacagatgtgagacactgcacccaaaccccaccacttttttttttcctttttctttttttgagacagtcttactccgttgcccaggctggagtgtagtggcatgatctcagctcactgcaacctccgcctcccgggttcaagcaattctcctgcctcagcctcccgagtagctgggattacagaggcctgccaccacacccgactaattttcgtatttttagtagagacggggtttctccatgttggccaggctgttcttgaactcctgacctcaagtgctccacctgcgttggcttcccaaagtgctgggatacaggagtgagccactgcgcctggctgatcccagcacttttcaaatgatgccgctcaaagccgtgacttggcctactttgaacagcaaacttgttgctgctgttgtcaacctgaaggcctctcaaatgccagcttcaagcagggtgtgaattggccagtgtcagatctcaggagtcctgtgttgagagtgtggctttcagctgcggggagctgcacttggtggggaaagccaggcaggtcaccctcacagccagataatgtggaggtcagaacccaaggaagggagtgagacctccactcccagtgggggacctggccacccatccttggggacctgagaaagcgtacttcaccttggggtgaaggctgggtggggccagagggaccagtgccctcctcagtgcttaggggcagagccacctgcagcaatggtatctgcatattagcccctctccaccttctttctcccgctgaatcatttccctcaaagcccaagagctgtcactgcttctttctccctgggaagaatgcgtggactctgcctggtgatagactgaagccagaacagtgccacaccctcgccttaattccttgctaggtgttctcagatttatgagacttcttagtcaaatatgagggaggttggatgtggtggcttgtgcctgtaatcccagcattttgggaagccgaggtgggaggatcccttgaagccaggagtttgagacaagcctgggcaacaaagcaagaccctatctctaaaaaaaaaaaaaaaaaaaaaaaaaaaaaaaaaaaaaaaaaatctaggagatgctctttaccctgcctggcctcaaactattaatagcttcctttgagcaacattatttattatgaactttcaaacacaaaaaagtagagagagtagaataacaaatccccatgagcccatcacccaacttcagtaattatcaattcatggccatcttgttcacccctgcctgcttccctgcttcccctcattctgcagaggttcttttcttttgagacagagtgttgctctgttgcccaggctggagtgcagtggtgcaacttcggctcactgcaacctccgcctcccaggttcaagtgattctcctgcttcagcctctcaagtagctgggattacagatgcccgccaccacacctggctaattttcgtatttttgttagagatggggtttcaccatgttggccaggctggtctcgaactcctgacctcaagtgatccgcccgccttggcctcccaaaatgctgggattacaggtgtgaaccacggtgcctggccactgtacaggttatttatagaagttggagagtgaagggttgagaaagccaaggggcagatgcgggtctggaggattttgtgcctaaggccctctctttgctcccagacagcatgaagtaacaatgaggcatccacctcttggttttgtggcctctgtggatgacgtctctcaccttgaaccagttcagagttggagtagcgcaggatcctgtcttcagaggaggggccgaagcgggttcctctgttgtcaagctctttggaggtgcctggctgctactactgtcccagagaggtgatgatgaatgatgggtgtgtccagtggcagtttgccccactgaggcaggggcttccactaggccctgacagagcccttccagcaggcagaaatccctgtgctaggcaagattcaaactccgtagcatgtctcctgctcccatctcttaggaatggagtccttcaggccttgagtcccacattttccatgatgctccattaagcagctgatagcacccccacctccagggaaagtgagttcagagtccttggtctaatgcatctgtgttgaaattgaggccttcccctgtgttcacctttctgctctttttcttttagcccaaggctatgaaggcctcattcggtgctgggcatggtcactcctagcattcctcactctgttgctaacagcaacagcaataataataagggttacaacttactccataccttactgtctgccaggcattaagctaagtgctttacatatattaagtcatttaatcctcataatgaccctatgaaagagataccatctcaacccaattgacagctggtttgcaagattaggagggatgaaggacccaggggacaatgcgagggaaaactctgaccccggggccccaggctggatgttctttatgcctgtgaaccacagcttatcacatgtctggagttagggaccccacttaaagtgagattttggctggaggtggtggatcatacctataatcccagcactttgggagaccaaggcagaaggactgcttgaggccaggagttcaaaaccagtgtaggtaacagctagaccctatctctacaaaaaatttaaaaattagctgggtgtggtggtatgtgcctcaagttccagctactcaggaggctgaggtgggaggatcacttgagcacaggagtttgaagttacagtgagctatgatggcaccactgcacttcagcctaggcaacagagggagaccctgtctttaaagtacatagaggtttttcacaccaacacatctctgcccagtgtgccaacatctgccacctgctataatagtactataacactcaatatgtaattaatgtagtctcagggatgttatgacaatatgattacaactatcacgtgtgtgcccagccaggctcaatgccccaggctgggcgaggtggggcaggggacacagcctaaaatgccaggcctcaggaagccatttggtttagcagacattgtttattaaaggagttacctatgccagatcgaaggcctaagatgattaagacactatgagtgccttcaagtggttggggacgttcatgattgtggtacagacaaataggctttcacatcattcttttatgtaatcatacaacagatatttgcacctacatgtgcagagcactgtgataggcctcagtgacacagaataatacggcaaagaccccacccgatgagccccctcccaccacccaccagtacagtagggggtggtttaatggagtgttcctggaatatgaagtgggggcaggcattaggggtggcaaagggacaagtgtttatctgatcagttatgtactgtttataataagtaaatcagcagagggggaataatacttagaacctatagagagtaaatctgacaagatgaaatgctgatgaaaatatggaggaaatgaaactctcatgggttttgcagggaatctaagtcagtgctgtgttgtgaatgtaggtgtaccctttgaattcatatgttgaatcctaacccccaaagcaatggcattaagaggtggggcctttggggctgggtatggtggctcatgactgtaatcccagcactttgggatgctggcagggggcagatcacttgaagccaggagtctgagatcagcctggccaacatggtgaaaccccatctgtactaaaaatacaaaaattagccaggtgtgatggcgtacatctgtaatttcagccactcgggaggctgagacaggagaatagcttgaacccagtaggtggagatttcagtgagccgagatcgtgccactgcactccagcctgggtgacagagcgagactccatctcaaaaaaataataaagatgtggggcctgtgggaggtggttaggtcatgagggtggagatcatgaatggggttagcaccttataaaacaggcttgagggagcccttctgtcccttctaccatgtgtggatgcagtgagaaggcaccgtatctctgaagcagagagcccgccctggacactggatctgctggcaccttgatcttggacttcccagcctctagaactgtgagaaataattttttgttgtttacaaattacccaggctaaggtgtttcattgtaacctgaatggaccaagctggtgtgaccctgttggaaaactggcagtatctaccaaaagccgaacatacgtataaactgatccagcagttccactcctgggtatgtacaccacagaaagctatgtccaccgagacattggcaagaatgtttctaaccacacgctgactgtagccccaaacctgaaacaacccaaatgtccatccaccaacccaaatgtccatccacagttgaagctacagtgaagtcacagggtcgaatactactgcacagcaacgaatatgaatgaaaatatcgctatgcacagcaacatggataaatttcacagacatgaggtcaagcaaaagaggtcagagtcctcatcatcaagagagaattcattgtatgattctcttcctacaaaaagtacagaaataagcaaaactgatccatggtgttagaagccaggggaacagttaacaggggagggatactggggaggggcatcctggagtgctggtctacctcatctgggtgttgatttcacgagtattgtcagtttgtttccagactccctgttggagatgtggaaataaaaaccacctaaacaagagcagagaggccatttggtcaaagtttgcaaaggagtcagccatgattgcttgtatttggcaggggtcaaaggcaggcagggactgtgaaatgttatagtggaaaaaaagggaaggctctgggtgtgctgtgattggagattgttggcatggggacagagcggactaactggaggggcatctttggttggttgggggggtatatttggctttctctggttggtctggagttggaagagggggtgtggtggctggggattgggaagaagctggcagccactaagttcagactgttctgggtccgattgctgctgaggctgtggtttggcttccttggcttcccaggctggtcatgggtttctggccagagtctattgtcatatgtggcctggccattgtccagttgtatgttcagtctcttggaaggaagggtattgactctgagaggggccaccatcgctggaatgggggacacacagtacttcctccagctgcctacacccccctagggtcagtggcgcctgcctgtgagggtgagcccaatggctagagggctctgctccaagtcattgcttactacacccacaaacattcttcgttctttaaggcctaacttaaagcccagatcctacaggaaaccttgattagacccctctctttattaagcttcctaagatcaaaccctgcttttgtgtaaatgctgacctccttgcctacattttaaaaacctagagctgggcatgatggccccagcctgtaatcccagtgattcaggagactgaggtgggaggattgctagaagccaggagttcgagaccagcctgggtaacatagctagaccacatctcttaaaataaaatagttaatttagccaggcatgatgatatatgcctgtagtcccaactacttggaaggctgaggtgtgaggatctttgagcccgggaggtcgaggctacagtaagctatgatctcaccactgtactccagcctgggtgacagagcgagacccagactcaaaaaataaaaataaaaaccctgaatatcttccttctacttcttcagtgctgtttttatttaaaaaaaaaaaaaaccagccaaaaccacaactttttactgaagtgtaatgtaaatgctgtaaaaggcagtgaaaggcacaagggaggtggaggggtaggaagggtggaagtggcgggaggaagtggcagggcaggcaaaatgaagggaagccctgggttcttgtcctgcatccgcagccagctcccactttcctcaccctccaggacctgtaaactgtgaggctggaccagttatgtcaaatctgtcctcccccagagctcagtccctctgcccttgggtgtccttggcacaaggcaggctaggctgcaccagcttcctccatctccgtcctgcctcccccatccccaggtgccattcccacaccatctgaatcactgatttcctcgcaatcagacgctatcttccagttaatcacttcgcttgtatttaacataagaaagaaaaaccctttcattatcacatacagctggaaatcggcttcttgcaggaggcgtatccaaaggaattggagaagagataaactggtaattggtgaaagaattactttaattttttttcctacttgctgtcatgatgatgtccttagaattgtgagcccgtggacacttctgtacaataaatctgctattattacttctagaa

SEQ ID NO: 195 - DNA, 3581 bp, Homo sapiens
  source 1..3581: mol_type = genomic DNA
caaactctgtaagaactgcctgacagaaagctggactcaaagctcctacccgagtgtgcagcaggatcgccccggtccgggaccccaggcgcacaccgcagagtccaaagtgccgcgcctgccggccgcacctgcctgccgcggccccgcgcgccgccccgctgcccacctgcccgcctgcccacctgcccaggtgcgagtgcagccccgcgcgccggcctgagagccctgtggacaacctcgtcattgtcaggcacagagcggtagaccctgcttctctaagtgggcagcggacagcggcacgcacatttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccatgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccctcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaagaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaataaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacatgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaagcactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaataaaatgattgttttactctgcattgaa

SEQ ID NO: 196 - DNA, 2351 bp, Homo sapiens
  source 1..2351: mol_type = genomic DNA
aatgcgacccgaccgcgctgaggctccaggaccgcccgccatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctgaagactgttctgaggccctgggtgtgtgtgtatctgtctgcctgtccatgtacttctgccctgcctcctcctttcgttgtaggaggaatctgtgctctacttacctctcaattcctggagatgccaacttcacagacacgtctgcagcagctggacatcacatttcatgtcctgcatggaaccagtggctgtgagtggcatgtccacttgctggattatcagccaggacactatagaacaggaccagctgagactaagaaggaccagcagagccagctcagctctgagccattcacacatcttcaccctcagtttcctcacttgaggagtgggatggggagaacagagagtagctgtgtttgaatccctgtaggaaatggtgaagcatagctctgggtctcctgggggagaccaggcttggctgcgggagagctggctgttgctggactacatgctggccactgctgtgaccacgacactgctggggcagcttcttccacagtgatgcctactgatgcttcagtgcctctgcacaccgcccattccacttcctccttccccacagggcaggtggggaagcagtttggcccagcccaaggagaccccaccttgagccttatttcctaatgggtccacctctcatctgcatctttcacacctcccagcttctgcccaaccttcagcagtgacaagtccccaagagactcgcctgagcagcttgggctgcttttcatttccacctgtcaggatgcctgtggtcatgctctcagctccacctggcatgagaagggatcctggcctctggcatattcatcaagtatgagttctggggatgagtcactgtaatgatgtgagcagggagccttcctccctgggccacctgcagagagctttcccaccaactttgtaccttgattgccttacaaagttatttgtttacaaacagcgaccatataaaagcctcctgccccaaagcttgtgggcacatgggcacatacagactcacatacagacacacacatatatgtacagacatgtactctcacacacacaggcaccagcatacacacgtttttctaggtacagctcccaggaacagctaggtgggaaagtcccatcactgagggagcctaaccatgtccctgaacaaaaattgggcactcatctattccttttctcttgtgtccctactcattgaaaccaaactctggaaaggacccaatgtaccagtatttatacctctaatgaagcacagagagaggaagagagctgcttaaactcacacaacaatgaactgcagacacagctgttctctccctctctccttcccagagcaatttatactttaccctcaggctgtcctctggggagaaggtgccatggtcttaggtgtctgtgccccaggacagaccctaggaccctaaatccaatagaaaatgcatatctttgctccactttcagccaggctggagcaaggtaccttttcttaggatcttgggagggaatggatgcccctctctgcatgatcttgttgaggcatttagctgccatgcacctgtccccctttaatactgggcattttaaagccatctcaagaggcatcttctacatgttttgtacgcattaaaataatttcaaagatatctgagaaaagccgatatttgccattcttcctatatcctggaatatatcttgcatcctgagtttataataataaataatattctaccttggaaa

SEQ ID NO: 197 - DNA, 2691 bp, Homo sapiens
  source 1..2691: mol_type = genomic DNA
aatgcgacccgaccgcgctgaggctccaggaccgcccgccatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggttggctagaaggccacggggtgggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctgaagactgttctgaggccctgggtgtgtgtgtatctgtctgcctgtccatgtacttctgccctgcctcctcctttcgttgtaggaggaatctgtgctctacttacctctcaattcctggagatgccaacttcacagacacgtctgcagcagctggacatcacatttcatgtcctgcatggaaccagtggctgtgagtggcatgtccacttgctggattatcagccaggacactatagaacaggaccagctgagactaagaaggaccagcagagccagctcagctctgagccattcacacatcttcaccctcagtttcctcacttgaggagtgggatggggagaacagagagtagctgtgtttgaatccctgtaggaaatggtgaagcatagctctgggtctcctgggggagaccaggcttggctgcgggagagctggctgttgctggactacatgctggccactgctgtgaccacgacactgctggggcagcttcttccacagtgatgcctactgatgcttcagtgcctctgcacaccgcccattccacttcctccttccccacagggcaggtggggaagcagtttggcccagcccaaggagaccccaccttgagccttatttcctaatgggtccacctctcatctgcatctttcacacctcccagcttctgcccaaccttcagcagtgacaagtccccaagagactcgcctgagcagcttgggctgcttttcatttccacctgtcaggatgcctgtggtcatgctctcagctccacctggcatgagaagggatcctggcctctggcatattcatcaagtatgagttctggggatgagtcactgtaatgatgtgagcagggagccttcctccctgggccacctgcagagagctttcccaccaactttgtaccttgattgccttacaaagttatttgtttacaaacagcgaccatataaaagcctcctgccccaaagcttgtgggcacatgggcacatacagactcacatacagacacacacatatatgtacagacatgtactctcacacacacaggcaccagcatacacacgtttttctaggtacagctcccaggaacagctaggtgggaaagtcccatcactgagggagcctaaccatgtccctgaacaaaaattgggcactcatctattccttttctcttgtgtccctactcattgaaaccaaactctggaaaggacccaatgtaccagtatttatacctctaatgaagcacagagagaggaagagagctgcttaaactcacacaacaatgaactgcagacacagctgttctctccctctctccttcccagagcaatttatactttaccctcaggctgtcctctggggagaaggtgccatggtcttaggtgtctgtgccccaggacagaccctaggaccctaaatccaatagaaaatgcatatctttgctccactttcagccaggctggagcaaggtaccttttcttaggatcttgggagggaatggatgcccctctctgcatgatcttgttgaggcatttagctgccatgcacctgtccccctttaatactgggcattttaaagccatctcaagaggcatcttctacatgttttgtacgcattaaaataatttcaaagatatctgagaaaagccgatatttgccattcttcctatatcctggaatatatcttgcatcctgagtttataataataaataatattctaccttggaaa

SEQ ID NO: 198 - DNA, 2532 bp, Homo sapiens
  source 1..2532: mol_type = genomic DNA
aatgcgacccgaccgcgctgaggctccaggaccgcccgccatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctgaagactgttctgaggccctgggtgtgtgtgtatctgtctgcctgtccatgtacttctgccctgcctcctcctttcgttgtaggaggaatctgtgctctacttacctctcaattcctggagatgccaacttcacagacacgtctgcagcagctggacatcacatttcatgtcctgcatggaaccagtggctgtgagtggcatgtccacttgctggattatcagccaggacactatagaacaggaccagctgagactaagaaggaccagcagagccagctcagctctgagccattcacacatcttcaccctcagtttcctcacttgaggagtgggatggggagaacagagagtagctgtgtttgaatccctgtaggaaatggtgaagcatagctctgggtctcctgggggagaccaggcttggctgcgggagagctggctgttgctggactacatgctggccactgctgtgaccacgacactgctggggcagcttcttccacagtgatgcctactgatgcttcagtgcctctgcacaccgcccattccacttcctccttccccacagggcaggtggggaagcagtttggcccagcccaaggagaccccaccttgagccttatttcctaatgggtccacctctcatctgcatctttcacacctcccagcttctgcccaaccttcagcagtgacaagtccccaagagactcgcctgagcagcttgggctgcttttcatttccacctgtcaggatgcctgtggtcatgctctcagctccacctggcatgagaagggatcctggcctctggcatattcatcaagtatgagttctggggatgagtcactgtaatgatgtgagcagggagccttcctccctgggccacctgcagagagctttcccaccaactttgtaccttgattgccttacaaagttatttgtttacaaacagcgaccatataaaagcctcctgccccaaagcttgtgggcacatgggcacatacagactcacatacagacacacacatatatgtacagacatgtactctcacacacacaggcaccagcatacacacgtttttctaggtacagctcccaggaacagctaggtgggaaagtcccatcactgagggagcctaaccatgtccctgaacaaaaattgggcactcatctattccttttctcttgtgtccctactcattgaaaccaaactctggaaaggacccaatgtaccagtatttatacctctaatgaagcacagagagaggaagagagctgcttaaactcacacaacaatgaactgcagacacagctgttctctccctctctccttcccagagcaatttatactttaccctcaggctgtcctctggggagaaggtgccatggtcttaggtgtctgtgccccaggacagaccctaggaccctaaatccaatagaaaatgcatatctttgctccactttcagccaggctggagcaaggtaccttttcttaggatcttgggagggaatggatgcccctctctgcatgatcttgttgaggcatttagctgccatgcacctgtccccctttaatactgggcattttaaagccatctcaagaggcatcttctacatgttttgtacgcattaaaataatttcaaagatatctgagaaaagccgatatttgccattcttcctatatcctggaatatatcttgcatcctgagtttataataataaataatattctaccttggaaa

SEQ ID NO: 199 - DNA, 2486 bp, Homo sapiens
  source 1..2486: mol_type = genomic DNA
aatgcgacccgaccgcgctgaggctccaggaccgcccgccatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctgggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctgaagactgttctgaggccctgggtgtgtgtgtatctgtctgcctgtccatgtacttctgccctgcctcctcctttcgttgtaggaggaatctgtgctctacttacctctcaattcctggagatgccaacttcacagacacgtctgcagcagctggacatcacatttcatgtcctgcatggaaccagtggctgtgagtggcatgtccacttgctggattatcagccaggacactatagaacaggaccagctgagactaagaaggaccagcagagccagctcagctctgagccattcacacatcttcaccctcagtttcctcacttgaggagtgggatggggagaacagagagtagctgtgtttgaatccctgtaggaaatggtgaagcatagctctgggtctcctgggggagaccaggcttggctgcgggagagctggctgttgctggactacatgctggccactgctgtgaccacgacactgctggggcagcttcttccacagtgatgcctactgatgcttcagtgcctctgcacaccgcccattccacttcctccttccccacagggcaggtggggaagcagtttggcccagcccaaggagaccccaccttgagccttatttcctaatgggtccacctctcatctgcatctttcacacctcccagcttctgcccaaccttcagcagtgacaagtccccaagagactcgcctgagcagcttgggctgcttttcatttccacctgtcaggatgcctgtggtcatgctctcagctccacctggcatgagaagggatcctggcctctggcatattcatcaagtatgagttctggggatgagtcactgtaatgatgtgagcagggagccttcctccctgggccacctgcagagagctttcccaccaactttgtaccttgattgccttacaaagttatttgtttacaaacagcgaccatataaaagcctcctgccccaaagcttgtgggcacatgggcacatacagactcacatacagacacacacatatatgtacagacatgtactctcacacacacaggcaccagcatacacacgtttttctaggtacagctcccaggaacagctaggtgggaaagtcccatcactgagggagcctaaccatgtccctgaacaaaaattgggcactcatctattccttttctcttgtgtccctactcattgaaaccaaactctggaaaggacccaatgtaccagtatttatacctctaatgaagcacagagagaggaagagagctgcttaaactcacacaacaatgaactgcagacacagctgttctctccctctctccttcccagagcaatttatactttaccctcaggctgtcctctggggagaaggtgccatggtcttaggtgtctgtgccccaggacagaccctaggaccctaaatccaatagaaaatgcatatctttgctccactttcagccaggctggagcaaggtaccttttcttaggatcttgggagggaatggatgcccctctctgcatgatcttgttgaggcatttagctgccatgcacctgtccccctttaatactgggcattttaaagccatctcaagaggcatcttctacatgttttgtacgcattaaaataatttcaaagatatctgagaaaagccgatatttgccattcttcctatatcctggaatatatcttgcatcctgagtttataataataaataatattctaccttggaaa

SEQ ID NO: 200 - DNA, 2648 bp, Homo sapiens
  source 1..2648: mol_type = genomic DNA
aatgcgacccgaccgcgctgaggctccaggaccgcccgccatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctgaagactgttctgaggccctgggtgtgtgtgtatctgtctgcctgtccatgtacttctgccctgcctcctcctttcgttgtaggaggaatctgtgctctacttacctctcaattcctggagatgccaacttcacagacacgtctgcagcagctggacatcacatttcatgtcctgcatggaaccagtggctgtgagtggcatgtccacttgctggattatcagccaggacactatagaacaggaccagctgagactaagaaggaccagcagagccagctcagctctgagccattcacacatcttcaccctcagtttcctcacttgaggagtgggatggggagaacagagagtagctgtgtttgaatccctgtaggaaatggtgaagcatagctctgggtctcctgggggagaccaggcttggctgcgggagagctggctgttgctggactacatgctggccactgctgtgaccacgacactgctggggcagcttcttccacagtgatgcctactgatgcttcagtgcctctgcacaccgcccattccacttcctccttccccacagggcaggtggggaagcagtttggcccagcccaaggagaccccaccttgagccttatttcctaatgggtccacctctcatctgcatctttcacacctcccagcttctgcccaaccttcagcagtgacaagtccccaagagactcgcctgagcagcttgggctgcttttcatttccacctgtcaggatgcctgtggtcatgctctcagctccacctggcatgagaagggatcctggcctctggcatattcatcaagtatgagttctggggatgagtcactgtaatgatgtgagcagggagccttcctccctgggccacctgcagagagctttcccaccaactttgtaccttgattgccttacaaagttatttgtttacaaacagcgaccatataaaagcctcctgccccaaagcttgtgggcacatgggcacatacagactcacatacagacacacacatatatgtacagacatgtactctcacacacacaggcaccagcatacacacgtttttctaggtacagctcccaggaacagctaggtgggaaagtcccatcactgagggagcctaaccatgtccctgaacaaaaattgggcactcatctattccttttctcttgtgtccctactcattgaaaccaaactctggaaaggacccaatgtaccagtatttatacctctaatgaagcacagagagaggaagagagctgcttaaactcacacaacaatgaactgcagacacagctgttctctccctctctccttcccagagcaatttatactttaccctcaggctgtcctctggggagaaggtgccatggtcttaggtgtctgtgccccaggacagaccctaggaccctaaatccaatagaaaatgcatatctttgctccactttcagccaggctggagcaaggtaccttttcttaggatcttgggagggaatggatgcccctctctgcatgatcttgttgaggcatttagctgccatgcacctgtccccctttaatactgggcattttaaagccatctcaagaggcatcttctacatgttttgtacgcattaaaataatttcaaagatatctgagaaaagccgatatttgccattcttcctatatcctggaatatatcttgcatcctgagtttataataataaataatattctaccttggaaa

SEQ ID NO: 201 - DNA, 2513 bp, Homo sapiens
  source 1..2513: mol_type = genomic DNA
aatgcgacccgaccgcgctgaggctccaggaccgcccgccatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctgaagactgttctgaggccctgggtgtgtgtgtatctgtctgcctgtccatgtacttctgccctgcctcctcctttcgttgtaggaggaatctgtgctctacttacctctcaattcctggagatgccaacttcacagacacgtctgcagcagctggacatcacatttcatgtcctgcatggaaccagtggctgtgagtggcatgtccacttgctggattatcagccaggacactatagaacaggaccagctgagactaagaaggaccagcagagccagctcagctctgagccattcacacatcttcaccctcagtttcctcacttgaggagtgggatggggagaacagagagtagctgtgtttgaatccctgtaggaaatggtgaagcatagctctgggtctcctgggggagaccaggcttggctgcgggagagctggctgttgctggactacatgctggccactgctgtgaccacgacactgctggggcagcttcttccacagtgatgcctactgatgcttcagtgcctctgcacaccgcccattccacttcctccttccccacagggcaggtggggaagcagtttggcccagcccaaggagaccccaccttgagccttatttcctaatgggtccacctctcatctgcatctttcacacctcccagcttctgcccaaccttcagcagtgacaagtccccaagagactcgcctgagcagcttgggctgcttttcatttccacctgtcaggatgcctgtggtcatgctctcagctccacctggcatgagaagggatcctggcctctggcatattcatcaagtatgagttctggggatgagtcactgtaatgatgtgagcagggagccttcctccctgggccacctgcagagagctttcccaccaactttgtaccttgattgccttacaaagttatttgtttacaaacagcgaccatataaaagcctcctgccccaaagcttgtgggcacatgggcacatacagactcacatacagacacacacatatatgtacagacatgtactctcacacacacaggcaccagcatacacacgtttttctaggtacagctcccaggaacagctaggtgggaaagtcccatcactgagggagcctaaccatgtccctgaacaaaaattgggcactcatctattccttttctcttgtgtccctactcattgaaaccaaactctggaaaggacccaatgtaccagtatttatacctctaatgaagcacagagagaggaagagagctgcttaaactcacacaacaatgaactgcagacacagctgttctctccctctctccttcccagagcaatttatactttaccctcaggctgtcctctggggagaaggtgccatggtcttaggtgtctgtgccccaggacagaccctaggaccctaaatccaatagaaaatgcatatctttgctccactttcagccaggctggagcaaggtaccttttcttaggatcttgggagggaatggatgcccctctctgcatgatcttgttgaggcatttagctgccatgcacctgtccccctttaatactgggcattttaaagccatctcaagaggcatcttctacatgttttgtacgcattaaaataatttcaaagatatctgagaaaagccgatatttgccattcttcctatatcctggaatatatcttgcatcctgagtttataataataaataatattctaccttggaaa

SEQ ID NO: 202 - DNA, 2624 bp, Homo sapiens
  source 1..2624: mol_type = genomic DNA
aatgcgacccgaccgcgctgaggctccaggaccgcccgccatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctgaagactgttctgaggccctgggtgtgtgtgtatctgtctgcctgtccatgtacttctgccctgcctcctcctttcgttgtaggaggaatctgtgctctacttacctctcaattcctggagatgccaacttcacagacacgtctgcagcagctggacatcacatttcatgtcctgcatggaaccagtggctgtgagtggcatgtccacttgctggattatcagccaggacactatagaacaggaccagctgagactaagaaggaccagcagagccagctcagctctgagccattcacacatcttcaccctcagtttcctcacttgaggagtgggatggggagaacagagagtagctgtgtttgaatccctgtaggaaatggtgaagcatagctctgggtctcctgggggagaccaggcttggctgcgggagagctggctgttgctggactacatgctggccactgctgtgaccacgacactgctggggcagcttcttccacagtgatgcctactgatgcttcagtgcctctgcacaccgcccattccacttcctccttccccacagggcaggtggggaagcagtttggcccagcccaaggagaccccaccttgagccttatttcctaatgggtccacctctcatctgcatctttcacacctcccagcttctgcccaaccttcagcagtgacaagtccccaagagactcgcctgagcagcttgggctgcttttcatttccacctgtcaggatgcctgtggtcatgctctcagctccacctggcatgagaagggatcctggcctctggcatattcatcaagtatgagttctggggatgagtcactgtaatgatgtgagcagggagccttcctccctgggccacctgcagagagctttcccaccaactttgtaccttgattgccttacaaagttatttgtttacaaacagcgaccatataaaagcctcctgccccaaagcttgtgggcacatgggcacatacagactcacatacagacacacacatatatgtacagacatgtactctcacacacacaggcaccagcatacacacgtttttctaggtacagctcccaggaacagctaggtgggaaagtcccatcactgagggagcctaaccatgtccctgaacaaaaattgggcactcatctattccttttctcttgtgtccctactcattgaaaccaaactctggaaaggacccaatgtaccagtatttatacctctaatgaagcacagagagaggaagagagctgcttaaactcacacaacaatgaactgcagacacagctgttctctccctctctccttcccagagcaatttatactttaccctcaggctgtcctctggggagaaggtgccatggtcttaggtgtctgtgccccaggacagaccctaggaccctaaatccaatagaaaatgcatatctttgctccactttcagccaggctggagcaaggtaccttttcttaggatcttgggagggaatggatgcccctctctgcatgatcttgttgaggcatttagctgccatgcacctgtccccctttaatactgggcattttaaagccatctcaagaggcatcttctacatgttttgtacgcattaaaataatttcaaagatatctgagaaaagccgatatttgccattcttcctatatcctggaatatatcttgcatcctgagtttataataataaataatattctaccttggaaa

SEQ ID NO: 203 - DNA, 2329 bp, Homo sapiens
  source 1..2329: mol_type = genomic DNA
aagccctctagaacaacccagccagaggagaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctgaagactgttctgaggccctgggtgtgtgtgtatctgtctgcctgtccatgtacttctgccctgcctcctcctttcgttgtaggaggaatctgtgctctacttacctctcaattcctggagatgccaacttcacagacacgtctgcagcagctggacatcacatttcatgtcctgcatggaaccagtggctgtgagtggcatgtccacttgctggattatcagccaggacactatagaacaggaccagctgagactaagaaggaccagcagagccagctcagctctgagccattcacacatcttcaccctcagtttcctcacttgaggagtgggatggggagaacagagagtagctgtgtttgaatccctgtaggaaatggtgaagcatagctctgggtctcctgggggagaccaggcttggctgcgggagagctggctgttgctggactacatgctggccactgctgtgaccacgacactgctggggcagcttcttccacagtgatgcctactgatgcttcagtgcctctgcacaccgcccattccacttcctccttccccacagggcaggtggggaagcagtttggcccagcccaaggagaccccaccttgagccttatttcctaatgggtccacctctcatctgcatctttcacacctcccagcttctgcccaaccttcagcagtgacaagtccccaagagactcgcctgagcagcttgggctgcttttcatttccacctgtcaggatgcctgtggtcatgctctcagctccacctggcatgagaagggatcctggcctctggcatattcatcaagtatgagttctggggatgagtcactgtaatgatgtgagcagggagccttcctccctgggccacctgcagagagctttcccaccaactttgtaccttgattgccttacaaagttatttgtttacaaacagcgaccatataaaagcctcctgccccaaagcttgtgggcacatgggcacatacagactcacatacagacacacacatatatgtacagacatgtactctcacacacacaggcaccagcatacacacgtttttctaggtacagctcccaggaacagctaggtgggaaagtcccatcactgagggagcctaaccatgtccctgaacaaaaattgggcactcatctattccttttctcttgtgtccctactcattgaaaccaaactctggaaaggacccaatgtaccagtatttatacctctaatgaagcacagagagaggaagagagctgcttaaactcacacaacaatgaactgcagacacagctgttctctccctctctccttcccagagcaatttatactttaccctcaggctgtcctctggggagaaggtgccatggtcttaggtgtctgtgccccaggacagaccctaggaccctaaatccaatagaaaatgcatatctttgctccactttcagccaggctggagcaaggtaccttttcttaggatcttgggagggaatggatgcccctctctgcatgatcttgttgaggcatttagctgccatgcacctgtccccctttaatactgggcattttaaagccatctcaagaggcatcttctacatgttttgtacgcattaaaataatttcaaagatatctgagaaaagccgatatttgccattcttcctatatcctggaatatatcttgcatcctgagtttataataataaataatattctaccttggaaa

SEQ ID NO: 204 - DNA, 2667 bp, Homo sapiens
  source 1..2667: mol_type = genomic DNA
aatgcgacccgaccgcgctgaggctccaggaccgcccgccatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctgaagactgttctgaggccctgggtgtgtgtgtatctgtctgcctgtccatgtacttctgccctgcctcctcctttcgttgtaggaggaatctgtgctctacttacctctcaattcctggagatgccaacttcacagacacgtctgcagcagctggacatcacatttcatgtcctgcatggaaccagtggctgtgagtggcatgtccacttgctggattatcagccaggacactatagaacaggaccagctgagactaagaaggaccagcagagccagctcagctctgagccattcacacatcttcaccctcagtttcctcacttgaggagtgggatggggagaacagagagtagctgtgtttgaatccctgtaggaaatggtgaagcatagctctgggtctcctgggggagaccaggcttggctgcgggagagctggctgttgctggactacatgctggccactgctgtgaccacgacactgctggggcagcttcttccacagtgatgcctactgatgcttcagtgcctctgcacaccgcccattccacttcctccttccccacagggcaggtggggaagcagtttggcccagcccaaggagaccccaccttgagccttatttcctaatgggtccacctctcatctgcatctttcacacctcccagcttctgcccaaccttcagcagtgacaagtccccaagagactcgcctgagcagcttgggctgcttttcatttccacctgtcaggatgcctgtggtcatgctctcagctccacctggcatgagaagggatcctggcctctggcatattcatcaagtatgagttctggggatgagtcactgtaatgatgtgagcagggagccttcctccctgggccacctgcagagagctttcccaccaactttgtaccttgattgccttacaaagttatttgtttacaaacagcgaccatataaaagcctcctgccccaaagcttgtgggcacatgggcacatacagactcacatacagacacacacatatatgtacagacatgtactctcacacacacaggcaccagcatacacacgtttttctaggtacagctcccaggaacagctaggtgggaaagtcccatcactgagggagcctaaccatgtccctgaacaaaaattgggcactcatctattccttttctcttgtgtccctactcattgaaaccaaactctggaaaggacccaatgtaccagtatttatacctctaatgaagcacagagagaggaagagagctgcttaaactcacacaacaatgaactgcagacacagctgttctctccctctctccttcccagagcaatttatactttaccctcaggctgtcctctggggagaaggtgccatggtcttaggtgtctgtgccccaggacagaccctaggaccctaaatccaatagaaaatgcatatctttgctccactttcagccaggctggagcaaggtaccttttcttaggatcttgggagggaatggatgcccctctctgcatgatcttgttgaggcatttagctgccatgcacctgtccccctttaatactgggcattttaaagccatctcaagaggcatcttctacatgttttgtacgcattaaaataatttcaaagatatctgagaaaagccgatatttgccattcttcctatatcctggaatatatcttgcatcctgagtttataataataaataatattctaccttggaaa

SEQ ID NO: 205 - DNA, 4055 bp, Homo sapiens
  source 1..4055: mol_type = genomic DNA
gaagtgcaccagcgagccggggcaggaagaggaggtttcgccaccggagcggcccggcgacgcgctgacagcttcccctgcccttcccgtcggtcgggccgccagccgccgcagccctcggcctgcacgcagccaccggccccgctcccggagcccagcgccgccgaggccgcagccgcccggccagtaaggcggcgccgccgcccggccaccgcgcgccctgcgcttccctccgcccgcgctgcggccatggcgcggcgctgactggcctggcccggccccgccgcgctcccgctcgccccgacccgcactcgggcccgcccgggctccggcctgccgccgcctcttccttctccagccggcaggcccgcgccgcttaggagggagagcccacccgcgccaggaggccgaacgcggactcgccacccggcttcagaatggcagaagatgatccatatttgggaaggcctgaacaaatgtttcatttggatccttctttgactcatacaatatttaatccagaagtatttcaaccacagatggcactgccaacagcagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgctggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacagcggcgtggagacatccttccgcaaactcagctttaccgagtctctgaccagtggtgcctcactgctaactctcaacaaaatgccccatgattatgggcaggaaggacctctagaaggcaaaatttagcctgctgacaatttcccacaccgtgtaaaccaaagccctaaaattccactgcgttgtccacaagacagaagctgaagtgcatccaaaggtgctcagagagccggcccgcctgaatcattctcgatttaactcgagaccttttcaacttggcttcctttcttggttcataaatgaattttagtttggttcacttacagatagtatctagcaatcacaacactggctgagcggatgcatctggggatgaggttgcttactaagctttgccagctgctgctggatcacagctgctttctgttgtcattgctgttgtccctctgctacgttcctattgtcattaaaggtatcacggtcgccacctggcattccttctgaccacagcatcattttgcattcaaattaagggttaagaaaagagatattttaaaatgagagtcacttgatgtgccattttaaaaaaaaaggcatattgctttttctaatgtggttatttctctgatttgcaaaaaaaaaaaaaaaaaaaatacttgtcaatatttaaacatggttacaatcattgctgaaaatggtattttcccccttttctgcattttgctattgtaaatatgttttttagatcaaatactttaaaggaaaaaatgttggatttataaatgctattttttattttacttttataataaaaggaaaagcaaattgatgacctca

SEQ ID NO: 206 - DNA, 4052 bp, Homo sapiens
  source 1..4052: mol_type = genomic DNA
gaagtgcaccagcgagccggggcaggaagaggaggtttcgccaccggagcggcccggcgacgcgctgacagcttcccctgcccttcccgtcggtcgggccgccagccgccgcagccctcggcctgcacgcagccaccggccccgctcccggagcccagcgccgccgaggccgcagccgcccggccagtaaggcggcgccgccgcccggccaccgcgcgccctgcgcttccctccgcccgcgctgcggccatggcgcggcgctgactggcctggcccggccccgccgcgctcccgctcgccccgacccgcactcgggcccgcccgggctccggcctgccgccgcctcttccttctccagccggcaggcccgcgccgcttaggagggagagcccacccgcgccaggaggccgaacgcggactcgccacccggcttcagaatggcagaagatgatccatatttgggaaggcctgaacaaatgtttcatttggatccttctttgactcatacaatatttaatccagaagtatttcaaccacagatggcactgccaacagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgctggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacagcggcgtggagacatccttccgcaaactcagctttaccgagtctctgaccagtggtgcctcactgctaactctcaacaaaatgccccatgattatgggcaggaaggacctctagaaggcaaaatttagcctgctgacaatttcccacaccgtgtaaaccaaagccctaaaattccactgcgttgtccacaagacagaagctgaagtgcatccaaaggtgctcagagagccggcccgcctgaatcattctcgatttaactcgagaccttttcaacttggcttcctttcttggttcataaatgaattttagtttggttcacttacagatagtatctagcaatcacaacactggctgagcggatgcatctggggatgaggttgcttactaagctttgccagctgctgctggatcacagctgctttctgttgtcattgctgttgtccctctgctacgttcctattgtcattaaaggtatcacggtcgccacctggcattccttctgaccacagcatcattttgcattcaaattaagggttaagaaaagagatattttaaaatgagagtcacttgatgtgccattttaaaaaaaaaggcatattgctttttctaatgtggttatttctctgatttgcaaaaaaaaaaaaaaaaaaaatacttgtcaatatttaaacatggttacaatcattgctgaaaatggtattttcccccttttctgcattttgctattgtaaatatgttttttagatcaaatactttaaaggaaaaaatgttggatttataaatgctattttttattttacttttataataaaaggaaaagcaaattgatgacctca

SEQ ID NO: 207 - DNA, 3856 bp, Homo sapiens
  source 1..3856: mol_type = genomic DNA
agtagctgagaggcacatgggattagcgacagcggggaaagacacatccggacctcgcaggggctagtcggccgaagggccgcggccgcccggcggtcatttctcttcacgtccctccgcggggcgggaacgcgaaccggaggggaactcttacaaacttttaaaatccccaaccccagccccacctgggggatggtgagaaggttggagtgcgctgcggcgcggaggagagagcttcagaatggcagaagatgatccatatttgggaaggcctgaacaaatgtttcatttggatccttctttgactcatacaatatttaatccagaagtatttcaaccacagatggcactgccaacagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgctggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacagcggcgtggagacatccttccgcaaactcagctttaccgagtctctgaccagtggtgcctcactgctaactctcaacaaaatgccccatgattatgggcaggaaggacctctagaaggcaaaatttagcctgctgacaatttcccacaccgtgtaaaccaaagccctaaaattccactgcgttgtccacaagacagaagctgaagtgcatccaaaggtgctcagagagccggcccgcctgaatcattctcgatttaactcgagaccttttcaacttggcttcctttcttggttcataaatgaattttagtttggttcacttacagatagtatctagcaatcacaacactggctgagcggatgcatctggggatgaggttgcttactaagctttgccagctgctgctggatcacagctgctttctgttgtcattgctgttgtccctctgctacgttcctattgtcattaaaggtatcacggtcgccacctggcattccttctgaccacagcatcattttgcattcaaattaagggttaagaaaagagatattttaaaatgagagtcacttgatgtgccattttaaaaaaaaaggcatattgctttttctaatgtggttatttctctgatttgcaaaaaaaaaaaaaaaaaaaatacttgtcaatatttaaacatggttacaatcattgctgaaaatggtattttcccccttttctgcattttgctattgtaaatatgttttttagatcaaatactttaaaggaaaaaatgttggatttataaatgctattttttattttacttttataataaaaggaaaagcaaattgatgacctca

SEQ ID NO: 208 - DNA, 3934 bp, Homo sapiens
  source 1..3934: mol_type = genomic DNA
agtagctgagaggcacatgggattagcgacagcggggaaagacacatccggacctcgcaggggctagtcggccgaagggccgcggccgcccggcggtcatttctcttcacgtccctccgcggggcgggaacgcgaaccggaggggaactcttacaaacttttaaaatccccaaccccagccccacctgggggatggtgagaaggttggagtgcgctgcggcgcggaggagagagggaagaaggaacacaacgtcctacatcaagaaaaaagacagaagcatatcttggagatatgaaaagaaccaccagcttcagaatggcagaagatgatccatatttgggaaggcctgaacaaatgtttcatttggatccttctttgactcatacaatatttaatccagaagtatttcaaccacagatggcactgccaacagcagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgctggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacagcggcgtggagacatccttccgcaaactcagctttaccgagtctctgaccagtggtgcctcactgctaactctcaacaaaatgccccatgattatgggcaggaaggacctctagaaggcaaaatttagcctgctgacaatttcccacaccgtgtaaaccaaagccctaaaattccactgcgttgtccacaagacagaagctgaagtgcatccaaaggtgctcagagagccggcccgcctgaatcattctcgatttaactcgagaccttttcaacttggcttcctttcttggttcataaatgaattttagtttggttcacttacagatagtatctagcaatcacaacactggctgagcggatgcatctggggatgaggttgcttactaagctttgccagctgctgctggatcacagctgctttctgttgtcattgctgttgtccctctgctacgttcctattgtcattaaaggtatcacggtcgccacctggcattccttctgaccacagcatcattttgcattcaaattaagggttaagaaaagagatattttaaaatgagagtcacttgatgtgccattttaaaaaaaaaggcatattgctttttctaatgtggttatttctctgatttgcaaaaaaaaaaaaaaaaaaaatacttgtcaatatttaaacatggttacaatcattgctgaaaatggtattttcccccttttctgcattttgctattgtaaatatgttttttagatcaaatactttaaaggaaaaaatgttggatttataaatgctattttttattttacttttataataaaaggaaaagcaaattgatgacctca

SEQ ID NO: 209 - DNA, 4125 bp, Homo sapiens
  source 1..4125: mol_type = genomic DNA
gaagtgcaccagcgagccggggcaggaagaggaggtttcgccaccggagcggcccggcgacgcgctgacagcttcccctgcccttcccgtcggtcgggccgccagccgccgcagccctcggcctgcacgcagccaccggccccgctcccggagcccagcgccgccgaggccgcagccgcccggccagtaaggcggcgccgccgcccggccaccgcgcgccctgcgcttccctccgcccgcgctgcggccatggcgcggcgctgactggcctggcccggccccgccgcgctcccgctcgccccgacccgcactcgggcccgcccgggctccggcctgccgccgcctcttccttctccagccggcaggcccgcgccgcttaggagggagagcccacccgcgccaggaggccgaacgcggactcgccacccggaaggaacacaacgtcctacatcaagaaaaaagacagaagcatatcttggagatatgaaaagaaccaccagcttcagaatggcagaagatgatccatatttgggaaggcctgaacaaatgtttcatttggatccttctttgactcatacaatatttaatccagaagtatttcaaccacagatggcactgccaacagcagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgctggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacagcggcgtggagacatccttccgcaaactcagctttaccgagtctctgaccagtggtgcctcactgctaactctcaacaaaatgccccatgattatgggcaggaaggacctctagaaggcaaaatttagcctgctgacaatttcccacaccgtgtaaaccaaagccctaaaattccactgcgttgtccacaagacagaagctgaagtgcatccaaaggtgctcagagagccggcccgcctgaatcattctcgatttaactcgagaccttttcaacttggcttcctttcttggttcataaatgaattttagtttggttcacttacagatagtatctagcaatcacaacactggctgagcggatgcatctggggatgaggttgcttactaagctttgccagctgctgctggatcacagctgctttctgttgtcattgctgttgtccctctgctacgttcctattgtcattaaaggtatcacggtcgccacctggcattccttctgaccacagcatcattttgcattcaaattaagggttaagaaaagagatattttaaaatgagagtcacttgatgtgccattttaaaaaaaaaggcatattgctttttctaatgtggttatttctctgatttgcaaaaaaaaaaaaaaaaaaaatacttgtcaatatttaaacatggttacaatcattgctgaaaatggtattttcccccttttctgcattttgctattgtaaatatgttttttagatcaaatactttaaaggaaaaaatgttggatttataaatgctattttttattttacttttataataaaaggaaaagcaaattgatgacctca

SEQ ID NO: 210 - DNA, 4122 bp, Homo sapiens
  source 1..4122: mol_type = genomic DNA
gaagtgcaccagcgagccggggcaggaagaggaggtttcgccaccggagcggcccggcgacgcgctgacagcttcccctgcccttcccgtcggtcgggccgccagccgccgcagccctcggcctgcacgcagccaccggccccgctcccggagcccagcgccgccgaggccgcagccgcccggccagtaaggcggcgccgccgcccggccaccgcgcgccctgcgcttccctccgcccgcgctgcggccatggcgcggcgctgactggcctggcccggccccgccgcgctcccgctcgccccgacccgcactcgggcccgcccgggctccggcctgccgccgcctcttccttctccagccggcaggcccgcgccgcttaggagggagagcccacccgcgccaggaggccgaacgcggactcgccacccggaaggaacacaacgtcctacatcaagaaaaaagacagaagcatatcttggagatatgaaaagaaccaccagcttcagaatggcagaagatgatccatatttgggaaggcctgaacaaatgtttcatttggatccttctttgactcatacaatatttaatccagaagtatttcaaccacagatggcactgccaacagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgctggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacagcggcgtggagacatccttccgcaaactcagctttaccgagtctctgaccagtggtgcctcactgctaactctcaacaaaatgccccatgattatgggcaggaaggacctctagaaggcaaaatttagcctgctgacaatttcccacaccgtgtaaaccaaagccctaaaattccactgcgttgtccacaagacagaagctgaagtgcatccaaaggtgctcagagagccggcccgcctgaatcattctcgatttaactcgagaccttttcaacttggcttcctttcttggttcataaatgaattttagtttggttcacttacagatagtatctagcaatcacaacactggctgagcggatgcatctggggatgaggttgcttactaagctttgccagctgctgctggatcacagctgctttctgttgtcattgctgttgtccctctgctacgttcctattgtcattaaaggtatcacggtcgccacctggcattccttctgaccacagcatcattttgcattcaaattaagggttaagaaaagagatattttaaaatgagagtcacttgatgtgccattttaaaaaaaaaggcatattgctttttctaatgtggttatttctctgatttgcaaaaaaaaaaaaaaaaaaaatacttgtcaatatttaaacatggttacaatcattgctgaaaatggtattttcccccttttctgcattttgctattgtaaatatgttttttagatcaaatactttaaaggaaaaaatgttggatttataaatgctattttttattttacttttataataaaaggaaaagcaaattgatgacctca

SEQ ID NO: 211 - DNA, 4006 bp, Homo sapiens
  source 1..4006: mol_type = genomic DNA
gaagtgcaccagcgagccggggcaggaagaggaggtttcgccaccggagcggcccggcgacgcgctgacagcttcccctgcccttcccgtcggtcgggccgccagccgccgcagccctcggcctgcacgcagccaccggccccgctcccggagcccagcgccgccgaggccgcagccgcccggccagtaaggcggcgccgccgcccggccaccgcgcgccctgcgcttccctccgcccgcgctgcggccatggcgcggcgctgactggcctggcccggccccgccgcgctcccgctcgccccgacccgcactcgggcccgcccgggctccggcctgccgccgcctcttccttctccagccggcaggcccgcgccgcttaggagggagagcccacccgcgccaggaggccgaacgcggactcgccacccggatgtttcatttggatccttctttgactcatacaatatttaatccagaagtatttcaaccacagatggcactgccaacagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgctggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacagcggcgtggagacatccttccgcaaactcagctttaccgagtctctgaccagtggtgcctcactgctaactctcaacaaaatgccccatgattatgggcaggaaggacctctagaaggcaaaatttagcctgctgacaatttcccacaccgtgtaaaccaaagccctaaaattccactgcgttgtccacaagacagaagctgaagtgcatccaaaggtgctcagagagccggcccgcctgaatcattctcgatttaactcgagaccttttcaacttggcttcctttcttggttcataaatgaattttagtttggttcacttacagatagtatctagcaatcacaacactggctgagcggatgcatctggggatgaggttgcttactaagctttgccagctgctgctggatcacagctgctttctgttgtcattgctgttgtccctctgctacgttcctattgtcattaaaggtatcacggtcgccacctggcattccttctgaccacagcatcattttgcattcaaattaagggttaagaaaagagatattttaaaatgagagtcacttgatgtgccattttaaaaaaaaaggcatattgctttttctaatgtggttatttctctgatttgcaaaaaaaaaaaaaaaaaaaatacttgtcaatatttaaacatggttacaatcattgctgaaaatggtattttcccccttttctgcattttgctattgtaaatatgttttttagatcaaatactttaaaggaaaaaatgttggatttataaatgctattttttattttacttttataataaaaggaaaagcaaattgatgacctca

SEQ ID NO: 212 - DNA, 3015 bp, Homo sapiens
  source 1..3015: mol_type = genomic DNA
aactttcctgccccttccccggccaagcccaactccggatctcgctctccaccggatctcacccgccacacccggacaggcggctggaggaggcgggcgtctaaaattctgggaagcagaacctggccggagccactagacagagccgggcctagcccagagacatggagagttgctacaacccaggtctggatggtattattgaatatgatgatttcaaattgaactcctccattgtggaacccaaggagccagccccagaaacagctgatggcccctacctggtgatcgtggaacagcctaagcagagaggcttccgatttcgatatggctgtgaaggcccctcccatggaggactgcccggtgcctccagtgagaagggccgaaagacctatcccactgtcaagatctgtaactacgagggaccagccaagatcgaggtggacctggtaacacacagtgacccacctcgtgctcatgcccacagtctggtgggcaagcaatgctcggagctggggatctgcgccgtttctgtggggcccaaggacatgactgcccaatttaacaacctgggtgtcctgcatgtgactaagaagaacatgatggggactatgatacaaaaacttcagaggcagcggctccgctctaggccccagggccttacggaggccgagcagcgggagctggagcaagaggccaaagaactgaagaaggtgatggatctgagtatagtgcggctgcgcttctctgccttccttagagccagtgatggctccttctccctgcccctgaagccagtcatctcccagcccatccatgacagcaaatctccgggggcatcaaacctgaagatttctcgaatggacaagacagcaggctctgtgcggggtggagatgaagtttatctgctttgtgacaaggtgcagaaagatgacattgaggttcggttctatgaggatgatgagaatggatggcaggcctttggggacttctctcccacagatgtgcataaacagtatgccattgtgttccggacacccccctatcacaagatgaagattgagcggcctgtaacagtgtttctgcaactgaaacgcaagcgaggaggggacgtgtctgattccaaacagttcacctattaccctctggtggaagacaaggaagaggtgcagcggaagcggaggaaggccttgcccaccttctcccagcccttcgggggtggctcccacatgggtggaggctctgggggtgcagccgggggctacggaggagctggaggaggtggcagcctcggtttcttcccctcctccctggcctacagcccctaccagtccggcgcgggccccatgggctgctacccgggaggcgggggcggggcgcagatggccgccacggtgcccagcagggactccggggaggaagccgcggagccaagcgccccctccaggaccccccagtgcgagccgcaggccccggagatgctgcagcgagctcgagagtacaacgcgcgcctgttcggcctggcgcagcgcagcgcccgagccctactcgactacggcgtcaccgcggacgcgcgcgcgctgctggcgggacagcgccacctgctgacggcgcaggacgagaacggagacacaccactgcacctagccatcatccacgggcagaccagtgtcattgagcagatagtctatgtcatccaccacgcccaggacctcggcgttgtcaacctcaccaaccacctgcaccagacgcccctgcacctggcggtgatcacggggcagacgagtgtggtgagctttctgctgcgggtaggtgcagacccagctctgctggatcggcatggagactcagccatgcatctggcgctgcgggcaggcgctggtgctcctgagctgctgcgtgcactgcttcagagtggagctcctgctgtgccccagctgttgcatatgcctgactttgagggactgtatccagtacacctggcagtccgagcccgaagccctgagtgcctggatctgctggtggacagtggggctgaagtggaggccacagagcggcaggggggacgaacagccttgcatctagccacagagatggaggagctggggttggtcacccatctggtcaccaagctccgggccaacgtgaacgctcgcacctttgcgggaaacacacccctgcacctggcagctggactggggtacccgaccctcacccgcctccttctgaaggctggtgctgacatccatgctgaaaacgaggagcccctgtgcccactgccttcaccccctacctctgatagcgactcggactctgaagggcctgagaaggacacccgaagcagcttccggggccacacgcctcttgacctcacttgcagcaccaaggtgaagaccttgctgctaaatgctgctcagaacaccatggagccacccctgaccccgcccagcccagcagggccgggactgtcacttggtgatacagctctgcagaacctggagcagctgctagacgggccagaagcccagggcagctgggcagagctggcagagcgtctggggctgcgcagcctggtagacacgtaccgacagacaacctcacccagtggcagcctcctgcgcagctacgagctggctggcggggacctggcaggtctactggaggccctgtctgacatgggcctagaggagggagtgaggctgctgaggggtccagaaacccgagacaagctgcccagcacagcagaggtgaaggaagacagtgcgtacgggagccagtcagtggagcaggaggcagagaagctgggcccaccccctgagccaccaggagggctctgccacgggcacccccagcctcaggtgcactgacctgctgcctgcccccagcccccttcccggaccccctgtacagcgtccccacctatttcaaatcttatttaacaccccacacccacccctcagttgggacaaataaaggattctcatgggaaggggaggacccctccttcccaactta

SEQ ID NO: 213 - DNA, 3012 bp, Homo sapiens
  source 1..3012: mol_type = genomic DNA
aactttcctgccccttccccggccaagcccaactccggatctcgctctccaccggatctcacccgccacacccggacaggcggctggaggaggcgggcgtctaaaattctgggaagcagaacctggccggagccactagacagagccgggcctagcccagagacatggagagttgctacaacccaggtctggatggtattattgaatatgatgatttcaaattgaactcctccattgtggaacccaaggagccagccccagaaacagctgatggcccctacctggtgatcgtggaacagcctaagcagagaggcttccgatttcgatatggctgtgaaggcccctcccatggaggactgcccggtgcctccagtgagaagggccgaaagacctatcccactgtcaagatctgtaactacgagggaccagccaagatcgaggtggacctggtaacacacagtgacccacctcgtgctcatgcccacagtctggtgggcaagcaatgctcggagctggggatctgcgccgtttctgtggggcccaaggacatgactgcccaatttaacaacctgggtgtcctgcatgtgactaagaagaacatgatggggactatgatacaaaaacttcagaggcagcggctccgctctaggccccagggccttacggaggccgagcagcgggagctggagcaagaggccaaagaactgaagaaggtgatggatctgagtatagtgcggctgcgcttctctgccttccttagagccagtgatggctccttctccctgcccctgaagccagtcatctcccagcccatccatgacagcaaatctccgggggcatcaaacctgaagatttctcgaatggacaagacagcaggctctgtgcggggtggagatgaagtttatctgctttgtgacaaggtgcagaaagatgacattgaggttcggttctatgaggatgatgagaatggatggcaggcctttggggacttctctcccacagatgtgcataaacagtatgccattgtgttccggacacccccctatcacaagatgaagattgagcggcctgtaacagtgtttctgcaactgaaacgcaagcgaggaggggacgtgtctgattccaaacagttcacctattaccctctggtggaagacaaggaagaggtgcagcggaagcggaggaaggccttgcccaccttctcccagcccttcgggggtggctcccacatgggtggaggctctgggggtgcagccgggggctacggaggagctggaggaggtggcagcctcggtttcttcccctcctccctggcctacagcccctaccagtccggcgcgggccccatgggctgctacccgggaggcgggggcggggcgcagatggccgccacggtgcccagcagggactccggggaggaagccgcggagccaagcgccccctccaggaccccccagtgcgagccgcaggccccggagatgctgcagcgagctcgagagtacaacgcgcgcctgttcggcctggcgcagcgcagcgcccgagccctactcgactacggcgtcaccgcggacgcgcgcgcgctgctggcgggacagcgccacctgctgacggcgcaggacgagaacggagacacaccactgcacctagccatcatccacgggcagaccagtgtcattgagcagatagtctatgtcatccaccacgcccaggacctcggcgttgtcaacctcaccaaccacctgcaccagacgcccctgcacctggcggtgatcacggggcagacgagtgtggtgagctttctgctgcgggtaggtgcagacccagctctgctggatcggcatggagactcagccatgcatctggcgctgcgggcaggcgctggtgctcctgagctgctgcgtgcactgcttcagagtggagctcctgctgtgccccagctgttgcatatgcctgactttgagggactgtatccagtacacctggcagtccgagcccgaagccctgagtgcctggatctgctggtggacagtggggctgaagtggaggccacagagcggcaggggggacgaacagccttgcatctagccacagagatggaggagctggggttggtcacccatctggtcaccaagctccgggccaacgtgaacgctcgcacctttgcgggaaacacacccctgcacctggcagctggactggggtacccgaccctcacccgcctccttctgaaggctggtgctgacatccatgctgaaaacgaggagcccctgtgcccactgccttcaccccctacctctgatagcgactcggactctgaagggcctgagaaggacacccgaagcagcttccggggccacacgcctcttgacctcacttgcagcaccaaggtgaagaccttgctgctaaatgctgctcagaacaccatggagccacccctgaccccgcccagcccagcagggccgggactgtcacttggtgatacagctctgcagaacctggagcagctgctagacgggccagaagcccagggcagctgggcagagctggcagagcgtctggggctgcgcagcctggtagacacgtaccgacagacaacctcacccagtggcagcctcctgcgcagctacgagctggctggcggggacctggcaggtctactggaggccctgtctgacatgggcctagaggagggagtgaggctgctgaggggtccagaaacccgagacaagctgcccagcacagaggtgaaggaagacagtgcgtacgggagccagtcagtggagcaggaggcagagaagctgggcccaccccctgagccaccaggagggctctgccacgggcacccccagcctcaggtgcactgacctgctgcctgcccccagcccccttcccggaccccctgtacagcgtccccacctatttcaaatcttatttaacaccccacacccacccctcagttgggacaaataaaggattctcatgggaaggggaggacccctccttcccaactta

SEQ ID NO: 214 - DNA, 3125 bp, Homo sapiens
  source 1..3125: mol_type = genomic DNA
ccgcaaccagagccgccgccacggtgagtggctggattcagacccctgggtggccgggacaagagaaaagagggaggagggcctttagcggacagcgcctggggctggagagcagcagctgcacacagccggaaagggcgcgcaggcgacgacactcggatccacgtcgacaccgttgtacaaagatacgcggacccgcgggcgtctaaaattctgggaagcagaacctggccggagccactagacagagccgggcctagcccagagacatggagagttgctacaacccaggtctggatggtattattgaatatgatgatttcaaattgaactcctccattgtggaacccaaggagccagccccagaaacagctgatggcccctacctggtgatcgtggaacagcctaagcagagaggcttccgatttcgatatggctgtgaaggcccctcccatggaggactgcccggtgcctccagtgagaagggccgaaagacctatcccactgtcaagatctgtaactacgagggaccagccaagatcgaggtggacctggtaacacacagtgacccacctcgtgctcatgcccacagtctggtgggcaagcaatgctcggagctggggatctgcgccgtttctgtggggcccaaggacatgactgcccaatttaacaacctgggtgtcctgcatgtgactaagaagaacatgatggggactatgatacaaaaacttcagaggcagcggctccgctctaggccccagggccttacggaggccgagcagcgggagctggagcaagaggccaaagaactgaagaaggtgatggatctgagtatagtgcggctgcgcttctctgccttccttagagccagtgatggctccttctccctgcccctgaagccagtcatctcccagcccatccatgacagcaaatctccgggggcatcaaacctgaagatttctcgaatggacaagacagcaggctctgtgcggggtggagatgaagtttatctgctttgtgacaaggtgcagaaagatgacattgaggttcggttctatgaggatgatgagaatggatggcaggcctttggggacttctctcccacagatgtgcataaacagtatgccattgtgttccggacacccccctatcacaagatgaagattgagcggcctgtaacagtgtttctgcaactgaaacgcaagcgaggaggggacgtgtctgattccaaacagttcacctattaccctctggtggaagacaaggaagaggtgcagcggaagcggaggaaggccttgcccaccttctcccagcccttcgggggtggctcccacatgggtggaggctctgggggtgcagccgggggctacggaggagctggaggaggtggcagcctcggtttcttcccctcctccctggcctacagcccctaccagtccggcgcgggccccatgggctgctacccgggaggcgggggcggggcgcagatggccgccacggtgcccagcagggactccggggaggaagccgcggagccgagcgccccctccaggaccccccagtgcgagccgcaggccccggagatgctgcagcgagctcgagagtacaacgcgcgcctgttcggcctggcgcagcgcagcgcccgagccctactcgactacggcgtcaccgcggacgcgcgcgcgctgctggcgggacagcgccacctgctgacggcgcaggacgagaacggagacacaccactgcacctagccatcatccacgggcagaccagtgtcattgagcagatagtctatgtcatccaccacgcccaggacctcggcgttgtcaacctcaccaaccacctgcaccagacgcccctgcacctggcggtgatcacggggcagacgagtgtggtgagctttctgctgcgggtaggtgcagacccagctctgctggatcggcatggagactcagccatgcatctggcgctgcgggcaggcgctggtgctcctgagctgctgcgtgcactgcttcagagtggagctcctgctgtgccccagctgttgcatatgcctgactttgagggactgtatccagtacacctggcggtccgagcccgaagccctgagtgcctggatctgctggtggacagtggggctgaagtggaggccacagagcggcaggggggacgaacagccttgcatctagccacagagatggaggagctggggttggtcacccatctggtcaccaagctccgggccaacgtgaacgctcgcacctttgcgggaaacacacccctgcacctggcagctggactggggtacccgaccctcacccgcctccttctgaaggctggtgctgacatccatgctgaaaacgaggagcccctgtgcccactgccttcaccccctacctctgatagcgactcggactctgaagggcctgagaaggacacccgaagcagcttccggggccacacgcctcttgacctcacttgcagcaccaaggtgaagaccttgctgctaaatgctgctcagaacaccatggagccacccctgaccccgcccagcccagcagggccgggactgtcacttggtgatacagctctgcagaacctggagcagctgctagacgggccagaagcccagggcagctgggcagagctggcagagcgtctggggctgcgcagcctggtagacacgtaccgacagacaacctcacccagtggcagcctcctgcgcagctacgagctggctggcggggacctggcaggtctactggaggccctgtctgacatgggcctagaggagggagtgaggctgctgaggggtccagaaacccgagacaagctgcccagcacagcagaggtgaaggaagacagtgcgtacgggagccagtcagtggagcaggaggcagagaagctgggcccaccccctgagccaccaggagggctctgccacgggcacccccagcctcaggtgcactgacctgctgcctgcccccagcccccttcccggaccccctgtacagcgtccccacctatttcaaatcttatttaacaccccacacccacccctcagttgggacaaataaaggattctcatgggaaggggaggacccctccttcccaacttatggca

SEQ ID NO: 215 - DNA, 3103 bp, Homo sapiens
  source 1..3103: mol_type = genomic DNA
aactttcctgccccttccccggccaagcccaactccggatctcgctctccaccggatctcacccgccacacccggacaggcggctggaggaggtcggaccctcccccaaatctgggcccccatctcccgcccacccccatttagatctgaccccctcccccacgccactcctcccaactttaggcgggcgtctaaaattctgggaagcagaacctggccggagccactagacagagccgggcctagcccagagacatggagagttgctacaacccaggtctggatggtattattgaatatgatgatttcaaattgaactcctccattgtggaacccaaggagccagccccagaaacagctgatggcccctacctggtgatcgtggaacagcctaagcagagaggcttccgatttcgatatggctgtgaaggcccctcccatggaggactgcccggtgcctccagtgagaagggccgaaagacctatcccactgtcaagatctgtaactacgagggaccagccaagatcgaggtggacctggtaacacacagtgacccacctcgtgctcatgcccacagtctggtgggcaagcaatgctcggagctggggatctgcgccgtttctgtggggcccaaggacatgactgcccaatttaacaacctgggtgtcctgcatgtgactaagaagaacatgatggggactatgatacaaaaacttcagaggcagcggctccgctctaggccccagggccttacggaggccgagcagcgggagctggagcaagaggccaaagaactgaagaaggtgatggatctgagtatagtgcggctgcgcttctctgccttccttagagccagtgatggctccttctccctgcccctgaagccagtcatctcccagcccatccatgacagcaaatctccgggggcatcaaacctgaagatttctcgaatggacaagacagcaggctctgtgcggggtggagatgaagtttatctgctttgtgacaaggtgcagaaagatgacattgaggttcggttctatgaggatgatgagaatggatggcaggcctttggggacttctctcccacagatgtgcataaacagtatgccattgtgttccggacacccccctatcacaagatgaagattgagcggcctgtaacagtgtttctgcaactgaaacgcaagcgaggaggggacgtgtctgattccaaacagttcacctattaccctctggtggaagacaaggaagaggtgcagcggaagcggaggaaggccttgcccaccttctcccagcccttcgggggtggctcccacatgggtggaggctctgggggtgcagccgggggctacggaggagctggaggaggtggcagcctcggtttcttcccctcctccctggcctacagcccctaccagtccggcgcgggccccatgggctgctacccgggaggcgggggcggggcgcagatggccgccacggtgcccagcagggactccggggaggaagccgcggagccaagcgccccctccaggaccccccagtgcgagccgcaggccccggagatgctgcagcgagctcgagagtacaacgcgcgcctgttcggcctggcgcagcgcagcgcccgagccctactcgactacggcgtcaccgcggacgcgcgcgcgctgctggcgggacagcgccacctgctgacggcgcaggacgagaacggagacacaccactgcacctagccatcatccacgggcagaccagtgtcattgagcagatagtctatgtcatccaccacgcccaggacctcggcgttgtcaacctcaccaaccacctgcaccagacgcccctgcacctggcggtgatcacggggcagacgagtgtggtgagctttctgctgcgggtaggtgcagacccagctctgctggatcggcatggagactcagccatgcatctggcgctgcgggcaggcgctggtgctcctgagctgctgcgtgcactgcttcagagtggagctcctgctgtgccccagctgttgcatatgcctgactttgagggactgtatccagtacacctggcagtccgagcccgaagccctgagtgcctggatctgctggtggacagtggggctgaagtggaggccacagagcggcaggggggacgaacagccttgcatctagccacagagatggaggagctggggttggtcacccatctggtcaccaagctccgggccaacgtgaacgctcgcacctttgcgggaaacacacccctgcacctggcagctggactggggtacccgaccctcacccgcctccttctgaaggctggtgctgacatccatgctgaaaacgaggagcccctgtgcccactgccttcaccccctacctctgatagcgactcggactctgaagggcctgagaaggacacccgaagcagcttccggggccacacgcctcttgacctcacttgcagcaccaaggtgaagaccttgctgctaaatgctgctcagaacaccatggagccacccctgaccccgcccagcccagcagggccgggactgtcacttggtgatacagctctgcagaacctggagcagctgctagacgggccagaagcccagggcagctgggcagagctggcagagcgtctggggctgcgcagcctggtagacacgtaccgacagacaacctcacccagtggcagcctcctgcgcagctacgagctggctggcggggacctggcaggtctactggaggccctgtctgacatgggcctagaggagggagtgaggctgctgaggggtccagaaacccgagacaagctgcccagcacagaggtgaaggaagacagtgcgtacgggagccagtcagtggagcaggaggcagagaagctgggcccaccccctgagccaccaggagggctctgccacgggcacccccagcctcaggtgcactgacctgctgcctgcccccagcccccttcccggaccccctgtacagcgtccccacctatttcaaatcttatttaacaccccacacccacccctcagttgggacaaataaaggattctcatgggaaggggaggacccctccttcccaactta

SEQ ID NO: 216 - DNA, 3416 bp, Homo sapiens
  source 1..3416: mol_type = genomic DNA
cttcctcttccgctaacttcccggcagcgcgccgctcagggtgggggccccgagggctggggccgtcggcttccccctggggatcccccgcttcagagaaagccaagcgttaggcgcagccaaagccgagaggcagcggaagctcccggcccggggtggcgctgggtcagcgggtaccttctcggcggtcccctggccggccgaactcgcgcctggtgtcctgtcaccccgctccccgccctgagtgagcctgtcccctctcaggggcgcgcccgagtcgctccgggttggctgcgccagtccagagttaaactttcagccaatgaaaaagggcgcgaggcgtgacgcacggaaacgtcatgggaattcccccctccggggggccgagaaggggctttcccggccctgagccctgctggcaggcgaggtgtcgcgaccggtcccaggtgggtcgggcgcggagagaagccgcaaccagagccgccgccacggcgggcgtctaaaattctgggaagcagaacctggccggagccactagacagagccgggcctagcccagagacatggagagttgctacaacccaggtctggatggtattattgaatatgatgatttcaaattgaactcctccattgtggaacccaaggagccagccccagaaacagctgatggcccctacctggtgatcgtggaacagcctaagcagagaggcttccgatttcgatatggctgtgaaggcccctcccatggaggactgcccggtgcctccagtgagaagggccgaaagacctatcccactgtcaagatctgtaactacgagggaccagccaagatcgaggtggacctggtaacacacagtgacccacctcgtgctcatgcccacagtctggtgggcaagcaatgctcggagctggggatctgcgccgtttctgtggggcccaaggacatgactgcccaatttaacaacctgggtgtcctgcatgtgactaagaagaacatgatggggactatgatacaaaaacttcagaggcagcggctccgctctaggccccagggccttacggaggccgagcagcgggagctggagcaagaggccaaagaactgaagaaggtgatggatctgagtatagtgcggctgcgcttctctgccttccttagagccagtgatggctccttctccctgcccctgaagccagtcatctcccagcccatccatgacagcaaatctccgggggcatcaaacctgaagatttctcgaatggacaagacagcaggctctgtgcggggtggagatgaagtttatctgctttgtgacaaggtgcagaaagatgacattgaggttcggttctatgaggatgatgagaatggatggcaggcctttggggacttctctcccacagatgtgcataaacagtatgccattgtgttccggacacccccctatcacaagatgaagattgagcggcctgtaacagtgtttctgcaactgaaacgcaagcgaggaggggacgtgtctgattccaaacagttcacctattaccctctggtggaagacaaggaagaggtgcagcggaagcggaggaaggccttgcccaccttctcccagcccttcgggggtggctcccacatgggtggaggctctgggggtgcagccgggggctacggaggagctggaggaggtggcagcctcggtttcttcccctcctccctggcctacagcccctaccagtccggcgcgggccccatgggctgctacccgggaggcgggggcggggcgcagatggccgccacggtgcccagcagggactccggggaggaagccgcggagccgagcgccccctccaggaccccccagtgcgagccgcaggccccggagatgctgcagcgagctcgagagtacaacgcgcgcctgttcggcctggcgcagcgcagcgcccgagccctactcgactacggcgtcaccgcggacgcgcgcgcgctgctggcgggacagcgccacctgctgacggcgcaggacgagaacggagacacaccactgcacctagccatcatccacgggcagaccagtgtcattgagcagatagtctatgtcatccaccacgcccaggacctcggcgttgtcaacctcaccaaccacctgcaccagacgcccctgcacctggcggtgatcacggggcagacgagtgtggtgagctttctgctgcgggtaggtgcagacccagctctgctggatcggcatggagactcagccatgcatctggcgctgcgggcaggcgctggtgctcctgagctgctgcgtgcactgcttcagagtggagctcctgctgtgccccagctgttgcatatgcctgactttgagggactgtatccagtacacctggcggtccgagcccgaagccctgagtgcctggatctgctggtggacagtggggctgaagtggaggccacagagcggcaggggggacgaacagccttgcatctagccacagagatggaggagctggggttggtcacccatctggtcaccaagctccgggccaacgtgaacgctcgcacctttgcgggaaacacacccctgcacctggcagctggactggggtacccgaccctcacccgcctccttctgaaggctggtgctgacatccatgctgaaaacgaggagcccctgtgcccactgccttcaccccctacctctgatagcgactcggactctgaagggcctgagaaggacacccgaagcagcttccggggccacacgcctcttgacctcacttgcagcaccaaggtgaagaccttgctgctaaatgctgctcagaacaccatggagccacccctgaccccgcccagcccagcagggccgggactgtcacttggtgatacagctctgcagaacctggagcagctgctagacgggccagaagcccagggcagctgggcagagctggcagagcgtctggggctgcgcagcctggtagacacgtaccgacagacaacctcacccagtggcagcctcctgcgcagctacgagctggctggcggggacctggcaggtctactggaggccctgtctgacatgggcctagaggagggagtgaggctgctgaggggtccagaaacccgagacaagctgcccagcacagaggtgaaggaagacagtgcgtacgggagccagtcagtggagcaggaggcagagaagctgggcccaccccctgagccaccaggagggctctgccacgggcacccccagcctcaggtgcactgacctgctgcctgcccccagcccccttcccggaccccctgtacagcgtccccacctatttcaaatcttatttaacaccccacacccacccctcagttgggacaaataaaggattctcatgggaaggggaggacccctccttcccaacttatggca

SEQ ID NO: 217 - DNA, 3293 bp, Homo sapiens
  source 1..3293: mol_type = genomic DNA
cttcctcttccgctaacttcccggcagcgcgccgctcagggtgggggccccgagggctggggccgtcggcttccccctggggatcccccgcttcagagaaagccaagcgttaggcgcagccaaagccgagaggcagcggaagctcccggcccggggtggcgctgggtcagcgggtaccttctcggcggtcccctggccggccgaactcgcgcctggtgtcctgtcaccccgctccccgccctgagtgagcctgtcccctctcaggggcgcgcccgagtcgctccgggttggctgcgccagtccagagttaaactttcagccaatgaaaaagggcgcgaggcgtgacgcacggaaacgtcatgggaattcccccctccggggggccgagaaggggctttcccggccctgagccctgctggcaggcgaggtgtcgcgaccggtcccaggtgggtcgggcgcggagagaagccgcaaccagagccgccgccacggcgggcgtctaaaattctgggaagcagaacctggccggagccactagacagagccgggcctagcccagagacatggagagttgctacaacccaggtctggatggtattattgaatatgatgatttcaaattgaactcctccattgtggaacccaaggagccagccccagaaacagctgatggcccctacctggtgatcgtggaacagcctaagcagagaggcttccgatttcgatatggctgtgaaggcccctcccatggaggactgcccggtgcctccagtgagaagggccgaaagacctatcccactgtcaagatctgtaactacgagggaccagccaagatcgaggtggacctggtaacacacagtgacccacctcgtgctcatgcccacagtctggtgggcaagcaatgctcggagctggggatctgcgccgtttctgtggggcccaaggacatgactgcccaatttaacaacctgggtgtcctgcatgtgactaagaagaacatgatggggactatgatacaaaaacttcagaggcagcggctccgctctaggccccagggccttacggaggccgagcagcgggagctggagcaagaggccaaagaactgaagaaggtgatggatctgagtatagtgcggctgcgcttctctgccttccttagagccagtgatggctccttctccctgcccctgaagccagtcatctcccagcccatccatgacagcaaatctccgggggcatcaaacctgaagatttctcgaatggacaagacagcaggctctgtgcggggtggagatgaagtttatctgctttgtgacaaggtgcagaaagatgacattgaggttcggttctatgaggatgatgagaatggatggcaggcctttggggacttctctcccacagatgtgcataaacagtatgccattgtgttccggacacccccctatcacaagatgaagattgagcggcctgtaacagtgtttctgcaactgaaacgcaagcgaggaggggacgtgtctgattccaaacagttcacctattaccctctggtggaaggtggcagcctcggtttcttcccctcctccctggcctacagcccctaccagtccggcgcgggccccatgggctgctacccgggaggcgggggcggggcgcagatggccgccacggtgcccagcagggactccggggaggaagccgcggagccgagcgccccctccaggaccccccagtgcgagccgcaggccccggagatgctgcagcgagctcgagagtacaacgcgcgcctgttcggcctggcgcagcgcagcgcccgagccctactcgactacggcgtcaccgcggacgcgcgcgcgctgctggcgggacagcgccacctgctgacggcgcaggacgagaacggagacacaccactgcacctagccatcatccacgggcagaccagtgtcattgagcagatagtctatgtcatccaccacgcccaggacctcggcgttgtcaacctcaccaaccacctgcaccagacgcccctgcacctggcggtgatcacggggcagacgagtgtggtgagctttctgctgcgggtaggtgcagacccagctctgctggatcggcatggagactcagccatgcatctggcgctgcgggcaggcgctggtgctcctgagctgctgcgtgcactgcttcagagtggagctcctgctgtgccccagctgttgcatatgcctgactttgagggactgtatccagtacacctggcggtccgagcccgaagccctgagtgcctggatctgctggtggacagtggggctgaagtggaggccacagagcggcaggggggacgaacagccttgcatctagccacagagatggaggagctggggttggtcacccatctggtcaccaagctccgggccaacgtgaacgctcgcacctttgcgggaaacacacccctgcacctggcagctggactggggtacccgaccctcacccgcctccttctgaaggctggtgctgacatccatgctgaaaacgaggagcccctgtgcccactgccttcaccccctacctctgatagcgactcggactctgaagggcctgagaaggacacccgaagcagcttccggggccacacgcctcttgacctcacttgcagcaccaaggtgaagaccttgctgctaaatgctgctcagaacaccatggagccacccctgaccccgcccagcccagcagggccgggactgtcacttggtgatacagctctgcagaacctggagcagctgctagacgggccagaagcccagggcagctgggcagagctggcagagcgtctggggctgcgcagcctggtagacacgtaccgacagacaacctcacccagtggcagcctcctgcgcagctacgagctggctggcggggacctggcaggtctactggaggccctgtctgacatgggcctagaggagggagtgaggctgctgaggggtccagaaacccgagacaagctgcccagcacagcagaggtgaaggaagacagtgcgtacgggagccagtcagtggagcaggaggcagagaagctgggcccaccccctgagccaccaggagggctctgccacgggcacccccagcctcaggtgcactgacctgctgcctgcccccagcccccttcccggaccccctgtacagcgtccccacctatttcaaatcttatttaacaccccacacccacccctcagttgggacaaataaaggattctcatgggaaggggaggacccctccttcccaacttatggca

SEQ ID NO: 218 - DNA, 4622 bp, Homo sapiens
  source 1..4622: mol_type = genomic DNA
gaacgtagctagctgcaagcagaggccggcatgaccaccgagcagcgacgcagcctgcaagccttccaggattatatccggaagaccctggaccctacctacatcctgagctacatggccccctggtttagggaggaagaggtgcagtatattcaggctgagaaaaacaacaagggcccaatggaggctgccacactttttctcaagttcctgttggagctccaggaggaaggctggttccgtggctttttggatgccctagaccatgcaggttattctggactttatgaagccattgaaagttgggatttcaaaaaaattgaaaagttggaggagtatagattacttttaaaacgtttacaaccagaatttaaaaccagaattatcccaaccgatatcatttctgatctgtctgaatgtttaattaatcaggaatgtgaagaaattctacagatttgctctactaaggggatgatggcaggtgcagagaaattggtggaatgccttctcagatcagacaaggaaaactggcccaaaactttgaaacttgctttggagaaagaaaggaacaagttcagtgaactgtggattgtagagaaaggtataaaagatgttgaaacagaagatcttgaggataagatggaaacttctgacatacagattttctaccaagaagatccagaatgccagaatcttagtgagaattcatgtccaccttcagaagtgtctgatacaaacttgtacagcccatttaaaccaagaaattaccaattagagcttgctttgcctgctatgaaaggaaaaaacacaataatatgtgctcctacaggttgtggaaaaacctttgtttcactgcttatatgtgaacatcatcttaaaaaattcccacaaggacaaaaggggaaagttgtcttttttgcgaatcagatcccagtgtatgaacagcagaaatctgtattctcaaaatactttgaaagacatggagttacaggcatttctggagcaacagctgagaatgtcccagtggaacagattgttgagaacaatgacatcatcattttaactccacagattcttgtgaacaaccttaaaaagggaacgattccatcactatccatctttactttgatgatatttgatgaatgccacaacactagtaaacaacacccgtacaatatgatcatgtttaattatctagatcagaaacttggaggatcttcaggcccactgccccaggtcattgggctgactgcctcggttggtgttggggatgccaaaaacacagatgaagccttggattatatctgcaagctgtgtgcttctcttgatgcgtcagtgatagcaacagtcaaacacaatctggaggaactggagcaagttgtttataagccccagaagtttttcaggaaagtggaatcacggattagcgacaaatttaaatacatcatagctcagctgatgagggacacagagagtctggcaaagagaatctgcaaagacctcgaaaacttatctcaaattcaaaatagggaatttggaacacagaaatatgaacaatggattgttacagttcagaaagcatgcatggtgttccagatgccagacaaagatgaagagagcaggatttgtaaagccctgtttttatacacttcacatttgcggaaatataatgatgccctcattatcagtgagcatgcacgaatgaaagatgctctggattacttgaaagacttcttcagcaatgtccgagcagcaggattcgatgagattgagcaagatcttactcagagatttgaagaaaagctgcaggaactagaaagtgtttccagggatcccagcaatgagaatcctaaacttgaagacctctgcttcatcttacaagaagagtaccacttaaacccagagacaataacaattctctttgtgaaaaccagagcacttgtggacgctttaaaaaattggattgaaggaaatcctaaactcagttttctaaaacctggcatattgactggacgtggcaaaacaaatcagaacacaggaatgaccctcccggcacagaagtgtatattggatgcattcaaagccagtggagatcacaatattctgattgccacctcagttgctgatgaaggcattgacattgcacagtgcaatcttgtcatcctttatgagtatgtgggcaatgtcatcaaaatgatccaaaccagaggcagaggaagagcaagaggtagcaagtgcttccttctgactagtaatgctggtgtaattgaaaaagaacaaataaacatgtacaaagaaaaaatgatgaatgactctattttacgccttcagacatgggacgaagcagtatttagggaaaagattctgcatatacagactcatgaaaaattcatcagagatagtcaagaaaaaccaaaacctgtacctgataaggaaaataaaaaactgctctgcagaaagtgcaaagccttggcatgttacacagctgacgtaagagtgatagaggaatgccattacactgtgcttggagatgcttttaaggaatgctttgtgagtagaccacatcccaagccaaagcagttttcaagttttgaaaaaagagcaaagatattctgtgcccgacagaactgcagccatgactggggaatccatgtgaagtacaagacatttgagattccagttataaaaattgaaagttttgtggtggaggatattgcaactggagttcagacactgtactcgaagtggaaggactttcattttgagaagataccatttgatccagcagaaatgtccaaatgatatcaggtcctcaatcttcagctacagggaatgagtaactttgagtggagaagaaacaaacatagtgggtataatcatggatcgcttgtacccctgtgaaaatatattttttaaaaatatctttagcagtttgtactatattatatatgcaaagcacaaatgagtgaatcacagcactgagtattttgtaggccaacagagctcatagtacttgggaaaaattaaaaagcctcatttctagccttctttttagagtcaactgccaacaaacacacagtaatcactctgtacacactgggatagatgaatgaatggaatgttgggaatttttatctccctttgtctccttaacctactgtaaactggcttttgcccttaacaatctactgaaattgttcttttgaaggttaccagtgactctggttgccaaatccactgggcacttcttaaccttctatttgacctctgcgcatttggccctgttgagcactcttcttgaagctctccctgggcttctctctcttctagttctattctagtctttttttattgagtcctcctctttgctgatcccttccaagggttcaatatatatacatgtatatactgtacatatgtatatgtaactaatatacatacatacaggtatgtatatgtaatggttatatgtactcatgttcctggtgtagcaacgtgtggtatggctacacagagaacatgagaacataaagccatttttatgcttactactaaaagctgtccactgtagagttgctgtatgtagcaatgtgtatccactctacagtggtcagcttttagtagagagcataaaaatgataaaatacttcttgaaaacttagtttactatacatcttgccctattaatatgttctcttaacgtgtgccattgttctctttgaccattttcctataatgatgttgatgttcaacacctggactgaatgtctgttctcagatcccttggatgttacagatgaggcagtctgactgtcctttctacttgaaagattagaatatgtatccaaatggcattcacgtgtcacttagcaaggtttgctgatgcttcaaagagcttagtttgcggtttcctggacgtggaaacaagtatctgagttccctggagatcaacgggatgaggtgttacagctgcctccctcttcatgcaatctggtgagcagtggtgcaggcggggagccagagaaacttgccagttatataacttctctttggcttttcttcatctgtaaaacaaggataatactgaactgtaagggttagtggagagtttttaattaaaagaatgtgtgaaaagtacatgacacagtagttgcttgataatagttactagtagtagtattcttactaagacccaatacaaatggattatttaaaccaagtttatgagttggttttttttcattttctatttgtattttattaagagtgtcttttcttatgtgattttttttaattgctatttgatatggtttggctatatgtccccacccaaatctcatcttgaattataatccccatgtgtcaagggagggacctgacgggaggtgattggatcacgggggcagttgtccccatgctgttcttgggatagtgagttagttctcatgagatctgatggttttataagtgtttgacaattcctcctttacacacactctctctctcatctgctgccatgtaagacttgcctgcttccccttctgccatgattgtaagtttcctgaggcctcctcagccatgtggaactgtgaatctattaagcctcttttctttataaatga

SEQ ID NO: 219 - DNA, 4457 bp, Homo sapiens
  source 1..4457: mol_type = genomic DNA
gaacgtagctagctgcaagcagaggccggcatgaccaccgagcagcgacgcagcctgcaagccttccaggattatatccggaagaccctggaccctacctacatcctgagctacatggccccctggtttagggaggaagaggtgcagtatattcaggctgagaaaaacaacaagggcccaatggaggctgccacactttttctcaagttcctgttggagctccaggaggaaggctggttccgtggctttttggatgccctagaccatgcaggttattctggactttatgaagccattgaaagttgggatttcaaaaaaattgaaaagttggaggagtatagattacttttaaaacgtttacaaccagaatttaaaaccagaattatcccaaccgatatcatttctgatctgtctgaatgtttaattaatcaggaatgtgaagaaattctacagatttgctctactaaggggatgatggcaggtgcagagaaattggtggaatgccttctcagatcagacaaggaaaactggcccaaaactttgaaacttgctttggagaaagaaaggaacaagttcagtgaactgtggattgtagagaaaggtataaaagatgttgaaacagaagatcttgaggataagatggaaacttctgacatacagattttctaccaagaagatccagaatgccagaatcttagtgagaattcatgtccaccttcagaagtgtctgatacaaacttgtacagcccatttaaaccaagaaattaccaattagagcttgctttgcctgctatgaaaggaaaaaacacaataatatgtgctcctacaggttgtggaaaaacctttgtttcactgcttatatgtgaacatcatcttaaaaaattcccacaaggacaaaaggggaaagttgtcttttttgcgaatcagatcccagtgtatgaacagcagaaatctgtattctcaaaatactttgaaagacatgggtatagagttacaggcatttctggagcaacagctgagaatgtcccagtggaacagattgttgagaacaatgacatcatcattttaactccacagattcttgtgaacaaccttaaaaagggaacgattccatcactatccatctttactttgatgatatttgatgaatgccacaacactagtaaacaacacccgtacaatatgatcatgtttaattatctagatcagaaacttggaggatcttcaggcccactgccccaggtcattgggctgactgcctcggttggtgttggggatgccaaaaacacagatgaagccttggattatatctgcaagctgtgtgcttctcttgatgcgtcagtgatagcaacagtcaaacacaatctggaggaactggagcaagttgtttataagccccagaagtttttcaggaaagtggaatcacggattagcgacaaatttaaatacatcatagctcagctgatgagggacacagagagtctggcaaagagaatctgcaaagacctcgaaaacttatctcaaattcaaaatagggaatttggaacacagaaatatgaacaatggattgttacagttcagaaagcatgcatggtgttccagatgccagacaaagatgaagagagcaggatttgtaaagccctgtttttatacacttcacatttgcggaaatataatgatgccctcattatcagtgagcatgcacgaatgaaagatgctctggattacttgaaagacttcttcagcaatgtccgagcagcaggattcgatgagattgagcaagatcttactcagagatttgaagaaaagctgcaggaactagaaagtgtttccagggatcccagcaatgagaatcctaaacttgaagacctctgcttcatcttacaagaagagtaccacttaaacccagagacaataacaattctctttgtgaaaaccagagcacttgtggacgctttaaaaaattggattgaaggaaatcctaaactcagttttctaaaacctggcatattgactggacgtggcaaaacaaatcagaacacaggcagaggaagagcaagaggtagcaagtgcttccttctgactagtaatgctggtgtaattgaaaaagaacaaataaacatgtacaaagaaaaaatgatgaatgactctattttacgccttcagacatgggacgaagcagtatttagggaaaagattctgcatatacagactcatgaaaaattcatcagagatagtcaagaaaaaccaaaacctgtacctgataaggaaaataaaaaactgctctgcagaaagtgcaaagccttggcatgttacacagctgacgtaagagtgatagaggaatgccattacactgtgcttggagatgcttttaaggaatgctttgtgagtagaccacatcccaagccaaagcagttttcaagttttgaaaaaagagcaaagatattctgtgcccgacagaactgcagccatgactggggaatccatgtgaagtacaagacatttgagattccagttataaaaattgaaagttttgtggtggaggatattgcaactggagttcagacactgtactcgaagtggaaggactttcattttgagaagataccatttgatccagcagaaatgtccaaatgatatcaggtcctcaatcttcagctacagggaatgagtaactttgagtggagaagaaacaaacatagtgggtataatcatggatcgcttgtacccctgtgaaaatatattttttaaaaatatctttagcagtttgtactatattatatatgcaaagcacaaatgagtgaatcacagcactgagtattttgtaggccaacagagctcatagtacttgggaaaaattaaaaagcctcatttctagccttctttttagagtcaactgccaacaaacacacagtaatcactctgtacacactgggatagatgaatgaatggaatgttgggaatttttatctccctttgtctccttaacctactgtaaactggcttttgcccttaacaatctactgaaattgttcttttgaaggttaccagtgactctggttgccaaatccactgggcacttcttaaccttctatttgacctctgcgcatttggccctgttgagcactcttcttgaagctctccctgggcttctctctcttctagttctattctagtctttttttattgagtcctcctctttgctgatcccttccaagggttcaatatatatacatgtatatactgtacatatgtatatgtaactaatatacatacatacaggtatgtatatgtaatggttatatgtactcatgttcctggtgtagcaacgtgtggtatggctacacagagaacatgagaacataaagccatttttatgcttactactaaaagctgtccactgtagagttgctgtatgtagcaatgtgtatccactctacagtggtcagcttttagtagagagcataaaaatgataaaatacttcttgaaaacttagtttactatacatcttgccctattaatatgttctcttaacgtgtgccattgttctctttgaccattttcctataatgatgttgatgttcaacacctggactgaatgtctgttctcagatcccttggatgttacagatgaggcagtctgactgtcctttctacttgaaagattagaatatgtatccaaatggcattcacgtgtcacttagcaaggtttgctgatgcttcaaagagcttagtttgcggtttcctggacgtggaaacaagtatctgagttccctggagatcaacgggatgaggtgttacagctgcctccctcttcatgcaatctggtgagcagtggtgcaggcggggagccagagaaacttgccagttatataacttctctttggcttttcttcatctgtaaaacaaggataatactgaactgtaagggttagtggagagtttttaattaaaagaatgtgtgaaaagtacatgacacagtagttgcttgataatagttactagtagtagtattcttactaagacccaatacaaatggattatttaaaccaagtttatgagttggttttttttcattttctatttgtattttattaagagtgtcttttcttatgtgattttttttaattgctatttgatatggtttggctatatgtccccacccaaatctcatcttgaattataatccccatgtgtcaagggagggacctgacgggaggtgattggatcacgggggcagttgtccccatgctgttcttgggatagtgagttagttctcatgagatctgatggttttataagtgtttgacaattcctcctttacacacactctctctctcatctgctgccatgtaagacttgcctgcttccccttctgccatgattgtaagtttcctgaggcctcctcagccatgtggaactgtgaatctattaagcctcttttctttataaatga

SEQ ID NO: 220 - DNA, 4641 bp, Homo sapiens
  source 1..4641: mol_type = genomic DNA
gaacgtagctagctgcaagcagaggccggcatgaccaccgagcagcgacgcagcctgcaagccttccaggattatatccggaagaccctggaccctacctacatcctgagctacatggccccctggtttagggaggaagaggtgcagtatattcaggctgagaaaaacaacaagggcccaatggaggctgccacactttttctcaagttcctgttggagctccaggaggaaggctggttccgtggctttttggatgccctagaccatgcaggttattctggactttatgaagccattgaaagttgggatttcaaaaaaattgaaaagttggaggagtatagattacttttaaaacgtttacaaccagaatttaaaaccagaattatcccaaccgatatcatttctgatctgtctgaatgtttaattaatcaggaatgtgaagaaattctacagttaccttgcttagatttgctctactaaggggatgatggcaggtgcagagaaattggtggaatgccttctcagatcagacaaggaaaactggcccaaaactttgaaacttgctttggagaaagaaaggaacaagttcagtgaactgtggattgtagagaaaggtataaaagatgttgaaacagaagatcttgaggataagatggaaacttctgacatacagattttctaccaagaagatccagaatgccagaatcttagtgagaattcatgtccaccttcagaagtgtctgatacaaacttgtacagcccatttaaaccaagaaattaccaattagagcttgctttgcctgctatgaaaggaaaaaacacaataatatgtgctcctacaggttgtggaaaaacctttgtttcactgcttatatgtgaacatcatcttaaaaaattcccacaaggacaaaaggggaaagttgtcttttttgcgaatcagatcccagtgtatgaacagcagaaatctgtattctcaaaatactttgaaagacatgggtatagagttacaggcatttctggagcaacagctgagaatgtcccagtggaacagattgttgagaacaatgacatcatcattttaactccacagattcttgtgaacaaccttaaaaagggaacgattccatcactatccatctttactttgatgatatttgatgaatgccacaacactagtaaacaacacccgtacaatatgatcatgtttaattatctagatcagaaacttggaggatcttcaggcccactgccccaggtcattgggctgactgcctcggttggtgttggggatgccaaaaacacagatgaagccttggattatatctgcaagctgtgtgcttctcttgatgcgtcagtgatagcaacagtcaaacacaatctggaggaactggagcaagttgtttataagccccagaagtttttcaggaaagtggaatcacggattagcgacaaatttaaatacatcatagctcagctgatgagggacacagagagtctggcaaagagaatctgcaaagacctcgaaaacttatctcaaattcaaaatagggaatttggaacacagaaatatgaacaatggattgttacagttcagaaagcatgcatggtgttccagatgccagacaaagatgaagagagcaggatttgtaaagccctgtttttatacacttcacatttgcggaaatataatgatgccctcattatcagtgagcatgcacgaatgaaagatgctctggattacttgaaagacttcttcagcaatgtccgagcagcaggattcgatgagattgagcaagatcttactcagagatttgaagaaaagctgcaggaactagaaagtgtttccagggatcccagcaatgagaatcctaaacttgaagacctctgcttcatcttacaagaagagtaccacttaaacccagagacaataacaattctctttgtgaaaaccagagcacttgtggacgctttaaaaaattggattgaaggaaatcctaaactcagttttctaaaacctggcatattgactggacgtggcaaaacaaatcagaacacaggaatgaccctcccggcacagaagtgtatattggatgcattcaaagccagtggagatcacaatattctgattgccacctcagttgctgatgaaggcattgacattgcacagtgcaatcttgtcatcctttatgagtatgtgggcaatgtcatcaaaatgatccaaaccagaggcagaggaagagcaagaggtagcaagtgcttccttctgactagtaatgctggtgtaattgaaaaagaacaaataaacatgtacaaagaaaaaatgatgaatgactctattttacgccttcagacatgggacgaagcagtatttagggaaaagattctgcatatacagactcatgaaaaattcatcagagatagtcaagaaaaaccaaaacctgtacctgataaggaaaataaaaaactgctctgcagaaagtgcaaagccttggcatgttacacagctgacgtaagagtgatagaggaatgccattacactgtgcttggagatgcttttaaggaatgctttgtgagtagaccacatcccaagccaaagcagttttcaagttttgaaaaaagagcaaagatattctgtgcccgacagaactgcagccatgactggggaatccatgtgaagtacaagacatttgagattccagttataaaaattgaaagttttgtggtggaggatattgcaactggagttcagacactgtactcgaagtggaaggactttcattttgagaagataccatttgatccagcagaaatgtccaaatgatatcaggtcctcaatcttcagctacagggaatgagtaactttgagtggagaagaaacaaacatagtgggtataatcatggatcgcttgtacccctgtgaaaatatattttttaaaaatatctttagcagtttgtactatattatatatgcaaagcacaaatgagtgaatcacagcactgagtattttgtaggccaacagagctcatagtacttgggaaaaattaaaaagcctcatttctagccttctttttagagtcaactgccaacaaacacacagtaatcactctgtacacactgggatagatgaatgaatggaatgttgggaatttttatctccctttgtctccttaacctactgtaaactggcttttgcccttaacaatctactgaaattgttcttttgaaggttaccagtgactctggttgccaaatccactgggcacttcttaaccttctatttgacctctgcgcatttggccctgttgagcactcttcttgaagctctccctgggcttctctctcttctagttctattctagtctttttttattgagtcctcctctttgctgatcccttccaagggttcaatatatatacatgtatatactgtacatatgtatatgtaactaatatacatacatacaggtatgtatatgtaatggttatatgtactcatgttcctggtgtagcaacgtgtggtatggctacacagagaacatgagaacataaagccatttttatgcttactactaaaagctgtccactgtagagttgctgtatgtagcaatgtgtatccactctacagtggtcagcttttagtagagagcataaaaatgataaaatacttcttgaaaacttagtttactatacatcttgccctattaatatgttctcttaacgtgtgccattgttctctttgaccattttcctataatgatgttgatgttcaacacctggactgaatgtctgttctcagatcccttggatgttacagatgaggcagtctgactgtcctttctacttgaaagattagaatatgtatccaaatggcattcacgtgtcacttagcaaggtttgctgatgcttcaaagagcttagtttgcggtttcctggacgtggaaacaagtatctgagttccctggagatcaacgggatgaggtgttacagctgcctccctcttcatgcaatctggtgagcagtggtgcaggcggggagccagagaaacttgccagttatataacttctctttggcttttcttcatctgtaaaacaaggataatactgaactgtaagggttagtggagagtttttaattaaaagaatgtgtgaaaagtacatgacacagtagttgcttgataatagttactagtagtagtattcttactaagacccaatacaaatggattatttaaaccaagtttatgagttggttttttttcattttctatttgtattttattaagagtgtcttttcttatgtgattttttttaattgctatttgatatggtttggctatatgtccccacccaaatctcatcttgaattataatccccatgtgtcaagggagggacctgacgggaggtgattggatcacgggggcagttgtccccatgctgttcttgggatagtgagttagttctcatgagatctgatggttttataagtgtttgacaattcctcctttacacacactctctctctcatctgctgccatgtaagacttgcctgcttccccttctgccatgattgtaagtttcctgaggcctcctcagccatgtggaactgtgaatctattaagcctcttttctttataaatga

SEQ ID NO: 221 - DNA, 4480 bp, Homo sapiens
  source 1..4480: mol_type = genomic DNA
gaacgtagctagctgcaagcagaggccggcatgaccaccgagcagcgacgcagcctgcaagccttccaggattatatccggaagaccctggaccctacctacatcctgagctacatggccccctggtttagggaggaagaggtgcagtatattcaggctgagaaaaacaacaagggcccaatggaggctgccacactttttctcaagttcctgttggagctccaggaggaaggctggttccgtggctttttggatgccctagaccatgcaggttattctggactttatgaagccattgaaagttgggatttcaaaaaaattgaaaagttggaggagtatagattacttttaaaacgtttacaaccagaatttaaaaccagaattatcccaaccgatatcatttctgatctgtctgaatgtttaattaatcaggaatgtgaagaaattctacaggtataaaagatgttgaaacagaagatcttgaggataagatggaaacttctgacatacagattttctaccaagaagatccagaatgccagaatcttagtgagaattcatgtccaccttcagaagtgtctgatacaaacttgtacagcccatttaaaccaagaaattaccaattagagcttgctttgcctgctatgaaaggaaaaaacacaataatatgtgctcctacaggttgtggaaaaacctttgtttcactgcttatatgtgaacatcatcttaaaaaattcccacaaggacaaaaggggaaagttgtcttttttgcgaatcagatcccagtgtatgaacagcagaaatctgtattctcaaaatactttgaaagacatgggtatagagttacaggcatttctggagcaacagctgagaatgtcccagtggaacagattgttgagaacaatgacatcatcattttaactccacagattcttgtgaacaaccttaaaaagggaacgattccatcactatccatctttactttgatgatatttgatgaatgccacaacactagtaaacaacacccgtacaatatgatcatgtttaattatctagatcagaaacttggaggatcttcaggcccactgccccaggtcattgggctgactgcctcggttggtgttggggatgccaaaaacacagatgaagccttggattatatctgcaagctgtgtgcttctcttgatgcgtcagtgatagcaacagtcaaacacaatctggaggaactggagcaagttgtttataagccccagaagtttttcaggaaagtggaatcacggattagcgacaaatttaaatacatcatagctcagctgatgagggacacagagagtctggcaaagagaatctgcaaagacctcgaaaacttatctcaaattcaaaatagggaatttggaacacagaaatatgaacaatggattgttacagttcagaaagcatgcatggtgttccagatgccagacaaagatgaagagagcaggatttgtaaagccctgtttttatacacttcacatttgcggaaatataatgatgccctcattatcagtgagcatgcacgaatgaaagatgctctggattacttgaaagacttcttcagcaatgtccgagcagcaggattcgatgagattgagcaagatcttactcagagatttgaagaaaagctgcaggaactagaaagtgtttccagggatcccagcaatgagaatcctaaacttgaagacctctgcttcatcttacaagaagagtaccacttaaacccagagacaataacaattctctttgtgaaaaccagagcacttgtggacgctttaaaaaattggattgaaggaaatcctaaactcagttttctaaaacctggcatattgactggacgtggcaaaacaaatcagaacacaggaatgaccctcccggcacagaagtgtatattggatgcattcaaagccagtggagatcacaatattctgattgccacctcagttgctgatgaaggcattgacattgcacagtgcaatcttgtcatcctttatgagtatgtgggcaatgtcatcaaaatgatccaaaccagaggcagaggaagagcaagaggtagcaagtgcttccttctgactagtaatgctggtgtaattgaaaaagaacaaataaacatgtacaaagaaaaaatgatgaatgactctattttacgccttcagacatgggacgaagcagtatttagggaaaagattctgcatatacagactcatgaaaaattcatcagagatagtcaagaaaaaccaaaacctgtacctgataaggaaaataaaaaactgctctgcagaaagtgcaaagccttggcatgttacacagctgacgtaagagtgatagaggaatgccattacactgtgcttggagatgcttttaaggaatgctttgtgagtagaccacatcccaagccaaagcagttttcaagttttgaaaaaagagcaaagatattctgtgcccgacagaactgcagccatgactggggaatccatgtgaagtacaagacatttgagattccagttataaaaattgaaagttttgtggtggaggatattgcaactggagttcagacactgtactcgaagtggaaggactttcattttgagaagataccatttgatccagcagaaatgtccaaatgatatcaggtcctcaatcttcagctacagggaatgagtaactttgagtggagaagaaacaaacatagtgggtataatcatggatcgcttgtacccctgtgaaaatatattttttaaaaatatctttagcagtttgtactatattatatatgcaaagcacaaatgagtgaatcacagcactgagtattttgtaggccaacagagctcatagtacttgggaaaaattaaaaagcctcatttctagccttctttttagagtcaactgccaacaaacacacagtaatcactctgtacacactgggatagatgaatgaatggaatgttgggaatttttatctccctttgtctccttaacctactgtaaactggcttttgcccttaacaatctactgaaattgttcttttgaaggttaccagtgactctggttgccaaatccactgggcacttcttaaccttctatttgacctctgcgcatttggccctgttgagcactcttcttgaagctctccctgggcttctctctcttctagttctattctagtctttttttattgagtcctcctctttgctgatcccttccaagggttcaatatatatacatgtatatactgtacatatgtatatgtaactaatatacatacatacaggtatgtatatgtaatggttatatgtactcatgttcctggtgtagcaacgtgtggtatggctacacagagaacatgagaacataaagccatttttatgcttactactaaaagctgtccactgtagagttgctgtatgtagcaatgtgtatccactctacagtggtcagcttttagtagagagcataaaaatgataaaatacttcttgaaaacttagtttactatacatcttgccctattaatatgttctcttaacgtgtgccattgttctctttgaccattttcctataatgatgttgatgttcaacacctggactgaatgtctgttctcagatcccttggatgttacagatgaggcagtctgactgtcctttctacttgaaagattagaatatgtatccaaatggcattcacgtgtcacttagcaaggtttgctgatgcttcaaagagcttagtttgcggtttcctggacgtggaaacaagtatctgagttccctggagatcaacgggatgaggtgttacagctgcctccctcttcatgcaatctggtgagcagtggtgcaggcggggagccagagaaacttgccagttatataacttctctttggcttttcttcatctgtaaaacaaggataatactgaactgtaagggttagtggagagtttttaattaaaagaatgtgtgaaaagtacatgacacagtagttgcttgataatagttactagtagtagtattcttactaagacccaatacaaatggattatttaaaccaagtttatgagttggttttttttcattttctatttgtattttattaagagtgtcttttcttatgtgattttttttaattgctatttgatatggtttggctatatgtccccacccaaatctcatcttgaattataatccccatgtgtcaagggagggacctgacgggaggtgattggatcacgggggcagttgtccccatgctgttcttgggatagtgagttagttctcatgagatctgatggttttataagtgtttgacaattcctcctttacacacactctctctctcatctgctgccatgtaagacttgcctgcttccccttctgccatgattgtaagtttcctgaggcctcctcagccatgtggaactgtgaatctattaagcctcttttctttataaatga

SEQ ID NO: 222 - DNA, 4613 bp, Homo sapiens
  source 1..4613: mol_type = genomic DNA
gaacgtagctagctgcaagcagaggccggcatgaccaccgagcagcgacgcagcctgcaagccttccaggattatatccggaagaccctggaccctacctacatcctgagctacatggccccctggtttagggaggaagaggtgcagtatattcaggctgagaaaaacaacaagggcccaatggaggctgccacactttttctcaagttcctgttggagctccaggaggaaggctggttccgtggctttttggatgccctagaccatgcaggttattctggactttatgaagccattgaaagttgggatttcaaaaaaattgaaaagttggaggagtatagattacttttaaaacgtttacaaccagaatttaaaaccagaattatcccaaccgatatcatttctgatctgtctgaatgtttaattaatcaggaatgtgaagaaattctacaggggatgatggcaggtgcagagaaattggtggaatgccttctcagatcagacaaggaaaactggcccaaaactttgaaacttgctttggagaaagaaaggaacaagttcagtgaactgtggattgtagagaaaggtataaaagatgttgaaacagaagatcttgaggataagatggaaacttctgacatacagattttctaccaagaagatccagaatgccagaatcttagtgagaattcatgtccaccttcagaagtgtctgatacaaacttgtacagcccatttaaaccaagaaattaccaattagagcttgctttgcctgctatgaaaggaaaaaacacaataatatgtgctcctacaggttgtggaaaaacctttgtttcactgcttatatgtgaacatcatcttaaaaaattcccacaaggacaaaaggggaaagttgtcttttttgcgaatcagatcccagtgtatgaacagcagaaatctgtattctcaaaatactttgaaagacatgggtatagagttacaggcatttctggagcaacagctgagaatgtcccagtggaacagattgttgagaacaatgacatcatcattttaactccacagattcttgtgaacaaccttaaaaagggaacgattccatcactatccatctttactttgatgatatttgatgaatgccacaacactagtaaacaacacccgtacaatatgatcatgtttaattatctagatcagaaacttggaggatcttcaggcccactgccccaggtcattgggctgactgcctcggttggtgttggggatgccaaaaacacagatgaagccttggattatatctgcaagctgtgtgcttctcttgatgcgtcagtgatagcaacagtcaaacacaatctggaggaactggagcaagttgtttataagccccagaagtttttcaggaaagtggaatcacggattagcgacaaatttaaatacatcatagctcagctgatgagggacacagagagtctggcaaagagaatctgcaaagacctcgaaaacttatctcaaattcaaaatagggaatttggaacacagaaatatgaacaatggattgttacagttcagaaagcatgcatggtgttccagatgccagacaaagatgaagagagcaggatttgtaaagccctgtttttatacacttcacatttgcggaaatataatgatgccctcattatcagtgagcatgcacgaatgaaagatgctctggattacttgaaagacttcttcagcaatgtccgagcagcaggattcgatgagattgagcaagatcttactcagagatttgaagaaaagctgcaggaactagaaagtgtttccagggatcccagcaatgagaatcctaaacttgaagacctctgcttcatcttacaagaagagtaccacttaaacccagagacaataacaattctctttgtgaaaaccagagcacttgtggacgctttaaaaaattggattgaaggaaatcctaaactcagttttctaaaacctggcatattgactggacgtggcaaaacaaatcagaacacaggaatgaccctcccggcacagaagtgtatattggatgcattcaaagccagtggagatcacaatattctgattgccacctcagttgctgatgaaggcattgacattgcacagtgcaatcttgtcatcctttatgagtatgtgggcaatgtcatcaaaatgatccaaaccagaggcagaggaagagcaagaggtagcaagtgcttccttctgactagtaatgctggtgtaattgaaaaagaacaaataaacatgtacaaagaaaaaatgatgaatgactctattttacgccttcagacatgggacgaagcagtatttagggaaaagattctgcatatacagactcatgaaaaattcatcagagatagtcaagaaaaaccaaaacctgtacctgataaggaaaataaaaaactgctctgcagaaagtgcaaagccttggcatgttacacagctgacgtaagagtgatagaggaatgccattacactgtgcttggagatgcttttaaggaatgctttgtgagtagaccacatcccaagccaaagcagttttcaagttttgaaaaaagagcaaagatattctgtgcccgacagaactgcagccatgactggggaatccatgtgaagtacaagacatttgagattccagttataaaaattgaaagttttgtggtggaggatattgcaactggagttcagacactgtactcgaagtggaaggactttcattttgagaagataccatttgatccagcagaaatgtccaaatgatatcaggtcctcaatcttcagctacagggaatgagtaactttgagtggagaagaaacaaacatagtgggtataatcatggatcgcttgtacccctgtgaaaatatattttttaaaaatatctttagcagtttgtactatattatatatgcaaagcacaaatgagtgaatcacagcactgagtattttgtaggccaacagagctcatagtacttgggaaaaattaaaaagcctcatttctagccttctttttagagtcaactgccaacaaacacacagtaatcactctgtacacactgggatagatgaatgaatggaatgttgggaatttttatctccctttgtctccttaacctactgtaaactggcttttgcccttaacaatctactgaaattgttcttttgaaggttaccagtgactctggttgccaaatccactgggcacttcttaaccttctatttgacctctgcgcatttggccctgttgagcactcttcttgaagctctccctgggcttctctctcttctagttctattctagtctttttttattgagtcctcctctttgctgatcccttccaagggttcaatatatatacatgtatatactgtacatatgtatatgtaactaatatacatacatacaggtatgtatatgtaatggttatatgtactcatgttcctggtgtagcaacgtgtggtatggctacacagagaacatgagaacataaagccatttttatgcttactactaaaagctgtccactgtagagttgctgtatgtagcaatgtgtatccactctacagtggtcagcttttagtagagagcataaaaatgataaaatacttcttgaaaacttagtttactatacatcttgccctattaatatgttctcttaacgtgtgccattgttctctttgaccattttcctataatgatgttgatgttcaacacctggactgaatgtctgttctcagatcccttggatgttacagatgaggcagtctgactgtcctttctacttgaaagattagaatatgtatccaaatggcattcacgtgtcacttagcaaggtttgctgatgcttcaaagagcttagtttgcggtttcctggacgtggaaacaagtatctgagttccctggagatcaacgggatgaggtgttacagctgcctccctcttcatgcaatctggtgagcagtggtgcaggcggggagccagagaaacttgccagttatataacttctctttggcttttcttcatctgtaaaacaaggataatactgaactgtaagggttagtggagagtttttaattaaaagaatgtgtgaaaagtacatgacacagtagttgcttgataatagttactagtagtagtattcttactaagacccaatacaaatggattatttaaaccaagtttatgagttggttttttttcattttctatttgtattttattaagagtgtcttttcttatgtgattttttttaattgctatttgatatggtttggctatatgtccccacccaaatctcatcttgaattataatccccatgtgtcaagggagggacctgacgggaggtgattggatcacgggggcagttgtccccatgctgttcttgggatagtgagttagttctcatgagatctgatggttttataagtgtttgacaattcctcctttacacacactctctctctcatctgctgccatgtaagacttgcctgcttccccttctgccatgattgtaagtttcctgaggcctcctcagccatgtggaactgtgaatctattaagcctcttttctttataaatga

SEQ ID NO: 223 - DNA, 4638 bp, Homo sapiens
  source 1..4638: mol_type = genomic DNA
gaacgtagctagctgcaagcagaggccggcatgaccaccgagcagcgacgcagcctgcaagccttccaggattatatccggaagaccctggaccctacctacatcctgagctacatggccccctggtttagggaggaagaggtgcagtatattcaggctgagaaaaacaacaagggcccaatggaggctgccacactttttctcaagttcctgttggagctccaggaggaaggctggttccgtggctttttggatgccctagaccatgcaggttattctggactttatgaagccattgaaagttgggatttcaaaaaaattgaaaagttggaggagtatagattacttttaaaacgtttacaaccagaatttaaaaccagaattatcccaaccgatatcatttctgatctgtctgaatgtttaattaatcaggaatgtgaagaaattctacagttaccttgcttagatttgctctactaaggggatgatggcaggtgcagagaaattggtggaatgccttctcagatcagacaaggaaaactggcccaaaactttgaaacttgctttggagaaagaaaggaacaagttcagtgaactgtggattgtagagaaaggtataaaagatgttgaaacagaagatcttgaggataagatggaaacttctgacatacagattttctaccaagaagatccagaatgccagaatcttagtgagaattcatgtccaccttcagtgtctgatacaaacttgtacagcccatttaaaccaagaaattaccaattagagcttgctttgcctgctatgaaaggaaaaaacacaataatatgtgctcctacaggttgtggaaaaacctttgtttcactgcttatatgtgaacatcatcttaaaaaattcccacaaggacaaaaggggaaagttgtcttttttgcgaatcagatcccagtgtatgaacagcagaaatctgtattctcaaaatactttgaaagacatgggtatagagttacaggcatttctggagcaacagctgagaatgtcccagtggaacagattgttgagaacaatgacatcatcattttaactccacagattcttgtgaacaaccttaaaaagggaacgattccatcactatccatctttactttgatgatatttgatgaatgccacaacactagtaaacaacacccgtacaatatgatcatgtttaattatctagatcagaaacttggaggatcttcaggcccactgccccaggtcattgggctgactgcctcggttggtgttggggatgccaaaaacacagatgaagccttggattatatctgcaagctgtgtgcttctcttgatgcgtcagtgatagcaacagtcaaacacaatctggaggaactggagcaagttgtttataagccccagaagtttttcaggaaagtggaatcacggattagcgacaaatttaaatacatcatagctcagctgatgagggacacagagagtctggcaaagagaatctgcaaagacctcgaaaacttatctcaaattcaaaatagggaatttggaacacagaaatatgaacaatggattgttacagttcagaaagcatgcatggtgttccagatgccagacaaagatgaagagagcaggatttgtaaagccctgtttttatacacttcacatttgcggaaatataatgatgccctcattatcagtgagcatgcacgaatgaaagatgctctggattacttgaaagacttcttcagcaatgtccgagcagcaggattcgatgagattgagcaagatcttactcagagatttgaagaaaagctgcaggaactagaaagtgtttccagggatcccagcaatgagaatcctaaacttgaagacctctgcttcatcttacaagaagagtaccacttaaacccagagacaataacaattctctttgtgaaaaccagagcacttgtggacgctttaaaaaattggattgaaggaaatcctaaactcagttttctaaaacctggcatattgactggacgtggcaaaacaaatcagaacacaggaatgaccctcccggcacagaagtgtatattggatgcattcaaagccagtggagatcacaatattctgattgccacctcagttgctgatgaaggcattgacattgcacagtgcaatcttgtcatcctttatgagtatgtgggcaatgtcatcaaaatgatccaaaccagaggcagaggaagagcaagaggtagcaagtgcttccttctgactagtaatgctggtgtaattgaaaaagaacaaataaacatgtacaaagaaaaaatgatgaatgactctattttacgccttcagacatgggacgaagcagtatttagggaaaagattctgcatatacagactcatgaaaaattcatcagagatagtcaagaaaaaccaaaacctgtacctgataaggaaaataaaaaactgctctgcagaaagtgcaaagccttggcatgttacacagctgacgtaagagtgatagaggaatgccattacactgtgcttggagatgcttttaaggaatgctttgtgagtagaccacatcccaagccaaagcagttttcaagttttgaaaaaagagcaaagatattctgtgcccgacagaactgcagccatgactggggaatccatgtgaagtacaagacatttgagattccagttataaaaattgaaagttttgtggtggaggatattgcaactggagttcagacactgtactcgaagtggaaggactttcattttgagaagataccatttgatccagcagaaatgtccaaatgatatcaggtcctcaatcttcagctacagggaatgagtaactttgagtggagaagaaacaaacatagtgggtataatcatggatcgcttgtacccctgtgaaaatatattttttaaaaatatctttagcagtttgtactatattatatatgcaaagcacaaatgagtgaatcacagcactgagtattttgtaggccaacagagctcatagtacttgggaaaaattaaaaagcctcatttctagccttctttttagagtcaactgccaacaaacacacagtaatcactctgtacacactgggatagatgaatgaatggaatgttgggaatttttatctccctttgtctccttaacctactgtaaactggcttttgcccttaacaatctactgaaattgttcttttgaaggttaccagtgactctggttgccaaatccactgggcacttcttaaccttctatttgacctctgcgcatttggccctgttgagcactcttcttgaagctctccctgggcttctctctcttctagttctattctagtctttttttattgagtcctcctctttgctgatcccttccaagggttcaatatatatacatgtatatactgtacatatgtatatgtaactaatatacatacatacaggtatgtatatgtaatggttatatgtactcatgttcctggtgtagcaacgtgtggtatggctacacagagaacatgagaacataaagccatttttatgcttactactaaaagctgtccactgtagagttgctgtatgtagcaatgtgtatccactctacagtggtcagcttttagtagagagcataaaaatgataaaatacttcttgaaaacttagtttactatacatcttgccctattaatatgttctcttaacgtgtgccattgttctctttgaccattttcctataatgatgttgatgttcaacacctggactgaatgtctgttctcagatcccttggatgttacagatgaggcagtctgactgtcctttctacttgaaagattagaatatgtatccaaatggcattcacgtgtcacttagcaaggtttgctgatgcttcaaagagcttagtttgcggtttcctggacgtggaaacaagtatctgagttccctggagatcaacgggatgaggtgttacagctgcctccctcttcatgcaatctggtgagcagtggtgcaggcggggagccagagaaacttgccagttatataacttctctttggcttttcttcatctgtaaaacaaggataatactgaactgtaagggttagtggagagtttttaattaaaagaatgtgtgaaaagtacatgacacagtagttgcttgataatagttactagtagtagtattcttactaagacccaatacaaatggattatttaaaccaagtttatgagttggttttttttcattttctatttgtattttattaagagtgtcttttcttatgtgattttttttaattgctatttgatatggtttggctatatgtccccacccaaatctcatcttgaattataatccccatgtgtcaagggagggacctgacgggaggtgattggatcacgggggcagttgtccccatgctgttcttgggatagtgagttagttctcatgagatctgatggttttataagtgtttgacaattcctcctttacacacactctctctctcatctgctgccatgtaagacttgcctgcttccccttctgccatgattgtaagtttcctgaggcctcctcagccatgtggaactgtgaatctattaagcctcttttctttataaatga

SEQ ID NO: 224 - DNA, 4628 bp, Homo sapiens
  source 1..4628: mol_type = genomic DNA
gaacgtagctagctgcaagcagaggccggcatgaccaccgagcagcgacgcagcctgcaagccttccaggattatatccggaagaccctggaccctacctacatcctgagctacatggccccctggtttagggaggaagaggtgcagtatattcaggctgagaaaaacaacaagggcccaatggaggctgccacactttttctcaagttcctgttggagctccaggaggaaggctggttccgtggctttttggatgccctagaccatgcaggttattctggactttatgaagccattgaaagttgggatttcaaaaaaattgaaaagttggaggagtatagattacttttaaaacgtttacaaccagaatttaaaaccagaattatcccaaccgatatcatttctgatctgtctgaatgtttaattaatcaggaatgtgaagaaattctacagatttgctctactaaggggatgatggcaggtgcagagaaattggtggaatgccttctcagatcagacaaggaaaactggcccaaaactttgaaacttgctttggagaaagaaaggaacaagttcagtgaactgtggattgtagagaaaggtataaaagatgttgaaacagaagatcttgaggataagatggaaacttctgacatacagattttctaccaagaagatccagaatgccagaatcttagtgagaattcatgtccaccttcagaagtgtctgatacaaacttgtacagcccatttaaaccaagaaattaccaattagagcttgctttgcctgctatgaaaggaaaaaacacaataatatgtgctcctacaggttgtggaaaaacctttgtttcactgcttatatgtgaacatcatcttaaaaaattcccacaaggacaaaaggggaaagttgtcttttttgcgaatcagatcccagtgtatgaacagcagaaatctgtattctcaaaatactttgaaagacatgggtatagagttacaggcatttctggagcaacagctgagaatgtcccagtggaacagattgttgagaacaatgacatcatcattttaactccacagattcttgtgaacaaccttaaaaagggaacgattccatcactatccatctttactttgatgatatttgatgaatgccacaacactagtaaacaacacccgtacaatatgatcatgtttaattatctagatcagaaacttggaggatcttcaggcccactgccccaggtcattgggctgactgcctcggttggtgttggggatgccaaaaacacagatgaagccttggattatatctgcaagctgtgtgcttctcttgatgcgtcagtgatagcaacagtcaaacacaatctggaggaactggagcaagttgtttataagccccagaagtttttcaggaaagtggaatcacggattagcgacaaatttaaatacatcatagctcagctgatgagggacacagagagtctggcaaagagaatctgcaaagacctcgaaaacttatctcaaattcaaaatagggaatttggaacacagaaatatgaacaatggattgttacagttcagaaagcatgcatggtgttccagatgccagacaaagatgaagagagcaggatttgtaaagccctgtttttatacacttcacatttgcggaaatataatgatgccctcattatcagtgagcatgcacgaatgaaagatgctctggattacttgaaagacttcttcagcaatgtccgagcagcaggattcgatgagattgagcaagatcttactcagagatttgaagaaaagctgcaggaactagaaagtgtttccagggatcccagcaatgagaatcctaaacttgaagacctctgcttcatcttacaagaagagtaccacttaaacccagagacaataacaattctctttgtgaaaaccagagcacttgtggacgctttaaaaaattggattgaaggaaatcctaaactcagttttctaaaacctggcatattgactggacgtggcaaaacaaatcagaacacaggaatgaccctcccggcacagaagtgtatattggatgcattcaaagccagtggagatcacaatattctgattgccacctcagttgctgatgaaggcattgacattgcacagtgcaatcttgtcatcctttatgagtatgtgggcaatgtcatcaaaatgatccaaaccagaggcagaggaagagcaagaggtagcaagtgcttccttctgactagtaatgctggtgtaattgaaaaagaacaaataaacatgtacaaagaaaaaatgatgaatgactctattttacgccttcagacatgggacgaagcagtatttagggaaaagattctgcatatacagactcatgaaaaattcatcagagatagtcaagaaaaaccaaaacctgtacctgataaggaaaataaaaaactgctctgcagaaagtgcaaagccttggcatgttacacagctgacgtaagagtgatagaggaatgccattacactgtgcttggagatgcttttaaggaatgctttgtgagtagaccacatcccaagccaaagcagttttcaagttttgaaaaaagagcaaagatattctgtgcccgacagaactgcagccatgactggggaatccatgtgaagtacaagacatttgagattccagttataaaaattgaaagttttgtggtggaggatattgcaactggagttcagacactgtactcgaagtggaaggactttcattttgagaagataccatttgatccagcagaaatgtccaaatgatatcaggtcctcaatcttcagctacagggaatgagtaactttgagtggagaagaaacaaacatagtgggtataatcatggatcgcttgtacccctgtgaaaatatattttttaaaaatatctttagcagtttgtactatattatatatgcaaagcacaaatgagtgaatcacagcactgagtattttgtaggccaacagagctcatagtacttgggaaaaattaaaaagcctcatttctagccttctttttagagtcaactgccaacaaacacacagtaatcactctgtacacactgggatagatgaatgaatggaatgttgggaatttttatctccctttgtctccttaacctactgtaaactggcttttgcccttaacaatctactgaaattgttcttttgaaggttaccagtgactctggttgccaaatccactgggcacttcttaaccttctatttgacctctgcgcatttggccctgttgagcactcttcttgaagctctccctgggcttctctctcttctagttctattctagtctttttttattgagtcctcctctttgctgatcccttccaagggttcaatatatatacatgtatatactgtacatatgtatatgtaactaatatacatacatacaggtatgtatatgtaatggttatatgtactcatgttcctggtgtagcaacgtgtggtatggctacacagagaacatgagaacataaagccatttttatgcttactactaaaagctgtccactgtagagttgctgtatgtagcaatgtgtatccactctacagtggtcagcttttagtagagagcataaaaatgataaaatacttcttgaaaacttagtttactatacatcttgccctattaatatgttctcttaacgtgtgccattgttctctttgaccattttcctataatgatgttgatgttcaacacctggactgaatgtctgttctcagatcccttggatgttacagatgaggcagtctgactgtcctttctacttgaaagattagaatatgtatccaaatggcattcacgtgtcacttagcaaggtttgctgatgcttcaaagagcttagtttgcggtttcctggacgtggaaacaagtatctgagttccctggagatcaacgggatgaggtgttacagctgcctccctcttcatgcaatctggtgagcagtggtgcaggcggggagccagagaaacttgccagttatataacttctctttggcttttcttcatctgtaaaacaaggataatactgaactgtaagggttagtggagagtttttaattaaaagaatgtgtgaaaagtacatgacacagtagttgcttgataatagttactagtagtagtattcttactaagacccaatacaaatggattatttaaaccaagtttatgagttggttttttttcattttctatttgtattttattaagagtgtcttttcttatgtgattttttttaattgctatttgatatggtttggctatatgtccccacccaaatctcatcttgaattataatccccatgtgtcaagggagggacctgacgggaggtgattggatcacgggggcagttgtccccatgctgttcttgggatagtgagttagttctcatgagatctgatggttttataagtgtttgacaattcctcctttacacacactctctctctcatctgctgccatgtaagacttgcctgcttccccttctgccatgattgtaagtttcctgaggcctcctcagccatgtggaactgtgaatctattaagcctcttttctttataaatga

SEQ ID NO: 225 - DNA, 3160 bp, Homo sapiens
  source 1..3160: mol_type = genomic DNA
aagtgagacggccgcgggctgaggggccaaggcggcgcggggcgcggtcggtcaggcctgcgccgcccggcggggagcgctcccgccccctgtgggaggctgggatgctgagtcagagcgggggcgggggcgggcgcaccgagaagccccggacgacccgaggccaagccatccaggaccccggctgcgtggacactgcctctgggtataacaagaggattgcctgatccagccaagatgcagagcacttctaatcatctgtggcttttatctgatattttaggccaaggagctactgcaaatgtctttcgtggaagacataagaaaactggtgatttatttgctatcaaagtatttaataacataagcttccttcgtccagtggatgttcaaatgagagaatttgaagtgttgaaaaaactcaatcacaaaaatattgtcaaattatttgctattgaagaggagacaacaacaagacataaagtacttattatggaattttgtccatgtgggagtttatacactgttttagaagaaccttctaatgcctatggactaccagaatctgaattcttaattgttttgcgagatgtggtgggtggaatgaatcatctacgagagaatggtatagtgcaccgtgatatcaagccaggaaatatcatgcgtgttataggggaagatggacagtctgtgtacaaactcacagattttggtgcagctagagaattagaagatgatgagcagtttgtttctctgtatggcacagaagaatatttgcaccctgatatgtatgagagagcagtgctaagaaaagatcatcagaagaaatatggagcaacagttgatctttggagcattggggtaacattttaccatgcagctactggatcactgccatttagaccctttgaagggcctcgtaggaataaagaagtgatgtataaaataattacaggaaagccttctggtgcaatatctggagtacagaaagcagaaaatggaccaattgactggagtggagacatgcctgtttcttgcagtctttctcggggtcttcaggttctacttacccctgttcttgcaaacatccttgaagcagatcaggaaaagtgttggggttttgaccagttttttgcagaaactagtgatatacttcaccgaatggtaattcatgttttttcgctacaacaaatgacagctcataagatttatattcatagctataatactgctactatatttcatgaactggtatataaacaaaccaaaattatttcttcaaatcaagaacttatctacgaagggcgacgcttagtcttagaacctggaaggctggcacaacatttccctaaaactactgaggaaaaccctatatttgtagtaagccgggaacctctgaataccataggattaatatatgaaaaaatttccctccctaaagtacatccacgttatgatttagacggggatgctagcatggctaaggcaataacaggggttgtgtgttatgcctgcagaattgccagtaccttactgctttatcaggaattaatgcgaaaggggatacgatggctgattgaattaattaaagatgattacaatgaaactgttcacaaaaagacagaagttgtgatcacattggatttctgtatcagaaacattgaaaaaactgtgaaagtatatgaaaagttgatgaagatcaacctggaagcggcagagttaggtgaaatttcagacatacacaccaaattgttgagactttccagttctcagggaacaatagaaaccagtcttcaggatatcgacagcagattatctccaggtggatcactggcagacgcatgggcacatcaagaaggcactcatccgaaagacagaaatgtagaaaaactacaagtcctgttaaattgcatgacagagatttactatcagttcaaaaaagacaaagcagaacgtagattagcttataatgaagaacaaatccacaaatttgataagcaaaaactgtattaccatgccacaaaagctatgacgcactttacagatgaatgtgttaaaaagtatgaggcatttttgaataagtcagaagaatggataagaaagatgcttcatcttaggaaacagttattatcgctgactaatcagtgttttgatattgaagaagaagtatcaaaatatcaagaatatactaatgagttacaagaaactctgcctcagaaaatgtttacagcttccagtggaatcaaacataccatgaccccaatttatccaagttctaacacattagtagaaatgactcttggtatgaagaaattaaaggaagagatggaaggggtggttaaagaacttgctgaaaataaccacattttagaaaggtttggctctttaaccatggatggtggccttcgcaacgttgactgtctttagctttctaatagaagtttaagaaaagtttccgtttgcacaagaaaataacgcttgggcattaaatgaatgcctttatagatagtcacttgtttctacaattcagtatttgatgtggtcgtgtaaatatgtacaatattgtaaatacataaaaaatatacaaatttttggctgctgtgaagatgtaattttatcttttaacatttataattatatgaggaaatttgacctcagtgatcacgagaagaaagccatgaccgaccaatatgttgacatactgatcctctactctgagtggggctaaataagttattttctctgaccgcctactggaaatatttttaagtggaaccaaaataggcatccttacaaatcaggaagactgacttgacacgtttgtaaatggtagaacggtggctactgtgagtggggagcagaaccgcaccactgttatactgggataacaatttttttgagaaggataaagtggcattattttattttacaaggtgcccagatcccagttatccttgtatccatgtaatttcagatgaattattaagcaaacattttaaagtgaattcattattaaaaactattcatttttttcctttggccataaatgtgtaattgtcattaaaattctaaggtcatttcaactgttttaagctgtatatttctttaattctgcttactatttcatggaaaaaaataaatttctcaattttaatgtaaa

SEQ ID NO: 226 - DNA, 3142 bp, Homo sapiens
  source 1..3142: mol_type = genomic DNA
atccccgctccgcgccgcgcggccggtcctcgacctgcatcccgtccggcggctttgggcataaagaggacgactccgttaccgggcctgggccctggctgcctggggaggcaggggcacctctttcccttagactcgctgttttctaccctctttccgagcgtcgacacaacgggcagtttttaaattataacaagaggattgcctgatccagccaagatgcagagcacttctaatcatctgtggcttttatctgatattttaggccaaggagctactgcaaatgtctttcgtggaagacataagaaaactggtgatttatttgctatcaaagtatttaataacataagcttccttcgtccagtggatgttcaaatgagagaatttgaagtgttgaaaaaactcaatcacaaaaatattgtcaaattatttgctattgaagaggagacaacaacaagacataaagtacttattatggaattttgtccatgtgggagtttatacactgttttagaagaaccttctaatgcctatggactaccagaatctgaattcttaattgttttgcgagatgtggtgggtggaatgaatcatctacgagagaatggtatagtgcaccgtgatatcaagccaggaaatatcatgcgtgttataggggaagatggacagtctgtgtacaaactcacagattttggtgcagctagagaattagaagatgatgagcagtttgtttctctgtatggcacagaagaatatttgcaccctgatatgtatgagagagcagtgctaagaaaagatcatcagaagaaatatggagcaacagttgatctttggagcattggggtaacattttaccatgcagctactggatcactgccatttagaccctttgaagggcctcgtaggaataaagaagtgatgtataaaataattacaggaaagccttctggtgcaatatctggagtacagaaagcagaaaatggaccaattgactggagtggagacatgcctgtttcttgcagtctttctcggggtcttcaggttctacttacccctgttcttgcaaacatccttgaagcagatcaggaaaagtgttggggttttgaccagttttttgcagaaactagtgatatacttcaccgaatggtaattcatgttttttcgctacaacaaatgacagctcataagatttatattcatagctataatactgctactatatttcatgaactggtatataaacaaaccaaaattatttcttcaaatcaagaacttatctacgaagggcgacgcttagtcttagaacctggaaggctggcacaacatttccctaaaactactgaggaaaaccctatatttgtagtaagccgggaacctctgaataccataggattaatatatgaaaaaatttccctccctaaagtacatccacgttatgatttagacggggatgctagcatggctaaggcaataacaggggttgtgtgttatgcctgcagaattgccagtaccttactgctttatcaggaattaatgcgaaaggggatacgatggctgattgaattaattaaagatgattacaatgaaactgttcacaaaaagacagaagttgtgatcacattggatttctgtatcagaaacattgaaaaaactgtgaaagtatatgaaaagttgatgaagatcaacctggaagcggcagagttaggtgaaatttcagacatacacaccaaattgttgagactttccagttctcagggaacaatagaaaccagtcttcaggatatcgacagcagattatctccaggtggatcactggcagacgcatgggcacatcaagaaggcactcatccgaaagacagaaatgtagaaaaactacaagtcctgttaaattgcatgacagagatttactatcagttcaaaaaagacaaagcagaacgtagattagcttataatgaagaacaaatccacaaatttgataagcaaaaactgtattaccatgccacaaaagctatgacgcactttacagatgaatgtgttaaaaagtatgaggcatttttgaataagtcagaagaatggataagaaagatgcttcatcttaggaaacagttattatcgctgactaatcagtgttttgatattgaagaagaagtatcaaaatatcaagaatatactaatgagttacaagaaactctgcctcagaaaatgtttacagcttccagtggaatcaaacataccatgaccccaatttatccaagttctaacacattagtagaaatgactcttggtatgaagaaattaaaggaagagatggaaggggtggttaaagaacttgctgaaaataaccacattttagaaaggtttggctctttaaccatggatggtggccttcgcaacgttgactgtctttagctttctaatagaagtttaagaaaagtttccgtttgcacaagaaaataacgcttgggcattaaatgaatgcctttatagatagtcacttgtttctacaattcagtatttgatgtggtcgtgtaaatatgtacaatattgtaaatacataaaaaatatacaaatttttggctgctgtgaagatgtaattttatcttttaacatttataattatatgaggaaatttgacctcagtgatcacgagaagaaagccatgaccgaccaatatgttgacatactgatcctctactctgagtggggctaaataagttattttctctgaccgcctactggaaatatttttaagtggaaccaaaataggcatccttacaaatcaggaagactgacttgacacgtttgtaaatggtagaacggtggctactgtgagtggggagcagaaccgcaccactgttatactgggataacaatttttttgagaaggataaagtggcattattttattttacaaggtgcccagatcccagttatccttgtatccatgtaatttcagatgaattattaagcaaacattttaaagtgaattcattattaaaaactattcatttttttcctttggccataaatgtgtaattgtcattaaaattctaaggtcatttcaactgttttaagctgtatatttctttaattctgcttactatttcatggaaaaaaataaatttctcaattttaatgtaaa

SEQ ID NO: 227 - DNA, 2541 bp, Homo sapiens
  source 1..2541: mol_type = genomic DNA
agcgccctcagcgcgctacggtccgcgggcaactccgcagaagccccagcccccaggaccccaggacccagtggcgcagccggcagccccggatccctgatctgcttgggcagctcctgcagaacctggaacagtgaatgggtgcagcaggccaggacaagctcttgtatctgaagcacaaactgaagaccccacgcccaggctgccaggggcaggacctcctgcatgccatggttctcctgaagctgggccaggaaactgaggccaggatctctctagaggcattgaaggccgatgcggtggcccggctggtggcccgccagtgggctggcgtggacagcaccgaggacccagaggagcccccagatgtgtcctgggctgtggcccgcttgtaccacctgctggctgaggagaagctgtgccccgcctcgctgcgggacgtggcctaccaggaagccgtccgcaccctcagctccagggacgaccaccggctgggggaacttcaggatgaggcccgaaaccggtgtgggtgggacattgctggggatccagggagcatccggacgctccagtccaatctgggctgcctcccaccatcctcggctttgccctctgggaccaggagcctcccacgccccattgacggtgtttcggactggagccaagggtgctccctgcgatccactggcagccctgcctccctggccagcaacttggaaatcagccagtcccctaccatgcccttcctcagcctgcaccgcagcccacatgggcccagcaagctctgtgacgacccccaggccagcttggtgcccgagcctgtccccggtggctgccaggagcctgaggagatgagctggccgccatcgggggagattgccagcccaccagagctgccaagcagcccacctcctgggcttcccgaagtggccccagatgcaacctccactggcctccctgatacccccgcagctccagaaaccagcaccaactacccagtggagtgcaccgaggggtctgcaggcccccagtctctccccttgcctattctggagccggtcaaaaacccctgctctgtcaaagaccagacgccactccaactttctgtagaagataccacctctccaaataccaagccgtgcccacctactcccaccaccccagaaacatcccctcctcctcctcctcctcctccttcatctactccttgttcagctcacctgaccccctcctccctgttcccttcctccctggaatcatcatcggaacagaaattctataactttgtgatcctccacgccagggcagacgaacacatcgccctgcgggttcgggagaagctggaggcccttggcgtgcccgacggggccaccttctgcgaggatttccaggtgccggggcgcggggagctgagctgcctgcaggacgccatagaccactcagctttcatcatcctacttctcacctccaacttcgactgtcgcctgagcctgcaccaggtgaaccaagccatgatgagcaacctcacgcgacaggggtcgccagactgtgtcatccccttcctgcccctggagagctccccggcccagctcagctccgacacggccagcctgctctccgggctggtgcggctggacgaacactcccagatcttcgccaggaaggtggccaacaccttcaagccccacaggcttcaggcccgaaaggccatgtggaggaaggaacaggacacccgagccctgcgggaacagagccaacacctggacggtgagcggatgcaggcggcggcactgaacgcagcctactcagcctacctccagagctacttgtcctaccaggcacagatggagcagctccaggtggcttttgggagccacatgtcatttgggactggggcgccctatggggctcgaatgccctttgggggccaggtgcccctgggagccccgccaccctttcccacttggccggggtgcccgcagccgccacccctgcacgcatggcaggctggcacccccccaccgccctccccacagccagcagcctttccacagtcactgcccttcccgcagtccccagccttccctacggcctcacccgcaccccctcagagcccagggctgcaacccctcattatccaccacgcacagatggtacagctggggctgaacaaccacatgtggaaccagagagggtcccaggcgcccgaggacaagacgcaggaggcagaatgaccgcgtgtccttgcctgaccacctggggaacacccctggacccaggcatcggccaggaccccatagagcaccccggtctgccctgtgccctgtggacagtggaagatgaggtcatctgccactttcaggacattgtccgggagcccttcatttaggacaaaacgggcgcgatgatgccctggctttcagggtggtcagaactggatacggtgtttacaattccaatctctctatttctgggtgaagggtcttggtggtgggggtattgctacggtcttttaattataataaatatttattgaatgct

SEQ ID NO: 228 - DNA, 2499 bp, Homo sapiens
  source 1..2499: mol_type = genomic DNA
agcgccctcagcgcgctacggtccgcgggcaactccgcagaagccccagcccccaggaccccaggacccagtggcgcagccggcagccccggatccctggtgcagcaggccaggacaagctcttgtatctgaagcacaaactgaagaccccacgcccaggctgccaggggcaggacctcctgcatgccatggttctcctgaagctgggccaggaaactgaggccaggatctctctagaggcattgaaggccgatgcggtggcccggctggtggcccgccagtgggctggcgtggacagcaccgaggacccagaggagcccccagatgtgtcctgggctgtggcccgcttgtaccacctgctggctgaggagaagctgtgccccgcctcgctgcgggacgtggcctaccaggaagccgtccgcaccctcagctccagggacgaccaccggctgggggaacttcaggatgaggcccgaaaccggtgtgggtgggacattgctggggatccagggagcatccggacgctccagtccaatctgggctgcctcccaccatcctcggctttgccctctgggaccaggagcctcccacgccccattgacggtgtttcggactggagccaagggtgctccctgcgatccactggcagccctgcctccctggccagcaacttggaaatcagccagtcccctaccatgcccttcctcagcctgcaccgcagcccacatgggcccagcaagctctgtgacgacccccaggccagcttggtgcccgagcctgtccccggtggctgccaggagcctgaggagatgagctggccgccatcgggggagattgccagcccaccagagctgccaagcagcccacctcctgggcttcccgaagtggccccagatgcaacctccactggcctccctgatacccccgcagctccagaaaccagcaccaactacccagtggagtgcaccgaggggtctgcaggcccccagtctctccccttgcctattctggagccggtcaaaaacccctgctctgtcaaagaccagacgccactccaactttctgtagaagataccacctctccaaataccaagccgtgcccacctactcccaccaccccagaaacatcccctcctcctcctcctcctcctccttcatctactccttgttcagctcacctgaccccctcctccctgttcccttcctccctggaatcatcatcggaacagaaattctataactttgtgatcctccacgccagggcagacgaacacatcgccctgcgggttcgggagaagctggaggcccttggcgtgcccgacggggccaccttctgcgaggatttccaggtgccggggcgcggggagctgagctgcctgcaggacgccatagaccactcagctttcatcatcctacttctcacctccaacttcgactgtcgcctgagcctgcaccaggtgaaccaagccatgatgagcaacctcacgcgacaggggtcgccagactgtgtcatccccttcctgcccctggagagctccccggcccagctcagctccgacacggccagcctgctctccgggctggtgcggctggacgaacactcccagatcttcgccaggaaggtggccaacaccttcaagccccacaggcttcaggcccgaaaggccatgtggaggaaggaacaggacacccgagccctgcgggaacagagccaacacctggacggtgagcggatgcaggcggcggcactgaacgcagcctactcagcctacctccagagctacttgtcctaccaggcacagatggagcagctccaggtggcttttgggagccacatgtcatttgggactggggcgccctatggggctcgaatgccctttgggggccaggtgcccctgggagccccgccaccctttcccacttggccggggtgcccgcagccgccacccctgcacgcatggcaggctggcacccccccaccgccctccccacagccagcagcctttccacagtcactgcccttcccgcagtccccagccttccctacggcctcacccgcaccccctcagagcccagggctgcaacccctcattatccaccacgcacagatggtacagctggggctgaacaaccacatgtggaaccagagagggtcccaggcgcccgaggacaagacgcaggaggcagaatgaccgcgtgtccttgcctgaccacctggggaacacccctggacccaggcatcggccaggaccccatagagcaccccggtctgccctgtgccctgtggacagtggaagatgaggtcatctgccactttcaggacattgtccgggagcccttcatttaggacaaaacgggcgcgatgatgccctggctttcagggtggtcagaactggatacggtgtttacaattccaatctctctatttctgggtgaagggtcttggtggtgggggtattgctacggtcttttaattataataaatatttattgaatgct

SEQ ID NO: 229 - DNA, 2107 bp, Homo sapiens
  source 1..2107: mol_type = genomic DNA
agcgccctcagcgcgctacggtccgcgggcaactccgcagaagccccagcccccaggaccccaggacccagtggcgcagccggcagccccggatccctgatctgcttgggcagctcctgcagaacctggaacagtgaatgggtaggggacactgggcgtgcagaaggcggggggcagtgtggaacatgccttcaccacctccagcttctgctgccggaggctgcacccacctggtgctccctgcgatccactggcagccctgcctccctggccagcaacttggaaatcagccagtcccctaccatgcccttcctcagcctgcaccgcagcccacatgggcccagcaagctctgtgacgacccccaggccagcttggtgcccgagcctgtccccggtggctgccaggagcctgaggagatgagctggccgccatcgggggagattgccagcccaccagagctgccaagcagcccacctcctgggcttcccgaagtggccccagatgcaacctccactggcctccctgatacccccgcagctccagaaaccagcaccaactacccagtggagtgcaccgaggggtctgcaggcccccagtctctccccttgcctattctggagccggtcaaaaacccctgctctgtcaaagaccagacgccactccaactttctgtagaagataccacctctccaaataccaagccgtgcccacctactcccaccaccccagaaacatcccctcctcctcctcctcctcctccttcatctactccttgttcagctcacctgaccccctcctccctgttcccttcctccctggaatcatcatcggaacagaaattctataactttgtgatcctccacgccagggcagacgaacacatcgccctgcgggttcgggagaagctggaggcccttggcgtgcccgacggggccaccttctgcgaggatttccaggtgccggggcgcggggagctgagctgcctgcaggacgccatagaccactcagctttcatcatcctacttctcacctccaacttcgactgtcgcctgagcctgcaccaggtgaaccaagccatgatgagcaacctcacgcgacaggggtcgccagactgtgtcatccccttcctgcccctggagagctccccggcccagctcagctccgacacggccagcctgctctccgggctggtgcggctggacgaacactcccagatcttcgccaggaaggtggccaacaccttcaagccccacaggcttcaggcccgaaaggccatgtggaggaaggaacaggacacccgagccctgcgggaacagagccaacacctggacggtgagcggatgcaggcggcggcactgaacgcagcctactcagcctacctccagagctacttgtcctaccaggcacagatggagcagctccaggtggcttttgggagccacatgtcatttgggactggggcgccctatggggctcgaatgccctttgggggccaggtgcccctgggagccccgccaccctttcccacttggccggggtgcccgcagccgccacccctgcacgcatggcaggctggcacccccccaccgccctccccacagccagcagcctttccacagtcactgcccttcccgcagtccccagccttccctacggcctcacccgcaccccctcagagcccagggctgcaacccctcattatccaccacgcacagatggtacagctggggctgaacaaccacatgtggaaccagagagggtcccaggcgcccgaggacaagacgcaggaggcagaatgaccgcgtgtccttgcctgaccacctggggaacacccctggacccaggcatcggccaggaccccatagagcaccccggtctgccctgtgccctgtggacagtggaagatgaggtcatctgccactttcaggacattgtccgggagcccttcatttaggacaaaacgggcgcgatgatgccctggctttcagggtggtcagaactggatacggtgtttacaattccaatctctctatttctgggtgaagggtcttggtggtgggggtattgctacggtcttttaattataataaatatttattgaatgct

SEQ ID NO: 230 - DNA, 2684 bp, Homo sapiens
  source 1..2684: mol_type = genomic DNA
agcgccctcagcgcgctacggtccgcgggcaactccgcagaagccccagcccccaggaccccaggacccagtggcgcagccggcagccccggatccctgatctgcttgggcagctcctgcagaacctggaacagtgaatgggtaggggacactgggcgtgcagaaggcggggggcagtgtggaacatgccttcaccacctccagcttctgctgccggaggctgcacccacctgtgcccatggcctgcacaggcccatcacttcctagcgccttcgacattctaggtgcagcaggccaggacaagctcttgtatctgaagcacaaactgaagaccccacgcccaggctgccaggggcaggacctcctgcatgccatggttctcctgaagctgggccaggaaactgaggccaggatctctctagaggcattgaaggccgatgcggtggcccggctggtggcccgccagtgggctggcgtggacagcaccgaggacccagaggagcccccagatgtgtcctgggctgtggcccgcttgtaccacctgctggctgaggagaagctgtgccccgcctcgctgcgggacgtggcctaccaggaagccgtccgcaccctcagctccagggacgaccaccggctgggggaacttcaggatgaggcccgaaaccggtgtgggtgggacattgctggggatccagggagcatccggacgctccagtccaatctgggctgcctcccaccatcctcggctttgccctctgggaccaggagcctcccacgccccattgacggtgtttcggactggagccaagggtgctccctgcgatccactggcagccctgcctccctggccagcaacttggaaatcagccagtcccctaccatgcccttcctcagcctgcaccgcagcccacatgggcccagcaagctctgtgacgacccccaggccagcttggtgcccgagcctgtccccggtggctgccaggagcctgaggagatgagctggccgccatcgggggagattgccagcccaccagagctgccaagcagcccacctcctgggcttcccgaagtggccccagatgcaacctccactggcctccctgatacccccgcagctccagaaaccagcaccaactacccagtggagtgcaccgaggggtctgcaggcccccagtctctccccttgcctattctggagccggtcaaaaacccctgctctgtcaaagaccagacgccactccaactttctgtagaagataccacctctccaaataccaagccgtgcccacctactcccaccaccccagaaacatcccctcctcctcctcctcctcctccttcatctactccttgttcagctcacctgaccccctcctccctgttcccttcctccctggaatcatcatcggaacagaaattctataactttgtgatcctccacgccagggcagacgaacacatcgccctgcgggttcgggagaagctggaggcccttggcgtgcccgacggggccaccttctgcgaggatttccaggtgccggggcgcggggagctgagctgcctgcaggacgccatagaccactcagctttcatcatcctacttctcacctccaacttcgactgtcgcctgagcctgcaccaggtgaaccaagccatgatgagcaacctcacgcgacaggggtcgccagactgtgtcatccccttcctgcccctggagagctccccggcccagctcagctccgacacggccagcctgctctccgggctggtgcggctggacgaacactcccagatcttcgccaggaaggtggccaacaccttcaagccccacaggcttcaggcccgaaaggccatgtggaggaaggaacaggacacccgagccctgcgggaacagagccaacacctggacggtgagcggatgcaggcggcggcactgaacgcagcctactcagcctacctccagagctacttgtcctaccaggcacagatggagcagctccaggtggcttttgggagccacatgtcatttgggactggggcgccctatggggctcgaatgccctttgggggccaggtgcccctgggagccccgccaccctttcccacttggccggggtgcccgcagccgccacccctgcacgcatggcaggctggcacccccccaccgccctccccacagccagcagcctttccacagtcactgcccttcccgcagtccccagccttccctacggcctcacccgcaccccctcagagcccagggctgcaacccctcattatccaccacgcacagatggtacagctggggctgaacaaccacatgtggaaccagagagggtcccaggcgcccgaggacaagacgcaggaggcagaatgaccgcgtgtccttgcctgaccacctggggaacacccctggacccaggcatcggccaggaccccatagagcaccccggtctgccctgtgccctgtggacagtggaagatgaggtcatctgccactttcaggacattgtccgggagcccttcatttaggacaaaacgggcgcgatgatgccctggctttcagggtggtcagaactggatacggtgtttacaattccaatctctctatttctgggtgaagggtcttggtggtgggggtattgctacggtcttttaattataataaatatttattgaatgct

SEQ ID NO: 231 - DNA, 5745 bp, Homo sapiens
  source 1..5745: mol_type = genomic DNA
cgtttcctcggcggcctcggagcgcgggtgcagcagttgtgtcccgacccctgggagcgccatggcagagctgtgccccctggccgaggagctgtcgtgctccatctgcctggagcccttcaaggagccggtcaccactccgtgcggccacaacttctgcgggtcgtgcctgaatgagacgtgggcagtccagggctcgccatacctgtgcccgcagtgccgcgccgtctaccaggcgcgaccgcagctgcacaagaacacggtgctgtgcaacgtggtggagcagttcctgcaggccgacctggcccgggagccacccgccgacgtctggacgccgcccgcccgcgcctctgcacccagcccgaatgcccaggtggcctgcgaccactgcctgaaggaggccgccgtgaagacgtgcttggtgtgcatggcctccttctgtcaggagcacctgcagccgcacttcgacagccccgccttccaggaccacccgctgcagccgcccgttcgcgacctgttgcgccgcaaatgttcccagcacaatcggctgcgggaatttttctgccccgagcacagcgagtgcatctgccacatctgcctggtggagcataagacctgctctcccgcgtccctgagccaggccagcgccgacctggaggccaccctgaggcacaaactaactgtcatgtacagtcagatcaacggggcgtcgagagcactggatgatgtgagaaacaggcagcaggatgtgcggatgactgcaaacagaaaggtggagcagctacaacaagaatacacggaaatgaaggctctcttggacgcctcagagaccacctcgacaaggaagataaaggaagaggagaagagggtcaacagcaagtttgacaccatttatcagattctcctcaagaagaagagtgagatccagaccttgaaggaggagattgaacagagcctgaccaagagggatgagttcgagtttctggagaaagcatcaaaactgcgaggaatctcaacaaagccagtctacatccccgaggtggaactgaaccacaagctgataaaaggcatccaccagagcaccatagacctcaaaaacgagctgaagcagtgcatcgggcggctccaggagcccacccccagttcaggtgaccctggagagcatgacccagcgtccacacacaaatccacacgccctgtgaagaaggtctccaaagaggaaaagaaatccaagaaacctccccctgtccctgccttacccagcaagcttcccacgtttggagccccggaacagttagtggatttaaaacaagctggcttggaggctgcagccaaagccaccagctcacatccgaactcaacatctctcaaggccaaggtgctggagaccttcctggccaagtccagacctgagctcctggagtattacattaaagtcatcctggactacaacaccgcccacaacaaagtggctctgtcagagtgctatacagtagcttctgtggctgagatgcctcagaactaccggccgcatccccagaggttcacatactgctctcaggtgctgggcctgcactgctacaagaaggggatccactactgggaggtggagctgcagaagaacaacttctgtggggtaggcatctgctacggaagcatgaaccggcagggcccagaaagcaggctcggccgcaacagcgcctcctggtgcgtggagtggttcaacaccaagatctctgcctggcacaataacgtggagaaaaccctgccctccaccaaggccacgcgggtgggcgtgcttctcaactgtgaccacggctttgtcatcttcttcgctgttgccgacaaggtccacctgatgtataagttcagggtggactttactgaggctttgtacccggctttctgggtattttctgctggtgccacactctccatctgctcccccaagtaggcaggctgtaggcacttgggctgactgcctgcagaagtcccaagaccctagtgaaaatacagcaggcagaactctccttggataattccccaagaggtcccaaggattgggagcatgggaggggagctggcgggagggtgggaggtgggatttagccaggaaaggggtgagagtgattgtgttgtgggcgaggaggcgtttccaccccctggtgcctatcagggcagggtgacctactccccattgttctggaaatctccaggctgctgggcagctgggcagagctctgggaagtgaagtcatgagtgcccgattcctcttagagaaaatccatagctactgtaggttctgtcttgggccacttggatctgaaggctgcccctttgctctctggggtagccttcagatcttggtgttttgaattcttactatagatgtttttaaagttccaaagtcattgagtttcaatgttacataaactccattacccgcatgttggggcttgatctcctggttattatctgtgcttgaggaaacacccacagcagtctctacccagaacagtttcctaaagaggcataccctcttcctccactggaaaatagtgcgttcccttcctaccctgcacacccatcgcccccacattgatggttttcaaacagcaaacttttcagctaaagcttcaaaccatgattggaatcagcctgtgttggatttgtgattcagggtcatggtgaccctgatccagtttgggtggaaatccttcctaagtatcataagaagcatcttggcagagatgctttggtggcagccatgagctttgctggaggccttgcttcccatagccttggctgtggggcaaggaactctgccaggcgagggggatgctgccctggatcaacagaagcctggtgggtttgctcgtgttagagtgtcctgccttcttactgacaactcttctcggtgatagcctctcttccctggattgtgacatatggaatgacagtgcaggtaccaccgaggctagcacagtcaagcctccagctaagctggatccctgaagcctgctatcatgcagacaggctatgcggctgcctcggaccatgctaggccacttgctggggtgtcaacctaccaccaaaggggtcttttagcaaacctcatggggaacaggaacattcctgttcatccctggccacaggctgcagacccagcactggcccttgcgtgagtcagagcctggggctggccctagccccttctactgacttcctcatttaagccaattatataagctcacattgatcagggagggagggaaagagctaaagagggtcacacaagtggctattttccctgcagtgtttctgtgtggtgaaaataacccagtccactaaggggcgggagtgaatggatggctggattttccccaagctccttatagcctaatgttgtcaggatgtgagtatgaggaatttagcctcttatagtgaaatgagtccaactctgggctttgcttagaggagagctcctgtcaggcttcctataatatgaaaagaagtcaccattggggaactagagaccccagaccttttcatatggatatttgagaatgtaatgcatctcaggcctcgtgctggaactctagggcactctaggcaggctcagaacacttgatattcctgacagctacacacctgacatgcaggtacatacctgatcggtgtcatctcctaacaaggattttcagttcctcgggagagcaataatctttgtaggaaagacatccctgcaataggtgatatgtggtccttagaagttttattcctttactacttggaagaaaagttctttggtgattcttctctgcttttgaagatgatcaaaagcatcttcattgattttctgaaacgaaagccttgtctgaaaccaattaatacttgggaaacagctgggcttggaggagtagaatgccagagataaatccatggctcctgctctggctctcttctgcagaaatgagggcaacagtgaggccacttccctggcaaatgtgcagctcaggatagggaagcataagaccctctgtttaaaagagagtcaagtaggtaaccaaagccaagctctgtgcaaggtgctttggagttgtaaattgaggagtgcatccttgctgtcttgaaccattctgtttgcaatggtgagaccttacataacctagccttgcagggccgccacacaaccctggagtcctagagttggaggaacctttgtatccatctgacttctcattttgcagaatatgatgagaaagtagaggatcgctctgttcaccactcttgctattccattagtggggagatgcctgctagcatgtgtgaggggaacactctgatacactgggaagtatcggaaattcccagaaacacaaacataaaataactctcctagacccaggtactggggactgtctcagtccgtgtggcatgataaataaaaggttaggatcaagtctttgtatttttcaagatgtggtagctgattattcctgttttaagtactctgaaattgatctgtgatcaataatactaatatgttatcttttaccgtattctgcctctcactattgattttaattagttaggagtatttgagctgttatttcttgagcttaatatttttttagagttaactctttaaggagataatcatggctgtagacaaggccagggctggctgacgtgccttagaaggtttgaatgcaataaagcggtgtttggcgttctcctgcattgtagtgcgggtacaaaatgctatttgttcgtcatactgttgtcagcagatgagccgcccactacagacggctactgcccagggacctgcccaggccccacccaagggctcccaagggttgagatttctgcagacctatagccagcacacttagtcctgccctatatagagttcctcttcgggaagcttttgataaggaattctcagaccgataggatgtctgtctgggctttgctgcgggacagtctaactgtgggggctaggggaaagcaggagagtatcgatcaaagagtaagccacacacggataatcagttactagggatggaggtgtgagggttcattatattattcattttactgttgtatatgtttgaaaatgtctataataaaaagctttaaaaaaaaaaaagaaacaaaaatagttcaggccagcaatgggccctccatgttcagtggattttatccttgtttcaattggggtaggagttccaggaggtgtggattggaggttgtctaaagaattgaactggcctttgtccctgcttcctgggagggagactttcagtccttgacatttccagggtaataggagtgtctttgttatttgtgaggccatgggatcacacctgagcttatgctaaaaggtgggatgactcagagctggggctggtcaccagacagactgaccatgtgattagaggtttgaggctttgagtcagcctgacctatggggcgggggactggaggctgagtttaatcatctggccagtgctttaatcagtacatcatgaaaccctaataaaaactctggacactaaagctcagcatagcttcctgggtggtgaagctgtggctatgccggcagggcagtgtgccctaattccacgaagagggggcatgggagcactgtgctcaagacccttccagacctttccctgtgtcttccttggcttgtggagctgatttgtatcctttataataaaactaatcataagtata

SEQ ID NO: 232 - DNA, 11335 bp, Homo sapiens
  source 1..11335: mol_type = genomic DNA
ccatactgaagccctcccaagtgcagccttgcgggcctcgcagagagcgccaaaccatccggccctgcgcgccacgcggaccggcatagccatctctctggagtggcagctgtgccaaggtggcaaggttaggcccgcaggccctcagcacgggggcgcagccggggcaagtcttcaggaagcagcgaggtgcttgaagggggtgctggaggcaggccaaacccagtcaggatgggtggcctggggccacgacgggcgggaacctcgagggagaggctagtggtaacaggccgagctggatggatgggtatggggagaggggcaggacgttcagccctgggattctggccgaccctcgccttccttctctgcagcttccccgcagccacctccccgtgcaagatcctcaagtgcaactctgagttctggagcgccacgtcgggcagccacgccccagcctcagacgacacccccgagttctgtgcagccttgcgcagctacgccctgtgcacgcggcggacggcccgcacctgccggggtgacctggcctaccactcggccgtccatggcatagaggacctcatgagccagcacaactgctccaaggatggccccacctcgcagccacgcctgcgcacgctcccaccggccggagacagccaggagcgctcggacagccccgagatctgccattacgagaagagctttcacaagcactcggccacccccaactacacgcactgtggcctcttcggggacccacacctcaggactttcaccgaccgcttccagacctgcaaggtgcagggcgcctggccgctcatcgacaataattacctgaacgtgcaggtcaccaacacgcctgtgctgcccggctcagcggccactgccaccagcaagctcaccatcatcttcaagaacttccaggagtgtgtggaccagaaggtgtaccaggctgagatggacgagctcccggccgccttcgtggatggctctaagaacggtggggacaagcacggggccaacagcctgaagatcactgagaaggtgtcaggccagcacgtggagatccaggccaagtacatcggcaccaccatcgtggtgcgccaggtgggccgctacctgacctttgccgtccgcatgccagaggaagtggtcaatgctgtggaggactgggacagccagggtctctacctctgcctgcggggctgccccctcaaccagcagatcgacttccaggccttccacaccaatgctgagggcaccggtgcccgcaggctggcagccgccagccctgcacccacagcccccgagaccttcccatacgagacagccgtggccaagtgcaaggagaagctgccggtggaggacctgtactaccaggcctgcgtcttcgacctcctcaccacgggcgacgtgaacttcacactggccgcctactacgcgttggaggatgtcaagatgctccactccaacaaagacaaactgcacctgtatgagaggactcgggacctgccaggcagggcggctgcggggctgcccctggccccccggcccctcctgggcgccctcgtcccgctcctggccctgctccctgtgttctgctagacgcgtagatgtggagggaggcgcgggctccgtcctctcggcttccccatgtgtgggctgggaccgcccacggggtgcagatctcctggcgtgtccaccatggccccgcagaacgccagggaccgcctgctgccaagggctcaggcacggacccctccccttctagtgcacgtgacaaggttgtggtgactggtgccatgatgtttgacagtagagctgtgtgagagggagagcagctcccctcgccccgcccctgcagtgtgaatgtgtgaaacatcccctcaggctgaagccccccacccccaccagagacacactgggaaccgtcagagtcagctccttccccctcgcaatgcactgaaaggcccggccgactgctgctcgccgatccgtggggccccctgtgcccgccacacgcacgcacacactcttacacgagagcacactcgatccccctaggccagcggggacaccccagccacacagggaggcatccttggggcttggccccaggcagggcaaccccggggcgctgcttggcaccttagcagactgctggaaccttttggccagtaggtcgtgcccgcctggtgccttctggcctgtggcctccctgcccatgttcacctggctgctgtgggtaccagtgcaggtcccggttttcaggcacctgctcagctgcccgtctctggcctgggcccctgccccttccaccctgtgcttagaaagtcgaagtgcttggttctaaatgtctaaacagagaagagatccttgacttctgttcctctctctcctgcagatgcaagagctcctgggcaggggtgcctgggccccagggtgtggcaggagacccagtggatggggccagctggcctgccctgatcctctgcttcctcctcacaaccccaagagcccccagcccggtccatccacgtctggagtctggggagaggagcagggtcttaggactctcagctctgagcatccctggcagggtcttcaacctctaatctcttcccttaagccctgtggccacacagccaggagagacttgccgctggctcccgcctcatttcagcccagggtgctcatccaggggcccagaacagtcccacctgtgctgctgtgcccacagcacaaagccaggcttcactcccaaaagtgcagccaggccctggagggtgatcctgccagcagccctacagctccacaccctacccacccatcggcagcccctctgctgttccccagggacctctcatacactggccaggaggctgcagaacgtgtgtctccccctccctccaagaggtcctgctccctctgccagaaccgtgtgtgggcgggtgggagggcgctcggggcccggcccctccctctccctgctggttttagttggtccctatgttggaagtaaaaagtgaagcactttattttggttgtgtttgctcacgttctgcttggaagtggggacccctcactgcgtccacgtgtctgcgacctgtgtggagtgtcaccgcgtgtacatactgtaaattatttattaatggctaaatgcaagtaaagtttggtttttttgttattttcttttagacgtgttggagtttgttttctgaacagactttggctcccttgaggcgggtggaggatggactttggtggtgggatcccctgcagcaagtgtgtgtgtgtgcgcgcatgtgtgtgtgcgcgcatgtgtgtatgcatgcctgtgtgtgtgcgtgcctgtgtgcgtgcatgtacgcatgtgtgcgcatgcctgtgtgtgcgcccatgcctatgtgtgtgcatgtaggcatgtgtgtgcgcatgtgtatgtgcgtgagaatggtgtgaaggcggatagaagatgcagcagtttaacagccaccacacattgcttttgtaaagtcacttttcctcctcacgatgagctgtctttggagcttggcaggcccagatcctcttgttctagtgacagtggaacacagtcagggagacaggtgactatgtggtggagagacatatggtgccctctgggatgagatgggtgggatagcctgagatctgggatgactccctctcagccactgctatcagcctggagccccagctgttgggactgcctgccaccctccctgccagtgcccatcttctcatagcccgggcggagcagtggtgctgcaggaggctgctgtccacgtcactgtgggaccaggtttattggcagctgtgatcacatttcatctgtacaaactacccagtgaatcaggaagaaattcaggcccattttgccgatgaaacagactctgagattaactgacttgcctagggctccttagcaacgggaaggacaacctgtgcacctaggttagtattgtcctgaagccccttctcttcaaactaaatgccatgaggcagaatagcagcacagttaagccagcctacatcctggcttctccacctccccagcagtgtgaccatgggcaagtcacttcacctctctgagcctgcatgctgttctctggaagatgggggggtgcctgtcccatacagttgtttggagagtcaggagttcattcctgtaaattgcttaggagagggcggggcacacatggcttgtctggtcgtggggagggagggggtggtattatcccaaagcaagataaatcagttaaggggagaaggaaaggggcgatacagaccctggactcttgggagaggtgaacctgcaggctagcaaggggaggagtcccaagagtggccaagttcattaatcccaggcagaccaggaaggctgaagaaaaaggagaactttattttatttttgaaatggagtctggcgtcttgctctgtcgccaggctggagtgcagtggcaccatcccagctcactgcagcccctgcctcccaggttcaagcaattctcctgcctcagcctcccaagtagctgggactacaggcacacaccaccacgcctggctaattttttgtattttagtagagatggtgtttcaccatgttgaccagtatggtcttgatctcctgacctcgtgatccgcctgccttggcctcccaaagtgttgggattacaggcgtgagccaccaccgtgcccggccaaaaaaggagaacgttctaaagccctgttcttgcagggctcccagagagaacacgggagttgggtttgggaggcaggaaccagactggaaaggacaaaacgtacccagcatcttccctggcggccggaaccgtcactacaggccccgtgcccttagcagtcccagggacagagcgactcagggaggcagcaaggaggagcggagccattggtgctcgtccggcacagagaacagttcctgagaagccagggcacacagaagaaaaagaaaaaagaaatctctactagacgaaaatgctaaatggcactagccacaaagtattcttggtttggaattttttttccaggaccctaaatgtaaccggctggaattctttcccaagtgccatgctacttactactggttgccctcacgagcatggaaatgccgtcgtgaatatggaacacagcatcctgagctaaaagattcccaagaagcagcttcatcttgcaggacgggctaggttgggagcctctggaggttcctggctgccagctccgcggaggcctgcggcctggaggtaaatgcattagcacacggaggtgggcgcctctccccgccccatcctgtctcactgatgtgttgccgagaactggagttcttgtttagctaaccagaatgtgcaaggctataggacaggagagaaatggcctttaaaacaaatgaatgagagtcacgatgaagggggcagacgggggggtgaaaatcattaccagaacacttgggcagagacccgtcagagccaggcaggagacagtcatcagagctgtgccagcaagtggcatggagcaggggtggcacttgggctcgggctgcaggcaaacttggcgtcaaaactggctcaacccgtcgcgagctgtgtggtctccttgggattttttgagccttagattccttatttgtaaaatggatttgtgagtctccacctcccgaggtcagtatggaaatccagtgagctaacactcgtcaatggcctcgcacatggccctggcaccacgcatggaggccgccaaaagtggccgcctcctctctgcaattgcatttatttatccatttgataactctattgggtgtctacccagcattgttcaggggggtgtgaggggctaccgcaatgacccagacagactgctgtgcattcttattggaaagacaaggcaataaaccacagttacaaaacccagggtcccatggtaacgagctccacagagaaaagggaagtgggccagaggttccgagagacacacattttggattgcaggagggtgagaaggggcattcatctgtggtggggttaaggggaggcctccgactagctcacctgcatgaatttcaggaacaagccatgcagagccggacagagcattctgtgcagagaaaacagtgtaacagccctagagcagcagcagcaaagggggagtttaagggatggtgggaagccactgtcactggacagtagcaagtggacagaccaggggtcccaggtgaggccacagagcagccaaagtactgcagggctctccggctccgggaggacttgatgtttgactccagttgccagaggaagccgttcacgggctttgagtgtaggaatgatgtgtccgacttgggtctttttattgttcttttttagagacagggtctcactctgtcacccaggctggagtgcagcggcacaatcacagctcactgcagcctccacttcctgggctcaagcaatcctcccacttcagccactcaagtagctgggattacaggtgcctgccgtcacacccggctaattgttaaatttttttggagagacaggatcttgctgttttcccaggctgatcttgaactcctgggctcaagcgagacgtctgtttcagcctcccaaagtgctgggataacaggtgtgagccactgggcctgccttggcttgggtattaaaggaattgttctagttactatatggaggtgacttggggccgctggggaagaaagaggaagtcaggagccagtctgaggcagtggcaatgcagcgattatcagaaataatgggctcaatgatagactggataaagaaaatgtggcacatatacgccatggagtactatgcagccataaaaagggatgagttcatgtcctttgcagggacatggatgaagctggaaaccatcattctcagcaaactaacacaagaacagaaaaccaaacaccgcatgttctcactcgtaagtgggagttgaacaatgagaacacatggacacagggaagggaacatcacacactggggcctgtcagtgggtggggggctgggggagggagagcatcaggagaaatacccaatgtagatgatgacgggttcataggtgcagcaaaccaccatggcacgtgtatacctatgtaacaaacttgtgcgttctgcacgtgtaccccataactgaaagtataatcataattttaagaaaagaaatgatggcctttggggtctaataagaaggtggggctgacagggcttggggagagagcgagcttgacaggagaccacgatgtcttccccaggggtctgagcacctggggaaatggtggtgccatttaccatgactggaaagatcatgggaggagcaggtttgggacactgaaggcccgagagcccctggcaaaccaccagtgtaagtccaagagtccaaaagctgaagaacttggagtccaatgtttgagggtaaaaaacagccagcatgagaaaaagatgaaggccagaagactcgtccagtctagcccttccacattcctctgcccgcttttatcctagcctcgctggcaggtgattagatggtgcccacccaggttaagcgtgggtctctgtctcccagttcactgactcaaatgttaatttcctttggcgacagcctcacagacacacccaggaacgatactttgcatcctccaatccaatcaagttggcactcaatattaaacagtttcggccgggagcggtggctcacgcctgtaatcccagcactttgggaggccaaagcgggcagatcactaagtcaggagatcgagaccatcctggctaacacggtgaaaccccatctctactaaaaatacaaaaaattagccaggcgtggtggtgggcgcctgtagtcccagctactcgggaggctgaggcaagagagtggcgtgaacccgggaggcagaacttgcagcaagcagagatcgcaccactgcactccagcctccagcctgggcgacagagcaagacaccgtctcaaaaaaaaaaaaaaaacaactaacagtttcgttcattaaggcaatgtccatggggcagctcctgcatgccacgccacccctgccctgacatactcccaggctgaccagccagaactggggagagggcatcttgaagaaacagtccaccctccgccctcgcatacctgtgtctcccagcagacaagacacacaagggcagggactgcactttcctgtgtgcaaatctcaggtatggtccagaaatcacatctctttggtctgattggccttacttaccaaacccttgaaacaatggaaagaagcttgctgcaatgggaagacccctgccccccaaaccctctaaccaggagtctacagacctcattccgtggaagttcccattgtcccctgggaaaccaccaccttctgaatcggcacagccaaggcccccaaccctgctatcaacagctttccttagccaccttccccatcctgcagggcttttcaacatttcgactttttaatccttccctccctaagatatagacatggtgttagtagcagaaccctgaaggcaaaggaacagaaactaacactctcagaaagtcctagcaggatggtctgaattaatagacagctgcaggcagccagtgtgcaaggcacgctcgtaatgtcagtggcgttccggaatgagggctgtgatcccaggcagcccagatcccaaggcgaaaacattcatgtcgcccagcatcaccctgtccttgcccagggtgaccctgagacggcacttggattcttccacatctgaactcaaacctgctccctcagctctccccagcaggaattggagccctctccaggccgttcagccaaagcagaatgtgtctggccaagcagatctgcccagagaagaacaaagacgtgttcagacactgtctggggaattggttttgagctcactctgagctccatctgtgcaacaaggccagcgtcagctgttacagagaaggggcttgtggcagtttattaccctggtgagctggctcggggccatgctgaccgcaggactgtaaaggacataaggcagttggcaatatgggaagcagcagtccccaaagcaccccgtcaaggaaacgatgcaagagctctgtccactggaccagccccaccctaccccgccacgccctggcctagggggagggaccttgtgtttcacgggcctggatggccagggatggacagtggcatgggcacagacacacccagaccgaaggaagggcccgggctccagaaccagacccgatccaggctcccagctctgtcacctactggctgcatgaaaagggtgggttttccttgttttgcagcctcagctatctcagctgtgagggaacaatgacgccacctccctcacgtggcggtcctgaagatgaaatgtaatgcatgcagcgcggcggccggcccaccgtgtgctcttagtaactggccgctgttctcttcagccctcccgttactgaccacctcgagggcaccgttcctccaagctgaccatttcctgccagtcacccactgcccagctcgcccagccatggagtgggctctggtccacgtgcatggtgagctgccatctttgtcgtcctctggacaccacgtctacctccttgggaaatagaaacgagtcctgcattgctttgtcaaatttcacaaaaaccctagaaagcagatattgccctggtttacagacggacagacagaagcaaagagaagccaactgacccactccaggttacaagcagggtctggtaggagagcacgcgtccaccccaccgctcactgcgcccacctactcctcgcttgagggcagagaacgagctggggcggggcgctccagggggcacaggcagcctgtgttcaaggcatgctcgtgagccccacagcaggggcggagtcaaaccagggctctggttctggggtgagcagagggtggggccaggcccccactgggaggctgactgaggaggggctcaaagggcaggcaggctccatcagagcggtttgatgagcagggccgctgagtggccctcaggtcaaggctggggccgggaccaccagggaccaggtctgcggggccaggggtccctccccagggtggagcaggaaaactccggcaaccgccttcttgagtggacttgaccttccctttctctgctaatgatctcagattaatcccttaaggcaagtgccccagggtgggtgttggttgcataaaacaagaggagtccttcttcccttccttcctggcacaaacatttgtaggccacccccgatgtggctggtcgtagtctggatttgggggcacattcaggcattcaacaaatacgtcatgaggccccgtgatgagctggggggctatgcttgatgtcggcagtacagctgtggtcgctcgaatgaatgtgcccgccctcacgggactgacctctagtggtgagagacagacactaaccgagtcacatgtgtgattacacgtgtaatcatacttacatatgtaacaacacgagttacacgtgcagtgtggggagggggcccttcacggtgaccagcccatcagggaaggggatgcgggtggcagaaggaagcggcacacccttgtcctcacggagctcacaggaggttctctgaccctcatcttaagccacaggctgtgtctgagcctcactgccctagctatcaactgggtgcaggactaggccttaatcactgggcagcgggaagaactacataagctcatactggcgcaaagccccttgggagaccgcagggaaagccattgcatgaggtggtgacaggctcggccttcagagtcgggctagaatctgagttctgacaatgatccccgccttgacccatctctcctcaggccgccctgagccttccagttaggcctgggctttgggcttccgtgtttgtctctggattgttcggttttagcaagagtcctgctaaattggtcttgctggaatcctccgccctccacatctgattgcccttcctgtctgatcgtggtcttcatgccccacggcctccaggggacgtctgatcacctggcctgccttcagcaagaatccccccaggtgtttcctcttaagaggttttccatccactgagcccaccctgctccttggccatcaattcccacttgcctgtactgtattcagaggggaccccaatctctcttcccagctgcagcggtccctgtccaacactgccccccacctcaaataaagtctttcttactgtctttaacatatgccgtcaataaagttttagcagatccaa

SEQ ID NO: 233 - DNA, 11191 bp, Homo sapiens
  source 1..11191: mol_type = genomic DNA
gcgccgggcgcccgcggccgaggtggacggagcctcccggctcgctgctcccgccggcggagcgaggctgccctttcttcgcagcgtgatttattttcttctttttttctgaactcttcttccagggagaggctagtggtaacaggccgagctggatggatgggtatggggagaggggcaggacgttcagccctgggattctggccgaccctcgccttccttctctgcagcttccccgcagccacctccccgtgcaagatcctcaagtgcaactctgagttctggagcgccacgtcgggcagccacgccccagcctcagacgacacccccgagttctgtgcagccttgcgcagctacgccctgtgcacgcggcggacggcccgcacctgccggggtgacctggcctaccactcggccgtccatggcatagaggacctcatgagccagcacaactgctccaaggatggccccacctcgcagccacgcctgcgcacgctcccaccggccggagacagccaggagcgctcggacagccccgagatctgccattacgagaagagctttcacaagcactcggccacccccaactacacgcactgtggcctcttcggggacccacacctcaggactttcaccgaccgcttccagacctgcaaggtgcagggcgcctggccgctcatcgacaataattacctgaacgtgcaggtcaccaacacgcctgtgctgcccggctcagcggccactgccaccagcaagctcaccatcatcttcaagaacttccaggagtgtgtggaccagaaggtgtaccaggctgagatggacgagctcccggccgccttcgtggatggctctaagaacggtggggacaagcacggggccaacagcctgaagatcactgagaaggtgtcaggccagcacgtggagatccaggccaagtacatcggcaccaccatcgtggtgcgccaggtgggccgctacctgacctttgccgtccgcatgccagaggaagtggtcaatgctgtggaggactgggacagccagggtctctacctctgcctgcggggctgccccctcaaccagcagatcgacttccaggccttccacaccaatgctgagggcaccggtgcccgcaggctggcagccgccagccctgcacccacagcccccgagaccttcccatacgagacagccgtggccaagtgcaaggagaagctgccggtggaggacctgtactaccaggcctgcgtcttcgacctcctcaccacgggcgacgtgaacttcacactggccgcctactacgcgttggaggatgtcaagatgctccactccaacaaagacaaactgcacctgtatgagaggactcgggacctgccaggcagggcggctgcggggctgcccctggccccccggcccctcctgggcgccctcgtcccgctcctggccctgctccctgtgttctgctagacgcgtagatgtggagggaggcgcgggctccgtcctctcggcttccccatgtgtgggctgggaccgcccacggggtgcagatctcctggcgtgtccaccatggccccgcagaacgccagggaccgcctgctgccaagggctcaggcacggacccctccccttctagtgcacgtgacaaggttgtggtgactggtgccatgatgtttgacagtagagctgtgtgagagggagagcagctcccctcgccccgcccctgcagtgtgaatgtgtgaaacatcccctcaggctgaagccccccacccccaccagagacacactgggaaccgtcagagtcagctccttccccctcgcaatgcactgaaaggcccggccgactgctgctcgccgatccgtggggccccctgtgcccgccacacgcacgcacacactcttacacgagagcacactcgatccccctaggccagcggggacaccccagccacacagggaggcatccttggggcttggccccaggcagggcaaccccggggcgctgcttggcaccttagcagactgctggaaccttttggccagtaggtcgtgcccgcctggtgccttctggcctgtggcctccctgcccatgttcacctggctgctgtgggtaccagtgcaggtcccggttttcaggcacctgctcagctgcccgtctctggcctgggcccctgccccttccaccctgtgcttagaaagtcgaagtgcttggttctaaatgtctaaacagagaagagatccttgacttctgttcctctctctcctgcagatgcaagagctcctgggcaggggtgcctgggccccagggtgtggcaggagacccagtggatggggccagctggcctgccctgatcctctgcttcctcctcacaaccccaagagcccccagcccggtccatccacgtctggagtctggggagaggagcagggtcttaggactctcagctctgagcatccctggcagggtcttcaacctctaatctcttcccttaagccctgtggccacacagccaggagagacttgccgctggctcccgcctcatttcagcccagggtgctcatccaggggcccagaacagtcccacctgtgctgctgtgcccacagcacaaagccaggcttcactcccaaaagtgcagccaggccctggagggtgatcctgccagcagccctacagctccacaccctacccacccatcggcagcccctctgctgttccccagggacctctcatacactggccaggaggctgcagaacgtgtgtctccccctccctccaagaggtcctgctccctctgccagaaccgtgtgtgggcgggtgggagggcgctcggggcccggcccctccctctccctgctggttttagttggtccctatgttggaagtaaaaagtgaagcactttattttggttgtgtttgctcacgttctgcttggaagtggggacccctcactgcgtccacgtgtctgcgacctgtgtggagtgtcaccgcgtgtacatactgtaaattatttattaatggctaaatgcaagtaaagtttggtttttttgttattttcttttagacgtgttggagtttgttttctgaacagactttggctcccttgaggcgggtggaggatggactttggtggtgggatcccctgcagcaagtgtgtgtgtgtgcgcgcatgtgtgtgtgcgcgcatgtgtgtatgcatgcctgtgtgtgtgcgtgcctgtgtgcgtgcatgtacgcatgtgtgcgcatgcctgtgtgtgcgcccatgcctatgtgtgtgcatgtaggcatgtgtgtgcgcatgtgtatgtgcgtgagaatggtgtgaaggcggatagaagatgcagcagtttaacagccaccacacattgcttttgtaaagtcacttttcctcctcacgatgagctgtctttggagcttggcaggcccagatcctcttgttctagtgacagtggaacacagtcagggagacaggtgactatgtggtggagagacatatggtgccctctgggatgagatgggtgggatagcctgagatctgggatgactccctctcagccactgctatcagcctggagccccagctgttgggactgcctgccaccctccctgccagtgcccatcttctcatagcccgggcggagcagtggtgctgcaggaggctgctgtccacgtcactgtgggaccaggtttattggcagctgtgatcacatttcatctgtacaaactacccagtgaatcaggaagaaattcaggcccattttgccgatgaaacagactctgagattaactgacttgcctagggctccttagcaacgggaaggacaacctgtgcacctaggttagtattgtcctgaagccccttctcttcaaactaaatgccatgaggcagaatagcagcacagttaagccagcctacatcctggcttctccacctccccagcagtgtgaccatgggcaagtcacttcacctctctgagcctgcatgctgttctctggaagatgggggggtgcctgtcccatacagttgtttggagagtcaggagttcattcctgtaaattgcttaggagagggcggggcacacatggcttgtctggtcgtggggagggagggggtggtattatcccaaagcaagataaatcagttaaggggagaaggaaaggggcgatacagaccctggactcttgggagaggtgaacctgcaggctagcaaggggaggagtcccaagagtggccaagttcattaatcccaggcagaccaggaaggctgaagaaaaaggagaactttattttatttttgaaatggagtctggcgtcttgctctgtcgccaggctggagtgcagtggcaccatcccagctcactgcagcccctgcctcccaggttcaagcaattctcctgcctcagcctcccaagtagctgggactacaggcacacaccaccacgcctggctaattttttgtattttagtagagatggtgtttcaccatgttgaccagtatggtcttgatctcctgacctcgtgatccgcctgccttggcctcccaaagtgttgggattacaggcgtgagccaccaccgtgcccggccaaaaaaggagaacgttctaaagccctgttcttgcagggctcccagagagaacacgggagttgggtttgggaggcaggaaccagactggaaaggacaaaacgtacccagcatcttccctggcggccggaaccgtcactacaggccccgtgcccttagcagtcccagggacagagcgactcagggaggcagcaaggaggagcggagccattggtgctcgtccggcacagagaacagttcctgagaagccagggcacacagaagaaaaagaaaaaagaaatctctactagacgaaaatgctaaatggcactagccacaaagtattcttggtttggaattttttttccaggaccctaaatgtaaccggctggaattctttcccaagtgccatgctacttactactggttgccctcacgagcatggaaatgccgtcgtgaatatggaacacagcatcctgagctaaaagattcccaagaagcagcttcatcttgcaggacgggctaggttgggagcctctggaggttcctggctgccagctccgcggaggcctgcggcctggaggtaaatgcattagcacacggaggtgggcgcctctccccgccccatcctgtctcactgatgtgttgccgagaactggagttcttgtttagctaaccagaatgtgcaaggctataggacaggagagaaatggcctttaaaacaaatgaatgagagtcacgatgaagggggcagacgggggggtgaaaatcattaccagaacacttgggcagagacccgtcagagccaggcaggagacagtcatcagagctgtgccagcaagtggcatggagcaggggtggcacttgggctcgggctgcaggcaaacttggcgtcaaaactggctcaacccgtcgcgagctgtgtggtctccttgggattttttgagccttagattccttatttgtaaaatggatttgtgagtctccacctcccgaggtcagtatggaaatccagtgagctaacactcgtcaatggcctcgcacatggccctggcaccacgcatggaggccgccaaaagtggccgcctcctctctgcaattgcatttatttatccatttgataactctattgggtgtctacccagcattgttcaggggggtgtgaggggctaccgcaatgacccagacagactgctgtgcattcttattggaaagacaaggcaataaaccacagttacaaaacccagggtcccatggtaacgagctccacagagaaaagggaagtgggccagaggttccgagagacacacattttggattgcaggagggtgagaaggggcattcatctgtggtggggttaaggggaggcctccgactagctcacctgcatgaatttcaggaacaagccatgcagagccggacagagcattctgtgcagagaaaacagtgtaacagccctagagcagcagcagcaaagggggagtttaagggatggtgggaagccactgtcactggacagtagcaagtggacagaccaggggtcccaggtgaggccacagagcagccaaagtactgcagggctctccggctccgggaggacttgatgtttgactccagttgccagaggaagccgttcacgggctttgagtgtaggaatgatgtgtccgacttgggtctttttattgttcttttttagagacagggtctcactctgtcacccaggctggagtgcagcggcacaatcacagctcactgcagcctccacttcctgggctcaagcaatcctcccacttcagccactcaagtagctgggattacaggtgcctgccgtcacacccggctaattgttaaatttttttggagagacaggatcttgctgttttcccaggctgatcttgaactcctgggctcaagcgagacgtctgtttcagcctcccaaagtgctgggataacaggtgtgagccactgggcctgccttggcttgggtattaaaggaattgttctagttactatatggaggtgacttggggccgctggggaagaaagaggaagtcaggagccagtctgaggcagtggcaatgcagcgattatcagaaataatgggctcaatgatagactggataaagaaaatgtggcacatatacgccatggagtactatgcagccataaaaagggatgagttcatgtcctttgcagggacatggatgaagctggaaaccatcattctcagcaaactaacacaagaacagaaaaccaaacaccgcatgttctcactcgtaagtgggagttgaacaatgagaacacatggacacagggaagggaacatcacacactggggcctgtcagtgggtggggggctgggggagggagagcatcaggagaaatacccaatgtagatgatgacgggttcataggtgcagcaaaccaccatggcacgtgtatacctatgtaacaaacttgtgcgttctgcacgtgtaccccataactgaaagtataatcataattttaagaaaagaaatgatggcctttggggtctaataagaaggtggggctgacagggcttggggagagagcgagcttgacaggagaccacgatgtcttccccaggggtctgagcacctggggaaatggtggtgccatttaccatgactggaaagatcatgggaggagcaggtttgggacactgaaggcccgagagcccctggcaaaccaccagtgtaagtccaagagtccaaaagctgaagaacttggagtccaatgtttgagggtaaaaaacagccagcatgagaaaaagatgaaggccagaagactcgtccagtctagcccttccacattcctctgcccgcttttatcctagcctcgctggcaggtgattagatggtgcccacccaggttaagcgtgggtctctgtctcccagttcactgactcaaatgttaatttcctttggcgacagcctcacagacacacccaggaacgatactttgcatcctccaatccaatcaagttggcactcaatattaaacagtttcggccgggagcggtggctcacgcctgtaatcccagcactttgggaggccaaagcgggcagatcactaagtcaggagatcgagaccatcctggctaacacggtgaaaccccatctctactaaaaatacaaaaaattagccaggcgtggtggtgggcgcctgtagtcccagctactcgggaggctgaggcaagagagtggcgtgaacccgggaggcagaacttgcagcaagcagagatcgcaccactgcactccagcctccagcctgggcgacagagcaagacaccgtctcaaaaaaaaaaaaaaaacaactaacagtttcgttcattaaggcaatgtccatggggcagctcctgcatgccacgccacccctgccctgacatactcccaggctgaccagccagaactggggagagggcatcttgaagaaacagtccaccctccgccctcgcatacctgtgtctcccagcagacaagacacacaagggcagggactgcactttcctgtgtgcaaatctcaggtatggtccagaaatcacatctctttggtctgattggccttacttaccaaacccttgaaacaatggaaagaagcttgctgcaatgggaagacccctgccccccaaaccctctaaccaggagtctacagacctcattccgtggaagttcccattgtcccctgggaaaccaccaccttctgaatcggcacagccaaggcccccaaccctgctatcaacagctttccttagccaccttccccatcctgcagggcttttcaacatttcgactttttaatccttccctccctaagatatagacatggtgttagtagcagaaccctgaaggcaaaggaacagaaactaacactctcagaaagtcctagcaggatggtctgaattaatagacagctgcaggcagccagtgtgcaaggcacgctcgtaatgtcagtggcgttccggaatgagggctgtgatcccaggcagcccagatcccaaggcgaaaacattcatgtcgcccagcatcaccctgtccttgcccagggtgaccctgagacggcacttggattcttccacatctgaactcaaacctgctccctcagctctccccagcaggaattggagccctctccaggccgttcagccaaagcagaatgtgtctggccaagcagatctgcccagagaagaacaaagacgtgttcagacactgtctggggaattggttttgagctcactctgagctccatctgtgcaacaaggccagcgtcagctgttacagagaaggggcttgtggcagtttattaccctggtgagctggctcggggccatgctgaccgcaggactgtaaaggacataaggcagttggcaatatgggaagcagcagtccccaaagcaccccgtcaaggaaacgatgcaagagctctgtccactggaccagccccaccctaccccgccacgccctggcctagggggagggaccttgtgtttcacgggcctggatggccagggatggacagtggcatgggcacagacacacccagaccgaaggaagggcccgggctccagaaccagacccgatccaggctcccagctctgtcacctactggctgcatgaaaagggtgggttttccttgttttgcagcctcagctatctcagctgtgagggaacaatgacgccacctccctcacgtggcggtcctgaagatgaaatgtaatgcatgcagcgcggcggccggcccaccgtgtgctcttagtaactggccgctgttctcttcagccctcccgttactgaccacctcgagggcaccgttcctccaagctgaccatttcctgccagtcacccactgcccagctcgcccagccatggagtgggctctggtccacgtgcatggtgagctgccatctttgtcgtcctctggacaccacgtctacctccttgggaaatagaaacgagtcctgcattgctttgtcaaatttcacaaaaaccctagaaagcagatattgccctggtttacagacggacagacagaagcaaagagaagccaactgacccactccaggttacaagcagggtctggtaggagagcacgcgtccaccccaccgctcactgcgcccacctactcctcgcttgagggcagagaacgagctggggcggggcgctccagggggcacaggcagcctgtgttcaaggcatgctcgtgagccccacagcaggggcggagtcaaaccagggctctggttctggggtgagcagagggtggggccaggcccccactgggaggctgactgaggaggggctcaaagggcaggcaggctccatcagagcggtttgatgagcagggccgctgagtggccctcaggtcaaggctggggccgggaccaccagggaccaggtctgcggggccaggggtccctccccagggtggagcaggaaaactccggcaaccgccttcttgagtggacttgaccttccctttctctgctaatgatctcagattaatcccttaaggcaagtgccccagggtgggtgttggttgcataaaacaagaggagtccttcttcccttccttcctggcacaaacatttgtaggccacccccgatgtggctggtcgtagtctggatttgggggcacattcaggcattcaacaaatacgtcatgaggccccgtgatgagctggggggctatgcttgatgtcggcagtacagctgtggtcgctcgaatgaatgtgcccgccctcacgggactgacctctagtggtgagagacagacactaaccgagtcacatgtgtgattacacgtgtaatcatacttacatatgtaacaacacgagttacacgtgcagtgtggggagggggcccttcacggtgaccagcccatcagggaaggggatgcgggtggcagaaggaagcggcacacccttgtcctcacggagctcacaggaggttctctgaccctcatcttaagccacaggctgtgtctgagcctcactgccctagctatcaactgggtgcaggactaggccttaatcactgggcagcgggaagaactacataagctcatactggcgcaaagccccttgggagaccgcagggaaagccattgcatgaggtggtgacaggctcggccttcagagtcgggctagaatctgagttctgacaatgatccccgccttgacccatctctcctcaggccgccctgagccttccagttaggcctgggctttgggcttccgtgtttgtctctggattgttcggttttagcaagagtcctgctaaattggtcttgctggaatcctccgccctccacatctgattgcccttcctgtctgatcgtggtcttcatgccccacggcctccaggggacgtctgatcacctggcctgccttcagcaagaatccccccaggtgtttcctcttaagaggttttccatccactgagcccaccctgctccttggccatcaattcccacttgcctgtactgtattcagaggggaccccaatctctcttcccagctgcagcggtccctgtccaacactgccccccacctcaaataaagtctttcttactgtctttaacatatgccgtcaataaagttttagcagatccaa

SEQ ID NO: 234 - DNA, 11309 bp, Homo sapiens
  source 1..11309: mol_type = genomic DNA
ctcccgctcgacgcccggctgcggagctgcctggccatctcctgccctcggggatgtcggcgcagtgcaccgcgtgggctcgcgtcccggcggccctttgcctcggcgcgccggcgtacgggaaggtgcggaccttggattgagggccgaggatgcccattgccaagccgccccggctttcctaagtggacgggagggcacggcgacgattcagcgccggctcgcgccgcagctcaacccaggggagaggctagtggtaacaggccgagctggatggatgggtatggggagaggggcaggacgttcagccctgggattctggccgaccctcgccttccttctctgcagcttccccgcagccacctccccgtgcaagatcctcaagtgcaactctgagttctggagcgccacgtcgggcagccacgccccagcctcagacgacacccccgagttctgtgcagccttgcgcagctacgccctgtgcacgcggcggacggcccgcacctgccggggtgacctggcctaccactcggccgtccatggcatagaggacctcatgagccagcacaactgctccaaggatggccccacctcgcagccacgcctgcgcacgctcccaccggccggagacagccaggagcgctcggacagccccgagatctgccattacgagaagagctttcacaagcactcggccacccccaactacacgcactgtggcctcttcggggacccacacctcaggactttcaccgaccgcttccagacctgcaaggtgcagggcgcctggccgctcatcgacaataattacctgaacgtgcaggtcaccaacacgcctgtgctgcccggctcagcggccactgccaccagcaagctcaccatcatcttcaagaacttccaggagtgtgtggaccagaaggtgtaccaggctgagatggacgagctcccggccgccttcgtggatggctctaagaacggtggggacaagcacggggccaacagcctgaagatcactgagaaggtgtcaggccagcacgtggagatccaggccaagtacatcggcaccaccatcgtggtgcgccaggtgggccgctacctgacctttgccgtccgcatgccagaggaagtggtcaatgctgtggaggactgggacagccagggtctctacctctgcctgcggggctgccccctcaaccagcagatcgacttccaggccttccacaccaatgctgagggcaccggtgcccgcaggctggcagccgccagccctgcacccacagcccccgagaccttcccatacgagacagccgtggccaagtgcaaggagaagctgccggtggaggacctgtactaccaggcctgcgtcttcgacctcctcaccacgggcgacgtgaacttcacactggccgcctactacgcgttggaggatgtcaagatgctccactccaacaaagacaaactgcacctgtatgagaggactcgggacctgccaggcagggcggctgcggggctgcccctggccccccggcccctcctgggcgccctcgtcccgctcctggccctgctccctgtgttctgctagacgcgtagatgtggagggaggcgcgggctccgtcctctcggcttccccatgtgtgggctgggaccgcccacggggtgcagatctcctggcgtgtccaccatggccccgcagaacgccagggaccgcctgctgccaagggctcaggcacggacccctccccttctagtgcacgtgacaaggttgtggtgactggtgccatgatgtttgacagtagagctgtgtgagagggagagcagctcccctcgccccgcccctgcagtgtgaatgtgtgaaacatcccctcaggctgaagccccccacccccaccagagacacactgggaaccgtcagagtcagctccttccccctcgcaatgcactgaaaggcccggccgactgctgctcgccgatccgtggggccccctgtgcccgccacacgcacgcacacactcttacacgagagcacactcgatccccctaggccagcggggacaccccagccacacagggaggcatccttggggcttggccccaggcagggcaaccccggggcgctgcttggcaccttagcagactgctggaaccttttggccagtaggtcgtgcccgcctggtgccttctggcctgtggcctccctgcccatgttcacctggctgctgtgggtaccagtgcaggtcccggttttcaggcacctgctcagctgcccgtctctggcctgggcccctgccccttccaccctgtgcttagaaagtcgaagtgcttggttctaaatgtctaaacagagaagagatccttgacttctgttcctctctctcctgcagatgcaagagctcctgggcaggggtgcctgggccccagggtgtggcaggagacccagtggatggggccagctggcctgccctgatcctctgcttcctcctcacaaccccaagagcccccagcccggtccatccacgtctggagtctggggagaggagcagggtcttaggactctcagctctgagcatccctggcagggtcttcaacctctaatctcttcccttaagccctgtggccacacagccaggagagacttgccgctggctcccgcctcatttcagcccagggtgctcatccaggggcccagaacagtcccacctgtgctgctgtgcccacagcacaaagccaggcttcactcccaaaagtgcagccaggccctggagggtgatcctgccagcagccctacagctccacaccctacccacccatcggcagcccctctgctgttccccagggacctctcatacactggccaggaggctgcagaacgtgtgtctccccctccctccaagaggtcctgctccctctgccagaaccgtgtgtgggcgggtgggagggcgctcggggcccggcccctccctctccctgctggttttagttggtccctatgttggaagtaaaaagtgaagcactttattttggttgtgtttgctcacgttctgcttggaagtggggacccctcactgcgtccacgtgtctgcgacctgtgtggagtgtcaccgcgtgtacatactgtaaattatttattaatggctaaatgcaagtaaagtttggtttttttgttattttcttttagacgtgttggagtttgttttctgaacagactttggctcccttgaggcgggtggaggatggactttggtggtgggatcccctgcagcaagtgtgtgtgtgtgcgcgcatgtgtgtgtgcgcgcatgtgtgtatgcatgcctgtgtgtgtgcgtgcctgtgtgcgtgcatgtacgcatgtgtgcgcatgcctgtgtgtgcgcccatgcctatgtgtgtgcatgtaggcatgtgtgtgcgcatgtgtatgtgcgtgagaatggtgtgaaggcggatagaagatgcagcagtttaacagccaccacacattgcttttgtaaagtcacttttcctcctcacgatgagctgtctttggagcttggcaggcccagatcctcttgttctagtgacagtggaacacagtcagggagacaggtgactatgtggtggagagacatatggtgccctctgggatgagatgggtgggatagcctgagatctgggatgactccctctcagccactgctatcagcctggagccccagctgttgggactgcctgccaccctccctgccagtgcccatcttctcatagcccgggcggagcagtggtgctgcaggaggctgctgtccacgtcactgtgggaccaggtttattggcagctgtgatcacatttcatctgtacaaactacccagtgaatcaggaagaaattcaggcccattttgccgatgaaacagactctgagattaactgacttgcctagggctccttagcaacgggaaggacaacctgtgcacctaggttagtattgtcctgaagccccttctcttcaaactaaatgccatgaggcagaatagcagcacagttaagccagcctacatcctggcttctccacctccccagcagtgtgaccatgggcaagtcacttcacctctctgagcctgcatgctgttctctggaagatgggggggtgcctgtcccatacagttgtttggagagtcaggagttcattcctgtaaattgcttaggagagggcggggcacacatggcttgtctggtcgtggggagggagggggtggtattatcccaaagcaagataaatcagttaaggggagaaggaaaggggcgatacagaccctggactcttgggagaggtgaacctgcaggctagcaaggggaggagtcccaagagtggccaagttcattaatcccaggcagaccaggaaggctgaagaaaaaggagaactttattttatttttgaaatggagtctggcgtcttgctctgtcgccaggctggagtgcagtggcaccatcccagctcactgcagcccctgcctcccaggttcaagcaattctcctgcctcagcctcccaagtagctgggactacaggcacacaccaccacgcctggctaattttttgtattttagtagagatggtgtttcaccatgttgaccagtatggtcttgatctcctgacctcgtgatccgcctgccttggcctcccaaagtgttgggattacaggcgtgagccaccaccgtgcccggccaaaaaaggagaacgttctaaagccctgttcttgcagggctcccagagagaacacgggagttgggtttgggaggcaggaaccagactggaaaggacaaaacgtacccagcatcttccctggcggccggaaccgtcactacaggccccgtgcccttagcagtcccagggacagagcgactcagggaggcagcaaggaggagcggagccattggtgctcgtccggcacagagaacagttcctgagaagccagggcacacagaagaaaaagaaaaaagaaatctctactagacgaaaatgctaaatggcactagccacaaagtattcttggtttggaattttttttccaggaccctaaatgtaaccggctggaattctttcccaagtgccatgctacttactactggttgccctcacgagcatggaaatgccgtcgtgaatatggaacacagcatcctgagctaaaagattcccaagaagcagcttcatcttgcaggacgggctaggttgggagcctctggaggttcctggctgccagctccgcggaggcctgcggcctggaggtaaatgcattagcacacggaggtgggcgcctctccccgccccatcctgtctcactgatgtgttgccgagaactggagttcttgtttagctaaccagaatgtgcaaggctataggacaggagagaaatggcctttaaaacaaatgaatgagagtcacgatgaagggggcagacgggggggtgaaaatcattaccagaacacttgggcagagacccgtcagagccaggcaggagacagtcatcagagctgtgccagcaagtggcatggagcaggggtggcacttgggctcgggctgcaggcaaacttggcgtcaaaactggctcaacccgtcgcgagctgtgtggtctccttgggattttttgagccttagattccttatttgtaaaatggatttgtgagtctccacctcccgaggtcagtatggaaatccagtgagctaacactcgtcaatggcctcgcacatggccctggcaccacgcatggaggccgccaaaagtggccgcctcctctctgcaattgcatttatttatccatttgataactctattgggtgtctacccagcattgttcaggggggtgtgaggggctaccgcaatgacccagacagactgctgtgcattcttattggaaagacaaggcaataaaccacagttacaaaacccagggtcccatggtaacgagctccacagagaaaagggaagtgggccagaggttccgagagacacacattttggattgcaggagggtgagaaggggcattcatctgtggtggggttaaggggaggcctccgactagctcacctgcatgaatttcaggaacaagccatgcagagccggacagagcattctgtgcagagaaaacagtgtaacagccctagagcagcagcagcaaagggggagtttaagggatggtgggaagccactgtcactggacagtagcaagtggacagaccaggggtcccaggtgaggccacagagcagccaaagtactgcagggctctccggctccgggaggacttgatgtttgactccagttgccagaggaagccgttcacgggctttgagtgtaggaatgatgtgtccgacttgggtctttttattgttcttttttagagacagggtctcactctgtcacccaggctggagtgcagcggcacaatcacagctcactgcagcctccacttcctgggctcaagcaatcctcccacttcagccactcaagtagctgggattacaggtgcctgccgtcacacccggctaattgttaaatttttttggagagacaggatcttgctgttttcccaggctgatcttgaactcctgggctcaagcgagacgtctgtttcagcctcccaaagtgctgggataacaggtgtgagccactgggcctgccttggcttgggtattaaaggaattgttctagttactatatggaggtgacttggggccgctggggaagaaagaggaagtcaggagccagtctgaggcagtggcaatgcagcgattatcagaaataatgggctcaatgatagactggataaagaaaatgtggcacatatacgccatggagtactatgcagccataaaaagggatgagttcatgtcctttgcagggacatggatgaagctggaaaccatcattctcagcaaactaacacaagaacagaaaaccaaacaccgcatgttctcactcgtaagtgggagttgaacaatgagaacacatggacacagggaagggaacatcacacactggggcctgtcagtgggtggggggctgggggagggagagcatcaggagaaatacccaatgtagatgatgacgggttcataggtgcagcaaaccaccatggcacgtgtatacctatgtaacaaacttgtgcgttctgcacgtgtaccccataactgaaagtataatcataattttaagaaaagaaatgatggcctttggggtctaataagaaggtggggctgacagggcttggggagagagcgagcttgacaggagaccacgatgtcttccccaggggtctgagcacctggggaaatggtggtgccatttaccatgactggaaagatcatgggaggagcaggtttgggacactgaaggcccgagagcccctggcaaaccaccagtgtaagtccaagagtccaaaagctgaagaacttggagtccaatgtttgagggtaaaaaacagccagcatgagaaaaagatgaaggccagaagactcgtccagtctagcccttccacattcctctgcccgcttttatcctagcctcgctggcaggtgattagatggtgcccacccaggttaagcgtgggtctctgtctcccagttcactgactcaaatgttaatttcctttggcgacagcctcacagacacacccaggaacgatactttgcatcctccaatccaatcaagttggcactcaatattaaacagtttcggccgggagcggtggctcacgcctgtaatcccagcactttgggaggccaaagcgggcagatcactaagtcaggagatcgagaccatcctggctaacacggtgaaaccccatctctactaaaaatacaaaaaattagccaggcgtggtggtgggcgcctgtagtcccagctactcgggaggctgaggcaagagagtggcgtgaacccgggaggcagaacttgcagcaagcagagatcgcaccactgcactccagcctccagcctgggcgacagagcaagacaccgtctcaaaaaaaaaaaaaaaacaactaacagtttcgttcattaaggcaatgtccatggggcagctcctgcatgccacgccacccctgccctgacatactcccaggctgaccagccagaactggggagagggcatcttgaagaaacagtccaccctccgccctcgcatacctgtgtctcccagcagacaagacacacaagggcagggactgcactttcctgtgtgcaaatctcaggtatggtccagaaatcacatctctttggtctgattggccttacttaccaaacccttgaaacaatggaaagaagcttgctgcaatgggaagacccctgccccccaaaccctctaaccaggagtctacagacctcattccgtggaagttcccattgtcccctgggaaaccaccaccttctgaatcggcacagccaaggcccccaaccctgctatcaacagctttccttagccaccttccccatcctgcagggcttttcaacatttcgactttttaatccttccctccctaagatatagacatggtgttagtagcagaaccctgaaggcaaaggaacagaaactaacactctcagaaagtcctagcaggatggtctgaattaatagacagctgcaggcagccagtgtgcaaggcacgctcgtaatgtcagtggcgttccggaatgagggctgtgatcccaggcagcccagatcccaaggcgaaaacattcatgtcgcccagcatcaccctgtccttgcccagggtgaccctgagacggcacttggattcttccacatctgaactcaaacctgctccctcagctctccccagcaggaattggagccctctccaggccgttcagccaaagcagaatgtgtctggccaagcagatctgcccagagaagaacaaagacgtgttcagacactgtctggggaattggttttgagctcactctgagctccatctgtgcaacaaggccagcgtcagctgttacagagaaggggcttgtggcagtttattaccctggtgagctggctcggggccatgctgaccgcaggactgtaaaggacataaggcagttggcaatatgggaagcagcagtccccaaagcaccccgtcaaggaaacgatgcaagagctctgtccactggaccagccccaccctaccccgccacgccctggcctagggggagggaccttgtgtttcacgggcctggatggccagggatggacagtggcatgggcacagacacacccagaccgaaggaagggcccgggctccagaaccagacccgatccaggctcccagctctgtcacctactggctgcatgaaaagggtgggttttccttgttttgcagcctcagctatctcagctgtgagggaacaatgacgccacctccctcacgtggcggtcctgaagatgaaatgtaatgcatgcagcgcggcggccggcccaccgtgtgctcttagtaactggccgctgttctcttcagccctcccgttactgaccacctcgagggcaccgttcctccaagctgaccatttcctgccagtcacccactgcccagctcgcccagccatggagtgggctctggtccacgtgcatggtgagctgccatctttgtcgtcctctggacaccacgtctacctccttgggaaatagaaacgagtcctgcattgctttgtcaaatttcacaaaaaccctagaaagcagatattgccctggtttacagacggacagacagaagcaaagagaagccaactgacccactccaggttacaagcagggtctggtaggagagcacgcgtccaccccaccgctcactgcgcccacctactcctcgcttgagggcagagaacgagctggggcggggcgctccagggggcacaggcagcctgtgttcaaggcatgctcgtgagccccacagcaggggcggagtcaaaccagggctctggttctggggtgagcagagggtggggccaggcccccactgggaggctgactgaggaggggctcaaagggcaggcaggctccatcagagcggtttgatgagcagggccgctgagtggccctcaggtcaaggctggggccgggaccaccagggaccaggtctgcggggccaggggtccctccccagggtggagcaggaaaactccggcaaccgccttcttgagtggacttgaccttccctttctctgctaatgatctcagattaatcccttaaggcaagtgccccagggtgggtgttggttgcataaaacaagaggagtccttcttcccttccttcctggcacaaacatttgtaggccacccccgatgtggctggtcgtagtctggatttgggggcacattcaggcattcaacaaatacgtcatgaggccccgtgatgagctggggggctatgcttgatgtcggcagtacagctgtggtcgctcgaatgaatgtgcccgccctcacgggactgacctctagtggtgagagacagacactaaccgagtcacatgtgtgattacacgtgtaatcatacttacatatgtaacaacacgagttacacgtgcagtgtggggagggggcccttcacggtgaccagcccatcagggaaggggatgcgggtggcagaaggaagcggcacacccttgtcctcacggagctcacaggaggttctctgaccctcatcttaagccacaggctgtgtctgagcctcactgccctagctatcaactgggtgcaggactaggccttaatcactgggcagcgggaagaactacataagctcatactggcgcaaagccccttgggagaccgcagggaaagccattgcatgaggtggtgacaggctcggccttcagagtcgggctagaatctgagttctgacaatgatccccgccttgacccatctctcctcaggccgccctgagccttccagttaggcctgggctttgggcttccgtgtttgtctctggattgttcggttttagcaagagtcctgctaaattggtcttgctggaatcctccgccctccacatctgattgcccttcctgtctgatcgtggtcttcatgccccacggcctccaggggacgtctgatcacctggcctgccttcagcaagaatccccccaggtgtttcctcttaagaggttttccatccactgagcccaccctgctccttggccatcaattcccacttgcctgtactgtattcagaggggaccccaatctctcttcccagctgcagcggtccctgtccaacactgccccccacctcaaataaagtctttcttactgtctttaacatatgccgtcaataaagttttagcagatccaa

SEQ ID NO: 235 - DNA, 11163 bp, Homo sapiens
  source 1..11163: mol_type = genomic DNA
agacctagacaactcggaagtgggtttttcagcctcctgcaccggtgtcgcgtctgagtgcgactgatgagccagggggcgtcggtggaagcttgggggagaggctagtggtaacaggccgagctggatggatgggtatggggagaggggcaggacgttcagccctgggattctggccgaccctcgccttccttctctgcagcttccccgcagccacctccccgtgcaagatcctcaagtgcaactctgagttctggagcgccacgtcgggcagccacgccccagcctcagacgacacccccgagttctgtgcagccttgcgcagctacgccctgtgcacgcggcggacggcccgcacctgccggggtgacctggcctaccactcggccgtccatggcatagaggacctcatgagccagcacaactgctccaaggatggccccacctcgcagccacgcctgcgcacgctcccaccggccggagacagccaggagcgctcggacagccccgagatctgccattacgagaagagctttcacaagcactcggccacccccaactacacgcactgtggcctcttcggggacccacacctcaggactttcaccgaccgcttccagacctgcaaggtgcagggcgcctggccgctcatcgacaataattacctgaacgtgcaggtcaccaacacgcctgtgctgcccggctcagcggccactgccaccagcaagctcaccatcatcttcaagaacttccaggagtgtgtggaccagaaggtgtaccaggctgagatggacgagctcccggccgccttcgtggatggctctaagaacggtggggacaagcacggggccaacagcctgaagatcactgagaaggtgtcaggccagcacgtggagatccaggccaagtacatcggcaccaccatcgtggtgcgccaggtgggccgctacctgacctttgccgtccgcatgccagaggaagtggtcaatgctgtggaggactgggacagccagggtctctacctctgcctgcggggctgccccctcaaccagcagatcgacttccaggccttccacaccaatgctgagggcaccggtgcccgcaggctggcagccgccagccctgcacccacagcccccgagaccttcccatacgagacagccgtggccaagtgcaaggagaagctgccggtggaggacctgtactaccaggcctgcgtcttcgacctcctcaccacgggcgacgtgaacttcacactggccgcctactacgcgttggaggatgtcaagatgctccactccaacaaagacaaactgcacctgtatgagaggactcgggacctgccaggcagggcggctgcggggctgcccctggccccccggcccctcctgggcgccctcgtcccgctcctggccctgctccctgtgttctgctagacgcgtagatgtggagggaggcgcgggctccgtcctctcggcttccccatgtgtgggctgggaccgcccacggggtgcagatctcctggcgtgtccaccatggccccgcagaacgccagggaccgcctgctgccaagggctcaggcacggacccctccccttctagtgcacgtgacaaggttgtggtgactggtgccatgatgtttgacagtagagctgtgtgagagggagagcagctcccctcgccccgcccctgcagtgtgaatgtgtgaaacatcccctcaggctgaagccccccacccccaccagagacacactgggaaccgtcagagtcagctccttccccctcgcaatgcactgaaaggcccggccgactgctgctcgccgatccgtggggccccctgtgcccgccacacgcacgcacacactcttacacgagagcacactcgatccccctaggccagcggggacaccccagccacacagggaggcatccttggggcttggccccaggcagggcaaccccggggcgctgcttggcaccttagcagactgctggaaccttttggccagtaggtcgtgcccgcctggtgccttctggcctgtggcctccctgcccatgttcacctggctgctgtgggtaccagtgcaggtcccggttttcaggcacctgctcagctgcccgtctctggcctgggcccctgccccttccaccctgtgcttagaaagtcgaagtgcttggttctaaatgtctaaacagagaagagatccttgacttctgttcctctctctcctgcagatgcaagagctcctgggcaggggtgcctgggccccagggtgtggcaggagacccagtggatggggccagctggcctgccctgatcctctgcttcctcctcacaaccccaagagcccccagcccggtccatccacgtctggagtctggggagaggagcagggtcttaggactctcagctctgagcatccctggcagggtcttcaacctctaatctcttcccttaagccctgtggccacacagccaggagagacttgccgctggctcccgcctcatttcagcccagggtgctcatccaggggcccagaacagtcccacctgtgctgctgtgcccacagcacaaagccaggcttcactcccaaaagtgcagccaggccctggagggtgatcctgccagcagccctacagctccacaccctacccacccatcggcagcccctctgctgttccccagggacctctcatacactggccaggaggctgcagaacgtgtgtctccccctccctccaagaggtcctgctccctctgccagaaccgtgtgtgggcgggtgggagggcgctcggggcccggcccctccctctccctgctggttttagttggtccctatgttggaagtaaaaagtgaagcactttattttggttgtgtttgctcacgttctgcttggaagtggggacccctcactgcgtccacgtgtctgcgacctgtgtggagtgtcaccgcgtgtacatactgtaaattatttattaatggctaaatgcaagtaaagtttggtttttttgttattttcttttagacgtgttggagtttgttttctgaacagactttggctcccttgaggcgggtggaggatggactttggtggtgggatcccctgcagcaagtgtgtgtgtgtgcgcgcatgtgtgtgtgcgcgcatgtgtgtatgcatgcctgtgtgtgtgcgtgcctgtgtgcgtgcatgtacgcatgtgtgcgcatgcctgtgtgtgcgcccatgcctatgtgtgtgcatgtaggcatgtgtgtgcgcatgtgtatgtgcgtgagaatggtgtgaaggcggatagaagatgcagcagtttaacagccaccacacattgcttttgtaaagtcacttttcctcctcacgatgagctgtctttggagcttggcaggcccagatcctcttgttctagtgacagtggaacacagtcagggagacaggtgactatgtggtggagagacatatggtgccctctgggatgagatgggtgggatagcctgagatctgggatgactccctctcagccactgctatcagcctggagccccagctgttgggactgcctgccaccctccctgccagtgcccatcttctcatagcccgggcggagcagtggtgctgcaggaggctgctgtccacgtcactgtgggaccaggtttattggcagctgtgatcacatttcatctgtacaaactacccagtgaatcaggaagaaattcaggcccattttgccgatgaaacagactctgagattaactgacttgcctagggctccttagcaacgggaaggacaacctgtgcacctaggttagtattgtcctgaagccccttctcttcaaactaaatgccatgaggcagaatagcagcacagttaagccagcctacatcctggcttctccacctccccagcagtgtgaccatgggcaagtcacttcacctctctgagcctgcatgctgttctctggaagatgggggggtgcctgtcccatacagttgtttggagagtcaggagttcattcctgtaaattgcttaggagagggcggggcacacatggcttgtctggtcgtggggagggagggggtggtattatcccaaagcaagataaatcagttaaggggagaaggaaaggggcgatacagaccctggactcttgggagaggtgaacctgcaggctagcaaggggaggagtcccaagagtggccaagttcattaatcccaggcagaccaggaaggctgaagaaaaaggagaactttattttatttttgaaatggagtctggcgtcttgctctgtcgccaggctggagtgcagtggcaccatcccagctcactgcagcccctgcctcccaggttcaagcaattctcctgcctcagcctcccaagtagctgggactacaggcacacaccaccacgcctggctaattttttgtattttagtagagatggtgtttcaccatgttgaccagtatggtcttgatctcctgacctcgtgatccgcctgccttggcctcccaaagtgttgggattacaggcgtgagccaccaccgtgcccggccaaaaaaggagaacgttctaaagccctgttcttgcagggctcccagagagaacacgggagttgggtttgggaggcaggaaccagactggaaaggacaaaacgtacccagcatcttccctggcggccggaaccgtcactacaggccccgtgcccttagcagtcccagggacagagcgactcagggaggcagcaaggaggagcggagccattggtgctcgtccggcacagagaacagttcctgagaagccagggcacacagaagaaaaagaaaaaagaaatctctactagacgaaaatgctaaatggcactagccacaaagtattcttggtttggaattttttttccaggaccctaaatgtaaccggctggaattctttcccaagtgccatgctacttactactggttgccctcacgagcatggaaatgccgtcgtgaatatggaacacagcatcctgagctaaaagattcccaagaagcagcttcatcttgcaggacgggctaggttgggagcctctggaggttcctggctgccagctccgcggaggcctgcggcctggaggtaaatgcattagcacacggaggtgggcgcctctccccgccccatcctgtctcactgatgtgttgccgagaactggagttcttgtttagctaaccagaatgtgcaaggctataggacaggagagaaatggcctttaaaacaaatgaatgagagtcacgatgaagggggcagacgggggggtgaaaatcattaccagaacacttgggcagagacccgtcagagccaggcaggagacagtcatcagagctgtgccagcaagtggcatggagcaggggtggcacttgggctcgggctgcaggcaaacttggcgtcaaaactggctcaacccgtcgcgagctgtgtggtctccttgggattttttgagccttagattccttatttgtaaaatggatttgtgagtctccacctcccgaggtcagtatggaaatccagtgagctaacactcgtcaatggcctcgcacatggccctggcaccacgcatggaggccgccaaaagtggccgcctcctctctgcaattgcatttatttatccatttgataactctattgggtgtctacccagcattgttcaggggggtgtgaggggctaccgcaatgacccagacagactgctgtgcattcttattggaaagacaaggcaataaaccacagttacaaaacccagggtcccatggtaacgagctccacagagaaaagggaagtgggccagaggttccgagagacacacattttggattgcaggagggtgagaaggggcattcatctgtggtggggttaaggggaggcctccgactagctcacctgcatgaatttcaggaacaagccatgcagagccggacagagcattctgtgcagagaaaacagtgtaacagccctagagcagcagcagcaaagggggagtttaagggatggtgggaagccactgtcactggacagtagcaagtggacagaccaggggtcccaggtgaggccacagagcagccaaagtactgcagggctctccggctccgggaggacttgatgtttgactccagttgccagaggaagccgttcacgggctttgagtgtaggaatgatgtgtccgacttgggtctttttattgttcttttttagagacagggtctcactctgtcacccaggctggagtgcagcggcacaatcacagctcactgcagcctccacttcctgggctcaagcaatcctcccacttcagccactcaagtagctgggattacaggtgcctgccgtcacacccggctaattgttaaatttttttggagagacaggatcttgctgttttcccaggctgatcttgaactcctgggctcaagcgagacgtctgtttcagcctcccaaagtgctgggataacaggtgtgagccactgggcctgccttggcttgggtattaaaggaattgttctagttactatatggaggtgacttggggccgctggggaagaaagaggaagtcaggagccagtctgaggcagtggcaatgcagcgattatcagaaataatgggctcaatgatagactggataaagaaaatgtggcacatatacgccatggagtactatgcagccataaaaagggatgagttcatgtcctttgcagggacatggatgaagctggaaaccatcattctcagcaaactaacacaagaacagaaaaccaaacaccgcatgttctcactcgtaagtgggagttgaacaatgagaacacatggacacagggaagggaacatcacacactggggcctgtcagtgggtggggggctgggggagggagagcatcaggagaaatacccaatgtagatgatgacgggttcataggtgcagcaaaccaccatggcacgtgtatacctatgtaacaaacttgtgcgttctgcacgtgtaccccataactgaaagtataatcataattttaagaaaagaaatgatggcctttggggtctaataagaaggtggggctgacagggcttggggagagagcgagcttgacaggagaccacgatgtcttccccaggggtctgagcacctggggaaatggtggtgccatttaccatgactggaaagatcatgggaggagcaggtttgggacactgaaggcccgagagcccctggcaaaccaccagtgtaagtccaagagtccaaaagctgaagaacttggagtccaatgtttgagggtaaaaaacagccagcatgagaaaaagatgaaggccagaagactcgtccagtctagcccttccacattcctctgcccgcttttatcctagcctcgctggcaggtgattagatggtgcccacccaggttaagcgtgggtctctgtctcccagttcactgactcaaatgttaatttcctttggcgacagcctcacagacacacccaggaacgatactttgcatcctccaatccaatcaagttggcactcaatattaaacagtttcggccgggagcggtggctcacgcctgtaatcccagcactttgggaggccaaagcgggcagatcactaagtcaggagatcgagaccatcctggctaacacggtgaaaccccatctctactaaaaatacaaaaaattagccaggcgtggtggtgggcgcctgtagtcccagctactcgggaggctgaggcaagagagtggcgtgaacccgggaggcagaacttgcagcaagcagagatcgcaccactgcactccagcctccagcctgggcgacagagcaagacaccgtctcaaaaaaaaaaaaaaaacaactaacagtttcgttcattaaggcaatgtccatggggcagctcctgcatgccacgccacccctgccctgacatactcccaggctgaccagccagaactggggagagggcatcttgaagaaacagtccaccctccgccctcgcatacctgtgtctcccagcagacaagacacacaagggcagggactgcactttcctgtgtgcaaatctcaggtatggtccagaaatcacatctctttggtctgattggccttacttaccaaacccttgaaacaatggaaagaagcttgctgcaatgggaagacccctgccccccaaaccctctaaccaggagtctacagacctcattccgtggaagttcccattgtcccctgggaaaccaccaccttctgaatcggcacagccaaggcccccaaccctgctatcaacagctttccttagccaccttccccatcctgcagggcttttcaacatttcgactttttaatccttccctccctaagatatagacatggtgttagtagcagaaccctgaaggcaaaggaacagaaactaacactctcagaaagtcctagcaggatggtctgaattaatagacagctgcaggcagccagtgtgcaaggcacgctcgtaatgtcagtggcgttccggaatgagggctgtgatcccaggcagcccagatcccaaggcgaaaacattcatgtcgcccagcatcaccctgtccttgcccagggtgaccctgagacggcacttggattcttccacatctgaactcaaacctgctccctcagctctccccagcaggaattggagccctctccaggccgttcagccaaagcagaatgtgtctggccaagcagatctgcccagagaagaacaaagacgtgttcagacactgtctggggaattggttttgagctcactctgagctccatctgtgcaacaaggccagcgtcagctgttacagagaaggggcttgtggcagtttattaccctggtgagctggctcggggccatgctgaccgcaggactgtaaaggacataaggcagttggcaatatgggaagcagcagtccccaaagcaccccgtcaaggaaacgatgcaagagctctgtccactggaccagccccaccctaccccgccacgccctggcctagggggagggaccttgtgtttcacgggcctggatggccagggatggacagtggcatgggcacagacacacccagaccgaaggaagggcccgggctccagaaccagacccgatccaggctcccagctctgtcacctactggctgcatgaaaagggtgggttttccttgttttgcagcctcagctatctcagctgtgagggaacaatgacgccacctccctcacgtggcggtcctgaagatgaaatgtaatgcatgcagcgcggcggccggcccaccgtgtgctcttagtaactggccgctgttctcttcagccctcccgttactgaccacctcgagggcaccgttcctccaagctgaccatttcctgccagtcacccactgcccagctcgcccagccatggagtgggctctggtccacgtgcatggtgagctgccatctttgtcgtcctctggacaccacgtctacctccttgggaaatagaaacgagtcctgcattgctttgtcaaatttcacaaaaaccctagaaagcagatattgccctggtttacagacggacagacagaagcaaagagaagccaactgacccactccaggttacaagcagggtctggtaggagagcacgcgtccaccccaccgctcactgcgcccacctactcctcgcttgagggcagagaacgagctggggcggggcgctccagggggcacaggcagcctgtgttcaaggcatgctcgtgagccccacagcaggggcggagtcaaaccagggctctggttctggggtgagcagagggtggggccaggcccccactgggaggctgactgaggaggggctcaaagggcaggcaggctccatcagagcggtttgatgagcagggccgctgagtggccctcaggtcaaggctggggccgggaccaccagggaccaggtctgcggggccaggggtccctccccagggtggagcaggaaaactccggcaaccgccttcttgagtggacttgaccttccctttctctgctaatgatctcagattaatcccttaaggcaagtgccccagggtgggtgttggttgcataaaacaagaggagtccttcttcccttccttcctggcacaaacatttgtaggccacccccgatgtggctggtcgtagtctggatttgggggcacattcaggcattcaacaaatacgtcatgaggccccgtgatgagctggggggctatgcttgatgtcggcagtacagctgtggtcgctcgaatgaatgtgcccgccctcacgggactgacctctagtggtgagagacagacactaaccgagtcacatgtgtgattacacgtgtaatcatacttacatatgtaacaacacgagttacacgtgcagtgtggggagggggcccttcacggtgaccagcccatcagggaaggggatgcgggtggcagaaggaagcggcacacccttgtcctcacggagctcacaggaggttctctgaccctcatcttaagccacaggctgtgtctgagcctcactgccctagctatcaactgggtgcaggactaggccttaatcactgggcagcgggaagaactacataagctcatactggcgcaaagccccttgggagaccgcagggaaagccattgcatgaggtggtgacaggctcggccttcagagtcgggctagaatctgagttctgacaatgatccccgccttgacccatctctcctcaggccgccctgagccttccagttaggcctgggctttgggcttccgtgtttgtctctggattgttcggttttagcaagagtcctgctaaattggtcttgctggaatcctccgccctccacatctgattgcccttcctgtctgatcgtggtcttcatgccccacggcctccaggggacgtctgatcacctggcctgccttcagcaagaatccccccaggtgtttcctcttaagaggttttccatccactgagcccaccctgctccttggccatcaattcccacttgcctgtactgtattcagaggggaccccaatctctcttcccagctgcagcggtccctgtccaacactgccccccacctcaaataaagtctttcttactgtctttaacatatgccgtcaataaagttttagcagatccaa

SEQ ID NO: 236 - DNA, 11213 bp, Homo sapiens
  source 1..11213: mol_type = genomic DNA
gtagctgagatgcagagagccatcctgcccagacctagacaactcggaagtgggtttttcagcctcctgcaccggtgtcgcgtctgagtgcgactgatgagccagggggcgtcggtggaagcttggggtcggcagtctggttggaaaggagaggctagtggtaacaggccgagctggatggatgggtatggggagaggggcaggacgttcagccctgggattctggccgaccctcgccttccttctctgcagcttccccgcagccacctccccgtgcaagatcctcaagtgcaactctgagttctggagcgccacgtcgggcagccacgccccagcctcagacgacacccccgagttctgtgcagccttgcgcagctacgccctgtgcacgcggcggacggcccgcacctgccggggtgacctggcctaccactcggccgtccatggcatagaggacctcatgagccagcacaactgctccaaggatggccccacctcgcagccacgcctgcgcacgctcccaccggccggagacagccaggagcgctcggacagccccgagatctgccattacgagaagagctttcacaagcactcggccacccccaactacacgcactgtggcctcttcggggacccacacctcaggactttcaccgaccgcttccagacctgcaaggtgcagggcgcctggccgctcatcgacaataattacctgaacgtgcaggtcaccaacacgcctgtgctgcccggctcagcggccactgccaccagcaagctcaccatcatcttcaagaacttccaggagtgtgtggaccagaaggtgtaccaggctgagatggacgagctcccggccgccttcgtggatggctctaagaacggtggggacaagcacggggccaacagcctgaagatcactgagaaggtgtcaggccagcacgtggagatccaggccaagtacatcggcaccaccatcgtggtgcgccaggtgggccgctacctgacctttgccgtccgcatgccagaggaagtggtcaatgctgtggaggactgggacagccagggtctctacctctgcctgcggggctgccccctcaaccagcagatcgacttccaggccttccacaccaatgctgagggcaccggtgcccgcaggctggcagccgccagccctgcacccacagcccccgagaccttcccatacgagacagccgtggccaagtgcaaggagaagctgccggtggaggacctgtactaccaggcctgcgtcttcgacctcctcaccacgggcgacgtgaacttcacactggccgcctactacgcgttggaggatgtcaagatgctccactccaacaaagacaaactgcacctgtatgagaggactcgggacctgccaggcagggcggctgcggggctgcccctggccccccggcccctcctgggcgccctcgtcccgctcctggccctgctccctgtgttctgctagacgcgtagatgtggagggaggcgcgggctccgtcctctcggcttccccatgtgtgggctgggaccgcccacggggtgcagatctcctggcgtgtccaccatggccccgcagaacgccagggaccgcctgctgccaagggctcaggcacggacccctccccttctagtgcacgtgacaaggttgtggtgactggtgccatgatgtttgacagtagagctgtgtgagagggagagcagctcccctcgccccgcccctgcagtgtgaatgtgtgaaacatcccctcaggctgaagccccccacccccaccagagacacactgggaaccgtcagagtcagctccttccccctcgcaatgcactgaaaggcccggccgactgctgctcgccgatccgtggggccccctgtgcccgccacacgcacgcacacactcttacacgagagcacactcgatccccctaggccagcggggacaccccagccacacagggaggcatccttggggcttggccccaggcagggcaaccccggggcgctgcttggcaccttagcagactgctggaaccttttggccagtaggtcgtgcccgcctggtgccttctggcctgtggcctccctgcccatgttcacctggctgctgtgggtaccagtgcaggtcccggttttcaggcacctgctcagctgcccgtctctggcctgggcccctgccccttccaccctgtgcttagaaagtcgaagtgcttggttctaaatgtctaaacagagaagagatccttgacttctgttcctctctctcctgcagatgcaagagctcctgggcaggggtgcctgggccccagggtgtggcaggagacccagtggatggggccagctggcctgccctgatcctctgcttcctcctcacaaccccaagagcccccagcccggtccatccacgtctggagtctggggagaggagcagggtcttaggactctcagctctgagcatccctggcagggtcttcaacctctaatctcttcccttaagccctgtggccacacagccaggagagacttgccgctggctcccgcctcatttcagcccagggtgctcatccaggggcccagaacagtcccacctgtgctgctgtgcccacagcacaaagccaggcttcactcccaaaagtgcagccaggccctggagggtgatcctgccagcagccctacagctccacaccctacccacccatcggcagcccctctgctgttccccagggacctctcatacactggccaggaggctgcagaacgtgtgtctccccctccctccaagaggtcctgctccctctgccagaaccgtgtgtgggcgggtgggagggcgctcggggcccggcccctccctctccctgctggttttagttggtccctatgttggaagtaaaaagtgaagcactttattttggttgtgtttgctcacgttctgcttggaagtggggacccctcactgcgtccacgtgtctgcgacctgtgtggagtgtcaccgcgtgtacatactgtaaattatttattaatggctaaatgcaagtaaagtttggtttttttgttattttcttttagacgtgttggagtttgttttctgaacagactttggctcccttgaggcgggtggaggatggactttggtggtgggatcccctgcagcaagtgtgtgtgtgtgcgcgcatgtgtgtgtgcgcgcatgtgtgtatgcatgcctgtgtgtgtgcgtgcctgtgtgcgtgcatgtacgcatgtgtgcgcatgcctgtgtgtgcgcccatgcctatgtgtgtgcatgtaggcatgtgtgtgcgcatgtgtatgtgcgtgagaatggtgtgaaggcggatagaagatgcagcagtttaacagccaccacacattgcttttgtaaagtcacttttcctcctcacgatgagctgtctttggagcttggcaggcccagatcctcttgttctagtgacagtggaacacagtcagggagacaggtgactatgtggtggagagacatatggtgccctctgggatgagatgggtgggatagcctgagatctgggatgactccctctcagccactgctatcagcctggagccccagctgttgggactgcctgccaccctccctgccagtgcccatcttctcatagcccgggcggagcagtggtgctgcaggaggctgctgtccacgtcactgtgggaccaggtttattggcagctgtgatcacatttcatctgtacaaactacccagtgaatcaggaagaaattcaggcccattttgccgatgaaacagactctgagattaactgacttgcctagggctccttagcaacgggaaggacaacctgtgcacctaggttagtattgtcctgaagccccttctcttcaaactaaatgccatgaggcagaatagcagcacagttaagccagcctacatcctggcttctccacctccccagcagtgtgaccatgggcaagtcacttcacctctctgagcctgcatgctgttctctggaagatgggggggtgcctgtcccatacagttgtttggagagtcaggagttcattcctgtaaattgcttaggagagggcggggcacacatggcttgtctggtcgtggggagggagggggtggtattatcccaaagcaagataaatcagttaaggggagaaggaaaggggcgatacagaccctggactcttgggagaggtgaacctgcaggctagcaaggggaggagtcccaagagtggccaagttcattaatcccaggcagaccaggaaggctgaagaaaaaggagaactttattttatttttgaaatggagtctggcgtcttgctctgtcgccaggctggagtgcagtggcaccatcccagctcactgcagcccctgcctcccaggttcaagcaattctcctgcctcagcctcccaagtagctgggactacaggcacacaccaccacgcctggctaattttttgtattttagtagagatggtgtttcaccatgttgaccagtatggtcttgatctcctgacctcgtgatccgcctgccttggcctcccaaagtgttgggattacaggcgtgagccaccaccgtgcccggccaaaaaaggagaacgttctaaagccctgttcttgcagggctcccagagagaacacgggagttgggtttgggaggcaggaaccagactggaaaggacaaaacgtacccagcatcttccctggcggccggaaccgtcactacaggccccgtgcccttagcagtcccagggacagagcgactcagggaggcagcaaggaggagcggagccattggtgctcgtccggcacagagaacagttcctgagaagccagggcacacagaagaaaaagaaaaaagaaatctctactagacgaaaatgctaaatggcactagccacaaagtattcttggtttggaattttttttccaggaccctaaatgtaaccggctggaattctttcccaagtgccatgctacttactactggttgccctcacgagcatggaaatgccgtcgtgaatatggaacacagcatcctgagctaaaagattcccaagaagcagcttcatcttgcaggacgggctaggttgggagcctctggaggttcctggctgccagctccgcggaggcctgcggcctggaggtaaatgcattagcacacggaggtgggcgcctctccccgccccatcctgtctcactgatgtgttgccgagaactggagttcttgtttagctaaccagaatgtgcaaggctataggacaggagagaaatggcctttaaaacaaatgaatgagagtcacgatgaagggggcagacgggggggtgaaaatcattaccagaacacttgggcagagacccgtcagagccaggcaggagacagtcatcagagctgtgccagcaagtggcatggagcaggggtggcacttgggctcgggctgcaggcaaacttggcgtcaaaactggctcaacccgtcgcgagctgtgtggtctccttgggattttttgagccttagattccttatttgtaaaatggatttgtgagtctccacctcccgaggtcagtatggaaatccagtgagctaacactcgtcaatggcctcgcacatggccctggcaccacgcatggaggccgccaaaagtggccgcctcctctctgcaattgcatttatttatccatttgataactctattgggtgtctacccagcattgttcaggggggtgtgaggggctaccgcaatgacccagacagactgctgtgcattcttattggaaagacaaggcaataaaccacagttacaaaacccagggtcccatggtaacgagctccacagagaaaagggaagtgggccagaggttccgagagacacacattttggattgcaggagggtgagaaggggcattcatctgtggtggggttaaggggaggcctccgactagctcacctgcatgaatttcaggaacaagccatgcagagccggacagagcattctgtgcagagaaaacagtgtaacagccctagagcagcagcagcaaagggggagtttaagggatggtgggaagccactgtcactggacagtagcaagtggacagaccaggggtcccaggtgaggccacagagcagccaaagtactgcagggctctccggctccgggaggacttgatgtttgactccagttgccagaggaagccgttcacgggctttgagtgtaggaatgatgtgtccgacttgggtctttttattgttcttttttagagacagggtctcactctgtcacccaggctggagtgcagcggcacaatcacagctcactgcagcctccacttcctgggctcaagcaatcctcccacttcagccactcaagtagctgggattacaggtgcctgccgtcacacccggctaattgttaaatttttttggagagacaggatcttgctgttttcccaggctgatcttgaactcctgggctcaagcgagacgtctgtttcagcctcccaaagtgctgggataacaggtgtgagccactgggcctgccttggcttgggtattaaaggaattgttctagttactatatggaggtgacttggggccgctggggaagaaagaggaagtcaggagccagtctgaggcagtggcaatgcagcgattatcagaaataatgggctcaatgatagactggataaagaaaatgtggcacatatacgccatggagtactatgcagccataaaaagggatgagttcatgtcctttgcagggacatggatgaagctggaaaccatcattctcagcaaactaacacaagaacagaaaaccaaacaccgcatgttctcactcgtaagtgggagttgaacaatgagaacacatggacacagggaagggaacatcacacactggggcctgtcagtgggtggggggctgggggagggagagcatcaggagaaatacccaatgtagatgatgacgggttcataggtgcagcaaaccaccatggcacgtgtatacctatgtaacaaacttgtgcgttctgcacgtgtaccccataactgaaagtataatcataattttaagaaaagaaatgatggcctttggggtctaataagaaggtggggctgacagggcttggggagagagcgagcttgacaggagaccacgatgtcttccccaggggtctgagcacctggggaaatggtggtgccatttaccatgactggaaagatcatgggaggagcaggtttgggacactgaaggcccgagagcccctggcaaaccaccagtgtaagtccaagagtccaaaagctgaagaacttggagtccaatgtttgagggtaaaaaacagccagcatgagaaaaagatgaaggccagaagactcgtccagtctagcccttccacattcctctgcccgcttttatcctagcctcgctggcaggtgattagatggtgcccacccaggttaagcgtgggtctctgtctcccagttcactgactcaaatgttaatttcctttggcgacagcctcacagacacacccaggaacgatactttgcatcctccaatccaatcaagttggcactcaatattaaacagtttcggccgggagcggtggctcacgcctgtaatcccagcactttgggaggccaaagcgggcagatcactaagtcaggagatcgagaccatcctggctaacacggtgaaaccccatctctactaaaaatacaaaaaattagccaggcgtggtggtgggcgcctgtagtcccagctactcgggaggctgaggcaagagagtggcgtgaacccgggaggcagaacttgcagcaagcagagatcgcaccactgcactccagcctccagcctgggcgacagagcaagacaccgtctcaaaaaaaaaaaaaaaacaactaacagtttcgttcattaaggcaatgtccatggggcagctcctgcatgccacgccacccctgccctgacatactcccaggctgaccagccagaactggggagagggcatcttgaagaaacagtccaccctccgccctcgcatacctgtgtctcccagcagacaagacacacaagggcagggactgcactttcctgtgtgcaaatctcaggtatggtccagaaatcacatctctttggtctgattggccttacttaccaaacccttgaaacaatggaaagaagcttgctgcaatgggaagacccctgccccccaaaccctctaaccaggagtctacagacctcattccgtggaagttcccattgtcccctgggaaaccaccaccttctgaatcggcacagccaaggcccccaaccctgctatcaacagctttccttagccaccttccccatcctgcagggcttttcaacatttcgactttttaatccttccctccctaagatatagacatggtgttagtagcagaaccctgaaggcaaaggaacagaaactaacactctcagaaagtcctagcaggatggtctgaattaatagacagctgcaggcagccagtgtgcaaggcacgctcgtaatgtcagtggcgttccggaatgagggctgtgatcccaggcagcccagatcccaaggcgaaaacattcatgtcgcccagcatcaccctgtccttgcccagggtgaccctgagacggcacttggattcttccacatctgaactcaaacctgctccctcagctctccccagcaggaattggagccctctccaggccgttcagccaaagcagaatgtgtctggccaagcagatctgcccagagaagaacaaagacgtgttcagacactgtctggggaattggttttgagctcactctgagctccatctgtgcaacaaggccagcgtcagctgttacagagaaggggcttgtggcagtttattaccctggtgagctggctcggggccatgctgaccgcaggactgtaaaggacataaggcagttggcaatatgggaagcagcagtccccaaagcaccccgtcaaggaaacgatgcaagagctctgtccactggaccagccccaccctaccccgccacgccctggcctagggggagggaccttgtgtttcacgggcctggatggccagggatggacagtggcatgggcacagacacacccagaccgaaggaagggcccgggctccagaaccagacccgatccaggctcccagctctgtcacctactggctgcatgaaaagggtgggttttccttgttttgcagcctcagctatctcagctgtgagggaacaatgacgccacctccctcacgtggcggtcctgaagatgaaatgtaatgcatgcagcgcggcggccggcccaccgtgtgctcttagtaactggccgctgttctcttcagccctcccgttactgaccacctcgagggcaccgttcctccaagctgaccatttcctgccagtcacccactgcccagctcgcccagccatggagtgggctctggtccacgtgcatggtgagctgccatctttgtcgtcctctggacaccacgtctacctccttgggaaatagaaacgagtcctgcattgctttgtcaaatttcacaaaaaccctagaaagcagatattgccctggtttacagacggacagacagaagcaaagagaagccaactgacccactccaggttacaagcagggtctggtaggagagcacgcgtccaccccaccgctcactgcgcccacctactcctcgcttgagggcagagaacgagctggggcggggcgctccagggggcacaggcagcctgtgttcaaggcatgctcgtgagccccacagcaggggcggagtcaaaccagggctctggttctggggtgagcagagggtggggccaggcccccactgggaggctgactgaggaggggctcaaagggcaggcaggctccatcagagcggtttgatgagcagggccgctgagtggccctcaggtcaaggctggggccgggaccaccagggaccaggtctgcggggccaggggtccctccccagggtggagcaggaaaactccggcaaccgccttcttgagtggacttgaccttccctttctctgctaatgatctcagattaatcccttaaggcaagtgccccagggtgggtgttggttgcataaaacaagaggagtccttcttcccttccttcctggcacaaacatttgtaggccacccccgatgtggctggtcgtagtctggatttgggggcacattcaggcattcaacaaatacgtcatgaggccccgtgatgagctggggggctatgcttgatgtcggcagtacagctgtggtcgctcgaatgaatgtgcccgccctcacgggactgacctctagtggtgagagacagacactaaccgagtcacatgtgtgattacacgtgtaatcatacttacatatgtaacaacacgagttacacgtgcagtgtggggagggggcccttcacggtgaccagcccatcagggaaggggatgcgggtggcagaaggaagcggcacacccttgtcctcacggagctcacaggaggttctctgaccctcatcttaagccacaggctgtgtctgagcctcactgccctagctatcaactgggtgcaggactaggccttaatcactgggcagcgggaagaactacataagctcatactggcgcaaagccccttgggagaccgcagggaaagccattgcatgaggtggtgacaggctcggccttcagagtcgggctagaatctgagttctgacaatgatccccgccttgacccatctctcctcaggccgccctgagccttccagttaggcctgggctttgggcttccgtgtttgtctctggattgttcggttttagcaagagtcctgctaaattggtcttgctggaatcctccgccctccacatctgattgcccttcctgtctgatcgtggtcttcatgccccacggcctccaggggacgtctgatcacctggcctgccttcagcaagaatccccccaggtgtttcctcttaagaggttttccatccactgagcccaccctgctccttggccatcaattcccacttgcctgtactgtattcagaggggaccccaatctctcttcccagctgcagcggtccctgtccaacactgccccccacctcaaataaagtctttcttactgtctttaacatatgccgtcaataaagttttagcagatccaa

SEQ ID NO: 237 - DNA, 11359 bp, Homo sapiens
  source 1..11359: mol_type = genomic DNA
actctgcagttgtctcccgagcgctggctgcgccgcccgagccgctgggccggggaagcactggccgttcgctcccgggccggctccgccaggcgctcgcaggcatgcagcccgggagcaggaggcgctccccgggccgctgctgagccggccggggcggcggggaccagcgccagcggagcccctcccaccttgccccggggcagacgagcggcgccccgacaccccctcttctcccgcagccccgccagcgccaccccccgcgggccgcaggggctcatgcagccgccaagggagaggctagtggtaacaggccgagctggatggatgggtatggggagaggggcaggacgttcagccctgggattctggccgaccctcgccttccttctctgcagcttccccgcagccacctccccgtgcaagatcctcaagtgcaactctgagttctggagcgccacgtcgggcagccacgccccagcctcagacgacacccccgagttctgtgcagccttgcgcagctacgccctgtgcacgcggcggacggcccgcacctgccggggtgacctggcctaccactcggccgtccatggcatagaggacctcatgagccagcacaactgctccaaggatggccccacctcgcagccacgcctgcgcacgctcccaccggccggagacagccaggagcgctcggacagccccgagatctgccattacgagaagagctttcacaagcactcggccacccccaactacacgcactgtggcctcttcggggacccacacctcaggactttcaccgaccgcttccagacctgcaaggtgcagggcgcctggccgctcatcgacaataattacctgaacgtgcaggtcaccaacacgcctgtgctgcccggctcagcggccactgccaccagcaagctcaccatcatcttcaagaacttccaggagtgtgtggaccagaaggtgtaccaggctgagatggacgagctcccggccgccttcgtggatggctctaagaacggtggggacaagcacggggccaacagcctgaagatcactgagaaggtgtcaggccagcacgtggagatccaggccaagtacatcggcaccaccatcgtggtgcgccaggtgggccgctacctgacctttgccgtccgcatgccagaggaagtggtcaatgctgtggaggactgggacagccagggtctctacctctgcctgcggggctgccccctcaaccagcagatcgacttccaggccttccacaccaatgctgagggcaccggtgcccgcaggctggcagccgccagccctgcacccacagcccccgagaccttcccatacgagacagccgtggccaagtgcaaggagaagctgccggtggaggacctgtactaccaggcctgcgtcttcgacctcctcaccacgggcgacgtgaacttcacactggccgcctactacgcgttggaggatgtcaagatgctccactccaacaaagacaaactgcacctgtatgagaggactcgggacctgccaggcagggcggctgcggggctgcccctggccccccggcccctcctgggcgccctcgtcccgctcctggccctgctccctgtgttctgctagacgcgtagatgtggagggaggcgcgggctccgtcctctcggcttccccatgtgtgggctgggaccgcccacggggtgcagatctcctggcgtgtccaccatggccccgcagaacgccagggaccgcctgctgccaagggctcaggcacggacccctccccttctagtgcacgtgacaaggttgtggtgactggtgccatgatgtttgacagtagagctgtgtgagagggagagcagctcccctcgccccgcccctgcagtgtgaatgtgtgaaacatcccctcaggctgaagccccccacccccaccagagacacactgggaaccgtcagagtcagctccttccccctcgcaatgcactgaaaggcccggccgactgctgctcgccgatccgtggggccccctgtgcccgccacacgcacgcacacactcttacacgagagcacactcgatccccctaggccagcggggacaccccagccacacagggaggcatccttggggcttggccccaggcagggcaaccccggggcgctgcttggcaccttagcagactgctggaaccttttggccagtaggtcgtgcccgcctggtgccttctggcctgtggcctccctgcccatgttcacctggctgctgtgggtaccagtgcaggtcccggttttcaggcacctgctcagctgcccgtctctggcctgggcccctgccccttccaccctgtgcttagaaagtcgaagtgcttggttctaaatgtctaaacagagaagagatccttgacttctgttcctctctctcctgcagatgcaagagctcctgggcaggggtgcctgggccccagggtgtggcaggagacccagtggatggggccagctggcctgccctgatcctctgcttcctcctcacaaccccaagagcccccagcccggtccatccacgtctggagtctggggagaggagcagggtcttaggactctcagctctgagcatccctggcagggtcttcaacctctaatctcttcccttaagccctgtggccacacagccaggagagacttgccgctggctcccgcctcatttcagcccagggtgctcatccaggggcccagaacagtcccacctgtgctgctgtgcccacagcacaaagccaggcttcactcccaaaagtgcagccaggccctggagggtgatcctgccagcagccctacagctccacaccctacccacccatcggcagcccctctgctgttccccagggacctctcatacactggccaggaggctgcagaacgtgtgtctccccctccctccaagaggtcctgctccctctgccagaaccgtgtgtgggcgggtgggagggcgctcggggcccggcccctccctctccctgctggttttagttggtccctatgttggaagtaaaaagtgaagcactttattttggttgtgtttgctcacgttctgcttggaagtggggacccctcactgcgtccacgtgtctgcgacctgtgtggagtgtcaccgcgtgtacatactgtaaattatttattaatggctaaatgcaagtaaagtttggtttttttgttattttcttttagacgtgttggagtttgttttctgaacagactttggctcccttgaggcgggtggaggatggactttggtggtgggatcccctgcagcaagtgtgtgtgtgtgcgcgcatgtgtgtgtgcgcgcatgtgtgtatgcatgcctgtgtgtgtgcgtgcctgtgtgcgtgcatgtacgcatgtgtgcgcatgcctgtgtgtgcgcccatgcctatgtgtgtgcatgtaggcatgtgtgtgcgcatgtgtatgtgcgtgagaatggtgtgaaggcggatagaagatgcagcagtttaacagccaccacacattgcttttgtaaagtcacttttcctcctcacgatgagctgtctttggagcttggcaggcccagatcctcttgttctagtgacagtggaacacagtcagggagacaggtgactatgtggtggagagacatatggtgccctctgggatgagatgggtgggatagcctgagatctgggatgactccctctcagccactgctatcagcctggagccccagctgttgggactgcctgccaccctccctgccagtgcccatcttctcatagcccgggcggagcagtggtgctgcaggaggctgctgtccacgtcactgtgggaccaggtttattggcagctgtgatcacatttcatctgtacaaactacccagtgaatcaggaagaaattcaggcccattttgccgatgaaacagactctgagattaactgacttgcctagggctccttagcaacgggaaggacaacctgtgcacctaggttagtattgtcctgaagccccttctcttcaaactaaatgccatgaggcagaatagcagcacagttaagccagcctacatcctggcttctccacctccccagcagtgtgaccatgggcaagtcacttcacctctctgagcctgcatgctgttctctggaagatgggggggtgcctgtcccatacagttgtttggagagtcaggagttcattcctgtaaattgcttaggagagggcggggcacacatggcttgtctggtcgtggggagggagggggtggtattatcccaaagcaagataaatcagttaaggggagaaggaaaggggcgatacagaccctggactcttgggagaggtgaacctgcaggctagcaaggggaggagtcccaagagtggccaagttcattaatcccaggcagaccaggaaggctgaagaaaaaggagaactttattttatttttgaaatggagtctggcgtcttgctctgtcgccaggctggagtgcagtggcaccatcccagctcactgcagcccctgcctcccaggttcaagcaattctcctgcctcagcctcccaagtagctgggactacaggcacacaccaccacgcctggctaattttttgtattttagtagagatggtgtttcaccatgttgaccagtatggtcttgatctcctgacctcgtgatccgcctgccttggcctcccaaagtgttgggattacaggcgtgagccaccaccgtgcccggccaaaaaaggagaacgttctaaagccctgttcttgcagggctcccagagagaacacgggagttgggtttgggaggcaggaaccagactggaaaggacaaaacgtacccagcatcttccctggcggccggaaccgtcactacaggccccgtgcccttagcagtcccagggacagagcgactcagggaggcagcaaggaggagcggagccattggtgctcgtccggcacagagaacagttcctgagaagccagggcacacagaagaaaaagaaaaaagaaatctctactagacgaaaatgctaaatggcactagccacaaagtattcttggtttggaattttttttccaggaccctaaatgtaaccggctggaattctttcccaagtgccatgctacttactactggttgccctcacgagcatggaaatgccgtcgtgaatatggaacacagcatcctgagctaaaagattcccaagaagcagcttcatcttgcaggacgggctaggttgggagcctctggaggttcctggctgccagctccgcggaggcctgcggcctggaggtaaatgcattagcacacggaggtgggcgcctctccccgccccatcctgtctcactgatgtgttgccgagaactggagttcttgtttagctaaccagaatgtgcaaggctataggacaggagagaaatggcctttaaaacaaatgaatgagagtcacgatgaagggggcagacgggggggtgaaaatcattaccagaacacttgggcagagacccgtcagagccaggcaggagacagtcatcagagctgtgccagcaagtggcatggagcaggggtggcacttgggctcgggctgcaggcaaacttggcgtcaaaactggctcaacccgtcgcgagctgtgtggtctccttgggattttttgagccttagattccttatttgtaaaatggatttgtgagtctccacctcccgaggtcagtatggaaatccagtgagctaacactcgtcaatggcctcgcacatggccctggcaccacgcatggaggccgccaaaagtggccgcctcctctctgcaattgcatttatttatccatttgataactctattgggtgtctacccagcattgttcaggggggtgtgaggggctaccgcaatgacccagacagactgctgtgcattcttattggaaagacaaggcaataaaccacagttacaaaacccagggtcccatggtaacgagctccacagagaaaagggaagtgggccagaggttccgagagacacacattttggattgcaggagggtgagaaggggcattcatctgtggtggggttaaggggaggcctccgactagctcacctgcatgaatttcaggaacaagccatgcagagccggacagagcattctgtgcagagaaaacagtgtaacagccctagagcagcagcagcaaagggggagtttaagggatggtgggaagccactgtcactggacagtagcaagtggacagaccaggggtcccaggtgaggccacagagcagccaaagtactgcagggctctccggctccgggaggacttgatgtttgactccagttgccagaggaagccgttcacgggctttgagtgtaggaatgatgtgtccgacttgggtctttttattgttcttttttagagacagggtctcactctgtcacccaggctggagtgcagcggcacaatcacagctcactgcagcctccacttcctgggctcaagcaatcctcccacttcagccactcaagtagctgggattacaggtgcctgccgtcacacccggctaattgttaaatttttttggagagacaggatcttgctgttttcccaggctgatcttgaactcctgggctcaagcgagacgtctgtttcagcctcccaaagtgctgggataacaggtgtgagccactgggcctgccttggcttgggtattaaaggaattgttctagttactatatggaggtgacttggggccgctggggaagaaagaggaagtcaggagccagtctgaggcagtggcaatgcagcgattatcagaaataatgggctcaatgatagactggataaagaaaatgtggcacatatacgccatggagtactatgcagccataaaaagggatgagttcatgtcctttgcagggacatggatgaagctggaaaccatcattctcagcaaactaacacaagaacagaaaaccaaacaccgcatgttctcactcgtaagtgggagttgaacaatgagaacacatggacacagggaagggaacatcacacactggggcctgtcagtgggtggggggctgggggagggagagcatcaggagaaatacccaatgtagatgatgacgggttcataggtgcagcaaaccaccatggcacgtgtatacctatgtaacaaacttgtgcgttctgcacgtgtaccccataactgaaagtataatcataattttaagaaaagaaatgatggcctttggggtctaataagaaggtggggctgacagggcttggggagagagcgagcttgacaggagaccacgatgtcttccccaggggtctgagcacctggggaaatggtggtgccatttaccatgactggaaagatcatgggaggagcaggtttgggacactgaaggcccgagagcccctggcaaaccaccagtgtaagtccaagagtccaaaagctgaagaacttggagtccaatgtttgagggtaaaaaacagccagcatgagaaaaagatgaaggccagaagactcgtccagtctagcccttccacattcctctgcccgcttttatcctagcctcgctggcaggtgattagatggtgcccacccaggttaagcgtgggtctctgtctcccagttcactgactcaaatgttaatttcctttggcgacagcctcacagacacacccaggaacgatactttgcatcctccaatccaatcaagttggcactcaatattaaacagtttcggccgggagcggtggctcacgcctgtaatcccagcactttgggaggccaaagcgggcagatcactaagtcaggagatcgagaccatcctggctaacacggtgaaaccccatctctactaaaaatacaaaaaattagccaggcgtggtggtgggcgcctgtagtcccagctactcgggaggctgaggcaagagagtggcgtgaacccgggaggcagaacttgcagcaagcagagatcgcaccactgcactccagcctccagcctgggcgacagagcaagacaccgtctcaaaaaaaaaaaaaaaacaactaacagtttcgttcattaaggcaatgtccatggggcagctcctgcatgccacgccacccctgccctgacatactcccaggctgaccagccagaactggggagagggcatcttgaagaaacagtccaccctccgccctcgcatacctgtgtctcccagcagacaagacacacaagggcagggactgcactttcctgtgtgcaaatctcaggtatggtccagaaatcacatctctttggtctgattggccttacttaccaaacccttgaaacaatggaaagaagcttgctgcaatgggaagacccctgccccccaaaccctctaaccaggagtctacagacctcattccgtggaagttcccattgtcccctgggaaaccaccaccttctgaatcggcacagccaaggcccccaaccctgctatcaacagctttccttagccaccttccccatcctgcagggcttttcaacatttcgactttttaatccttccctccctaagatatagacatggtgttagtagcagaaccctgaaggcaaaggaacagaaactaacactctcagaaagtcctagcaggatggtctgaattaatagacagctgcaggcagccagtgtgcaaggcacgctcgtaatgtcagtggcgttccggaatgagggctgtgatcccaggcagcccagatcccaaggcgaaaacattcatgtcgcccagcatcaccctgtccttgcccagggtgaccctgagacggcacttggattcttccacatctgaactcaaacctgctccctcagctctccccagcaggaattggagccctctccaggccgttcagccaaagcagaatgtgtctggccaagcagatctgcccagagaagaacaaagacgtgttcagacactgtctggggaattggttttgagctcactctgagctccatctgtgcaacaaggccagcgtcagctgttacagagaaggggcttgtggcagtttattaccctggtgagctggctcggggccatgctgaccgcaggactgtaaaggacataaggcagttggcaatatgggaagcagcagtccccaaagcaccccgtcaaggaaacgatgcaagagctctgtccactggaccagccccaccctaccccgccacgccctggcctagggggagggaccttgtgtttcacgggcctggatggccagggatggacagtggcatgggcacagacacacccagaccgaaggaagggcccgggctccagaaccagacccgatccaggctcccagctctgtcacctactggctgcatgaaaagggtgggttttccttgttttgcagcctcagctatctcagctgtgagggaacaatgacgccacctccctcacgtggcggtcctgaagatgaaatgtaatgcatgcagcgcggcggccggcccaccgtgtgctcttagtaactggccgctgttctcttcagccctcccgttactgaccacctcgagggcaccgttcctccaagctgaccatttcctgccagtcacccactgcccagctcgcccagccatggagtgggctctggtccacgtgcatggtgagctgccatctttgtcgtcctctggacaccacgtctacctccttgggaaatagaaacgagtcctgcattgctttgtcaaatttcacaaaaaccctagaaagcagatattgccctggtttacagacggacagacagaagcaaagagaagccaactgacccactccaggttacaagcagggtctggtaggagagcacgcgtccaccccaccgctcactgcgcccacctactcctcgcttgagggcagagaacgagctggggcggggcgctccagggggcacaggcagcctgtgttcaaggcatgctcgtgagccccacagcaggggcggagtcaaaccagggctctggttctggggtgagcagagggtggggccaggcccccactgggaggctgactgaggaggggctcaaagggcaggcaggctccatcagagcggtttgatgagcagggccgctgagtggccctcaggtcaaggctggggccgggaccaccagggaccaggtctgcggggccaggggtccctccccagggtggagcaggaaaactccggcaaccgccttcttgagtggacttgaccttccctttctctgctaatgatctcagattaatcccttaaggcaagtgccccagggtgggtgttggttgcataaaacaagaggagtccttcttcccttccttcctggcacaaacatttgtaggccacccccgatgtggctggtcgtagtctggatttgggggcacattcaggcattcaacaaatacgtcatgaggccccgtgatgagctggggggctatgcttgatgtcggcagtacagctgtggtcgctcgaatgaatgtgcccgccctcacgggactgacctctagtggtgagagacagacactaaccgagtcacatgtgtgattacacgtgtaatcatacttacatatgtaacaacacgagttacacgtgcagtgtggggagggggcccttcacggtgaccagcccatcagggaaggggatgcgggtggcagaaggaagcggcacacccttgtcctcacggagctcacaggaggttctctgaccctcatcttaagccacaggctgtgtctgagcctcactgccctagctatcaactgggtgcaggactaggccttaatcactgggcagcgggaagaactacataagctcatactggcgcaaagccccttgggagaccgcagggaaagccattgcatgaggtggtgacaggctcggccttcagagtcgggctagaatctgagttctgacaatgatccccgccttgacccatctctcctcaggccgccctgagccttccagttaggcctgggctttgggcttccgtgtttgtctctggattgttcggttttagcaagagtcctgctaaattggtcttgctggaatcctccgccctccacatctgattgcccttcctgtctgatcgtggtcttcatgccccacggcctccaggggacgtctgatcacctggcctgccttcagcaagaatccccccaggtgtttcctcttaagaggttttccatccactgagcccaccctgctccttggccatcaattcccacttgcctgtactgtattcagaggggaccccaatctctcttcccagctgcagcggtccctgtccaacactgccccccacctcaaataaagtctttcttactgtctttaacatatgccgtcaataaagttttagcagatccaa

SEQ ID NO: 238 - DNA, 21 bp, synthetic construct
  source 1..21: mol_type = other DNA
  misc_feature 1..19: note = RNA
  misc_feature 20..21: note = DNA
cgctcatcgacaataattatt

SEQ ID NO: 239 - DNA, 21 bp, synthetic construct
  source 1..21: mol_type = other DNA
  misc_feature 1..19: note = RNA
  misc_feature 20..21: note = DNA
taattattgtcgatgagcggc

SEQ ID NO: 240 - DNA, 20 bp, synthetic construct
  source 1..20: mol_type = other DNA
cttctcgtaatggcagatct

SEQ ID NO: 241 - DNA, 20 bp, synthetic construct
  source 1..20: mol_type = other DNA
gcacttgaggatcttgcacg

SEQ ID NO: 242 - DNA, 20 bp, synthetic construct
  source 1..20: mol_type = other DNA
gaggtcctctatgccatgga

SEQ ID NO: 243 - DNA, 20 bp, synthetic construct
  source 1..20: mol_type = other DNA
ccatacccatccatccagct

SEQ ID NO: 244 - protein, 4 aa, unidentified
  source 1..4: note = DEAD box protein family motif sequence
DEAD

SEQ ID NO: 245 - DNA, 21 bp, synthetic construct
  source 1..21: mol_type = other DNA
  misc_feature 1..19: note = RNA
  misc_feature 20..21: note = DNA
gaagctagaacttaccaaatt

SEQ ID NO: 246 - DNA, 21 bp, synthetic construct
  source 1..21: mol_type = other DNA
  misc_feature 1..19: note = RNA
  misc_feature 20..21: note = DNA
tttggtaagttctagcttcct

SEQ ID NO: 247 - RNA, 20 nt, synthetic construct
  source 1..20: mol_type = other RNA
ttgccactacagctaccacc

SEQ ID NO: 248 - RNA, 20 nt, synthetic construct
  source 1..20: mol_type = other RNA
ccaatgtactcagggaaaca

SEQ ID NO: 249 - RNA, 20 nt, synthetic construct
  source 1..20: mol_type = other RNA
agtccaccaaagtgtttgct

SEQ ID NO: 250 - RNA, 20 nt, synthetic construct
  source 1..20: mol_type = other RNA
aaaggagtctgcttacttac

SEQ ID NO: 251 - DNA, 20 bp, synthetic construct
  source 1..20: mol_type = other DNA
ttgccactacagctaccacc

SEQ ID NO: 252 - DNA, 20 bp, synthetic construct
  source 1..20: mol_type = other DNA
ccaatgtactcagggaaaca

SEQ ID NO: 253 - DNA, 20 bp, synthetic construct
  source 1..20: mol_type = other DNA
agtccaccaaagtgtttgct

SEQ ID NO: 254 - DNA, 20 bp, synthetic construct
  source 1..20: mol_type = other DNA
aaaggagtctgcttacttac

SEQ ID NO: 255 - DNA, 1473 bp, Homo sapiens
  source 1..1473: mol_type = genomic DNA
cttcaacactgtggaagctttgttctttcgctcttttgcaataaatcttgctactgctcactctttgggtccacactgcctttatgagctgtaacactcaccgcgaaaggtctgcagcttcactcctgaagccagcgagcccacgagcccacagggaggaatgaacaactccagatgcaccgccttaagagctgtaacactcacggcgaaggtctgcagcttcactcctgagccagcgagaccaggaacccaccagaaggaagaaactccgaacacatccgaacatcagaaggaacaaactccagacggcgccaccttaagagctgtaacactcaccgccagggtccggggcttcgttcttgaagtcagtgagaccaagaacccaccaattccggacgcagtaccatgatcaacaggatgttactagcaacttccttggagcgatgtggttgatatcaataacttttctctccattggttatggtgacatggtacctaacacatactgtggaaaaggagtctgcttacttactggaattatgggtgctggttgcacagccctggtggtagctgtagtggcaaggaagctagaacttaccaaagcagaaaaacacgtgcacaatttcatgatggatactcagctgactaaaagagtaaaaaatgcagctgccaatgtactcagggaaacatggctaatttacaaaaatacaaagctagtgaaaaagatagatcatgcaaaagtaagaaaacatcaacgaaaattcctgcaagctattcatcaattaagaagtgtaaaaatggagcagaggaaactgaatgaccaagcaaacactttggtggacttggcaaagacccagaacatcatgtatgatatgatttctgacttaaacgaaaggagtgaagacttcgagaagaggattgttaccctggaaacaaaactagagactttgattggtagcatccacgccctccctgggctcataagccagaccatcaggcagcagcagagagatttcattgaggctcagatggagagctacgacaagcacgtcacttacaatgctgagcggtcccggtcctcgtccaggaggcggcggtcctcttccacagcaccaccaacttcatcagagagtagctagaagagaataagttaaccacaaaataagactttttgccatcatatggtcaatattttagcttttattgtaaagcccctatggttctaatcagcgttatccgggttctgatgtcagaatcctgggaacctgaacactaagttttaggccaaaatgagtgaaaactctttttttttctttcagatgcacagggaatgcacctattattgctatatagattgttcctcctgtaatttcactaactttttattcatgcacttcaaacaaactttactactacattatatgatatataataaaaaaagttaatttctgcacataaaaaaaaaaaaaaaaa

SEQ ID NO: 256 - DNA, 2112 bp, Homo sapiens
  source 1..2112: mol_type = genomic DNA
cggcggcagcagcccatgcctccggtgcaacagctgcgcctcctccggtgccccggcggcgggggcgggagataacctgtccctgctgctccgcacctcctcgcccggcggcgccttccggacccgcacctcctcgccgctgtcgggctcgtcctgctgctgctgctgctgctcgtcgcgccggggcagccagctcaatgtgagcgagctgacgccgtccagccatgccagtgcgctccggcagcagtacgcgcagcagtccgcgcagcagtcggcgtccgcctcccagtaccaccagtgccacagcctgcagcccgccgccagccccacgggcagcctcggcagtctgggctccgggcccccgctctcgcaccaccaccaccacccgcacccggcgcaccaccagcaccaccagccccaggcgcgccgcgagagcaaccccttcaccgaaatagccatgagcagctgcaggtacaacgggggcgtcatgcggccgctcagcaacttgagcgcgtcccgccggaacctgcacgagatggactcagaggcgcagcccctgcagccccccgcgtctgtcggaggaggtggcggcgcgtcctccccgtctgcagccgctgccgccgccgccgctgtttcgtcctcagcccccgagatcgtggtgtctaagcccgagcacaacaactccaacaacctggcgctctatggaaccggcggcggaggcagcactggaggaggcggcggcggtggcgggagcgggcacggcagcagcagtggcaccaagtccagcaaaaagaaaaaccagaacatcggctacaagctgggccaccggcgcgccctgttcgaaaagcgcaagcggctcagcgactacgcgctcatcttcggcatgttcggcatcgtggtcatggtcatcgagaccgagctgtcgtggggcgcctacgacaaggcgtcgctgtattccttagctctgaaatgccttatcagtctctccacgatcatcctgctcggtctgatcatcgtgtaccacgccagggaaatacaggtaccatgatcaacaggatgttactagcaacttccttggagcgatgtggttgatatcaataacttttctctccattggttatggtgacatggtacctaacacatactgtggaaaaggagtctgcttacttactggaattatgggtgctggttgcacagccctggtggtagctgtagtggcaaggaagctagaacttaccaaagcagaaaaacacgtgcacaatttcatgatggatactcagctgactaaaagagtaaaaaatgcagctgccaatgtactcagggaaacatggctaatttacaaaaatacaaagctagtgaaaaagatagatcatgcaaaagtaagaaaacatcaacgaaaattcctgcaagctattcatcaattaagaagtgtaaaaatggagcagaggaaactgaatgaccaagcaaacactttggtggacttggcaaagacccagaacatcatgtatgatatgatttctgacttaaacgaaaggagtgaagacttcgagaagaggattgttaccctggaaacaaaactagagactttgattggtagcatccacgccctccctgggctcataagccagaccatcaggcagcagcagagagatttcattgaggctcagatggagagctacgacaagcacgtcacttacaatgctgagcggtcccggtcctcgtccaggaggcggcggtcctcttccacagcaccaccaacttcatcagagagtagctagaagagaataagttaaccacaaaataagactttttgccatcatatggtcaatattttagcttttattgtaaagcccctatggttctaatcagcgttatccgggttctgatgtcagaatcctgggaacctgaacactaagttttaggccaaaatgagtgaaaactctttttttttctttcagatgcacagggaatgcacctattattgctatatagattgttcctcctgtaatttcactaactttttattcatgcacttcaaacaaactttactactacattatatgatatataataaaaaaagttaatttctgcacataaaaaaaaaaaaaaaaa